(19) United States
(12) Patent Application Publication
(10) Pub. No.: US 2025/0064856 A1
(43) Pub. Date: Feb. 27, 2025

(54) METHODS FOR ADMINISTERING PLATELET DERIVATIVE COMPOSITIONS TO SUBJECTS AFFLICTED WITH HERMANSKY PUDLAK SYNDROME OR BERNARD SOULIER SYNDROME

(71) Applicant: Cellphire, Inc., Rockville, MD (US)

(72) Inventors: Keith Andrew MOSKOWITZ, Westfield, IN (US); William Matthew DICKERSON, Washington, DC (US); Maria Teresa ABREU-BLANCO, Rockville, MD (US); Lisa Ellen BOOTH, Gaithersburg, MD (US); Emma Caitlin LESHAN, Bethesda, MD (US); Glen Michael FITZPATRICK, North Potomac, MD (US)

(73) Assignee: Cellphire, Inc., Rockville, MD (US)

(21) Appl. No.: 18/943,815

(22) Filed: Nov. 11, 2024

Related U.S. Application Data
(63) Continuation-in-part of application No. PCT/US2023/066965, filed on May 12, 2023.
Application No. PCT/US2023/066965 is a continuation-in-part of application No. PCT/US2022/079280, filed on Nov. 4, 2022.
Application No. PCT/US2022/079280 is a continuation-in-part of application No. 17/673,773, filed on Feb. 16, 2022.
Application No. 17/673,773 is a continuation-in-part of application No. PCT/US2022/016866, filed on Feb. 17, 2022.
Application No. PCT/US2022/016866 is a continuation-in-part of application No. 17/673,773, filed on Feb. 16, 2022.
Application No. 17/673,773 is a continuation-in-part of application No. 16/994,377, filed on Aug. 14, 2020, now Pat. No. 11,701,388.
Application No. 17/673,773 is a continuation-in-part of application No. PCT/US2020/046525, filed on Aug. 14, 2020.
(60) Provisional application No. 63/599,917, filed on Nov. 16, 2023.
(60) Provisional application No. 63/364,620, filed on May 12, 2022.
(60) Provisional application No. 63/376,986, filed on Sep. 23, 2022.
(60) Provisional application No. 63/490,196, filed on Mar. 14, 2023.
(60) Provisional application No. 63/275,937, filed on Nov. 4, 2021.
(60) Provisional application No. 63/276,420, filed on Nov. 5, 2021.
(60) Provisional application No. 63/264,226, filed on Nov. 17, 2021.
(60) Provisional application No. 63/264,227, filed on Nov. 17, 2021.
(60) Provisional application No. 63/364,620, filed on May 12, 2022.
(60) Provisional application No. 63/371,849, filed on Aug. 18, 2022.
(60) Provisional application No. 63/376,986, filed on Sep. 23, 2022.
(60) Provisional application No. 63/150,338, filed on Feb. 17, 2021.
(60) Provisional application No. 63/275,937, filed on Nov. 4, 2021.
(60) Provisional application No. 63/276,420, filed on Nov. 5, 2021.
(60) Provisional application No. 63/264,227, filed on Nov. 17, 2021.
(60) Provisional application No. 63/065,337, filed on Aug. 13, 2020.
(60) Provisional application No. 62/887,923, filed on Aug. 16, 2019.
(60) Provisional application No. 63/065,337, filed on Aug. 13, 2020.
(60) Provisional application No. 62/887,923, filed on Aug. 16, 2019.
(60) Provisional application No. 63/264,227, filed on Nov. 17, 2021.
(60) Provisional application No. 63/276,420, filed on Nov. 5, 2021.
(60) Provisional application No. 63/275,937, filed on Nov. 4, 2021.
(60) Provisional application No. 63/150,338, filed on Feb. 17, 2021.

(51) Int. Cl. A61K 35/19; A61P 7/02

(52) U.S. Cl. CPC A61K 35/19; A61P 7/02

(57) ABSTRACT

Provided herein are methods for administering platelet derivatives, such as freeze-dried platelet derivatives (FDPDs) to a subject having Hermansky Pudlak Syndrome (HPS) or Bernard Soulier Syndrome (BSS), comprising administering an effective dose of the platelet derivatives in a platelet derivative composition to the subject. The platelet derivatives can have numerous characteristics provided herein, that make them well suited to restore hemostatic functions in the subject. In some embodiments, the platelet derivatives are from a pool of donors. Furthermore, provided herein are platelet derivatives, and methods using the same, that have numerous beneficial properties, as provided.

### CROSS-REFERENCE TO RELATED APPLICATIONS

This application claims priority to U.S. Provisional Application Ser. No. 63/599,917, filed on Nov. 16, 2023, and to International Application No. PCT/US2023/066965, filed on May 12, 2023. International Application No. PCT/US2023/066965 claims priority to U.S. Provisional Application Ser. No. 63/364,620, filed on May 12, 2022, U.S. Provisional Application Ser. No. 63/376,986, filed on Sep. 23, 2022, International Application No. PCT/US2022/079280, filed on Nov. 4, 2022, and U.S. Provisional Application Ser. No. 63/490,196, filed on Mar. 14, 2023. International Application No. PCT/US2022/079280 claims priority to U.S. Provisional Application Ser. No. 63/275,937, filed on Nov. 4, 2021, U.S. Provisional Application Ser. No. 63/276,420, filed on Nov. 5, 2021, U.S. Provisional Application Ser. No. 63/264,226, filed on Nov. 17, 2021, U.S. Provisional Application Ser. No. 63/264,227, filed on Nov. 17, 2021, U.S. application Ser. No. 17/673,773, filed on Feb. 16, 2022, International Application No. PCT/US2022/016866, filed on Feb. 17, 2022, U.S. Provisional Application Ser. No. 63/364,620, filed on May 12, 2022, U.S. Provisional Application Ser. No. 63/371,849, filed on Aug. 18, 2022, and U.S. Provisional Application Ser. No. 63/376,986, filed on Sep. 23, 2022. International Application No. PCT/US2022/016866 claims priority to U.S. Provisional Application Ser. No. 63/150,338, filed on Feb. 17, 2021, U.S. Provisional Application Ser. No. 63/275,937, filed on Nov. 4, 2021, U.S. Provisional Application Ser. No. 63/276,420, filed on Nov. 5, 2021, U.S. Provisional Application Ser. No. 63/264,227, filed on Nov. 17, 2021, and U.S. application Ser. No. 17/673,773, filed on Feb. 16, 2022. The U.S. application Ser. No. 17/673,773 claims priority to U.S. Provisional Application Ser. No. 63/150,338, filed on Feb. 17, 2021, U.S. Provisional Application Ser. No. 63/275,937 filed on Nov. 4, 2021, U.S. Provisional Application Ser. No. 63/276,420 filed on Nov. 5, 2021, U.S. Provisional Application Ser. No. 63/264,227 filed on Nov. 17, 2021, International Application No. PCT/US2020/046525 filed on Aug. 14, 2020, and is a continuation in part of U.S. application Ser. No. 16/994,377, filed on Aug. 14, 2020, which is a patented case with U.S. Pat. No. 11,701,388 dated Jul. 18, 2023. The U.S. application Ser. No. 16/994,377, filed on Aug. 14, 2020, claims priority to U.S. Provisional Application Ser. No. 62/887,923, filed on Aug. 16, 2019, and U.S. Provisional Application Ser. No. 63/065,337, filed on Aug. 13, 2020. International Application No. PCT/US2020/046525 filed on Aug. 14, 2020, claims priority to U.S. Provisional Application Ser. No. 62/887,923, filed on Aug. 16, 2019, and U.S. Provisional Application Ser. No. 63/065,337, filed on Aug. 13, 2020. Each of the applications listed in this paragraph is incorporated herein by reference in its entirety.

### TECHNICAL FIELD

The present disclosure generally relates to blood products, including those containing platelet derivatives, methods of producing such blood products, and methods of treating a subject using such blood products.

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Inactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Hermansky-Pudlak syndrome (HPS) is a rare autosomal recessive disorder characterized by abnormal biogenesis of lysosome-related organelles which manifests with oculocutaneous albinism and excessive bleeding of variable severity. HPS was initially described by Frantisek Hermansky and Paulus Pudlak in 1959 (Hermansky, F, and Pudlak, P. “Albinism associated with hemorrhagic diathesis and unusual pigmented reticular cells in the bone marrow: report of two cases with histochemical studies.” Blood vol. 14, 2 (1959): 162-9). These authors reported two patients with oculocutaneous albinism, bleeding diathesis due to platelet dysfunction, and reticular cells with pigment deposits in the bone marrow. Since the initial report, 10 genetic types of HPS have been identified and each is associated with a defect in biogenesis of lysosome-related organelles complex (BLOC)-1 (HPS-7, HPS-8 and HPS-9), BLOC-2 (HPS-3, HPS-5 and HPS-6), BLOC-3 (HPS-1 and HPS-4) or adapter protein (AP)-3 complex (HPS-2 and HPS-10).

Mutations observed in HPS are known to cause impairment of specialized secretory cells, including melanocytes, platelets, and lung alveolar type II epithelial cells. These patients demonstrate prolonged bleeding after surgical procedures and easy bruising (Pierson, Diane M., et al. “Pulmonary fibrosis in Hermansky-Pudlak syndrome.” Respiration 73.3 (2006): 382-395). Platelet transfusion is one of the treatment options for subjects with HPS. However, transfusion with platelets is associated with risks including transfusion-associated acute lung injury, allergic reactions, clinically relevant immunomodulation, post-transfusion purpura, infectious risk, and alloimmunization with subsequent ineffectiveness of platelet transfusion. Further, since conventional platelets cannot be frozen, they have the highest risk of bacterial sepsis of any blood product (Corash, Laurence. “Bacterial contamination of platelet components: potential solutions to prevent transfusion-related sepsis.” Expert review of hematology 4.5 (2011): 509-525). Accordingly, there is a need for an improved platelet product for effectively managing or treating the symptoms of a subject having HPS.

Bernard Soulier syndrome (BSS) is a rare autosomal recessive disorder with an estimated prevalence of 1 case in 1 million people. The defect in BSS platelets is due to abnormalities in the GPIb-IX-V complex affecting binding to von Willebrand factor (vWF) when exposed due to vascular injury on subendothelial surfaces, and thus interfering with primary hemostatic plug formation. Gp1b-IX-V mutations also interfere with normal clot formation by reducing the ability of thrombin at low concentrations to activate platelets. Mutations associated with BSS have been reported for GP1BA, GP1BB and GP9 genes (Peitsidis et al. Bernard Soulier syndrome in pregnancy: a systemic review. Haemophilia 2010.16: 584-59). Mutations in GP1BA gene account for half of BSS, including mutations within leucine rich repeats (LRR) which binds to vWF, indicating that the integrity of the LRR's is essential for normal function of the GPIb-IX complex (Lanza F., Bernard-Soulier syndrome (Hemorrhagiparous thrombocytic dystrophy). Orphanet Journal of Rare Diseases. 2006 I:46). BSS is characterized by thrombocytopenia, enlarged platelets, and prolonged bleeding time. A constant feature of BSS patients is the presence of a small number of very large platelets with a round shape, platelets count typically rage from 20,000-100,000/μL. Another distinctive abnormality of BSS platelets is a defect in ristocetin-induced aggregation, which is not corrected by addition of normal plasma. Aggregation response to agonists such as ADP or collagen are normal. A defect in prothrombin consumption is observed in BSS patients and is attributed to decrease in GPIb-fibrin-dependent thrombin generation (Beguin et al. Fibrin-dependent platelet procoagulant activity requires GPIb receptors and von Willebrand factor. Blood. 1999 2:564-70). Clinical manifestations of BSS include epistaxis, gingival and mucocutaneus bleeding, and hemorrhage after trauma. Some patients experience gastrointestinal haemorrhage and hematuria.

### SUMMARY

To overcome the above-mentioned and additional problems in the art, the present disclosure provides, at least in part, blood products, such as, platelet derivatives, freeze-dried platelet-derived hemostats, freeze-dried platelets or freeze-dried platelet derivatives for use in administering to a subject having HPS or BSS. Also provided herein, are methods for administering the freeze-dried platelets or freeze-dried platelet derivatives in a subject having HPS or BSS.

Accordingly, provided herein is a method for administering platelet derivatives, such as freeze-dried platelet derivatives (FDPDs) or freeze-dried platelet-derived hemostats (HDPDSs), to a subject having Hermansky Pudlak Syndrome (HPS) or Bernard Soulier Syndrome (BSS), comprising administering an effective dose of the platelet derivatives in a platelet derivative composition to the subject. The platelet derivatives, FPHs, and FDPDs can have numerous characteristics provided herein, that make them well suited to restore hemostatic functions in the subject.

In another aspect, provided herein is a method for administering freeze-dried platelet derivatives to a subject having Hermansky Pudlak Syndrome (HPS) or BSS, comprising:
administering an effective dose of the freeze-dried platelet derivatives in a platelet derivative composition to the subject,
wherein the platelet derivative composition comprises a population of freeze-dried platelet derivatives (FDPDs) comprising CD 41-positive platelet derivatives, wherein less than 5% of the CD 41-positive platelet derivatives are microparticles,
and wherein the FDPDs
   a) have the ability to generate thrombin in vitro in the presence of tissue factor and phospholipids;
   b) have the ability to occlude a collagen-coated microchannel in vitro; or
   c) both a) and b).

In some embodiments, the administering increases the levels of at least one platelet biomarker selected from CD62P, PAC-1, and CD63 for endogenous platelets of the subject (recipient) as compared to before the administering. In illustrative embodiments, levels of both CD62P and PAC-1 are increased in the subject after the administering. In illustrative embodiments, the subject has HPS.

In some embodiments that include a subject having BSS, administering increases or restores the ability to generate thrombin, and/or increases or restore the clot formation ability of the subject.

In some embodiments, the method further comprises before the administering, rehydrating the freeze-dried platelet derivatives to form a rehydrated platelet derivative composition, and wherein the administering is administering an effective dose of the rehydrated platelets from the rehydrated platelet derivative composition to the subject.

Provided herein, is a method for administering a platelet derivative composition to a subject having Hermansky Pudlak Syndrome (HPS) or Bernard Soulier Syndrome (BSS), comprising:

administering an effective dose of platelet derivatives in the platelet derivative composition to the subject, wherein the platelet derivative composition comprises a population of platelet derivatives, and wherein anti-Human Leukocyte Antigen (HLA), and/or anti-Human Platelet Antigen (HPA) antibodies are present in blood of the subject.

Provided herein, is a method for administering a platelet derivative composition to a subject, comprising:

administering an effective dose of platelet derivatives in the platelet derivative composition to the subject, wherein the platelet derivative composition comprises a population of platelet derivatives, and wherein anti-Human Leukocyte Antigen (HLA), and/or anti-Human Platelet Antigen (HPA) antibodies are present in blood of the subject.

In another aspect, provided herein, is a platelet derivative composition for use in administering to a subject having Hermansky Pudlak Syndrome (HPS) or Bernard Soulier Syndrome (BSS), wherein the administering comprises administering an effective dose of platelet derivatives in the platelet derivative composition to the subject, wherein the platelet derivatives
a) have the ability to generate thrombin in vitro in the presence of tissue factor and phospholipids;
b) have the ability to occlude a collagen-coated microchannel in vitro; or
c) both a) and b), and
   wherein anti-Human Leukocyte Antigen (HLA), and/or anti-Human Platelet Antigen (HPA) antibodies are present in blood of the subject.

In some embodiments, methods herein further comprises determining the presence of cross-reactive antibodies against the platelet derivatives in blood of the subject, and in illustrative embodiments, the subject has the presence of cross-reactive antibodies against the platelet derivatives in the blood.

Further details regarding aspects and embodiments of the present disclosure are provided throughout this patent application. The preceding paragraphs in this Summary section is not an exhaustive list of aspects and embodiments disclosed herein. Sections and section headers are for ease of reading and are not intended to limit combinations of disclosure, such as methods, compositions, and kits or functional elements therein across sections.

### DESCRIPTION OF THE DRAWINGS

FIG. 1A shows a reduced lag time of thrombin production with the addition of freeze-dried platelet derivatives (FDPDs) to the platelet-rich plasma (PRP) obtained from the blood of an Hermansky Pudlak Syndrome (HPS) patient.

FIG. 1B shows a reduced time of peak of thrombin production with the addition of FDPDs to the PRP obtained from the blood of an HPS patient.

FIG. 2 shows that the addition of FDPDs to HPS patient blood restores clot amplitude to near normal amplitude in an ex vivo experiment.

FIG. 3A shows an improvement of clot amplitude in HPS patients with the addition of FDPDs to the blood of HPS patient in comparison to the addition of apheresis platelets (APU) in an ex vivo experiment.

FIG. 3B shows increased reduction of k time with the addition of FDPDs to the blood of HPS patient in comparison to the addition of apheresis platelets (APU) in an ex vivo experiment.

FIG. 4A shows that the addition of FDPDs to the blood of HPS patients (HPS-8, HPS-9, and HPS-11) restores the percentage of positivity of endogenous platelets that are PAC-1 positive back to normal levels in an ex vivo experiment.

FIG. 4B shows the data from FIG. 4A as the mean and standard error of the mean of the blood of HPS patients' data.

FIG. 4C shows the mean fluorescent intensity (MFI) representation of the data from FIG. 4B.

FIG. 5A shows that the addition of FDPDs to the blood of HPS patients (HPS-8, HPS-9, and HPS-11) restores the percentage of positivity of endogenous platelets that are CD62P back to normal levels in an ex vivo experiment.

FIG. 5B shows the data of FIG. 5A as the mean and standard error of the mean of the HPS patients' data.

FIG. 5C shows the MFI representation of the same data as FIG. 5B.

FIG. 6A shows that the addition of FDPDs to HPS blood increases the number of cells that are positive for CD63 in an ex vivo experiment.

FIG. 6B shows the MFI representation of the same data as FIG. 6A.

FIG. 7 shows the detection of the level of anti-platelets IgG antibodies in different serums.

FIG. 8 shows that addition of FDPDs to PRP from HPS patients improves the time to occlude.

FIG. 9A shows the MFI representation of the surface marker CD42b (GPIbα) on control platelets and surrogate BSS model platelets (0.025 μg/μl AK2).

FIG. 9B shows the MFI representation of the surface marker CD41 (GPIIb) on control platelets and surrogate BSS model platelets (0.025 μg/μl AK2).

FIG. 9C shows the MFI representation of the surface marker CD62P (P-Selectin) on control platelets and surrogate BSS model platelets (0.025 μg/μl AK2).

FIG. 10 shows the aggregation data of surrogate BSS model platelets (0.025 μg/μl AK2) and the control platelets (control) in the presence of ristocetin.

FIG. 11A shows the occlusion time of the surrogate BSS model platelets (0.025 μg/μl AK2), surrogate BSS model platelets in the presence of FPH (0.025 μg/μl AK2+FPH), and the control platelets in a T-TAS assay.

FIG. 11B shows the area under the curve of the surrogate BSS model platelets (0.025 μg/μl AK2), surrogate BSS model platelets in the presence of FPH (0.025 μg/μl AK2+FPH), and the control platelets in a T-TAS assay.

FIG. 12 shows the average Mean Fluorescence Intensity (MFI) corresponding to the presence of IgG on the surface of FPH, from the three FPH lots, with error bars representing standard deviation.

FIG. 13A shows the peak thrombin for the different HPS serum samples when incubated with FPH.

FIG. 13B shows the lag time, time until thrombin first generated for the different HPS samples when incubated with FPH.

FIG. 13C shows the endogenous thrombin potential or the net amount of thrombin that HPS plasma samples can generate when incubated with FPH.

FIG. 13D shows the time to peak or the time for maximum concentration of thrombin generated.

FIG. 13E shows the velocity index or the slope of the thrombin potential between the lag time and the time to peak.

FIG. 14A shows the occlusion start time in the presence of FPH and HPS serum samples, in T-TAS assay.

FIG. 14B shows the occlusion time in the presence of FPH and HPS serum samples, in T-TAS assay.

FIG. 14C shows the area under the curve (AUC) in the presence of FPH and HPS serum samples, in T-TAS assay.

### DEFINITIONS

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment. It is to be understood that the terminology used herein is for the purpose of describing particular aspects and embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

As used herein, the term “platelets” has its ordinary meaning in the art.

As used herein, “cryopreserved platelets” are frozen platelets that when thawed are in a liquid state regardless of whether any liquid is added to the frozen platelets after thawing. Accordingly, cryopreserved platelets are not fresh platelets and they are not freeze-dried platelet derivatives. During processing cryopreserved platelets are not dried. The term “cryopreserved platelets” does not imply any minimum length of time such platelets are present in a frozen state. However, cryopreserved platelets are typically stable for at least 1, 2, 3, 4, 5, 6, 9, or 12 months, and in illustrative embodiments are stable for at least 18, 24, 36, or 48 hours. Cryopreserved platelets are typically suspended in a cryoprotectant in a frozen state, until thawing before use.

As used herein, “hemostatic properties” include the following properties: (a) the ability to generate thrombin in a thrombin generation assay, for example in the presence of tissue factor and phospholipids; (b) the ability to occlude a collagen-coated microchannel in vitro, for example under conditions in which fresh platelets can occlude a collagen-coated microchannel in vitro; (c) the capability of thrombin-induced trapping in the presence of thrombin. As demonstrated in Examples herein, platelet derivatives, such as a freeze-dried platelet derivatives (e.g., thrombosomes) herein, in illustrative embodiments are hemostats, and thus have one, two, or all of the aforementioned hemostatic properties.

As used herein, “platelet derivatives” are particles that have some characteristics of fresh platelets but are surrounded by a compromised plasma membrane (i.e., lack an integrated membrane around them), and as such include pores that are larger than pores found in living platelets. Thus, in illustrative embodiments, platelet derivatives herein exhibit an increased permeability to IgG antibodies. In illustrative embodiments, platelet derivatives, or aggregates thereof found in platelet compositions, are at least 0.5 μm or between 0.5 μm and 25 μm in diameter as determined by dynamic light scattering. Thus, such subsets of platelet-derivative particles are distinguishable from platelet-derivative microparticles, which have a diameter of less than 0.5 μm. Dry platelet derivatives are typically present in a dried substance that includes other components present along with the platelet derivatives when they were dried. Furthermore, a dry platelet derivative(s) with at least one hemostatic property can be referred to as a dry platelet derivative hemostat(s) (PDH).

In illustrative embodiments, platelet derivatives herein have a reduced ability to, or are unable to transduce signals from the external environment into a response inside the particle that are typically transduced in living platelets. However, platelet derivatives herein (e.g., thrombosomes) can retain some metabolic activity, for example, as evidenced by lactate dehydrogenase (LDH) activity and/or esterase activity.

As used herein, “particle size” refers to the diameter of a particle, unless indicated otherwise. In some embodiments of any of the aspects and embodiments herein that include a platelet derivative composition in a powdered form, the size of the particles is determined after rehydrating the platelet derivative composition with an appropriate solution. In some embodiments, the amount of solution for rehydrating a platelet derivative composition is equal to the amount of buffer or preparation agent present at the step of freeze-drying. The particle size distribution and microparticle content of a composition can be measured by any appropriate method, for example, by flow cytometry using sizing standards, or in illustrative embodiments by dynamic light scattering (DLS). As used herein, a content (e.g., ratio or percent) of microparticles in illustrative embodiments refers to the microparticle content based on the scattering intensity of all particles from about 1 nm to about 60,000 nm in radius in the composition. In some cases, the viscosity of a sample used for DLS can be at about 1.060 cP (or adjusted to be so), as this is the approximate viscosity of plasma. It will be understood that the measured size of particles can vary depending on the technology used to perform the measurement. Particle sizes provided herein are typically as determined by DLS unless the context indicates otherwise. For example if a percent surface marker content of a composition that includes particles, is recited as a percent of particles or platelet derivatives within a certain size range, flow cytometry is typically used to measure both biomarker content and particle size. In some embodiments, the platelet derivative composition as per any aspects, or embodiments comprises a population of platelet derivatives greater than 0.5 μm, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, illustrative or target platelet derivatives have a diameter in the range of 0.5-2.5 μm using flow cytometry, or a diameter of 0.5-25 μm using DLS, and microparticles have a diameter less than 0.5 μm by either method.

As used herein, “thrombosomes” (sometimes also herein called “Tsomes” or “Ts”, particularly in the Examples and FIGS.) are platelet derivatives that have been contacted with an incubating agent (e.g., any of the incubating agents described herein) and lyopreserved (e.g., freeze-dried). Thus, thrombosomes are illustrative or target freeze-dried platelet derivatives (FDPDs). Illustrative or target freeze-dried platelet derivative compositions herein (e.g. thrombosomes) typically have at least 1 hemostatic property, and thus can function as hemostatic agents and can be referred to hemostat(s) or hemostatic product(s). Illustrative or target FDPDs and compositions herein comprising the same that have at least 1 hemostatic property can also be referred to as freeze-dried platelet derived hemostat(s) or freeze-dried platelet hemostat(s) (both of which can be abbreviated FDPDH, FDPH or FPH). In some cases, illustrative or target FDPDs such as thrombosomes can be prepared from pooled platelets. Illustrative or target FDPDs such as thrombosomes can have a shelf life of 2-3 years in dry form at ambient temperature and can be rehydrated with sterile water within minutes (e.g. 1, 2, 3, 4, 5, 10 15, 20, 25, or 30 minutes) for immediate infusion. One example of thrombosomes are THROMBOSOMES® freeze-dried platelet derivatives (Cellphire Inc., Rockville, MD), which are in clinical trials for the treatment of acute hemorrhage in thrombocytopenic patients and are a product of Cellphire, Inc. In non-limiting illustrative embodiments, FDPD compositions, or illustrative or target freeze-dried platelet-derivative (i.e. “FDPD”) compositions herein, such as those prepared according to Example 2 herein, are compositions that include a population of platelet derivatives having a reduced propensity to aggregate such that no more than 10% of the platelet derivatives in the population aggregate under aggregation conditions comprising an agonist but no platelets, and in illustrative embodiments, no divalent cations. Furthermore, such illustrative or target platelet derivatives typically have the ability to generate thrombin in an in vitro thrombin generation assay and/or have the ability to occlude a collagen-coated microchannel in vitro.

In illustrative embodiments, illustrative or target platelet derivatives are CD41 positive and/or CD42 positive. Platelets derivatives (e.g., thrombosomes) herein, in some embodiments, are dry platelet derivatives, or dry platelet derived particles. A skilled artisan will understand that most properties of such dry platelet derivatives are analyzed after the platelet derivatives are rehydrated. Dry platelet derivatives are typically present in a dried substance that includes other components (e.g., saccharides such as, for example, trehalose and/or polysucrose) present along with the platelet derivatives when they were dried. In illustrative platelet derivative compositions herein, less than 5% of the particles are microparticles having a diameter of less than 0.5 μm. In illustrative platelet derivative compositions herein, at least 90% of the particles therein are at least 0.5 μm in diameter. Furthermore, in illustrative embodiments, between 75% and 95% of the platelet derivatives or particles therein are CD41 positive, between 75% and 95% of the platelet derivatives or particles therein are CD42 positive, and less than 5% of the CD 41-positive platelet derivatives or particles therein are microparticles having a diameter of less than 0.5 μm. It will be understood that in such percent calculations, particles are only intended to cover those that can be detected for example by the instrument (e.g., flow cytometer) used to detect CD41 or CD42 or any surface marker.

In some examples of such illustrative embodiments, the platelet derivatives have a potency of at least 1.5 thrombin generation potency units (TGPU) per 10⁶ platelet derivatives. In non-limiting illustrative embodiments, FDPD compositions, or illustrative FDPD compositions herein, such as those prepared according to Example 2 herein, are compositions that include illustrative or target platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and typically such compositions have the ability to generate thrombin in an in vitro thrombin generation assay and/or have the ability to occlude a collagen-coated microchannel in vitro. In illustrative embodiments, the platelet derivatives in such compositions have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the illustrative or target platelet derivatives are between 0.5 and 2.5 μm in diameter by flow cytometry or between 0.5 and 25.0 μm in diameter by dynamic light scattering.

It is to be understood that the terminology used herein is for the purpose of describing particular aspects and embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication. Any Headings herein are for convenience only and are not intended to be limiting, and it will be understood that the disclosure including aspects and embodiments provided within one section herein can be combined with the disclosure includes aspects and embodiments provided within any other section herein.

### DETAILED DESCRIPTION

To overcome the above-mentioned and additional problems in the art, the present disclosure provides, at least in part, blood products, such as, platelet derivatives, freeze-dried platelets or freeze-dried platelet derivatives for use in administering to a subject having HPS or BSS. Also provided herein, are methods for administering the freeze-dried platelets or freeze-dried platelet derivatives in a subject having HPS or BSS.

Accordingly, provided herein is a method for administering platelet derivatives, freeze-dried platelet derivatives (FDPDs), or freeze-dried platelet-derived hemostats (FPHs) to a subject having Hermansky Pudlak Syndrome (HPS), comprising administering an effective dose of the freeze-dried platelet derivatives in a platelet derivative composition to the subject. The FDPDs can have numerous characteristics provided herein, that make them well suited to restore hemostatic functions in a recipient.

Accordingly, provided herein is a method for administering platelet derivatives, freeze-dried platelet derivatives (FDPDs), or freeze-dried platelet-derived hemostats (FPHs) to a subject having Bernard Soulier Syndrome (BSS), comprising administering an effective dose of the freeze-dried platelet derivatives in a platelet derivative composition to the subject. The FDPDs can have numerous characteristics provided herein, that make them well suited to restore hemostatic functions in a recipient.

In some embodiments, the administering increases the levels of at least one platelet biomarker selected from CD62P, PAC-1, and CD63 for endogenous platelets of the subject (recipient) as compared to before the administering. In illustrative embodiments, levels of both CD62P and PAC-1 are increased in the subject after the administering. Such embodiments can include those in which a subject has BSS, and in illustrative embodiments, include subjects having HPS.

In some embodiments, the administering restores the thrombin generation activity, and/or the clot forming ability. For example, the administering restores/decreases the occlusion time for occluding a collagen-coated channel, such as in a T-TAS assay.

Platelet Derivatives Maintain its Functions in the Presence of Anti-HLA and/or Anti-HPA Antibodies

Methods herein, or compositions for use in methods herein, include administering an effective amount of platelet derivatives, or freeze-dried platelet derivatives in a platelet derivative composition to a subject, in illustrative embodiments, to a subject (i.e., recipient) that has anti-Human Leukocyte Antigen (HLA) and/or anti-Human Platelet Antigen (HPA) antibodies in the blood. For example, the subject can have anti-HLA antibodies in the blood. In some embodiments, the subject can have anti-HPA antibodies in the blood. The subject can have both anti-HLA and anti-HPA antibodies in the blood. The anti-HLA antibodies can be against HLA Class-1 antigens, and/or HLA Class-2 antigens. For example, in case of anti-HLA antibodies against Class-1 antigens, the antibodies can be against one or more of HLA-A, HLA-B, and HLA-C, including various sub-types. In case of anti-HLA antibodies against Class-2 antigens, the antibodies can be against one or more of DR, DQ, DPA, and DPB, including various sub-types. The anti-HPA antibodies can be against one or more antigens on the platelets, for example, glycoproteins, including different types or sub-types thereof. In illustrative embodiments, the anti-HPA antibodies can be against different glycoproteins, including GPIIb/IIIa, GPIa/IIa, GPIIIa, GPIbα, GPIIb, GPIa, GPIbβ, CD109, and GPIX. Typically, HPA can be categorized as different HPA systems, namely, HPA-1, HPA-2, HPA-3, HPA-4, HPA-5, and HPA-15. In some embodiments, the subject can have cross-reactive antibodies against the platelet derivatives, or the freeze-dried platelet derivatives. The cross-reactive antibodies can be present against one or more antigen that is present on the platelet derivatives, typically, including GPIIb/IIIa, GPIa/IIa, GPIIIa, GPIbα, GPIIb, GPIa, GPIbβ, CD109, and GPIX.

Methods herein, or compositions for use in methods herein for administering platelet derivatives to a subject, for example, a recipient can include a step of determining the presence of anti-HLA and/or anti-HPA antibodies, including cross-reactive antibodies against the platelet derivatives in a sample obtained from a subject. For example, the sample from the subject can include blood sample, typically, plasma or serum sample. The information regarding the type of antibodies (for example, alloantibodies) produced in a subject can be obtained by known techniques including, but not limited to Luminex-based phenotypic beads such as, beads coated with specific Class 1 or Class 2 HLA antigens, or HPA antigens. In some embodiments, the Luminex-based beads having only single type of antigen, such as, single-antigen bead assay (SAB) can also be used. In SAB, Luminex beads are coated with a single HLA/HPA on their surface and incubated with the serum of the recipient. If the recipient has antibodies against the specific HLA/HPA that is coated on the beads, they will bind to the bead that is coated with the respective antigen. The beads are then washed and incubated with phycoerythrin (PE)-labeled anti-human IgG antibodies. This antibody will bind to the Fc region of antibodies bound to beads. The mixture is then washed and analyzed using a Luminex instrument. Each bead is identified through a unique combination of two fluorescent dyes (red and infrared) impregnated into the bead. The anti-human IgG complex will emit fluorescence upon exposure to laser light. This fluorescence is detected on a Luminex platform and antibody reactivity is recorded as mean fluorescence intensity.

Another non-limiting example of a method for analyzing a recipient's alloantibodies includes generating a calculated panel-reactive antibody (cPRA or PRA) percentage. A cPRA can be generated by a commonly known test that employs Luminex-based phenotypic beads to screen for HLA antibodies, typically, HLA Class-1 antibodies. A cPRA provides a percentage of antibody in a test serum, typically from the recipient that are reactive against a panel of known HLA antigens. Therefore, higher cPRA percentage denotes higher number of antibodies in the serum of the recipient that are reactive against a known panel of HLA Class-1 antigens. Accordingly, in some embodiments, cPRA can be used to study the presence of antibodies against known HLA. Accordingly, subjects, patients, or recipients in methods herein can have a PRA score of greater than 10, 15, 20, 25, 30, 40, 50, 60, 70, 75 80, 85, or 90%, or between 15% on the low end and 50, 60, 70, 75 80, 85, or 90% on the high end. In some embodiments, the subject, patient or recipient is refractory to platelet transfusions. In one illustrative embodiment, the subject, patient, or recipient has a PRA score of greater than 15% and less than 70%.

The sample obtained from the subject can be positive for anti-HLA and/or anti-HPA antibodies, and further for the presence of antibodies against platelet antigen on the platelet derivatives based upon the detection of antibodies against one or more HLA, HPA, or platelet antigen on the platelet derivatives. The amount of the antibodies can vary as per the subject, such that, based on the method used to quantify the amount of antibodies against HLA, HPA, or platelet antigen on the platelet derivatives, the subject, or recipient can be categorized as high risk, moderate risk, or low risk. As per one of the methods for quantifying the anti-HLA, and/or anti-HPA antibodies, involving flow cytometry, the mean fluorescence intensity (MFI) of the antibodies over 5,000 were categorized as high risk, between 3,000 and 5,000 were categorized as medium risk, and between 1,000 and 3,000 were categorized as low risk. A non-limiting method of determining the presence of anti-HLA, and/or anti-HPA antibodies in a subject can include performing antibody screening for the presence of anti-HLA antibodies against HLA Class-1 and Class-2 antigens. For samples that are positive for the presence of anti-HLA antibodies, the category of the patients can be established based on the titer of antibodies, for example, high risk, moderate risk, or low risk. Once the anti-HLA antibodies screening is completed, the samples can further be screened for the presence of anti-HPA antibodies. For samples that are positive for the presence of anti-HPA antibodies, the category of the patients can be established based on the titer of antibodies, for example, high risk, moderate risk, or low risk.

In some embodiments, platelet derivatives herein can have a comparable hemostatic activity and/or thrombin generation activity in the presence versus absence of anti-HLA, and/or anti-HPA antibodies. The platelet derivatives herein can have a comparable hemostatic activity and/or thrombin generation activity in the presence versus absence of cross-reactive antibodies against the freeze-dried platelet derivatives. For example, in ex vivo, or in vitro experiments, the hemostatic activity and/or thrombin generation activity of platelet derivatives remain unaltered, or significantly unchanged in the presence versus absence of anti-HLA, and/or anti-HPA antibodies. In some embodiments, one or more of thrombin generation activity of the platelet derivatives, for example, in in vitro thrombin generation assays, including peak thrombin, lag time until thrombin generation, endogenous thrombin potential, and time to peak thrombin generation remains comparable, unaltered, or significantly unchanged in the presence versus absence of anti-HLA, and/or anti-HPA antibodies, including the presence or absence of cross-reactive antibodies against the platelet derivatives. For example, peak thrombin, lag time until thrombin generation, endogenous thrombin potential, and time to peak thrombin generation remains comparable, unaltered, or significantly unchanged such that, platelet derivatives have the property of yielding +/−50%, +/−40%, +/−30%, +/−25%, +/−20%, +/15%, +/−10%, +/−5%, or less in one or more of the peak thrombin, lag time until thrombin generation, endogenous thrombin potential, and time to peak thrombin generation in the presence versus absence of anti-HLA, and/or anti-HPA antibodies, including the presence or absence of cross-reactive antibodies against the platelet derivatives. For example, the variation in the yield of platelet derivatives in the presence versus absence of anti-HLA, and/or anti-HPA antibodies, including the presence versus absence of cross-reactive antibodies can be in the range of 1-50%, 1-45%, 1-40%, 1-35%, 1-30%, 1-25%, 1-20%, 1-15%, 1-10%, 1-5%, 5-50%, 10-50%, 15-50%, or 20-50% in one or more of the peak thrombin, lag time until thrombin generation, endogenous thrombin potential, and time to peak thrombin generation. In some embodiments, one or more of hemostatic activity of the platelet derivatives, for example, in an in vitro T-TAS assay including occlusion start time, occlusion time, and area under the curve (AUC) remains comparable, unaltered, or significantly unchanged in the presence versus absence of anti-HLA, and/or anti-HPA antibodies, including the presence or absence of cross-reactive antibodies against the platelet derivatives. For example, occlusion start time, occlusion time, and area under the curve (AUC) remains comparable, unaltered, or significantly unchanged such that, platelet derivatives have the property of yielding +/−50%, +/−40%, +/−30%, +/−25%, +/−20%, +/15%, +/−10%, +/−5%, or less in one or more of the occlusion start time, occlusion time, and the AUC in the presence or absence of anti-HLA, and/or anti-HPA antibodies, including the presence versus absence of cross-reactive antibodies against the platelet derivatives. For example, the variation in the yield of platelet derivatives in the presence versus absence of anti-HLA, and/or anti-HPA antibodies, including the presence versus absence of cross-reactive antibodies can be in the range of 1-50%, 1-45%, 1-40%, 1-35%, 1-30%, 1-25%, 1-20%, 1-15%, 1-10%, 1-5%, 5-50%, 10-50%, 15-50%, or 20-50% in one or more of occlusion start time, occlusion time, and the AUC.

### Platelet Derivatives Affect Platelet Biomarkers of Endogenous Platelets

Methods herein, or compositions for use in methods herein, include administering an effective amount of platelet derivatives, or freeze-dried platelet derivatives in a platelet derivative composition to a subject in need thereof. In illustrative embodiments, the subject in need thereof, has Hermansky Pudlak Syndrome (HPS) or has Bernard Soulier Syndrome (BSS). In illustrative embodiments, the platelet derivative composition comprises a population having a reduced propensity to aggregate such that no more than 10%, 8%, 7%, or 5% of the platelet derivatives in the population aggregate under aggregation conditions comprising an agonist but no platelets. In some embodiments, administering can include administering the platelet derivatives, freeze-dried platelet-derived hemostats (FPHs), or freeze-dried platelet derivatives for treating a subject. The treating, for example, can include a partial, or a complete restoration of platelet functions in the subject. In some embodiments, the administering restores one or more normal platelet functions or properties, for example expression of certain platelet biomarkers as provided herein, but at a dose that is different from, in certain embodiments, less than a dose required to reduce or stop bleeding in the subject, and thus in some embodiments, such dose does not reduce or in illustrative stop bleeding in the subject, for example at a primary site of bleeding in the subject, or at all or substantially all sites of bleeding. In some embodiments, the administering does not reduce or stop bleeding in the subject within 4, 8, 12, or 24 hours, or 1 day, 2 days, 3 days or 7 days from the administering. However, in some further examples of such embodiments, the bleeding is stopped after 4, 8, 12 or 24 hours, or 1 day, 2 days, 3 days or 7 days after the administering.

For example, administering platelet derivatives, freeze-dried platelet-derived hemostats (FPHs) or freeze-dried platelet derivatives (FDPDs) herein to a subject, patient, and/or recipient can have certain effects on the levels of at least one platelet biomarker of the endogenous platelets in the subject, patient, and/or recipient of the FPHs or FDPDs. A skilled artisan will understand that the endogenous platelets are the platelets of a subject, and does not include platelets, or platelet derivatives that are administered to the subject. The platelet biomarkers that can be affected upon administering, in some embodiments can include any platelet biomarker that is associated with the activation of the platelets, or endogenous platelets in a subject.

In some embodiments, the platelet biomarkers whose levels are affected by administration of FPHs or FDPDs can be any one of PAC-1, CD62P, CD63, or combinations thereof. In some embodiments, the platelet biomarkers can be at least two, or all the three of the platelet biomarkers PAC-1, CD62P, CD63. Such embodiments include those in which the subject has BSS, and in illustrative embodiments, include those in which the subject has HPS. In illustrative embodiments, the platelet biomarker can be PAC-1, CD62P, or both. In some embodiments, the administering can lead to restoring the levels of at least one platelet biomarkers of the endogenous platelets in the subject to levels similar to the corresponding platelet biomarkers of platelets in a healthy subject. Similar levels as used herein, can cover a range within lower values and higher values of the level of a corresponding platelet biomarker from a healthy subject. In other words, similar levels can include levels higher or lower than a control level or a normal level of the corresponding platelet biomarker in a healthy subject. For example, the administering herein, can restore the levels to between 30% higher to 30% lower, between 25% higher to 25% lower, between 20% higher to 20% lower, between 15% higher to 15% lower, between 10% higher to 10% lower, or between 5% higher to 5% lower levels of the endogenous platelets in the subject as compared to normal levels for these biomarkers. For example, the administering herein, can restore the levels within at least 5%, 10%, 15%, 20%, 25%, or 30% levels of the endogenous platelets in the subject as compared to the normal levels. In some embodiments, the administering herein can lead to improving, increasing, increasing platelets testing positive for the at least one platelet biomarker, affecting activation of at least one platelet biomarker, or restoring the at least one platelet biomarker as discussed herein. For example, the administering can lead to an increase or an improvement in the levels of at least one platelet biomarker of platelets, in illustrative embodiments, endogenous platelets, such that the levels are increased by at least 5%, 10%, 15%, 20%, 25%, 30%, 35%, 40%, 45%, 50%, 55%, 60%, or more as compared to the levels in the subject before administering the platelet derivatives, or freeze-dried platelet derivatives. In some embodiments, the administering can lead to an increase or an improvement in the levels of at least one platelet biomarker in the range of 2 to 70%, 2 to 65%, 2 to 60%, 2 to 55%, 2 to 50%, 2 to 45%, 2 to 40%, 2 to 35%, 2 to 30%, 5 to 70%, 10 to 70%, 15 to 70%, 20 to 70%, 25 to 70%, 30 to 70%, or 35 to 75%, as compared to the levels in the subject before administering the platelet derivatives, or freeze-dried platelet derivatives.

A subject having HPS can have HPS-related biomarker abnormalities. Thus, the subject can be treated by administering the platelet derivatives, FPHs or freeze-dried platelet derivatives to the subject, such that at least one HPS-related biomarker abnormality or BSS-related biomarker abnormality observed in the subject is improved in the subject after the administering as compared to before the administering step. In some embodiments, the HPS-related biomarker abnormalities include a change in the biomarker levels, for example in endogenous platelets of a subject as compared to a subject who does not have HPS. For example, a non-limiting list of such biomarker abnormalities can include an abnormal decrease in the levels of CD62P, PAC-1, and CD63 or in some embodiments, CD62P and PAC-1. In some embodiments, an HPS-related hemostatic abnormality can include any hemostatic abnormality that can result from HPS in a subject. For example, the HPS-related hemostatic abnormality can include a decreased clotting ability in a subject that can lead to increased bleeding time in case of an injury. In some embodiments, administering can lead to restoring the levels of at least one HPS-related biomarker to normal levels after the administering as compared to before the administering step. In some embodiments, the FDPD administration can improve, control, and/or restore the levels of biomarkers and/or hemostasis in the recipient subject, such that the subject stops taking another therapeutic for treating HPS and/or an HPS-related biomarker abnormality.

In some embodiments, administering platelet derivatives, or freeze-dried platelet derivatives can lead to an increase in the levels of at least one platelet biomarker of endogenous platelets in a recipient subject/patient compared to the levels of a corresponding platelet biomarker in a subject having HPS, but not administered with the platelet derivatives, or freeze-dried platelet derivatives. In some embodiments, administering herein, can lead to an increase in, or restore, the levels of a platelet biomarker of endogenous platelets in a recipient subject/patient as compared to a subject having HPS but administered normal platelets, in illustrative embodiments, normal apheresis platelets, and not administered, or before administering the platelet derivatives, or the freeze-dried platelet derivatives as disclosed herein.

### Platelet Derivatives Affect Thrombin Generation and Clotting Ability

Administering platelet derivatives, freeze-dried platelet derivatives (FDPDs), or freeze-dried platelet-derived hemostats (FPHs) herein to a subject (i.e. recipient), in illustrative embodiments, to a subject (i.e. recipient) having HPS or BSS can affect at least one HPS-related, or BSS, respectively, hemostatic abnormality. In some embodiments, the administering can be used to treat abnormalities caused by HPS or BSS in the subject. The treatment herein can be a partial treatment or a complete treatment. For example, administering herein can lead to an improvement in one, two, three, at least one, all but one, or all HPS-related or BSS-related hemostatic abnormality. Furthermore, administration of the FDPDs can maintain the normal levels of hemostasis in the subject. For example, after administration of the FDPDs to the subject, the levels of hemostasis can be maintained such that the subject stops taking another therapeutic that the subject is taking for treating the HPS-related hemostatic irregularity(s) or BS-related hemostatic irregularity. In some embodiments, administering of FDPDs herein can lead to an improvement in thrombin generation in the subject as compared to the subject before the administering. For example, administering herein can increase or improve thrombin generation in the subject in vivo, and/or in an ex vivo assay using the subject's blood, by at least 5%, 10%, 15%, 20%, 25%, 30%, 35%, 40%, 45%, 50%, 55%, 60%, or more. In other embodiments, administering the FDPDs herein, can increase thrombin generation in the subject in vivo, and/or in an ex vivo assay using the subject's blood, in the range of 2 to 75%, 5 to 75%, 10 to 75%, 15 to 75%, 20 to 75%, 25 to 75%, 30 to 75%, 35 to 75%, 40 to 75%, 45 to 75%, 50 to 75%, 5 to 70%, 5 to 65%, 5 to 60%, 5 to 55%, 5 to 50%, or 5 to 45%.

A skilled artisan can use any known test(s) to assess thrombin generation in a subject. For example, thrombin generation can be assessed by a thrombin generation assay, and the assay can be performed by semi-automated methods for example using a calibrated automated thrombogram, or using fully automated systems. Thrombin generation assay is a type of coagulation test and is based on the potential of a plasma to generate thrombin over time, following addition of activators like phospholipids, tissue factor, and calcium. The results of the assay can typically be calculated as a thrombogram, or thrombin generation curve using computer software after calculation of thrombogram parameters.

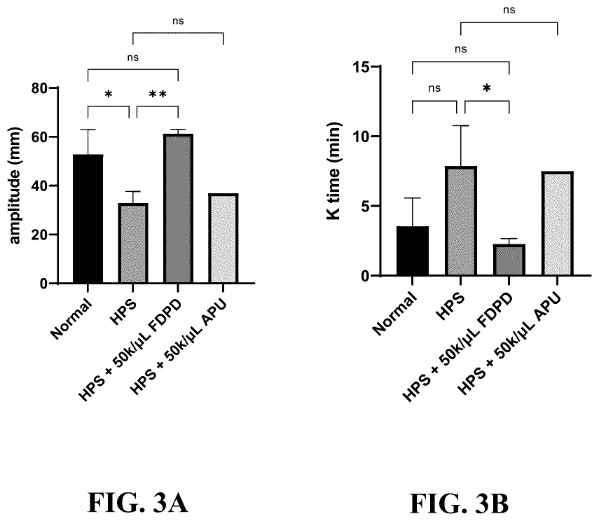

In some embodiments, administering FDPDs herein can lead to an improvement in clot formation in the subject as compared to the subject before the administration of FDPDs. In some embodiments, the subject has HPS. In some embodiments, the subject has BSS. For example, administering FDPDs herein can increase or improve clot formation in the subject by at least 5%, 10%, 15%, 20%, 25%, 30%, 35%, 40%, 45%, 50%, 55%, 60%, or more. In other embodiments, administering herein, can increase clot formation in the subject in the range of 2 to 75%, 5 to 75%, 10 to 75%, 15 to 75%, 20 to 75%, 25 to 75%, 30 to 75%, 35 to 75%, 40 to 75%, 45 to 75%, 50 to 75%, 5 to 70%, 5 to 65%, 5 to 60%, 5 to 55%, 5 to 50%, or 5 to 45%. Clot formation can be monitored by known techniques, for example, a thromboelastography method (TEG). As shown in Example 3 (FIG. 3A and FIG. 3B) it was surprisingly observed that providing FDPDs ex vivo HPS patient blood restores clot amplitude better than the same dose of apheresis platelets. Clot formation parameters are measured including time to clot initiation, the rate of clot formation, the angle of clot, maximum amplitude of the size of clot, and the percent platelet activation normalized to a known sample. TEG can also be used to analyze platelet function, and fibrinolysis along with clot formation. In some embodiments, administering can lead to an improvement in the clot amplitude by at least 2 fold, 3 fold, 4 fold, 5 fold, 6 fold or more, for example when analyzed using TEG. In some embodiments, 2-8 fold, 3-8 fold, or 3-7 fold improvement in clot amplitude. In some embodiments, administering FDPDs can lead to an improvement in the clot amplitude by at least 2 fold, 3 fold, 4 fold, 5 fold, 6 fold or more, as compared to the administering with platelets, such as apheresis platelets but not FDPDs, for example when analyzed using TEG. In some embodiments, administering FDPDs can show 2-8 fold, 3-8 fold, or 3-7 fold improvement in clot amplitude, as compared to the administering with platelets, such as apheresis platelets but not FDPDs, for example when analyzed using TEG. In some embodiments, administering can lead to an improvement or increase in both thrombin generation and clot formation. In some embodiments, the administering leads to an improvement or increase in thrombin generation and/or clot formation in the subject as compared to the subject after being administered apheresis platelets, but before the administering of the platelet derivatives, or the freeze-dried platelet derivatives.

In some embodiments, administering platelet derivatives herein can exhibit a reduction in the occlusion time for occluding a collagen-coated channel, for example, in a T-TAS assay as compared to the platelets obtained from a patient having BSS. For example, administering platelet derivatives herein can exhibit a reduction in the occlusion time to restore the occlusion time similar to that exhibited by platelets from a healthy subject. In some embodiments, administering platelet derivatives herein can exhibit a 1.5 fold, 2 fold, 3 fold, or higher reduction in the occlusion time, for example, in a T-TAS assay. As shown in Example 10 it was surprisingly observed that providing platelet derivatives in surrogate BSS model platelets decreased the occlusion time and restored it similar to that of the control. Further, it was also observed that the area under curve (AUC) was increased after providing the platelet derivatives herein. In some embodiments herein, the subject is a mammal, in illustrative embodiments, the mammal is a human.

### Platelet Derivatives for Treating Disorders

In some embodiments, a platelet derivative composition, in illustrative embodiment a freeze-dried platelet derivative, including, but not limited to, those of any of the aspects or embodiments herein, or the platelet derivative composition prepared by any of the process described in the aspects or embodiments herein, can be administered, or delivered to a subject having an indication and thus afflicted with a disorder or disease that could benefit from delivery of such platelet derivative compositions. In some embodiments, such indication can be any one or a combination of Von Willebrand disease, immune thrombocytopenia (ITP), intracranial hemorrhage (ICH), traumatic brain injury (TBI), Hermansky Pudlak Syndrome (HPS), chemotherapy induced thrombocytopenia (CIT), Scott syndrome, Evans syndrome, hematopoietic stem cell transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich syndrome, venous thromboembolism, MYH9 related disease, acute lymphoblastic lymphoma (ALL), acute coronary syndrome, chronic lymphocytic leukemia (CLL), acute promyelocytic leukemia, cerebral venous sinus thrombosis (CVST), liver cirrhosis, factor v deficiency (Owren Parahemophilia), thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's disease (Factor VII Deficiency), hemophilia C (Factor XI Deficiency), familial hemophagocytic lymphohistiocytosis, acute lung injury, hemolytic uremic syndrome, menorrhagia, chronic myeloid leukemia. In illustrative embodiments, a platelet derivative composition of any of the aspects or embodiments herein, or the platelet derivative composition prepared by any of the process described in the aspects or embodiments herein can be administered, or delivered to a subject afflicted by Immune thrombocytopenia. In certain illustrative embodiments, a platelet derivative composition of any of the aspects or embodiments herein, or the platelet derivative composition prepared by any of the process described in the aspects or embodiments herein can be administered, or delivered to a subject afflicted by Von Willebrand disease. In some embodiments, a method of treating of any of the aspects or embodiments herein, can include a method of treating a subject afflicted with any of the indications as described herein. In any of the methods herein wherein platelet derivatives are administered to a subject having any of the listed indications/disorders, the subject can have an anti-coagulant or antiplatelet agent in their body, such as in their blood, and can be, or have been within 1 month, 1 week, 5 days, 4 days, 3, days, 2 days, 1 day, 12 hours, 8 hours, or 4 hours, taking or administered an anti-coagulant and/or an anti-platelet agent.

In some embodiments, a platelet derivative composition of any of the aspects or embodiments herein, or the platelet derivative composition prepared by any of the process described in the aspects or embodiments can be used to treat a subject having any one or a combination of any of the indications as described herein. In certain embodiments, a platelet derivative composition as described herein can be used, for example as a medicament or in the manufacture of a kit, for treating a subject having any one or a combination of indication as disclosed herein.

In some embodiments, an indication or indications can include those type of indications which require a much higher dose of the platelet derivatives herein, or would require an unsafe dose of, or cannot be treated with, unmodified, cold stored, naturally-occurring, endogenous, autologous, allogeneic, or normal platelets, platelets having the characteristics of in-vivo platelets, or conventional platelets (e.g. platelets collected by a conventional method for collecting platelets such as, for example, a platelet-rich plasma-method, a buff coat-method, or by apheresis), but are treatable using a platelet derivative composition of any of the aspects or embodiments herein, or the platelet derivative composition prepared by any of the processes disclosed herein, in illustrative embodiments freeze-dried platelet derivatives. In some embodiments, such an indication can be an indication that is associated with defective platelet production in a subject. In some embodiments, such an indication can be an indication that is associated with a defective platelet activity in a subject. In some embodiments, such an indication can be any of the indication as described herein. In some embodiments, indications that are typically cannot be treated with conventional platelets, but are treatable with a platelet derivative composition as disclosed herein are Von Willebrand disease, immune thrombocytopenia (ITP), intracranial hemorrhage (ICH), traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, or combinations thereof. In some embodiments, such an indication can be Von Willebrand disease. In some embodiments, such an indication can be Immune thrombocytopenia. In some embodiments, such an indication can be Chemotherapy induced thrombocytopenia (CIT). In some embodiments, such an indication can be fetal and neonatal alloimmune thrombocytopenia.

In some embodiments, an indication or indications can include those type of indications which typically, can be well-suited for treatment using a platelet derivative composition as described herein. In some embodiments, such an indication or indications can include Von Willebrand disease, immune thrombocytopenia, intracranial hemorrhage (ICH), traumatic brain injury (TBI), Hermansky Pudlak Syndrome (HPS), Scott syndrome, Evans syndrome, Bernard Soulier syndrome, Glanzmann thrombasthenia, coronary thrombosis (myocardial infarction), arterial thromboembolism, Wiskott Aldrich syndrome, venous thromboembolism, and acute coronary syndrome.

In some embodiments, platelet derivatives as described herein can have several applications in terms of treating a subject suffering with a disorder selected from the group consisting of alopecia areata, Von Willebrand Disease, hemophilia, thrombasthenia, thrombocytopenia, thrombocytopenic purpura, trauma, or a combination thereof. In some embodiments, the platelet derivatives can be used to treat clotting-related disorders. The platelet derivatives as disclosed herein can be used both as a topical application and systemic administration. In some embodiments, there is provided a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenic purpura, trauma, or a combination thereof. In some embodiments, a platelet derivative composition is passed through a filter of 18 μm before administering to the subject. A skilled artisan would be able to appreciate that the platelet derivative composition in the form of a powder which would be commercialized in vials would be rehydrated with an appropriate amount of a solution before administering to a subject. In some embodiments, such a rehydrated platelet derivative composition is passed through a filter of 18 μm before administering to the subject. In some embodiments, the platelet derivative composition as disclosed herein can be used in treating a coagulopathy in a subject that has been administered or is being administered an antiplatelet agent. In some embodiments, the platelet derivative composition of any of the aspects or embodiments herein is provided for use an anti-platelet reversal agent. In some embodiments, the platelet derivative composition of any of the aspects or embodiments herein can be used in treating a coagulopathy in a subject that has been administered or is being administered an anticoagulant agent.

In some embodiments, the platelet derivatives disclosed herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject and/or a wound of the subject.

In some embodiments, the administering can include administering topically. Administering can include administering parenterally. Administering can include administering intravenously. Administering can include administering intramuscularly. Administering can include administering intrathecally. Administering can include administering subcutaneously. Administering can include administering intraperitoneally.

Platelet derivative compositions comprising platelet derivatives as described herein can be used as a medicament for treating a subject. Further, there is also provided herein, methods of treating a subject by administering to a subject a therapeutically effective amount or dose of a platelet derivative composition comprising platelet derivatives as described herein. In some embodiments, the subject is suffering from a condition, or a disease selected from the group consisting of thrombocytopenia, hematologic malignancy, bone marrow aplasia, myeloproliferative disorders, myelodysplastic syndromes, and platelet refractoriness. In some embodiments, the subject is suffering from one or more diseases or condition as described herein. In some embodiments, a therapeutically effective dose of platelet derivatives is based on units of thrombin generation activity administered per kilogram of body weight of the subject and/or the number of platelet derivatives delivered to the subject. In some embodiments of any aspect or embodiment herein the effective dose is based on the weight of the subject. It can be contemplated by a person of skill in the art that the effective dose can be based on any criteria that suits the requirement of the medical intervention of the subject.

A person of skill in the art can contemplate the effective dose of platelet derivatives that can be required to treat a subject in need thereof. The need may differ based on the condition of the subject. The effective dosage can be categorized into a) low dosage; b) medium dosage; and c) high dosage. In some embodiments, a medicament or a method of treating a subject can have the effective dose as low, medium, or high dosage of platelet derivatives that can broadly range from 1.0×10⁷ on the low end of the range to 1.0×10¹⁰/kg, 1.0×10¹¹/kg or 1.0×10¹²/kg of the subject on the high end of the range.

In some embodiments, a platelet derivative composition as described herein can be administered or delivered to a subject, such as a subject afflicted with any one or combination of indications as described herein, and the dose of a platelet derivative composition can be in the range of 1.0×10⁷ to 1.0×10¹² particles/kg of the subject. For example, in some embodiments, a dose of a composition comprising platelets or platelet derivatives (e.g., FDPDs) can include between about or exactly 1.0×10⁷ on the low end of the range to 1.0×10¹² particles (e.g. FDPDs)/kg of a subject on the high end of the range, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹¹ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁹ to 5.0×10⁹ particles (e.g. FDPDs)/kg of a subject, 5.0×10⁷ to 1.0×10¹¹ particles (e.g. FDPDs)/kg of a subject, 5.0×10⁹ to 1.0×10¹⁰ particles (e.g. FDPDs)/kg of a subject), or 1.0×10¹⁰ to 1.0×10¹¹ or 1.0×10¹² particles (e.g. FDPDs)/kg of a subject on the high end of the range. In some embodiments, the dose can be in the range of 250 and 5000 TGPU per kg of the subject.

In some embodiments, a platelet derivative composition, such as that provided in any aspect or embodiment herein, can be administered or delivered to a subject afflicted with any one or combination of indications/diseases as disclosed herein, and the dose of a platelet derivative composition can be in the range of 1.0×10⁷ on the low end of the range to 1.0×10¹² particles/kg of the subject. For example, in some embodiments, a dose of a composition comprising platelets or platelet derivatives (e.g., FDPDs) can include between about or exactly 1.0×10⁷ on the low end of the range to 1.0×10¹² particles (e.g. FDPDs)/kg of a subject, 1.0×10⁷ to 1.0×10¹¹ particles (e.g. FDPDs)/kg of a subject, 1.0×10⁷ to 1.0×10¹⁰ particles (e.g. FDPDs)/kg of a subject, 1.6×10⁷ to 1.0×10¹⁰ particles (e.g. FDPDs/kg of subject, 1.6×10⁷ to 5.1×10⁹ particles (e.g. FDPDs/kg of a subject, 1.6×10⁷ to 3.0×10⁹ particles (e.g. FDPDs)/kg of a subject, 1.6×10⁷ to 1.0×10⁹ particles (e.g. FDPDs)/kg of a subject, 1.6×10⁷ to 5.0×10⁸ particles (e.g. FDPDs)/kg of a subject, 1.6×10⁷ to 1.0×10⁸ particles (e.g. FDPDs)/kg of a subject, 1.6×10⁷ to 5.0×10⁷ particles (e.g. FDPDs)/kg of a subject, 5.0×10⁷ to 1.0×10⁸ particles (e.g. FDPDs)/kg of a subject, 1.0×10⁸ to 5.0×10⁸ particles (e.g. FDPDs)/kg of a subject, 5.0×10⁸ to 1.0×10⁹ particles (e.g. FDPDs)/kg of a subject, 1.0×10⁷ to 5.0×10⁹ particles (e.g. FDPDs)/kg of a subject, or 5.0×10⁹ to 1.0×10¹⁰ particles (e.g. FDPDs)/kg of a subject). In some embodiments, a dose of a composition comprising platelets or platelet derivatives (e.g., FDPDs) can be a range of between about or exactly 1.0×10⁸, 5.0×10⁸, 1.0×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ on the low end of the range to 1.0×10¹² particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 1×10⁹, 1.6×10⁹, 2×10⁹, 2.5×10⁹, 3.0×10⁹, 4.0×10⁹, 5.0×10⁹, 1.0×10¹⁰, 2.5×10¹⁰, or 5.0×10¹⁰ on the low end of the range to 5.0×10¹¹ particles/kg on the high end of the range. In some embodiments, the dose can be about or exactly 1×10⁹, 1.6×10⁹, 2×10⁹, 2.5×10⁹, 3.0×10⁹, 4.0×10⁹, 5.0×10⁹, 1.0×10¹⁰, 2.5×10¹⁰, or 5.0×10¹⁰ on the low end of the range to 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ on the low end of the range to 1.0×10¹¹ particles or 1.0×10¹² (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be a range of between about or exactly 3.0×10⁹, 4.0×10⁹, or 5.0×10⁹ on the low end of the range to 1.0×10¹⁰ particles (e.g. FDPDs)/kg of a subject on the high end of the range. In some embodiments, and in illustrative embodiments wherein a subject has indications as described herein, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end of the range and 1.5×10¹⁰, 1.4×10¹⁰, 1.3×10¹⁰, or 1.2×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg of the subject on the high end of the range.

In some embodiments of any aspect or embodiment herein a therapeutically effective dose or effective dose or amount of the platelet derivatives in a platelet derivative composition is in the range of 1.5×10⁷ to 5.0×10¹⁰/kg, 2.0×10⁷ to 1.0×10¹⁰/kg, 2.5×10⁷ to 5.0×10⁹/kg, 2.75×10⁷ to 3.0×10⁹/kg, 2.8×10⁷ to 4.0×10⁹/kg, 3.0×10⁷ to 4.0×10⁹/kg, 5×10⁷ to 4.0×10⁹/kg, 6×10⁷ to 3.0×10⁹/kg, 9×10⁷ to 3.0×10⁹/kg, 1.0×10⁸ to 2.0×10⁹/kg, or 1.3×10⁸ to 1.8×10⁹/kg of the subject. In some embodiments, a therapeutically effective dose or effective dose or amount of the platelet derivatives in a platelet derivative composition is in the range of 5.0×10⁷ to 1.0×10⁹/kg, 1.0×10⁸ to 5.0×10⁸/kg, 1.2×10⁸ to 2.5×10⁸/kg, 1.6×10⁸ to 2.2×10⁸/kg, or 1.7×10⁸ to 2.0×10⁸/kg of the subject. In some embodiments, the platelet derivatives in a platelet derivative composition is 1.1×10⁸/kg, 1.2×10⁸/kg, 1.3×10⁸/kg, 1.4×10⁸/kg, 1.5×10⁸/kg, 1.6×10⁸/kg, 1.7×10⁸/kg, 1.8×10⁸/kg, 1.9×10⁸/kg, 2.0×10⁸/kg, 2.1×10⁸/kg, 2.2×10⁸/kg. 2.3×10⁸/kg, 2.4×10⁸/kg, or 2.5×10⁸/kg of the subject.

In some embodiments of any aspect or embodiment herein a therapeutically effective dose or effective dose or amount of the platelet derivatives in a platelet derivative composition is in the range of 5.1×10⁸ to 9.9×10⁸/kg, 5.5×10⁸ to 9.5×10⁸/kg, 5.8×10⁸ to 9.3×10⁸/kg, 6.1×10⁸ to 9.0×10⁸/kg, 6.5×10⁸ to 8.8×10⁸/kg, 6.8×10⁸ to 8.5×10⁸/kg, 7.0×10⁸ to 8.4×10⁸/kg, 7.5×10⁸ to 8.3×10⁸/kg, 7.8×10⁸ to 8.2×10⁸/kg, or 7.9×10⁸ to 8.1×10⁸/kg of the subject. In some embodiments, a therapeutically effective dose or effective dose or amount of the platelet derivatives in a platelet derivative composition is 7.5×10⁸/kg, 7.6×10⁸/kg, 7.7×10⁸/kg, 7.8×10⁸/kg, 7.9×10⁸/kg, 8.0×10⁸/kg, 8.1×10⁸/kg, 8.2×10⁸/kg, 8.3×10⁸/kg, 8.4×10⁸/kg, or 8.5×10⁸/kg of the subject.

In some embodiments of any aspect or embodiment herein a therapeutically effective dose or effective dose or amount of the platelet derivatives in a platelet derivative composition is in the range of 1.0×10⁹ to 1.0×10¹⁰/kg, 1.1×10⁹ to 8.0×10⁹/kg, 1.2×10⁹ to 7.0×10⁹/kg, 1.2×10⁹ to 6.0×10⁹/kg, 1.2×10⁹ to 5.0×10⁹/kg, 1.3×10⁹ to 4.0×10⁹/kg, 1.3×10⁹ to 3.0×10⁹/kg, 1.3×10⁹ to 2.5×10⁹/kg, 1.4×10⁹ to 1.9×10⁹/kg, 1.50×10⁹ to 1.75×10⁹/kg, or 1.55×10⁹ to 1.70×10⁹/kg of the subject. In some embodiments, a therapeutically effective dose or effective dose or amount of the platelet derivatives in a platelet derivative composition is 1.1×10⁹/kg, 1.2×10⁹/kg, 1.3×10⁹/kg, 1.4×10⁹/kg 1.5×10⁹/kg, 1.55×10⁹/kg, 1.56×10⁹/kg, 1.57×10⁹/kg, 1.58×10⁹/kg, 1.59×10⁹/kg, 1.6×10⁹/kg, 1.61×10⁹/kg, 1.62×10⁹/kg, 1.63×10⁹/kg, 1.64×10⁹/kg, 1.65×10⁹/kg, 1.66×10⁹/kg, 1.7×10⁹/kg, 1.8×10⁹/kg, 1.9×10⁹/kg, or 2.0×10⁹/kg of the subject.

In some embodiments, a medicament or a method of treating a subject can have a low, medium, or high dosage of platelet derivatives that has a potency in the range of 250 to 5000 TGPU per kg of the subject.

In some embodiments of any aspect or embodiment herein a therapeutically effective dose or an effective dose or amount of the platelet derivatives is an amount that has a potency in the range of 250 to 5000 TGPU per kg, 270 to 4500 TGPU per kg, 280 to 4300 TGPU per kg, 290 to 4100 TGPU per kg, 300 to 3800 TGPU per kg, 310 to 3500 TGPU per kg, or 320 to 3000 TGPU per kg of the subject. In some embodiments, a therapeutically effective dose or effective dose or amount of the platelet derivatives is an amount that has a potency in the range of 275 to 500 TGPU per kg, 280 to 450 TGPU per kg, 290 to 400 TGPU per kg, 300 to 375 TGPU per kg, 310 to 350 TGPU per kg, or 320 to 340 TGPU per kg of the subject. In some embodiments of any aspect or embodiment herein a therapeutically effective dose or effective dose or amount of the platelet derivatives is an amount that has a potency of 270 TGPU per kg, 280 TGPU per kg, 290 TGPU per kg, 300 TGPU per kg, 310 TGPU per kg, 320 TGPU per kg, 330 TGPU per kg, 340 TGPU per kg, or 350 TGPU per kg of the subject.

In some embodiments of any aspect or embodiment herein a therapeutically effective dose or effective dose or amount of the platelet derivatives is an amount that has a potency in the range of 1001 to 2000 TGPU per kg, 1200 to 2000 TGPU per kg, 1300 to 1950 TGPU per kg, 1400 to 1900 TGPU per kg, 1500 to 1900 TGPU per kg, 1600 to 1900 TGPU per kg, 1700 to 1900 TGPU per kg, 1750 to 1875 TGPU per kg, or 1800 to 1850 TGPU per kg of the subject. In some embodiments, a therapeutically effective dose or effective dose or amount of the platelet derivatives is an amount that has a potency of 1780 TGPU per kg, 1790 TGPU per kg, 1800 TGPU per kg, 1810 TGPU per kg, 1820 TGPU per kg, 1830 TGPU per kg, 1840 TGPU per kg, or 1850 TGPU per kg of the subject.

In some embodiments of any aspect or embodiment herein a therapeutically effective dose or effective dose or amount of the platelet derivatives is an amount that has a potency in the range of 2001 to 3500 TGPU per kg, 2300 to 3300 TGPU per kg, 2500 to 3100 TGPU per kg, 2600 to 3100 TGPU per kg, 2700 to 3100 TGPU per kg, 2800 to 3100 TGPU per kg, 2850 to 3050 TGPU per kg, 2900 to 3000 TGPU per kg, or 2940 to 2990 TGPU per kg of the subject. In some embodiments, a therapeutically effective dose or effective dose or amount of the platelet derivatives is an amount that has a potency of 2910 TGPU per kg, 2920 TGPU per kg, 2930 TGPU per kg, 2940 TGPU per kg, 2950 TGPU per kg, 2960 TGPU per kg, 2970 TGPU per kg, 2980 TGPU per kg, 2990 TGPU per kg, 3000 TGPU per kg, or 3100 TGPU per kg of the subject.

In certain embodiments, any of the dose ranges provided above, and in illustrative embodiments those that include less than 1×10¹¹ particles/kg, or any of the ranges provided herein, for example those provided in the paragraph immediately above, can be administered more than 1 time to a subject. For example, a dose range of between 1.0×10⁷ particles to about 1.0×10¹⁰ particles, can be administered between 2 and 10 times, or between 2 and 8 times, or between 2 and 6 times, or between 3 and 8 times, or between 3 and 6 times, or between 4 and 6 times in a timeframe between within 1, 2, 3, 4, 5, or 7 days from the first dose. And in some embodiments a single (e.g., 1×10¹⁰), double (e.g., 2×10¹⁰), triple (e.g., 3×10¹⁰), or higher dose can be administered.

In a method of treating a subject with platelet derivatives as described herein, there can be several endpoints that determine if the subject is treated. A method of treating can have one or more primary endpoints. A method can additionally have one or more secondary endpoints. In some embodiments, in a method of treatment or a composition for use as a medicament as described herein, a method or a medicament leads to cessation or decrease in bleeding at a primary bleeding site at 12 hours, 24 hours, 48 hours, 72 hours, 4 days, 5 days, 6 days, and/or 7 days after administering the platelet derivative composition. In illustrative embodiments, a method or a medicament as described herein leads to cessation or decrease in bleeding at some, all, or substantially all bleeding sites in addition to and/or other than at a primary bleeding site at 24 hours after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition. In some embodiments, the bleeding in a subject is a non-compressible bleeding or a non-compressible hemorrhage. A non-compressible hemorrhage is a type of hemorrhage that is inaccessible to a tourniquet or pressure dressing. In some embodiments, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject. In some illustrative embodiments, the increase can be at least 5000 platelets/μl.

In some embodiments, a method or a medicament leads to changes, or in other embodiments, does not lead to changes, in one or more markers of endothelial cell injury in the subject from a pre-administration time through 12 hours to 35 days, 24 hours to 32 days, 24 hours to 30 days, or 48 hours to 28 days after administering the platelet derivative composition. In illustrative embodiments, a method or a medicament leads to changes, or in other embodiments, does not lead to changes, in one or more markers of endothelial cell injury in the subject from a pre-administration time through baseline to 72 hours after administering the platelet derivative composition. In some embodiments, the method or the medicament leads to changes in two or more markers, three or markers, four or more markers, five or more markers, or all of the markers selected from the group consisting of Syndecan-1, hyaluronan, thrombomodulin, vascular endothelial growth factor (VEGF), interleukin 6, and sVE cadherin. In some embodiments, the changes can be an increase or a decrease in the markers of endothelial cell injury in the subject as compared to a control.

In some embodiments, a method or a medicament leads to acceptable measures of coagulation in the subject at between 12 hours to 35 days, 24 hours to 32 days, 24 hours to 30 days, or 24 hours to 28 days after administering the platelet derivative composition. In illustrative embodiments, a method or a medicament leads to acceptable measures of coagulation in the subject at 72 hours after administering the platelet derivative composition. In some embodiments, the acceptable measure of coagulation includes one or more, two or more, three or more, four or more, five or more, or all of prothrombin time (PT), international normalized ratio (INR), fibrinogen, D-dimer, activated partial thromboplastin time (aPTT), and thromboelastography (TEG) or rotational thromboelastometry (ROTEM). In some embodiments, a method or a medicament leads to an increase or a decrease in the acceptable measure of coagulation in the subject as compared to a control.

In some embodiments, a method or a medicament leads to acceptable measures of hematology in the subject from a pre-administration time through 12 hours to 35 days, 24 hours to 32 days, 24 hours to 30 days, or 48 hours to 28 days after administering the platelet derivative composition. In some embodiments, the acceptable measures of hematology are one or more, two or more, three or more, four or more, five or more, or all selected from the group consisting of Prothrombin Fragment 1+2, thrombin generation assay (TGA), Thrombopoietin, activated Protein C, tissue plasminogen activator (TPA), and/or plasminogen activator inhibitor (PAI). In some embodiments, the acceptable measures of hematology can be an increase or a decrease in the subject as compared to a control.

In some embodiments, a method of treatment or a composition for use as a medicament as described herein, a method or a medicament leads to survival of the subject without WHO Grade 2A or greater bleeding during the first 3, 4, 5, 6, 7, 8, 9, or 10 days after administering of a platelet derivative composition.

In some embodiments, administering confers an improved survival at 10, 15, 20, 25, 30, 35, 40, or 50 days after administering the platelet derivatives. In some embodiments, administering leads to a decrease in administration of secondary blood products, platelets, or platelet derivatives to the subject for the first 5, 6, 7, 8, 9, or 10 days after the administering of the platelet derivatives.

A person of skill in the art can contemplate treating a subject or using platelet derivatives as described herein as a medicament in several doses in a span of time for treating the subject. In some embodiments, administering of platelet derivatives as described herein is performed in a maximum of 2, 3, 4, 5, 6, 7, 8, 9, or 10 doses in a 72-hour period of treatment. Further, in some embodiments, the subject involved in the treatment or a medication process satisfies certain criteria involving one or more of: minimum age; minimum weight; total circulating platelets (TCP); confirmed diagnosis of HPS, BSS, hematologic malignancy, myeloproliferative disorder, myelodysplastic syndrome or aplasia; undergoing chemotherapy, immunotherapy, radiation therapy or hematopoietic stem cell transplantation; refractory to platelet transfusion defined as two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of HPS, BSS, hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

A person of skill in the art can contemplate treating a subject or using platelet derivatives as described herein as a medicament in several doses in a span of time for treating the subject. In some embodiments, administering of platelet derivatives, as disclosed herein, is performed for at least, or at a maximum of 2, 3, 4, 5, 6, 7, 8, 9, or 10 doses in a 72-hour period of treatment. In some embodiments, administering of platelet derivatives as disclosed herein, can be performed as a continuous infusion procedure. For example, a specific dose of platelet derivatives can be decided as per the requirement of a subject and the specific dose can be provided to the subject as a continuous infusion procedure with or without an interval. The dose can be any of the doses as disclosed herein. For example, from 1.0×10⁷ on the low end of the range to 1.0×10¹⁰/kg, 1.0×10¹¹/kg or 1.0×10¹²/kg of the subject on the high end of the range. A particular dose can be any dose as disclosed herein, and the dose can vary during the time interval for which the platelet derivatives are administered to a subject or a recipient in need thereof. In some embodiments, administering can be performed as a continuous infusion procedure until the bleeding potential of the subject is reduced as compared to the bleeding potential before the administering. In some embodiments, administering can be performed as a continuous infusion until the bleeding in the subject is stopped. In some embodiments, administering of platelet derivatives as described herein can be performed at regular intervals. For example, a single, double, triple or more doses of platelet derivatives as described herein and as per the requirement of a subject, for example to reduce or stop bleeding of the subject, can be administered to the recipient subject every 10 minutes, 20 minutes, 30 minutes, 1 hour, 2 hours, 3 hours, 4 hours, 5 hours, 6 hours, 7 hours, 8 hours, 9 hours, 10 hours, 11 hours, or 12 hours depending on whether bleeding is reduced to a satisfactory level, for example such that it is no longer considered life-threatening, or no longer considered severe or serious, or continued until the bleeding is mild, or stops. In some embodiments, the doses can be administered at a regular interval for 36 hours, 48 hours, or 72 hours from the start of the first dose. In some embodiments, administering of platelet derivatives as described herein can be performed as a mixed procedure in which the continuous infusion can be interrupted with a specific dose of platelet derivatives followed by a specific interval as per the requirement. In some embodiments, administering of platelet derivatives as described herein can be performed in a maximum of 2, 3, 4, 5, 6, 7, 8, 9, or 10 doses in a 24-hour period. In some embodiments, administering of platelet derivatives as described herein is performed in a maximum of 2, 3, 4, 5, 6, 7, 8, 9, or 10 doses in a 72-hour period of treatment. In some embodiments, administering of platelet derivatives can be performed at a frequency of at least one dose every 15 minutes or more frequently. For example, administering can be performed at a frequency of at least one dose every 15 minutes or more frequently starting from the first dose until the bleeding potential of the subject is reduced as compared to the bleeding potential before the administering. In some embodiments, the administering can be performed until the bleeding stops. In some embodiments, the administering can be performed for at least 1, 10, 15, 30, 45, or 60 minutes. In some embodiments, the administering can be performed at a frequency of at least one dose in every 20 minutes, 30 minutes, 45 minutes, 60 minutes, 2 hours, 3 hours, 4 hours, 5 hours, 6 hours, 7 hours, 8 hours, 9 hours, 10 hours, 12 hours, 15 hours, 18 hours, 24 hours, 30 hours, or 36 hours or more frequently. Further, in some embodiments, the subject, patient, or recipient administered FDPDs herein, or involved in the treatment or a medication process satisfies certain criteria involving one or more of: minimum age; minimum weight; total circulating platelets (TCP); confirmed diagnosis of hematologic malignancy, myeloproliferative disorder, myelodysplastic syndrome or aplasia; undergoing chemotherapy, immunotherapy, radiation therapy or hematopoietic stem cell transplantation; refractory to platelet transfusion, for example having two 1-hour CCI of <5000 on consecutive transfusions of liquid stored platelets; and WHO Bleeding Score of 2 excluding cutaneous bleeding. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding. In some embodiments, the subject at the time of administering has one, two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets. In some embodiments, the administering is performed during surgery.

Delivery to a Subject being Treated with an Anti-Platelet and/or Anti-Coagulant Agent

In some aspects and embodiments, platelet derivatives provided herein can be used to treat a coagulopathy in a subject that has been administered or is being administered an antiplatelet agent and/or an anticoagulant agent and/or aspirin, and in illustrative embodiments, such subject is afflicted with HPS or BSS. For example, the subject in some embodiments has been, or is being administered, an antiplatelet agent, aspirin, and an anticoagulant. Accordingly, in related aspects and embodiments platelet derivatives as provided herein can be used as an anti-platelet reversal agent, or an anti-coagulant reversal agent. The antiplatelet class of drugs, which an illustrative class of antiplatelet agents, is widely used to prevent unwanted clotting episodes that lead to heart failure, stroke, and the like. In many cases, an antiplatelet drug may need to be reversed or stopped. In the case of advanced notice, as in a pre-planned surgery situation, the antiplatelet drug dose can sometimes be stopped before the surgery, preventing unwanted bleeding during surgery. In the case where an antiplatelet agent needs reversing quickly, reversal agents are typically not readily available, are expensive, or carry significant risk to the patient. In the case of need for rapid antiplatelet reversal, a platelet transfusion is typically administered, though the response to this is often only partial reversal. The caveat of this course of reversal is that the newly-infused platelets themselves are susceptible to circulating drug antiplatelet activity whereas, in some embodiments, compositions as described herein (e.g., including thrombosomes) are not. In some embodiments, compositions as described herein (e.g., including thrombosomes) are an active reversal agent. In some embodiments, the hemostatic activity of compositions as described herein (e.g., including thrombosomes) does not succumb to antiplatelet drugs. In some embodiments, an antiplatelet agent can be selected from the group consisting of aspirin, cangrelor, ticagrelor, clopidogrel, prasugrel, eptifibatide, tirofiban, abciximab, and combinations thereof.

In some embodiments of any of the methods for treating or methods of administering aspects herein, the subject is being treated or was treated with an anti-coagulant. In certain embodiments, the anticoagulant is dabigatran, argatroban, hirudin, rivaroxaban, apixaban, edoxaban, fondaparinux, warfarin, heparin, a low molecular weight heparin, a supplement, or a combination thereof. In some embodiments of any of the methods for administering or for treating aspects herein, wherein the subject is being treated with an anticoagulant, the anticoagulant is dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a health and wellness supplement with anti-coagulant properties, or a combination thereof.

In some embodiments, there is provided a method of administering to a subject, or a method of treating coagulopathy in a subject, wherein the subject has been treated or is being treated with an antiplatelet agent or an anti-coagulant, the method comprising administering to the subject in need thereof an effective amount of a composition comprising platelet derivatives as described herein and an incubating agent comprising one or more salts, a buffer, optionally a cryoprotectant, and optionally an organic solvent. In some embodiments, there is provided a method of restoring normal hemostasis in a subject, the method comprising administering to the subject in need thereof an effective amount of a composition comprising platelet derivatives as described herein and an incubating agent comprising one or more salts, a buffer, optionally a cryoprotectant, and optionally an organic solvent. In some embodiments, there is provided a method of preparing a subject for surgery, the method comprising administering to the subject in need thereof an effective amount of a composition comprising platelet derivative composition as described herein and an incubating agent comprising one or more salts, a buffer, optionally a cryoprotectant, and optionally an organic solvent.

In some embodiments, the subject that has been administered or is being administered an antiplatelet agent or an anticoagulant agent in a subject having an indication and thus afflicted with a disorder or disease, that is any one or a combination of Von Willebrand disease, immune thrombocytopenia, intracranial hemorrhage (ICH), traumatic brain injury (TBI), Hermansky Pudlak Syndrome (HPS), chemotherapy induced thrombocytopenia (CIT), Scott syndrome, Evans syndrome, Hematopoietic Stem Cell Transplantation, fetal and neonatal alloimmune thrombocytopenia, Bernard Soulier syndrome, acute myeloid leukemia, Glanzmann thrombasthenia, myelodysplastic syndrome, hemorrhagic shock, coronary thrombosis (myocardial infarction), ischemic stroke, arterial thromboembolism, Wiskott Aldrich Syndrome, venous thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, aplastic anemia, chronic liver disease, acute radiation syndrome, Dengue hemorrhagic fever, pre-eclampsia, snakebite envenomation, HELLP syndrome, haemorrhagic cystitis, multiple myeloma, disseminated intravascular coagulation, heparin induced thrombocytopenia, pre-eclampsia, labor and delivery, hemophilia, cerebral (fatal) malaria, Alexander's Disease (Factor VII deficiency), hemophilia c (factor xi deficiency), familial hemophagocytic lymphohistiocytosis, acute lung injury, hemolytic uremic syndrome, menorrhagia, chronic myeloid leukemia.

Provided herein in one aspect is a method of treating a coagulopathy in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject. In some embodiments, the coagulopathy is caused by a subject being afflicted with HPS or BSS.

In one aspect, provided herein is a method of treating a coagulopathy in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of restoring normal hemostasis in a subject, the method including administering to the subject in need thereof an effective amount of a composition including platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of restoring normal hemostasis in a subject, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition comprises platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of preparing a subject for surgery, the method including administering to the subject in need thereof an effective amount of a composition including platelets, or in illustrative embodiments platelet derivatives, and in further illustrative embodiments FDPDs. Various properties of exemplary embodiments of such FDPDs are provided herein, thereby treating the coagulopathy. In illustrative embodiments, the composition comprising the platelet derivatives is administered such that the bleeding potential of the subject is reduced, and in illustrative embodiments such that normal hemostasis is restored in the subject. Implementations can include one or more of the following features. The surgery can be an emergency surgery. The surgery can be a scheduled surgery.

In one aspect, provided herein is a method of preparing a subject for surgery, the method including administering to the subject in need thereof an effective amount of a composition prepared by a process including incubating platelets with an incubating agent including one or more salts, a buffer, optionally a cryoprotectant, and optionally an organic solvent, and in illustrative embodiments freeze-drying the incubated platelets, to form the composition, wherein the composition includes platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject. Various properties of exemplary embodiments of such FDPDs are provided herein. Implementations can include one or more of the following features. The surgery can be an emergency surgery. The surgery can be a scheduled surgery.

In one aspect, provided herein is a method of ameliorating the effects of an antiplatelet agent in a subject, the method including administering to the subject in need thereof an effective amount of a composition platelets, or in illustrative embodiments platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of ameliorating the effects of an antiplatelet agent in a subject, the method including administering to the subject in need thereof an effective amount of a composition prepared by a process including incubating platelets with an incubating agent including one or more salts, a buffer, optionally a cryoprotectant, and optionally an organic solvent, to form the composition, wherein the composition includes platelet derivatives, and in further illustrative embodiments FDPDs, thereby treating the coagulopathy. In illustrative embodiments, the composition comprising the platelet derivatives is administered such that the bleeding potential of the subject is reduced, and in illustrative embodiments such that normal hemostasis is restored in the subject.

In one aspect, provided herein is a method of treating a coagulopathy in a subject, or of restoring hemostasis in a subject, or of reducing bleeding potential of a subject that is being administered, or has been administered, an antiplatelet agent, the method comprising: administering to the subject in need thereof an effective amount of a composition comprising platelet derivatives, thereby treating the coagulopathy. In illustrative embodiments, the platelet derivatives are freeze-dried platelet derivatives (FDPDs). In further illustrative embodiments, the composition comprising the platelet derivatives is administered such that the bleeding potential of the subject is reduced, and in illustrative embodiments such that normal hemostasis is restored in the subject.

In another aspect, provided herein is a method of treating a coagulopathy in a subject, or of restoring hemostasis in a subject, or of reducing bleeding potential of a subject, wherein the subject is being administered, or has been administered, an antiplatelet agent, the method comprising administering to the subject in need thereof an effective amount of the composition comprising FDPDs, wherein the composition comprising FDPDs comprises a population of FDPDs having a reduced propensity to aggregate such that no more than 10% of the FDPDs in the population aggregate under aggregation conditions comprising an agonist but no platelets, thereby treating the coagulopathy. In further illustrative embodiments, the composition comprising the FDPDs is administered such that the bleeding potential of the subject is reduced, and in illustrative embodiments such that normal hemostasis is restored in the subject.

In another aspect, provided herein is a method of preventing or mitigating the potential for a coagulopathy in a subject, the method comprises: (a) determining that information regarding whether the subject was administered an antiplatelet agent is unavailable; and (b) administering to the subject an effective amount of a composition comprising freeze-dried platelet derivatives (FDPDs). In some embodiments of such a method, information regarding whether the subject was administered an antiplatelet agent is unavailable for a reason comprising that the subject cannot be identified. In some embodiments of the method, information regarding whether the subject was administered an antiplatelet agent is unavailable for a reason comprising that the medical history of the subject is unavailable. In further embodiments information regarding whether the subject was administered an antiplatelet agent is unavailable for a reason comprising that the subject is in need of emergency treatment.

In another aspect, provided herein is a method of treating a coagulopathy in a subject or of reducing the bleeding potential of a subject, or of restoring hemostasis in a subject, wherein the method comprises: administering to the subject in need thereof an effective amount of a composition comprising platelet derivatives, in illustrative embodiments, FDPDs, wherein the subject before the administering the composition comprising platelet derivatives, was administered an antiplatelet agent and a second agent that decreases platelet function, thereby treating the coagulopathy. In further illustrative embodiments, the composition comprising the platelet derivatives is administered such that the bleeding potential of the subject is reduced, and in illustrative embodiments such that normal hemostasis is restored in the subject. In illustrative embodiments, before the administering of the composition comprising FDPDs the subject was in need thereof because of an increased risk of bleeding due to, or as a result of being administered the anti-platelet agent and the second agent.

In another aspect, provided herein is a composition comprising freeze-dried platelet derivatives (FDPDs) for treating a coagulopathy in a subject, wherein the treating comprises:

administering to the subject in need thereof, an effective amount of the composition comprising FDPDs such that the bleeding potential, or risk of bleeding of the subject is reduced, wherein the subject was administered an antiplatelet agent and a second agent that decreases platelet function, and wherein the subject is in need thereof because of an increased potential for, or risk of bleeding due to, or as a result of being administered the antiplatelet agent and the second agent, thereby treating the coagulopathy.

In another aspect, provided herein is a composition comprising freeze-dried platelet derivatives (FDPDs) for treating a coagulopathy in a subject having an increased potential for, or risk of bleeding as a result of being administered or having been administered an anticoagulant, wherein the treating comprises: administering to the subject having the increased potential for, or risk of bleeding, an effective amount of the composition comprising FDPDs such that the bleeding potential or risk of bleeding of the subject is reduced, wherein the composition comprising FDPDs comprises a population of FDPDs having a reduced propensity to aggregate such that no more than 10% of the FDPDs in the population aggregate under aggregation conditions comprising an agonist but no platelets, thereby treating the coagulopathy.

In some embodiments of any of the method or use embodiments herein, a dose, and in illustrative embodiments an effective amount of a composition comprising platelets or platelet derivatives (e.g., FDPDs) administered to a subject or patient, can be in a range of between about or exactly 1.0×10⁸, 5.0×10⁸, 1.0×10⁹, 3.0×10⁹, 4.0×10⁹, 5.0×10⁹, 1.0×10¹⁰, or 5.0×10¹⁰ to 1.0×10¹² particles (e.g. platelet derivatives or FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ to 5.0×10¹¹ particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, 5.0×10⁹, 1.0×10¹⁰, 2.5×10¹⁰, or 5.0×10¹⁰ to 1.0×10¹¹ particles (e.g. FDPDs)/kg of a subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be a range of between about or exactly 3.0×10⁹, 4.0×10⁹, or 5.0×10⁹ to 1.0×10¹⁰ particles (e.g. FDPDs)/kg of a subject. In one illustrative embodiment, and in illustrative embodiments wherein a subject has blood comprising i) an anti-platelet agent and a second agent that decreases platelet function; ii) two anti-platelet agents; and/or iii) has been administered dual anti-platelet therapy, a dose or an effective amount of a composition comprising FDPDs is between 5.0×10¹⁰ to 1.0×10¹2/kg of the subject, 5.0×10¹⁰ to 5.0×10¹¹/kg of the subject, 5.0×10¹⁰ to 1.0×10¹¹/kg of the subject, 5.0×10⁹ to 1.0×10¹¹/kg of the subject, 5.0×10⁹ to 5.0×10¹⁰/kg of the subject, or 5.0×10⁹ to 1.0×10¹⁰/kg of the subject. In some embodiments, and in illustrative embodiments wherein a subject has blood comprising two anti-platelet agents and/or has been administered dual anti-platelet therapy, a dose of a composition comprising platelets or platelet derivatives (e.g., FDPDs) can be in a range of greater than 1.5×10⁹ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or greater than 1.0×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end of the range and 1.5×10¹⁰, 1.4×10¹⁰, 1.3×10¹⁰, 1.2×10¹⁰, or 1.1×10¹⁰ FDPDs/kg of the subject on the high end; or 1.1×10¹⁰ FDPDs/kg of the subject on the low end and less than 1.5×10¹⁰, 1.4×10¹⁰, 1.3×10¹⁰, or 1.2×10¹⁰ FDPDs/kg of the subject on the high end.

In some embodiments, for example of aspects wherein a subject was administered the antiplatelet agent and the second agent that decreases platelet function, such a method further comprises before the administering the composition comprising FDPDs, determining that the subject was administered the antiplatelet agent and the second agent that decreases platelet function. In some embodiments, the antiplatelet agent is a first antiplatelet agent and the second agent is a second antiplatelet agent. In some embodiments, the first antiplatelet agent and the second anti-platelet agent are each different antiplatelet agents selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate. In some embodiments, the first antiplatelet agent and the second anti-platelet agent have different mechanisms of action. In some embodiments, the first antiplatelet agent and the second anti-platelet agent are each different antiplatelet agents selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate.

In some embodiments of any of the aspects herein, before, immediately before, at the moment before, at the moment of, and/or at an initial time of, the administering of the composition comprising platelet derivatives, for example FDPDs, the subject was or is at an increased risk of bleeding due to being administered or having been administered the anti-platelet agent. Furthermore, the subject can be at an increased risk of bleeding at 7, 6, 5, 4, 3, 2, or 1 day, 12 hours, 8 hours, 4 hours, 2 hours, 1 hour or 45, 30, 15, 10, 5, 4, 3, 2, or 1 minute before the administering of the composition comprising the platelet derivatives. In some optional embodiments, this is confirmed by laboratory testing. However, in some embodiments no laboratory testing of bleeding risk or any clotting parameter is performed 7, 6, 5, 4, 3, 2, or 1 day or sooner before and/or after the administering of the composition comprising the platelet derivatives. Bleeding risk is typically decreased after administration of an effective dose of the composition comprising platelet derivatives, in illustrative embodiments FDPDs. Furthermore, the subject may remain at an increased risk of bleeding even after the administering of the composition comprising platelet derivatives (e.g. FDPDs), for example for 1, 2, 3, 4, 5, 10, 15, 20, 30, or 45 minutes, or 1, 2, 3, 4, 5, or 8 hours, or longer after the administering, depending on how long it takes for the FDPDs to decrease the risk in the subject after they are administered. Furthermore, in some embodiments, the administration of the composition comprising the platelet derivatives (e.g. FDPDs) decreases but does not completely resolve the increased risk of bleeding in the subject.

In some embodiments, for example of aspects wherein a subject was administered the antiplatelet agent and the second agent that decreases platelet function, administration of the second agent is stopped, for example before administrating the composition comprising the platelet derivatives. In other embodiments of such aspects, administration of the second agent is continued, for example after administering the composition comprising the platelet derivatives.

In certain embodiments of any of the aspects provided herein, the method further comprises before administering the composition comprising platelet derivatives, determining in a pre-administering evaluation, that the subject has an abnormal value for one or more clotting parameters. The pre-administration evaluation, in illustrative embodiments, is an in vitro laboratory test.

In certain embodiments of any of the aspects provided herein, the antiplatelet agent is selected from aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, and sarpogrelate. In other embodiments, the antiplatelet agent is selected from cangrelor, ticagrelor, abciximab, terutroban, picotamide, elinogrel, ibuprofen, vorapaxar, atopaxar, cilostazol, prostaglandin E1, epoprostenol, dipyridamole, treprostinil sodium, and sarpogrelate.

### Quantity of Platelet or Platelet Derivatives, and Erythrocytes

The composition comprising platelet derivatives and aqueous medium can have varying quantity of platelets and/or platelet derivatives, optionally along with erythrocytes. In some embodiments, a composition as described herein can have a platelet count of at least 10⁶ (e.g., at least 5×10⁶, 10⁷, 5×10⁷, 10⁸, 5×10⁸, 10⁹, 5×10⁹, or 10¹⁰). In some embodiments, a composition as described herein can have a platelet count of at least about 200×10³ platelets/μL (e.g., at least about 300×10³, 400×10³, 500×10³, 750×10³, 1000×10³, 1500×10³, 2000×10³, or 2500×10³ platelets/μL). In some embodiments, a composition as described herein can have a platelet count of at least about 2000×10³ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). In some embodiments, a composition as described herein can have a platelet count less than or equal to 1000×10⁴ platelets/μL. In some embodiments, the platelets or platelet derivatives in the composition are at least 100×10³ platelets/μL, or 200×10³ platelets/μL, or 400×10³ platelets/μL, or 1000×10³ platelets/μL, or 1250×10³ platelets/μL, or 1500×10³ platelets/μL, or 1750×10³ platelets/μL, 2000×10³ platelets/μL, or 2250×10³ platelets/μL, or 2500×10³ platelets/μL, or 2750×10³ platelets/μL, or 3000×10³ platelets/μL, 3250×10³ platelets/μL, 3500×10³ platelets/μL, 3750×10³ platelets/μL, 4000×10³ platelets/μL, or 4250×10³ platelets/μL, or 4500×10³ platelets/μL, or 4750×10³ platelets/μL, or 5000×10³ platelets/μL, or 5250×10³ platelets/μL, or 5500×10³ platelets/μL, or 5750×10³ platelets/μL, or 6000×10³ platelets/μL, or 7000×10³ platelets/μL, or 8000×10³ platelets/μL, or 9000×10³ platelets/μL, or 10,000×10³ platelets/μL, or 11,000×10³ platelets/μL, or 12,000×10³ platelets/μL, or 13,000×10³ platelets/μL, or 14,000×10³ platelets/μL, or 15,000×10³ platelets/μL, or 16,000×10³ platelets/μL, or 17,000×10³ platelets/μL, or 18,000×10³ platelets/μL, or 19,000×10³ platelets/μL, or 20,000×10³ platelets/μL. In some embodiments, the platelets or platelet derivatives in the composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL.

In some embodiments, a composition as provided herein can include erythrocytes. In some embodiments, a composition as provided herein can have an erythrocyte count of less than about 10¹⁰ (e.g., less than 5×10⁹, 10⁹, 5×10⁸, 10⁸, 5×10⁷, 10⁷, 5×10⁶, or 10⁶). In some embodiments, the erythrocyte count can be less than 0.2×10⁶/μL (e.g., less than 0.1×10⁶/μL, 0.5×10⁵/μL, or 0.1×10⁵/μL). In some embodiments, the erythrocytes in the composition is in the range of 0.1×10⁷ erythrocytes/μL to 0.2×10⁶ erythrocytes/μL, or 0.5×10⁵ erythrocytes/μL to 0.1×10⁶ erythrocytes/μL.

### Reduced Plasma Protein Content and Antibodies

Transfusion-related acute lung injury (TRALI) is a condition believed to be caused by the presence of antibodies (e.g., Human Leukocyte Antigen (HLA), Human Neutrophil Antigen (HNA), or granulocyte antibodies) in a transfused blood product, which can react with antigens in a transfusion recipient. Thus, the use of plasma-based blood products from donors considered to be high-risk or who test positive for Human Leukocyte Antigen (HLA) Class I, Class II, and neutrophil-specific antibodies can cause issues in transfusion or production of human-derived platelet products (e.g., compositions comprising platelets and/or platelet derivatives (e.g., thrombosomes)) and in illustrative embodiments, are therefore omitted from a donor pool.

The use of tangential flow filtration (TFF) or multi-pass centrifugation can reduce the amount of antibody in a blood product, for example, to limits not detectable by current, FDA-approved, testing methods. In some cases, reduction of certain plasma components (e.g., HLA antibodies) can allow for this donor population to be accepted for production of blood products (e.g., compositions comprising platelets and/or platelet derivatives (e.g., thrombosomes)). In some embodiments described herein, a blood product can be a composition that includes platelets or platelet derivatives and an aqueous medium.

Thrombosomes or cryopreserved platelet production can be limited by the availability of licensed apheresis collections performed at blood donor centers around the United States. Competition for these products can be fierce, and distribution for blood product manufacturing needs is usually prioritized below the needs of patient care. Blood product manufacturing (e.g., scale-up), could be aided by apheresis collections from otherwise deferred donors. One way this could be accomplished is by reducing free antibody levels in donor plasma to meet current, FDA approved, testing thresholds by utilizing tangential flow filtration (TFF) or centrifugation and plasma removal. Centrifugation of the raw materials (e.g., donor plasma), while typically more time consuming than TFF, can have a similar effect on the raw material. In some cases, removal of the donor plasma and replacement with buffer can allow the inventors to manufacture and characterize a final product (e.g., compositions comprising platelets and/or platelet derivatives (e.g., thrombosomes)) with a reduced protein (e.g., antibody (e.g., HLA antibody or HNA antibody)) content (e.g., as measured by absorbance at 280 nm). Such a product can increase the safety for a recipient of the product by reducing the transfusion related cause for TRALI.

In some embodiments, the materials and methods provided herein can allow previously deferred donors (such as those who screen positive for HLA antibodies or whose donor history presents a risk for positive HLA) to be allowed into the donor pool of raw materials used to manufacture blood products (e.g., compositions comprising platelets and/or platelet derivatives (e.g., thrombosomes)). In some embodiments described herein, a blood product can be a composition that includes platelets and an aqueous medium. Additionally, a reduction in HLA antibodies from the raw materials (e.g., donor apheresis material (e.g., platelets or pooled platelets)) can allow for a final product (e.g., compositions comprising platelets and/or platelet derivatives (e.g., thrombosomes)) to be labeled as HLA-reduced, increasing the safety of a product for a recipient.

In some embodiments, a blood product (e.g., compositions comprising platelets and/or platelet derivatives (e.g., thrombosomes)) as provided herein can have no detectable level of HLA antibodies. In some embodiments, a blood product (e.g., compositions comprising platelets and/or platelet derivatives (e.g., thrombosomes)) as provided herein can have no detectable level of an antibody selected from the group consisting of HLA Class I antibodies, HLA Class II antibodies, and HNA antibodies. In some embodiments, a blood product (e.g., compositions comprising platelets and/or platelet derivatives (e.g., thrombosomes)) as provided herein can have no detectable level of HLA Class I antibodies. In some embodiments, a blood product (e.g., compositions comprising platelets and/or platelet derivatives (e.g., thrombosomes)) as provided herein can have no detectable level of HLA Class II antibodies. In some embodiments, a blood product (e.g., compositions comprising platelets and/or platelet derivatives (e.g., thrombosomes)) as provided herein can have no detectable level of HNA antibodies. In some embodiments, detection of antibodies can be carried out using a regulatory agency approved (e.g., FDA cleared) assay. A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software. In some embodiments described herein, a blood product can be a composition that includes platelets and an aqueous medium.

In some embodiments, a blood product (e.g., compositions comprising platelets and/or platelet derivatives (e.g., thrombosomes)) as provided herein can have a level of an antibody selected from the group consisting of HLA Class I antibodies, HLA Class II antibodies, and HNA antibodies below a reference level. In some embodiments, a blood product (e.g., compositions comprising platelets and/or platelet derivatives (e.g., thrombosomes)) as provided herein can have a level of HLA Class I antibodies below a reference level. In some embodiments, a blood product (e.g., compositions comprising platelets and/or platelet derivatives (e.g., thrombosomes)) as provided herein can have a level of HLA Class II antibodies below a reference level. In some embodiments, a blood product (e.g., a composition comprising platelets and/or platelet derivatives (e.g., thrombosomes)) as provided herein can have a level of HNA antibodies below a reference level. A reference level can be any appropriate reference level. In some embodiments described herein, a blood product can be a composition that includes platelets and an aqueous medium.

In some embodiments, a blood product (e.g., a composition comprising platelets and/or platelet derivatives (e.g., thrombosomes)) as provided herein test negative for an antibody selected from the group consisting of HLA Class I antibodies, HLA Class II antibodies, and HNA antibodies in a regulatory agency approved assay (e.g., an FDA cleared assay). In some embodiments, a blood product (e.g., a composition comprising platelets and/or platelet derivatives (e.g., thrombosomes)) as provided herein can test negative for HLA Class I antibodies in a regulatory agency approved assay (e.g., an FDA cleared assay). In some embodiments, a blood product (e.g., a composition comprising platelets and/or platelet derivatives (e.g., thrombosomes)) as provided herein can test negative for HLA Class II antibodies a in regulatory agency approved assay (e.g., an FDA cleared assay). In some embodiments, a blood product (e.g., a composition comprising platelets and/or platelet derivatives (e.g., thrombosomes)) as provided herein can test negative for HNA antibodies in a regulatory agency approved assay (e.g., an FDA cleared assay). In some embodiments described herein, a blood product can be a composition that includes platelets and an aqueous medium. A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some aspects, provided herein are compositions comprising platelets and/or platelet derivatives (e.g., thrombosomes) and an aqueous medium. In some embodiments, the aqueous medium can include a preparation agent (e.g., any of the preparation agents described herein). In some embodiments, an aqueous medium as provided herein can have a level of an antibody selected from the group consisting of HLA Class I antibodies, HLA Class II antibodies, and HNA antibodies below a reference level. In some embodiments, an aqueous medium as provided herein can have a level of HLA Class I antibodies below a reference level. In some embodiments, an aqueous medium as provided herein can have a level of HLA Class II antibodies below a reference level. In some embodiments, an aqueous medium as provided herein can have a level of HNA antibodies below a reference level. A reference level can be any appropriate reference level. In some embodiments, an aqueous medium as provided herein can test negative for an antibody selected from the group consisting of HLA Class I antibodies, HLA Class II antibodies, and HNA antibodies in a regulatory agency approved assay (e.g., an FDA cleared assay). In some embodiments, an aqueous medium as provided herein can test negative for HLA Class I antibodies in a regulatory agency approved assay (e.g., an FDA cleared assay). In some embodiments, an aqueous medium as provided herein can test negative for HLA Class II antibodies in a regulatory agency approved assay (e.g., an FDA cleared assay). In some embodiments, an aqueous medium as provided herein can test negative for HNA antibodies in a regulatory agency approved assay (e.g., an FDA cleared assay). A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some embodiments, an aqueous medium can have a reduced amount of residual plasma compared to donor apheresis plasma (e.g., single-donor apheresis plasma or pooled donor apheresis plasma) can be a percentage of residual plasma (e.g., less than or equal to about 50%, 40%, 30%, 20%, 150%, 10%, 9%, 8%, 7%, 6%, 5%, 4%, 3%, 2%, 10%, 0.9%, 0.8%, 0.7%, 0.6%, 0.50%, 0.4%, 0.30%, 0.2%, or 0.10% of residual plasma). In some embodiments, an aqueous medium can have a reduced amount of residual plasma compared to donor apheresis plasma (e.g., single-donor apheresis plasma or pooled donor apheresis plasma) can be a percentage range of residual plasma (e.g., about 5% to about 50%, about 5% to about 40%, about 5% to about 30%, about 5% to about 20%, about 5% to about 15%, about 5% to about 10%, about 10% to about 20%, about 7% to about 15%, about 7% to about 10%, about 8% to about 15%, about 8% to about 10%, about 0.1% to about 5%, about 0.1% to about 3%, about 0.1% to about 1%, about 0.5% to about 3%, about 0.5% to about 1%, or about 1% to about 3% of residual plasma). In some embodiments, an aqueous medium can have a protein concentration less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the protein concentration of donor apheresis plasma (e.g., single-donor apheresis plasma or pooled donor apheresis plasma). In some embodiments, an aqueous medium can have a protein concentration of about 5% to about 50% (e.g., about 5% to about 40%, about 5% to about 30%, about 5% to about 20%, about 5% to about 15%, about 5% to about 10%, about 10% to about 20%, about 7% to about 15%, about 7% to about 10%, about 8% to about 15%, or about 8% to about 10%) of the protein concentration of donor apheresis plasma (e.g., single-donor apheresis plasma or pooled donor apheresis plasma). In some embodiments, an aqueous medium can have a protein concentration of about 0.10% to about 50% (e.g., about 0.10% to about 30%, about 0.10% to about 1%, about 0.5% to about 3%, about 0.5% to about 1%, about 1% to about 2%, or about 1% to about 3%) of the protein concentration of donor apheresis plasma (e.g., single-donor apheresis plasma or pooled donor apheresis plasma). A protein concentration can be measured by any appropriate method. Apart from the relative protein concentration of proteins in the aqueous medium, the protein concentration in the aqueous medium can also be measured in absolute terms. Accordingly, in some embodiments, a protein concentration can be measured by absorbance at 280 nm (A280). In some embodiments, an aqueous medium can have an A280 that is less that is less than 2.0 AU (e.g., less than 1.97, 1.95, 1.93, 1.90, 1.87, 1.85, 1.83, 1.80, 1.77, 1.75, 1.73, 1.70, 1.66, 1.6, 1.5, 1.4, 1.3, 1.2, 1.1, 1.0, 0.9, 0.8, 0.7, 0.6, 0.5, 0.4, 0.3, 0.2, or 0.1 AU) with a path length of 0.5 cm. In some embodiments, the protein concentration in the aqueous medium is less than or equal to 15%, 14%, 13%, 12%, 11%, 10, 9%, 8%, 7%, 6%, 5%, 4%, 3%, 2%, 1%, 0.1%, or 0.01%. In some illustrative embodiments, the protein concentration is less than 3% or 4%. In some embodiments, the protein concentration is in the range of 0.01-15%, 0.1-15%, 1-15%, 1-10%, 0.1-10%, 0.01-10%, 0.1-5%, 0.1-4%, 0.1-3%, 0.1-2%, 0.1-1%, 1-5%, 1-4%, 1-3%, 1-2%, 3-12%, or 5-10%. In some embodiments, an aqueous medium can have a HLA Class I antibody concentration less than about 70% (e.g., less than about 60%, 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the HLA Class I antibody concentration of donor apheresis plasma (e.g., single-donor apheresis plasma or pooled donor apheresis plasma). A HLA Class I antibody concentration can be measured by any appropriate method. In some embodiments, the HLA class I antibody concentration in the aqueous medium can be quantitated in absolute terms such that the aqueous medium can have HLA Class I antibody concentration less than about 70% (e.g., less than about or less than exactly 60%, 50%, 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) in the aqueous medium. In some embodiments, the HLA Class I antibody in the aqueous medium is low enough such that the composition comprising platelet derivatives and aqueous medium is negative for HLA Class I antibodies based on a regulatory agency approved test for HLA Class I antibodies.

In some embodiments, an aqueous medium can have a HLA Class II antibody concentration less than about 50% (e.g., less than about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the HLA Class II antibody concentration of donor apheresis plasma (e.g., single-donor apheresis plasma or pooled donor apheresis plasma). A HLA Class II antibody concentration can be measured by any appropriate method. In some embodiments, the HLA class II antibody concentration in the aqueous medium can be quantitated in absolute terms such that the aqueous medium can have HLA Class I antibody concentration less than about 50% (e.g., less than about or less than exactly 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) in the aqueous medium. In some embodiments, the HLA Class II antibody in the aqueous medium is low enough such that the composition comprising platelet derivatives and aqueous medium is negative for HLA Class II antibodies based on a regulatory agency approved test for HLA Class II antibodies.

In some embodiments, an aqueous medium can have a HNA antibody concentration less than about 50% (e.g., less than about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the HNA antibody concentration of donor apheresis plasma (e.g., single-donor apheresis plasma or pooled donor apheresis plasma). A HNA antibody concentration can be measured by any appropriate method. In some embodiments, the HNA antibody concentration in the aqueous medium can be quantitated in absolute terms such that the aqueous medium can have HNA antibody concentration less than about 50% (e.g., less than about or less than exactly 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) in the aqueous medium. In some embodiments, the HNA antibody in the aqueous medium is low enough such that the composition comprising platelet derivatives and aqueous medium is negative for HNA antibodies based on a regulatory agency approved test for HNA antibodies.

In some cases, flow cytometry can be used to evaluate compositions as provided herein. In some embodiments, an antibody selected from the group consisting of HLA Class I antibodies, HLA Class II antibodies, and HNA antibodies, as determined for a composition comprising platelets and an aqueous medium by flow cytometry using beads coated with Class I HLAs, Class II HLAs, or HNAs, respectively, is less than 10% (e.g., less than about or less than exactly 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class I antibodies, as determined for a composition comprising platelets and an aqueous medium by flow cytometry using beads coated with Class I HLAs, is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class II antibodies, as determined for a composition comprising platelets and an aqueous medium by flow cytometry using beads coated with Class II HLAs is less than 10% (e.g., less than about or less than exactly 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HNA antibodies, as determined for a composition comprising platelets and an aqueous medium by flow cytometry using beads coated with HNAs is less than 10% (e.g., less than about or less than exactly 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%).

In some embodiments, an antibody selected from the group consisting of HLA Class I antibodies, HLA Class II antibodies, and HNA antibodies, as determined for an aqueous medium by flow cytometry using beads coated with Class I HLAs, Class II HLAs, or HNAs, respectively, is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class I antibodies, as determined for an aqueous medium by flow cytometry using beads coated with Class I HLAs, is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HLA Class II antibodies, as determined for an aqueous medium by flow cytometry using beads coated with Class II HLAs is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%). In some embodiments, the percentage of beads positive for HNA antibodies, as determined for an aqueous medium by flow cytometry using beads coated with HNAs is less than 10% (e.g., less than 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%).

In some embodiments, the protein concentration is low enough to exclude the HLA Class I, HLA Class II, and HNA antibodies from the donor pheresis plasma such that the composition is negative for: a) HLA Class I antibodies based on a regulatory agency approved test for HLA Class I antibodies; b) HLA Class II antibodies based on a regulatory agency approved test for HLA Class II antibodies; and c) HNA antibodies based on a regulatory agency approved test for HNA antibodies. The platelet derivative composition of the present disclosure, and the process for obtaining the same, thus provides the flexibility to utilize the apheresis plasma from different multiple donors, and yet obtain a final product (platelet derivative composition) that is negative for the HLA Class I, HLA Class II, and HNA antibodies.

In some aspects, provided herein are platelet derivative compositions comprising platelet derivatives in the form of a solid, a composition with less than 1% water, and/or a powder. The composition in solid form, in illustrative embodiments dried form, for example with less than 1% water, can be one amongst different kinds in which the composition would be packed and commercialized. Thus, it is contemplated that the composition in the dried form would preserve the characteristics with respect to the low content, or even absence of detectable HLA Class I, HLA Class II, and HNA antibodies as described with respect to the aqueous medium in the embodiments described herein. In some embodiments, the platelet derivative composition in the form of a powder is negative for HLA Class I antibodies based on a regulatory agency approved test for HLA Class I antibodies. In some embodiments, the platelet derivative composition in the form of a powder is negative for HLA Class II antibodies based on a regulatory agency approved test for HLA Class II antibodies. In some embodiments, the platelet derivative composition in the form of a powder is negative for HNA antibodies based on a regulatory agency approved test for HNA antibodies. In some embodiments, the platelet derivative composition in the form of a powder is negative for HLA Class I antibodies, HLA Class II antibodies, and HNA antibodies based on a regulatory agency approved test for HLA Class I antibodies, HLA Class II antibodies, and HNA antibodies, respectively.

### Preparation Agent and Additional Components

In some embodiments, a composition provided herein can include one or more additional components. In some embodiments, a composition provided herein can include a preparation agent (e.g., any of the preparation agents described herein). In some embodiments, the composition can include a buffering agent, a base, a loading agent, optionally a salt, and optionally at least one organic solvent. A buffering agent can be any appropriate buffering agent. In some embodiments, a buffering agent can be HEPES (4-(2-hydroxyethyl)-1-piperazineethanesulfonic acid). A base can be any appropriate base. In some embodiments, a base can be sodium bicarbonate. A loading agent can be any appropriate loading agent. In some embodiments, a loading agent can be a monosaccharide, a polysaccharide, or a combination thereof. In some embodiments, a loading agent can be selected from the group consisting of sucrose, maltose, trehalose, glucose, mannose, and xylose. In some embodiments, a loading agent can be trehalose. In some embodiments, a polysaccharide can be polysucrose. A salt can be any appropriate salt. In some embodiments, a salt can be sodium chloride, potassium chloride, or a combination thereof. An organic solvent can be any appropriate organic solvent. In some embodiments, an organic solvent can be selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

A preparation agent can include any appropriate components. In some embodiments, the preparation agent may comprise a liquid medium. In some embodiments the preparation agent may comprise one or more salts selected from phosphate salts, sodium salts, potassium salts, calcium salts, magnesium salts, and any other salt that can be found in blood or blood products, or that is known to be useful in drying platelets, or any combination of two or more of these.

In some embodiments, the preparation agent comprises one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the preparation agent includes from about 0.5 mM to about 100 mM of the one or more salts. In some embodiments, the preparation agent includes from about 0.5 mM to about 100 mM (e.g., about 0.5 to about 2 mM, about 2 mM to about 90 mM, about 2 mM to about 6 mM, about 50 mM to about 100 mM, about 60 mM to about 90 mM, about 70 to about 85 mM) of the one or more salts. In some embodiments, the preparation agent includes about 5 mM, about 75 mM, or about 80 mM of the one or more salts. In some embodiments, the preparation agent comprises one or more salts selected from calcium salts, magnesium salts, and a combination of the two, in a concentration of about 0.5 mM to about 2 mM.

Preferably, these salts are present in the composition comprising platelets or platelet derivatives, such as freeze-dried platelets, at an amount that is about the same as is found in whole blood.

In some embodiments, the preparation agent further comprises a carrier protein. In some embodiments, the carrier protein comprises human serum albumin, bovine serum albumin, or a combination thereof. In some embodiments, the carrier protein is present in an amount of about 0.05% to about 1.0% (w/v).

The preparation agent may be any buffer that is non-toxic to the platelets and provides adequate buffering capacity to the solution at the temperatures at which the solution will be exposed during the process provided herein. Thus, the buffer may comprise any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TBS). Likewise, it may comprise one or more of the following buffers: propane-1,2,3-tricarboxylic (tricarballylic); benzenepentacarboxylic; maleic; 2,2-dimethylsuccinic; EDTA; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES). In some embodiments, the preparation agent includes one or more buffers, e.g., N-2-hydroxyethylpiperazine-N′-2-ethanesulfonic acid (HEPES), or sodium-bicarbonate (NaHCO₃). In some embodiments, the preparation agent includes from about 5 to about 100 mM of the one or more buffers. In some embodiments, the preparation agent includes from about 5 to about 50 mM (e.g., from about 5 mM to about 40 mM, from about 8 mM to about 30 mM, about 10 mM to about 25 mM) about of the one or more buffers. In some embodiments, the preparation agent includes about 10 mM, about 20 mM, about 25 mM, or about 30 mM of the one or more buffers.

In some embodiments, the preparation agent includes one or more saccharides, such as monosaccharides and disaccharides, including sucrose, maltose, trehalose, glucose, mannose, dextrose, and xylose. In some embodiments, the saccharide is a monosaccharide. In some embodiments, the saccharide is a disaccharide. In some embodiments, the saccharide is a monosaccharide, a disaccharide, or a combination thereof. In some embodiments, the saccharide is a non-reducing disaccharide. In some embodiments, the saccharide is sucrose, maltose, trehalose, glucose (e.g., dextrose), mannose, or xylose. In some embodiments, the saccharide comprises trehalose. In some embodiments, the preparation agent comprises a starch. In some embodiments, the preparation agent includes polysucrose, a polymer of sucrose and epichlorohydrin. In some embodiments, the preparation agent includes from about 10 mM to about 1,000 mM of the one or more saccharides. In some embodiments, the preparation agent includes from about 50 to about 500 mM of the one or more saccharides. In some embodiments, one or more saccharides is present in an amount of from 10 mM 10 to 500 mM. In some embodiments, one or more saccharides is present in an amount of from 50 mM to 200 mM. In some embodiments, one or more saccharides is present in an amount from 100 mM to 150 mM. In some embodiments, the one or more saccharides are the lyophilizing agent; for example, in some embodiments, the lyophilizing agent comprises trehalose, polysucrose, or a combination thereof. In some embodiments, the preparation agent comprises trehalose in the range of 0.4-35%, or 1-35%, or 2-30%, or 1-10%, or 1-5%, or 0.5-5%. In an exemplary embodiment, the composition comprises 3.5% trehalose. In some embodiments, the preparation agent comprises polysucrose in the range of 2-8%, or 2.25-7.75%, or 2.5-7.5%, or 2.5-6.5%, wherein the composition is in a rehydrated form. In an exemplary embodiment, the composition comprises 3% polysucrose. In another exemplary embodiment, the composition comprises 6% polysucrose. Different ionic forms of polysucrose can be used in the preparation agent that would be used to lyophilize the platelet derivatives. The ionic forms of polysucrose can be exploited to increase the efficiency of the lyophilization process. The ionic forms can be optimized to accommodate higher concentrations of platelet concentrations in the solution for performing lyophilization process. In some embodiments of the composition, wherein the composition comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. Polysucrose of different molecular weight can be used to increase the efficiency of the lyophilization process. In some embodiments of the composition, polysucrose has a molecular weight in the range of 70,000 MW to 400,000 MW. In some embodiments, polysucrose has a molecular weight in the range of 80,000 MW to 350,000 MW, or 100,000 MW to 300,00 MW. In some exemplary embodiments, polysucrose has a molecular weight in the range of 120,000 MW to 200,000 MW. In some exemplary embodiments, polysucrose has a molecular weight of 150,000 MW, or 160,000 MW, or 170,000 MW, or 180,000 MW, 190,000 MW, or 200,000 MW.

In some embodiments the composition comprising platelets or platelet derivatives, (e.g., thrombosomes), may comprise one or more of water or a saline solution. In some embodiments the composition comprising platelets or platelet derivatives, such as freeze-dried platelets, may comprise DMSO.

In some embodiments, the preparation agent comprises an organic solvent, such as an alcohol (e.g., ethanol). In such a preparation agent, the amount of solvent can range from 0.1% to 5.0% (v/v). In some embodiments, the organic solvent can range from about 0.1% (v/v) to about 5.0% (v/v), such as from about 0.3% (v/v) to about 3.0% (v/v), or from about 0.5% (v/v) to about 2% (v/v).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. In some embodiments the organic solvent is selected from the group consisting of ethanol, acetic acid, acetone, acetonitrile, dimethylformamide, dimethyl sulfoxide (DMSO), dioxane, methanol, n-propanol, isopropanol, tetrahydrofuran (THF), N-methyl pyrrolidone, dimethylacetamide (DMAC), or combinations thereof. In some embodiments, the organic solvent comprises ethanol, DMSO, or a combination thereof. The presence of organic solvents, such as ethanol, can be beneficial in the processing of platelets, platelet derivatives, or thrombosomes (e.g., freeze-dried platelet derivatives).

In some embodiments the preparation agent does not include an organic solvent. In some embodiments, the preparation agent comprises an organic solvent. In some embodiments the preparation agent comprises DMSO.

A preparation agent can have any appropriate pH. For example, in some embodiments, a preparation agent can have a pH of about 5.5 to about 8.0 (e.g., about 6.5 to about 6.9, or about 6.6 to about 6.8). In some embodiments, the preparation agent has a pH in the range of 5.5 to 8.0, or 6.0 to 8.0, or 6.0 to 7.5. In an exemplary embodiment, the preparation agent has a pH of 6.5. In another exemplary embodiment, the preparation agent has a pH of 7.4.

In some embodiments, one or more other components may be combined with in the platelets (e.g., as part of a preparation agent). Exemplary components may include Prostaglandin E1 or Prostacyclin and or EDTA/EGTA to prevent platelet aggregation and activation.

In some embodiments, a preparation agent can be Buffer A, as shown in Example 1 of U.S. Pat. No. 11,529,587 and Example 1 of PCT app no. PCT/US2022/079280, incorporated by reference herein in their entirety. In some embodiments, a preparation agent can comprise Buffer A, as shown in this Example 1 wherein one or more components (e.g., ethanol) is present in an amount up to three times the amount shown in this Example 1. Non-limiting examples of preparation agent compositions that may be used are shown in Tables P1-P6.

TABLE P1
Buffer

| | Concentration |
|---|---|
| Component | (mM unless otherwise specified) |
| NaCl | 75.0 |
| KCl | 4.8 |
| HEPES | 9.5 |
| NaHCCO₃ | 12.0 |
| Dextrose | 3 |
| Trehalose | 100 |
| Ethanol (optional) | 1% (v/v) |

Table P1. A preparation agent that can be used

TABLE P2
Buffer

| | Concentration |
|---|---|
| Component | (mM unless otherwise specified) |
| NaCl | 75.0 |
| KCl | 4.8 |
| HEPES | 9.5 |
| NaHCCO₃ | 12.0 |
| Dextrose | 3 |
| Trehalose | 100 |
| Ethanol (optional) | 1% (v/v) |

Table P2. A preparation agent that can be used

TABLE P3
Buffer A1 (pH 6.5)

| | Concentration |
|---|---|
| Component | (mM unless specified otherwise) |
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |

Table P3. A preparation agent that can be used.

TABLE P4
Buffer B

| | Concentration |
|---|---|
| Component | (mM unless otherwise specified) |
| Buffer and Salts | Table 4 (below) |
| BSA | 0.35% |
| Dextrose | 5 |
| pH | 7.4 |

Table P4. Buffer B can used when incubating platelets, e.g., for flow cytometry. Such an incubation can be done at room temperature in the dark. Albumin is an optional component of Buffer B.

TABLE P5
Concentration of HEPES and of Salts in Buffer B

| | Concentration |
|---|---|
| Component | (mM unless otherwise specified) |
| HEPES | 25 |
| NaCl | 119 |
| KCl | 5 |
| CaCl₂ | 2 |
| MgCl₂ | 2 |
| glucose | 6 g/l |

Table P5 shows the concentrations of HEPES and salts in Buffer B. The pH can be adjusted to 7.4 with NaOH. Albumin is an optional component of Buffer B.

TABLE P6
Tyrode's HEPES Buffer (plus PGE1)

| Component | Concentration (mM) |
|---|---|
| CaCl₂ | 1.8 |
| MgCl₂ | 1.1 |
| KCl | 2.7 |
| NaCl | 137 |
| NaH₂PO₄ | 0.4 |
| HEPES | 10 |
| D-glucose | 5.6 |
| pH | 6.5 |
| Prostagalandin E1 | 1 μg/ml |
| (PGE1) | |

Table P6 is another exemplary preparation agent.

### Rehydration of the Composition Comprising Platelet Derivatives

In some aspects, the platelet derivative composition of the present disclosure is in the form of a powder. In some aspects, the process for preparing the platelet derivative composition results in the final product which is in a dry powdered form. The platelet derivative composition in its dry form comprises platelet derivatives. The platelet derivative composition in its dry form comprises platelet derivatives, and/or freeze-dried platelets. It is well-known to a skilled artisan that the platelet derivatives in the dried form shall preserve the characteristics which it is intended to observe once the platelet derivatives are rehydrated for clinical application and/or studying the characteristics such as, the presence of platelet activation markers.

In some embodiments, rehydrating the composition comprising platelets or platelet derivatives comprises adding to the platelets an aqueous liquid. In some embodiments, the aqueous liquid is water. In some embodiments, the aqueous liquid is an aqueous solution (e.g., a buffer). In some embodiments, the aqueous liquid is a saline solution. In some embodiments, the aqueous liquid is a suspension.

In some embodiments, the platelets or platelet derivatives (e.g., thrombosomes) have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets or platelet derivatives (e.g., thrombosomes), have less than about 10%, such as less than about 8%, such as less than about 6%, such as less than about 4%, such as less than about 2%, such as less than about 0.5% crosslinking of platelet membranes via proteins and/or lipids present on the membranes. Since the process for preparing the platelet derivatives herein do not include the use of any cross-linking inducing agents, such as, non-limiting to aldehydes, typically, paraformaldehyde, the platelet derivatives herein do not have any exogenous cross-linking on the membranes. Therefore, typically, the platelet derivatives herein have no exogenous cross-linking on the membranes.

In some embodiments, rehydrating the composition comprising platelets or platelet derivatives comprises adding to the platelets or platelet derivatives sterile water (e.g., sterile water for injection) over about 10 minutes at about room temperature. In general, the rehydration volume is about equal to the volume used to fill each vial of platelet derivative composition prior to drying, for example, freeze-drying.

### Process for Preparing a Platelet Derivative Composition

In some embodiments, the platelets or pooled platelets can be initially diluted, further diluted (e.g. if initially diluted in an acidified buffer) or suspended in a preparation agent as described herein before being loaded onto a TFF unit to exchange the solution, buffer or diluted preparation agent with a preparation agent in the TFF unit. A skilled artisan would understand that before being loaded onto a TFF unit, the input composition can be initially diluted to a desirable dilution in order to carry out the TFF process in an effective manner. In some embodiments, the platelets or pooled platelets comprised in a composition can be diluted with an acidified washing buffer for example, and/or with a preparation agent as described herein before loading onto a TFF unit. In some embodiments, the platelets or pooled platelets are diluted 1:0.5, 1:1, 1:2, 1:4, 1:5, or 1:10, in a preparation agent, which in illustrative embodiments is the preparation in which the platelets will be freeze dried. In illustrative embodiments, the platelets or the pooled platelets can be diluted or suspended in a preparation agent comprising trehalose and in illustrative embodiments polysucrose before being loaded onto a TFF unit, followed by performing TFF with the preparation agent in the TFF unit. In illustrative embodiments, the platelets or the pooled platelets can be diluted or suspended in a preparation agent comprising 0.4 to 35% trehalose and 2% to 8% polysucrose, before being loaded onto a TFF unit, followed by performing TFF with the preparation agent in the TFF unit. A skilled artisan would understand that performing TFF is a continuous process of fluid exchange between the preparation agent and the platelets or the pooled platelets. The preparation agent that is used to dilute or suspend the platelets or the pooled platelets before being loaded onto a TFF unit can be the same preparation agent that is used for performing the TFF or it can be a different solution (e.g., acidified washing buffer) typically that is compatible with processing viable platelets. In some embodiments, the preparation agent used to dilute or suspend the platelets or the pooled platelets before being loaded onto a TFF unit can have the same ingredients but differ in the concentration of the ingredients than the preparation agent used for performing the TFF. A skilled artisan would understand the extent of the difference, if at all needed, based upon the dilution required to perform the TFF.

In some embodiments, the platelets or pooled platelets may be acidified to a pH of about 5.5 to about 8.0 prior to TFF or being diluted with the preparation agent. In some embodiments, the method comprises acidifying the platelets to a pH of about 6.5 to about 6.9. In some embodiments, the method comprises acidifying the platelets to a pH of about 6.6 to about 6.8. In some embodiments, the method comprises acidifying the platelets to a pH of about 6.6 to 7.5. In some embodiments, the acidifying comprises adding to the pooled platelets a solution comprising Acid Citrate Dextrose (ACD).

In some embodiments, the platelets are isolated prior to the step comprising tangential flow filtration (TFF) or being diluted with the preparation agent. In some embodiments, the method further comprises isolating platelets by using centrifugation. In some embodiments, the centrifugation occurs at a relative centrifugal force (RCF) of about 1000×g to about 2000×g. In some embodiments, the centrifugation occurs at relative centrifugal force (RCF) of about 1300×g to about 1800×g. In some embodiments, the centrifugation occurs at relative centrifugal force (RCF) of about 1500×g. In some embodiments, the centrifugation occurs for about 1 minute to about 60 minutes. In some embodiments, the centrifugation occurs for about 10 minutes to about 30 minutes. In some embodiments, the centrifugation occurs for about 30 minutes.

In some embodiments, platelets are isolated, for example in a liquid medium, prior to treating a subject.

In some embodiments, platelets are donor-derived platelets. In some embodiments, platelets are obtained by a process that comprises an apheresis step. In some embodiments, platelets are pooled platelets.

In some embodiments, platelets are pooled from a plurality of donors. Such platelets pooled from a plurality of donors may be also referred herein to as pooled platelets. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35. Pooled platelets can be used to make any of the compositions described herein. The platelets can be pooled wherein the platelets are donated by human subjects. In some other embodiments, the donor can be a non-human animal. In some embodiments, the donor can be a canine subject. In some embodiments, the donor can be an equine subject. In some embodiments, the donor can be a feline subject.

In some embodiments, platelets are derived in vitro. In some embodiments, platelets are derived or prepared in a culture. In some embodiments, preparing the platelets comprises deriving or growing the platelets from a culture of megakaryocytes. In some embodiments, preparing the platelets comprises deriving or growing the platelets (or megakaryocytes) from a culture of human pluripotent stem cells (PSCs), including embryonic stem cells (ESCs) and/or induced pluripotent stem cells (iPSCs).

Accordingly, in some embodiments, platelets or platelet derivatives (e.g., thrombosomes) are prepared prior to treating a subject as described herein. In some embodiments, the platelets or platelet derivatives (e.g., thrombosomes) are lyophilized. In some embodiments, the platelets or platelet derivatives (e.g., thrombosomes) are cryopreserved. For example, in some embodiments, the platelets or platelet derivatives can be cryopreserved in plasma and DMSO (e.g., 3-9% DMSO (e.g., 6% DMSO)). In some embodiments, the platelets or platelet derivatives are cryopreserved as described in U.S. Patent Application Publication No. 2020/0046771 A1, published on Feb. 13, 2020, incorporated herein by reference in its entirety.

In some embodiments, platelets (e.g., apheresis platelet, platelets isolated from whole blood, pooled platelets, or a combination thereof) form a suspension in a preparation agent comprising a liquid medium at a concentration from 10,000 platelets/μL to 10,000,000 platelets/μL, such as 50,000 platelets/μL to 2,000,000 platelets/μL, such as 100,000 platelets/μL to 500,000 platelets/μL, such as 150,000 platelets/μL to 300,000 platelets/μL, such as 200,000 platelets/μL.

In some embodiments, the method further comprises drying the platelets or platelet derivatives (e.g., thrombosomes). In some embodiments, the drying step comprises lyophilizing the platelets or platelet derivatives (e.g., thrombosomes). In some embodiments, the drying step comprises freeze-drying the platelets or platelet derivatives (e.g., thrombosomes). In some embodiments, the method further comprises rehydrating the platelets or platelet derivatives (e.g., thrombosomes) obtained from the drying step.

In some embodiments, the platelets or platelet derivatives (e.g., thrombosomes) are cold stored, cryopreserved, or lyophilized (e.g., to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table LA. Additional exemplary lyophilization methods can be found in U.S. Pat. Nos. 7,811,558, 8,486,617, and 8,097,403. An exemplary spray-drying method includes: combining nitrogen, as a drying gas, with a preparation agent according to the present disclosure, then introducing the mixture into GEA Mobile Minor spray dryer from GEA Processing Engineering, Inc. (Columbia MD, USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 1 0 to 35 minutes. The final step in spray drying is preferentially collecting the dried mixture. The dried composition in some embodiments is stable for at least six months at temperatures that range from −20° C. or lower to 90° C. or higher.

TABLE LA
Exemplary Lyophilization Protocol

| | Step | Temp. Set | | Type | Duration | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P | | | | | | 0 |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the platelets or platelet derivatives (e.g., thrombosomes) that are obtained as disclosed herein, such as the step of freeze-drying the platelets and/or platelet derivatives that are obtained as disclosed herein, comprises incubating the platelet and/or platelet derivatives with a lyophilizing agent (e.g., a non-reducing disaccharide). Accordingly, in some embodiments, the methods for preparing platelets and/or platelet derivatives further comprises incubating the platelets with a lyophilizing agent. In some embodiments the lyophilizing agent is a saccharide. In some embodiments the saccharide is a disaccharide, such as a non-reducing disaccharide.

In some embodiments, the platelets and/or platelet derivatives are incubated with a lyophilizing agent for a sufficient amount of time and at a suitable temperature to incubate the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrolidone (PVP), starch, and hydroxyethyl starch (HES). In some embodiments, exemplary lyophilizing agents can include a high molecular weight polymer. By “high molecular weight” it is meant a polymer having an average molecular weight of about or above 70 kDa and up to 1,000,000 kDa. Non-limiting examples are polymers of sucrose and epichlorohydrin (e.g., polysucrose). In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%. In some embodiments, polysucrose is used in the range of 2% to 8%%, or 2.25-7.75%, or 2.5-7.5%, or 2.5-6.5%. In an exemplary embodiment, the composition comprises 3% polysucrose. In another exemplary embodiment, the composition comprises 6% polysucrose. In some embodiments of the composition, wherein the composition comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 Da to 400,000 Da. In some embodiments, polysucrose has a molecular weight in the range of 80,000 Da to 350,000 Da, or 100,000 Da to 300.00 Da. In some exemplary embodiments, polysucrose has a molecular weight in the range of 120,000 Da to 200,000 Da. In some exemplary embodiments, polysucrose has a molecular weight of 150,000 Da, or 160,000 Da, or 170,000 Da, or 180,000 Da, 190,000 Da, or 200,000 Da.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In embodiments, it is present in an amount from 40 mM to 100 mM. In some embodiments, the composition comprises trehalose in the range of 0.4-35%, or 1-35%, or 2-30%, or 1-10%, or 1-5%, or 0.5-5%. In an exemplary embodiment, the composition comprises 3.5% trehalose.

In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

In some cases, preparation of thrombosomes further comprises one or more of the procedures described in U.S. Pat. No. 8,486,617 (such as, e.g., Examples 1-5) and 8,097,403 (such as, e.g., Examples 1-3), incorporated herein by reference in their entirety. In some cases, a starting material (e.g., one or more donor platelet units) are initially pooled into a common vessel. In some embodiments, a starting material can comprise one or more donor platelet units. In some embodiments, a starting material can comprise donor plasma. The starting material may or may not be acidified with an anti-coagulation buffer (i.e. ACD-A) before centrifugation. Plasma can be aspirated off of the platelet pellet after centrifugation. Cell compatible buffer containing cryoprotectants (e.g., a loading buffer, which can be similar to or the same as a preparation agent) can be added to the platelet pellet before resuspending the cells into suspension. Platelets may or may not be diluted to a pre-determined concentration (e.g., 2200 k/ul to 2800 k/ul) with buffer if desired. Platelets in buffer may be incubated between 0 minutes and 240 minutes at an incubation temperature from 18° C. to 37° C. A lyoprotectant bulking agent (e.g., polysucrose) can be added to the platelets in buffer to achieve a final bulking agent concentration from 1% to 10% w/v (with preference at 6% w/v). The centrifuged processed platelets can then be filled into vials, lyophilized and thermally treated.

### Platelet Derivatives and Microparticles

Platelet derivatives herein have been observed to have numerous surprising properties, as disclosed in further detail herein. It will be understood, as illustrated in the Examples of U.S. Pat. No. 11,529,587 and PCT app no. PCT/US2022/079280, that although platelet derivatives in some aspects and embodiments are in a solid, such as a powder form, the properties of such platelet derivatives can be identified, confirmed, and/or measured when a composition comprising such platelet derivatives is in liquid form.

A skilled artisan would be well-versed with different techniques that are available for measuring particle sizes of platelets, platelet derivatives or FDPDs, and microparticles. One such technique, in a non-limiting manner, that can be used for measuring particle sizes is flow cytometry. Flow Cytometry is a technique for quantifying characteristics of cells such as cell number, size and complexity, fluorescence, phenotype, and viability. In general, the forward scatter in a flow cytometry is located in line with the laser intercept and is typically considered a measure of the relative cell size. The side scatter is typically located perpendicular to the laser beam intercept and is used to measure the relative complexity of the cell. Commercially available sizing beads can be used to obtain the forward scatter values to calibrate the instrument in order to measure the sizes of the particles.

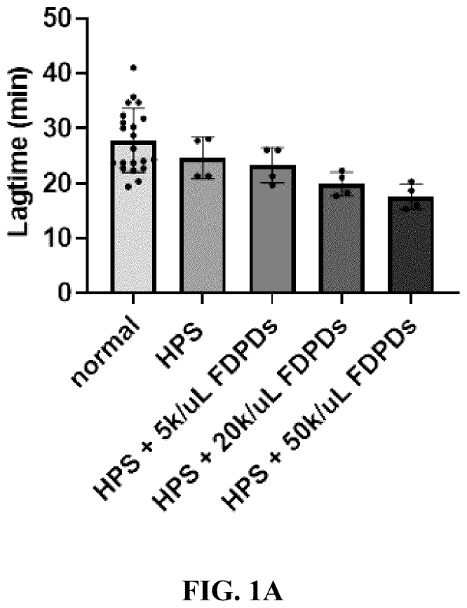

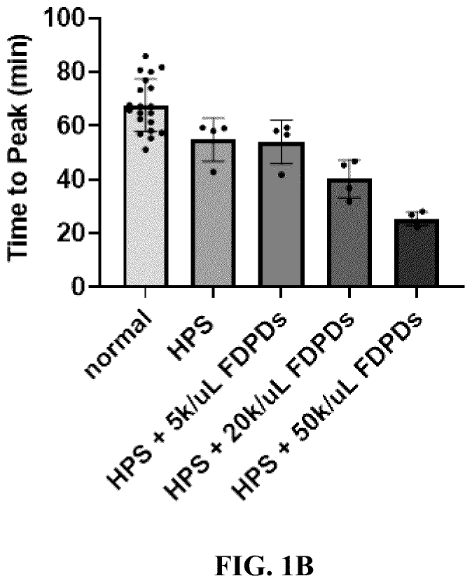

Liquid and dried compositions provided herein, in illustrative embodiments those prepared using freeze drying, and more specifically in some embodiments, prepared using methods provided herein, include particles that can be categorized broadly into populations based on at least one physical property, for example, but not limiting to, the size of the particles obtained. In some embodiments, the particles can be categorized into two populations based on size, typically in embodiments where exosomes are not present in detectable quantities, are not resolvable by the instrument analyzing particle size, and/or are not considered particles: For example, a first population comprising larger particles similar, or much more similar in size to in-dated stored platelets, which can be referred to herein as platelet derivatives, FDPDs, platelet-sized particles, a population of platelet derivatives with a size distribution centered around ˜1,000 nm radius, or ˜1,000 nm radius particles, and a second population comprising relatively smaller particles, which can be referred to herein as microparticles, a population of microparticles with a size distribution centered around ˜50 nm radius, or ˜50 nm radius particles (See e.g., FIG. 24A, FIG. 27B, or FIG. 28B, of U.S. Pat. No. 11,529,587 and of PCT app no. PCT/US2022/079280, where the main population of human in-dated stored platelets is centered at around 1,100 or 1,500 nm radius respectively, with a smaller (i.e. microparticle) peak at around 100 nm radius; and the particles in the FDPD composition (i.e. thrombosomes), which have a ˜platelet-sized major peak with a radius of approximately 1,100 nm (FIG. 24A) or 1,000 nm (FIG. 27B and FIG. 28B) (i.e. platelet derivatives) and a microparticle radius peak at approximately 50-75 nm).

A skilled artisan would further understand that the sizes determined for such populations of particles may not always be accurate enough to provide an exact cut-off value/range between these two particle size peaks. However, the difference in the sizes of the two populations can be resolved reproducibly using known methods, for example, using flow cytometry, or by using a particle/cell counter. And approximate size values or size range values can be obtained using such techniques optionally with sizing standards. In some embodiments, a composition comprising platelet derivatives or FDPDs as described herein or prepared according to methods described herein can have a population comprising platelet derivatives or FDPDs that includes between 95.1% to 99.9% of total particles in the composition, and the rest of the measurable particles, for example above 1 nm radius, can be microparticles. In some embodiments, platelet derivatives or FDPDs in such a composition can have a diameter of at least 0.4 μm (i.e., radius of at least 200 nm), and the microparticles in such a composition can have a diameter less than 0.4 μm (i.e., radius less than 200 nm). In other embodiments, platelet derivatives or FDPDs in such a composition can have a diameter of at least 0.5 μm (i.e., radius of at least 250 nm), and the microparticles in such a composition can have a diameter less than 0.5 μm (i.e., radius of less than 250 nm). In some embodiments, the platelet derivatives, or FDPDs can have a diameter of at least 0.4 μm, for example in the range of 0.5 μm to 22 μm (i.e., radius in the range of 200 nm or 250 nm to 11,000 nm), and the microparticles can have a diameter less than 0.5 μm (i.e., less than 250 nm radius), for example in the range of 0.04 μm to 0.350 μm (i.e., radius in the range of 20 nm to 175 nm). In some embodiments, the platelet derivatives, or FDPDs can have a diameter in the range of 1 μm to 18 μm (i.e., radius in the range of 500 nm to 9,000 nm), and the microparticles can have a diameter in the range of 0.06 μm to 0.2 μm (i.e., radius in the range of 30 nm to 100 nm). In some embodiments, the composition comprises platelet derivatives or FDPDs, and microparticles as the only or essentially the only particles present in the composition, optionally or typically other than exosomes, in embodiments where exosomes are not present in detectable quantities, are not resolvable by the instrument analyzing particle size, and/or are not considered particles. Of course, a composition as described herein may comprise any specific percentage number, or fraction thereof, of platelet derivatives, FDPDs or microparticles within the ranges discussed herein.

In some embodiments, the platelets or platelet derivatives (e.g., thrombosomes) have a particle size, for example a diameter, max dimension, or radius of at least about 0.5 μm (e.g., at least about at least about 0.6 μm, at least about 0.7 μm, at least about 0.8 μm, at least about 0.9 μm, at least about 1.0 μm, at least about 1.2 μm, at least about 1.5 μm, at least about 2.0 μm, at least about 2.5 μm, or at least about 5.0 μm). In some embodiments, the particle size, for example the diameter, max dimension, or radius, is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). From this disclosure, it will be apparent that microparticles typically have a size of less than 250 nm radius (i.e. less than 500 nm diameter). In some embodiments, the particle size is from about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets or platelet derivatives (e.g., thrombosomes), have a particle size of at least 0.5 μm, for example in the range of about 0.5 μm to about 25.0 μm, 20.0 μm, 15.0 μm, 12.5 μm, 10.0 μm, or 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of the platelets or platelet derivatives (e.g., thrombosomes), are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of the platelets or platelet derivatives (e.g., thrombosomes) are in the range of about 0.5 μm to about 5.0 μm (e.g., from about 0.5 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some illustrative embodiments, a microparticle can be a particle having a particle size (e.g., diameter, max dimension) of less than about 0.5 μm (less than about 0.45 μm or 0.4 μm) In some cases, a microparticle can be a particle having a particle size of about 0.01 μm to about 0.5 μm (e.g., about 0.02 μm to about 0.5 μm).

Compositions comprising platelets or platelet derivatives (e.g., thrombosomes), such as those prepared according to methods described herein, can have a microparticle content that contributes to less than about 5.0% (e.g., less than about 4.5%, 4.0%, 3.5%, 3.0%, 2.5%, 2.0%, 1.5%, 1.0%, 0.5%, or 0.1%) of the total scattering intensity of all particles from about 1 nm to about 60,000 nm in radius in the composition. In some embodiments, the platelet derivative composition comprises a population of platelet derivatives comprising CD41-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD42-positive platelet derivatives, wherein less than 15%, 10%, 7.5%, 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some embodiments, the platelet derivative composition comprises a population of platelet derivatives comprising CD61-positive platelet derivatives, wherein less than 15%, 10%, 7.5, 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm, which in certain illustrative embodiments are less than 0.5 μm. In some illustrative embodiments, the microparticles have a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. As used herein, a content of microparticles “by scattering intensity” refers to the microparticle content based on the scattering intensity of all particles from about 1 nm to about 60,000 nm in radius in the composition. The microparticle content can be measured by any appropriate method, for example, by dynamic light scattering (DLS). In some cases, the viscosity of a sample used for DLS can be at about 1.060 cP (or adjusted to be so), as this is the approximate viscosity of plasma. In some embodiments, the platelet derivative composition as per any aspects, or embodiments comprises a population of platelet derivatives, and microparticles, wherein the numerical ratio of platelet derivatives to the microparticles is at least 90:1, 91:1, 92:1, 93:1, 94:1, 95:1, 96:1, 97:1, 98:1, or 99:1. In some embodiments, the platelet derivatives have a diameter in the range of 0.5-2.5 μm, and the microparticles have a diameter less than 0.5 μm.

Platelets or platelet derivatives (e.g., thrombosomes) as described herein can have cell surface markers. The presence of cell surface markers can be determined using any appropriate method. In some embodiments, the presence of cell surface markers can be determined using binding proteins (e.g., antibodies) specific for one or more cell surface markers and flow cytometry (e.g., as a percent positivity, e.g., using approximately 2.7×10⁵ thrombosomes/μL; and about 4.8 μL of an anti-CD41 antibody, about 3.3 μL of an anti-CD42 antibody, about 1.3 μL of annexin V, or about 2.4 μL of an anti-CD62 antibody). Non-limiting examples of cell-surface markers include CD41 (also called glycoprotein IIb or GPIIb, which can be assayed using e.g., an anti-CD41 antibody), CD42 (which can be assayed using, e.g., an anti-CD42 antibody), CD62 (also called CD62P or P-selectin, which can be assayed using, e.g., an anti-CD62 antibody), phosphatidylserine (which can be assayed using, e.g., annexin V (AV)), and CD47 (which is used in self-recognition; absence of this marker, in some cases, can lead to phagocytosis). The percent positivity of any cell surface marker can be any appropriate percent positivity. For example, platelets or platelet derivatives (e.g., thrombosomes), such as those prepared by methods described herein, can have an average CD41 percent positivity of at least 55% (e.g., at least 60%, at least 65%, at least 67%, at least 70%, at least 75%, at least 80%, at least 85%, at least 90%, or at least 95%). In some embodiments, platelets or platelet derivatives herein can have an average CD41 percent positivity in the range of 70%-99%, 70%-95%, 70%-90%, 70%-86%, or 75%-86%. In some embodiments, at least 90%, 91%, 92%, 93%, 94%, 95%, 96%, 97%, 98%, 99% platelet derivatives that are positive for CD41 have a size in the range of 0.5-2.5 μm. In some embodiments, at least 90%, 91%, 92%, 93%, 94%, 95%, 96%, 97%, 98%, or 99% platelet derivatives that are positive for CD 41 have a size in the range of 0.4-2.8 μm. In some embodiments, at least 90%, 91%, 92%, 93%, 94%, 95%, 96%, 97%, 98%, or 99% platelet derivatives that are positive for CD 41 have a size in the range of 0.3-3 μm.

As another example, platelets or platelet derivatives (e.g., thrombosomes), such as those described herein, can have an average CD42 percent positivity of at least 65% (e.g., at least 67%, at least 70%, at least 75%, at least 80%, at least 85%, at least 90%, or at least 95%). In some embodiments, platelets or platelet derivatives can have an average CD42 percent positivity of at least 76%, 77%, 78%, or 79%. In some embodiments, platelets or platelet derivatives can have an average CD42 percent positivity in the range of 76-95%, 76-94%, 77-93%, or 78-90%. In some embodiments, at least 90%, 91%, 92%, 93%, 94%, 95%, 96%, 97%, 98%, or 99% platelet derivatives that are positive for CD 42 have a size in the range of 0.5-2.5 μm. In some embodiments, at least 90%, 91%, 92%, 93%, 94%, 95%, 96%, 97%, 98%, or 99% platelet derivatives that are positive for CD 42 have a size in the range of 0.4-2.8 μm. In some embodiments, at least 90%, 91%, 92%, 93%, 94%, 95%, 96%, 97%, 98%, or 99% platelet derivatives that are positive for CD 42 have a size in the range of 0.3-3 μm.

As another example, platelets or platelet derivatives (e.g., thrombosomes), such as those prepared by methods described herein, can have an average CD62 percent positivity of at least 10% (e.g., at least 15%, at least 20%, at least 25%, at least 30%, at least 35%, at least 40%, at least 45%, at least 50%, at least 55%, at least 60%, at least 65%, at least 70%, at least 75%, at least 80%, at least 82%, at least 83%, at least 84%, at least 85%, at least 90%, or at least 95%). In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.5-2.5 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.4-2.8 μm. In some embodiments, at least 70%, 75%, 80%, 85%, 90%, 91%, 92%, 93%, 94%, 95%, 96%, 97%, 98%, or 99% platelet derivatives that are positive for CD 62 have a size in the range of 0.3-3 μm.

As yet another example, platelets or platelet derivatives (e.g., thrombosomes), such as those prepared by methods described herein, can have an average annexin V positivity of at least 25% (e.g., at least 30%, at least 35%, at least 40%, at least 45%, at least 50%, at least 55%, at least 60%, at least 65%, at least 70%, at least 75%, at least 80%, at least 85%, at least 90%, at least 95%, at least 97%, or at least 99%). In some embodiments, at least 90%, 91%, 92%, 93%, 94%, 95%, 96%, 97%, 98%, 99% platelet derivatives that are positive for annexin V have a size in the range of 0.5-2.5 μm. In some embodiments, at least 90%, 91%, 92%, 93%, 94%, 95%, 96%, 97%, 98%, or 99% platelet derivatives that are positive for annexin V have a size in the range of 0.4-2.8 μm. In some embodiments, at least 90%, 91%, 92%, 93%, 94%, 95%, 96%, 97%, 98%, or 99% platelet derivatives that are positive for annexin V have a size in the range of 0.3-3 μm. In some embodiments, the presence of phosphatidyl serine in/on the platelet derivatives herein is higher than the presence of phosphatidyl serine in/on the platelets, such as, fresh platelets or apheresis platelets. For example, platelet derivatives herein exhibit at least 5 fold, 10 fold, 20 fold, 25 fold, 30 fold, 40 fold, or 50 fold higher presence of phosphatidyl serine as compared to the platelets.

In some embodiments, the platelet derivatives as described herein are activated to a maximum extent such that in the presence of an agonist, the platelet derivatives are not able to show an increase in the platelet activation markers on them as compared to the level of the platelet activation markers which were present prior to the exposure with the agonist. In some embodiments, the platelet derivatives as described herein show an inability to increase expression of a platelet activation marker in the presence of an agonist as compared to the expression of the platelet activation marker in the absence of an agonist. In some embodiments, the agonist is selected from the group consisting of collagen, epinephrine, ristocetin, arachidonic acid, adenosine di-phosphate, and thrombin receptor associated protein (TRAP). In some embodiments, the platelet activation marker is selected from the group consisting of Annexin V, and CD 62. In some embodiments, the platelet derivatives as described herein show an inability to increase expression of Annexin V in the presence of TRAP. An increased amount of the platelet activation markers on the platelets indicates the state of activeness of the platelets. However, in some embodiments, the platelet derivatives as described herein are not able to increase the amount of the platelet activation markers on them even in the presence of an agonist. This property indicates that the platelet derivatives as described herein are activated to a maximum extent. In some embodiments, the property can be beneficial where maximum activation of platelets is required, because the platelet derivatives as described herein is able to show a state of maximum activation in the absence of an agonist.

As another example, platelets or platelet derivatives (e.g., thrombosomes), such as those prepared by methods described herein, can have an average CD47 percent positivity of at least about 8% (e.g., at least about 10%, 15%, 20%, 25%, 30%, 35%, 40%, 45%, 50%, or 55%).

Glycoprotein VI (GPVI) is a platelet receptor for collagen, and the binding of collagen to GVPI activates the platelet. Receptor binding can be noticeably reduced in thrombosomes compared to fresh platelets. Without being bound by any particular theory, it is believed that the manufacturing process is blocking or destroying some copies of this receptor in thrombosomes, possibly to a reduction in collagen binding in thrombosomes relative to fresh platelets.

Platelets or platelet derivatives (e.g., thrombosomes) as described herein can have fibrinogen associated with the cell membrane. Aggregation of activated platelets is mediated by the formation of the GPIIb/IIIa complex, which can bind to fibrinogen (also called Factor 1) and form a clot. GPIIb/IIIa is a platelet fibrinogen receptor also known as CD41/CD61 complex. The GPIIb/IIIa clone PAC-1 binds to the active form of the GPIIb/IIIa. Without being bound by any particular theory, it is believed that the presence of fibrinogen on the cell membrane may be indicative of platelets or platelet derivatives (e.g., thrombosomes) capable of forming clots. Similarly, without being bound by any particular theory, it is believed that a lack of binding by anti-PAC1 antibodies to the platelets or platelet derivatives (e.g., thrombosomes), such as those prepared by methods described herein, can be indicative of fibrinogen bound to the active form of GPIIb/GPIIIa, as PAC-1 binds to the same complex. In some cases, platelets or platelets derivatives (e.g., thrombosomes), such as those prepared by methods described herein, can have a greater amount of bound fibrinogen when they retain a higher amount of residual plasma. In some embodiments of a platelet derivative composition as described herein, the platelet derivatives can have an amount of fibrinogen on their surface that is greater than that present on the surface of resting platelets, activated platelets, or lyophilized fixed platelets. In some embodiments, the platelet derivatives have at least 25%, 30%, 35%, 40%, 45%, 50%, 55%, 60%, 65%, 70%, 75%, 80%, 85%, 90%, 95%, or 100% higher fibrinogen on their surface as compared to resting platelets, or activated platelets, or fixed platelets. In some embodiments, the platelet derivatives when analyzed for the binding of anti-fibrinogen antibody to the platelet derivatives using flow cytometry exhibit at least 2 folds higher mean fluorescent intensity (MFI) in the absence of an agonist as compared to the MFI of binding of anti-fibrinogen antibody to the lyophilized fixed platelets. In some embodiments, the platelet derivatives when analyzed for the binding of anti-fibrinogen antibody to the platelet derivatives using flow cytometry exhibit at least 10, 15, 20, 25, 30, 35, or 40 folds higher mean fluorescent intensity (MFI) in the absence of an agonist as compared to the MFI of binding of anti-fibrinogen antibody to the fixed platelets. In some embodiments, the greater amount of fibrinogen present on the surface of the platelet derivatives as described herein as compared to that of lyophilized fixed platelets is beneficial. Without being bound by any particular theory, it is believed that the higher amount of fibrinogen on the cell membrane of the platelet derivatives (e.g., thrombosomes) as compared to that of lyophilized fixed platelets can make the platelet derivatives superior in terms of its ability to form clots as compared to lyophilized fixed platelets.

Von Willebrand factor (vWF) is a multimeric glycoprotein that plays a major role in blood coagulation. vWF serves as a bridging molecule that promotes platelet binding to sub-endothelium and other platelets, thereby promoting platelet adherence and aggregation. vWF also binds to collagens to facilitate clot formation at sites of injury. In some embodiments, the platelet derivatives as described herein have the presence of von Willebrand factor (vWF) on their surface at a level that is greater than that on the surface of resting platelets, activated platelets, or lyophilized fixed platelets. In some embodiments, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

Thrombospondin is a glycoprotein secreted from the α-granules of platelets upon activation. In the presence of divalent cations, the secreted protein binds to the surface of the activated platelets and is responsible for the endogenous lectin-like activity associated with activated platelets. In some embodiments, the platelet derivatives have the presence of thrombospondin (TSP-1) on their surface at a level that is greater than that presence on the surface of resting platelets, activated platelets, or lyophilized fixed platelets. In some embodiments, the platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

Platelet derivatives (e.g., FDPDs) that are included in methods, collections, and compositions herein, a) have the ability to generate thrombin in vitro, in illustrative embodiments, in the presence of tissue factor and phospholipids; b) have the ability to occlude a collagen-coated microchannel in vitro; or c) both a) and b). Thus, in some embodiments platelet derivatives (e.g., FDPDs) used in methods, collections, and compositions herein can be capable of generating thrombin, for example, when in the presence of a reagent containing tissue factor and phospholipids in vitro. For example, in some cases, platelets or platelet derivatives (e.g., thrombosomes) (e.g., at a concentration of about 4.8×10³ particles/μL) as described herein can generate a thrombin peak height (TPH) of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of a reagent containing tissue factor (e.g., at 0.25 pM, 0.5 pM, 1 pM, 2 pM, 5 pM or 10 pM) and optionally phospholipids. For example, in some cases, platelets or platelet derivatives (e.g., thrombosomes) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of a reagent containing tissue factor and (e.g., at 0.25 pM, 0.5 pM, 1 pM, 2 pM, 5 pM or 10 pM) and optionally phospholipids. In some cases, platelets or platelet derivatives (e.g., thrombosomes) (e.g., at a concentration of about 4.8×10³ particles/μL) as described herein can generate a TPH of at least 25 nM (e.g., at least 30 nM, 35 nM, 40 nM, 45 nM, 50 nM, 52 nM, 54 nM, 55 nM, 56 nM, 58 nM, 60 nM, 65 nM, 70 nM, 75 nM, or 80 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., thrombosomes). In some cases, platelets or platelet derivatives (e.g., thrombosomes) (e.g., at a concentration of about 4.8×10³ particles/μL) as described herein can generate a TPH of about 25 nM to about 100 nM (e.g., about 25 nM to about 50 nM, about 25 to about 75 nM, about 50 to about 100 nM, about 75 to about 100 nM, about 35 nM to about 95 nM, about 45 to about 85 nM, about 55 to about 75 nM, or about 60 to about 70 nM) when in the presence of PRP Reagent (cat #TS30.00 from Thrombinoscope), for example, using conditions comprising 20 μL of PRP Reagent and 80 μL of a composition comprising about 4.8×10³ particles/μL of platelets or platelet derivatives (e.g., thrombosomes).

Platelet derivatives (e.g., FDPDs) that are included in methods, collections, and compositions herein, a) have the ability to generate thrombin in vitro in the presence of tissue factor and phospholipids; b) have the ability to occlude a collagen-coated microchannel in vitro; or c) both a) and b). In some embodiments, platelet derivatives (e.g., thrombosomes) can have a potency of at least 1.2 (e.g., at least 1.3, 1.4, 1.5, 1.6, 1.7, 1.8, 1.9, 2.0, 2.1, 2.2, 2.3, 2.4, or 2.5) thrombin generation potency units (TGPU) per 10⁶ particles. For example, in some cases, platelets or platelet derivatives (e.g., thrombosomes) can have a potency of between 1.2 and 2.5 TGPU per 10⁶ particles (e.g., between 1.2 and 2.0, between 1.3 and 1.5, between 1.5 and 2.25, between 1.5 and 2.0, between 1.5 and 1.75, between 1.75 and 2.5, between 2.0 and 2.5, or between 2.25 and 2.5 TGPU per 10⁶ particles). TGPU can be calculated as follows: TGPU/million particles=[TPH in nM]*[Potency Coefficient in IU/(nM)]/[0.576 million particles in the well]. Similarly, the Potency Coefficient for a sample of thrombin can be calculated as follows: Potency Coefficient=Calculated Calibrator Activity (IU)/Effective Calibrator Activity (nM). In some cases, the calibrator activity can be based on a WHO international thrombin standard.

In some embodiments, platelet derivatives (e.g., FDPDs) as described herein can be capable of occluding a collagen-coated microchannel in vitro. For example, such occluding can be determined, for example, by using a total thrombus-formation analysis system (T-TAS®). In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in a T-TAS occlusion time (e.g., time to reach kPa of 80) of less than 30, 25, 20, 15, or 14 minutes, or between 5 on the low end of the range, and 15, 20, or 25 on the high end, or between 10 on the low end of the range, and 15, 20, or 25 on the high end, or between 15 on the low end of the range and 20 or 25 on the high end, for example, in platelet-reduced citrated whole blood. In some cases, platelets or platelet derivatives as described herein, when at a concentration of at least 70×10³ particles/μL (e.g., at least 73×10³, 100×10³, 150×10³, 173×10³, 200×10³, 250×10³, or 255×10³ particles/μL) can result in an area under the curve (AUC) of at least 1300 (e.g., at least 1380, 1400, 1500, 1600, or 1700), for example, in platelet-reduced citrated whole blood. Microchannels or capillaries having different dimensions can be used in a T-TAS system for determining the occlusion times of FDPDs under different experimental conditions as provided by numerous commercial suppliers (See e.g., Zacros, Tokyo, JP). For example, a T-TAS PL chip, AR chip, or HD chip can be used for an occlusion (e.g., T-TAS) assay, as are commercially available. For example, the PL chip can have capillary dimensions of 40 μm×40 μm; or an AR chip can have capillary dimensions of 0.3 mm×80 μm; or an HD chip can have capillary dimensions of 0.3 mm×50 μm. Therefore, it is envisioned that a T-TAS assay can be performed to test the ability to occlude a collagen-coated microchannel, utilizing a microchannel or capillary with dimensions in the range of 0.02-0.5, 0.1-0.5, 0.2-0.4, 0.1-0.3, or 0.2-0.3 mm×25-200, 25-100, 50-100, 40-90, 40-80, or 50-80 μm.

Platelets or platelet derivatives (e.g., thrombosomes) as described herein can be capable of thrombin-induced trapping in the presence of thrombin. In some cases, platelets or platelet derivatives (e.g., thrombosomes) as described herein can have a percent thrombin-induced trapping of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of thrombin. In some cases, platelets or platelet derivatives (e.g., thrombosomes) as described herein can have a percent thrombin-induced trapping of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of thrombin. Thrombin-induced trapping can be determined by any appropriate method, for example, light transmission aggregometry. Without being bound by any particular theory, it is believed that the thrombin-induced trapping is a result of the interaction of fibrinogen present on the surface of the platelet derivatives with thrombin.

Platelets or platelet derivatives (e.g., thrombosomes) as described herein can be capable of co-aggregating, for example, in the presence of an aggregation agonist, and fresh platelets. Non-limiting examples of aggregation agonists include, collagen, epinephrine, ristocetin, arachidonic acid, adenosine di-phosphate, and thrombin receptor associated protein (TRAP). In some cases, platelets or platelet derivatives (e.g., thrombosomes) as described herein can have a percent co-aggregation of at least 5% (e.g., at least 10%, 15%, 20%, 25%, 30%, 35%, 40%, 45%, 50%, 55%, 60%, 65%, 67%, 70%, 75%, 85%, 90%, or 99%) in the presence of an aggregation agonist, and fresh platelets. In some cases, platelets or platelet derivatives (e.g., thrombosomes) as described herein can have a percent co-aggregation of about 25% to about 100% (e.g., about 25% to about 50%, about 25% to about 75%, about 50% to about 100%, about 75% to about 100%, about 40% to about 95%, about 55% to about 80%, or about 65% to about 75%) in the presence of an aggregation agonist. Percent co-aggregation can be determined by any appropriate method, for example, light transmission aggregometry.

Platelet derivative compositions in certain illustrative embodiments herein, comprise a population of platelet derivatives having a reduced propensity to aggregate under aggregation conditions comprising an agonist but no fresh platelets, and in illustrative embodiments in the absence of divalent cations, compared to the propensity of fresh platelets and/or activated to aggregate under these conditions. Platelets or platelet derivatives (e.g., thrombosomes) as described herein in illustrative embodiments, display a reduced propensity to aggregate under aggregation conditions comprising an agonist but no fresh platelets and no divalent cations, compared to the propensity of fresh platelets and/or activated to aggregate under these conditions. It is noteworthy that aggregation of platelet derivatives is different from co-aggregation in that aggregation conditions typically do not include fresh platelets, whereas co-aggregation conditions include fresh platelets. Exemplary aggregation and co-aggregation conditions are provided in the Examples of U.S. Pat. No. 11,529,587 and of PCT App No. PCT/US2022/079280. Thus, in some embodiments, the platelet derivatives as described herein have a higher propensity to co-aggregate in the presence of fresh platelets and presence of an agonist, while having a reduced propensity to aggregate in the absence of fresh platelets, in the absence of divalent cations, and in the presence of an agonist, compared to the propensity of fresh platelets to aggregate under these conditions. In some embodiments, a platelet derivative composition comprises a population of platelet derivatives having a reduced propensity to aggregate, wherein no more than 1%, 2%, 3%, 4%, 5%, 7.5%, 10%, 12.5%, 15%, 17.5%, 20%, 22.5%, or 25% of the platelet derivatives in the population aggregate under aggregation conditions comprising an agonist but no divalent cations, no platelets, in illustrative embodiments no fresh platelets. In some embodiments, the population of platelet derivatives aggregate in the range of 0-1%, 0-2%, 0-3%, 0-4%, 0-5%, 0-7.5%, 0-10%, 2-30%, 5-25%, 10-30%, 10-25%, or 12.5-25%, and in illustrative embodiments 0-1% or 0 to about 1%, of the platelet derivatives under aggregation conditions comprising an agonist but no platelets, in illustrative embodiments no fresh platelets. In these and other illustrative embodiments the agonist is other than arachidonic acid. It will be understood that if an aggregation reaction control sample produces a background aggregation above 0% then aggregation values/ranges for FDPDs disclosed herein would be increased by the background value obtained with the control sample. Thus, if a background aggregation produced using a control sample was 1%, then the above ranges would be increased by 1% (e.g., 1-2%, 1-3%, 1-4%, 1-5%, 1-6%, 1-8.5%, and 1-11% etc.). Accordingly, in some embodiments, the values and ranges provided herein for aggregation are values above background values, for example obtained using a control sample or no sample, and thus can be referred to control-corrected, or control-adjusted aggregation values. In some embodiments, a platelet derivative composition comprises a population of platelet derivatives having a reduced propensity to aggregate, such that less than 1/5, 1/10, or 1/20 of the platelet derivatives in the population aggregate under aggregation conditions comprising an agonist but no platelets, compared to platelet rich plasma, in illustrative embodiments prepared from fresh platelets.

Platelet derivatives (e.g., thrombosomes) herein, in some embodiments, are dry platelet derivatives, or dry platelet derived particles. In some embodiments, such dry platelet derivatives are freeze-dried (i.e., lyophilized) platelets or platelet derivatives. One such non-limiting example of dry platelet derivatives are thrombosomes. Dry platelet derivatives are typically in the form of a platelet derivative powder. The dry platelet derivative powder when rehydrated typically forma a rehydrated platelet derivative composition comprising particles. In some embodiments, compositions comprising a population of platelet derivatives, dry platelet derivatives, platelet derivative powder, or rehydrated platelet derivatives can be characterized by the presence of CD41 on or in at least 55%, 60%, 65% or higher platelet derivatives in the population. In some embodiments, compositions comprising a population of platelet derivatives, dry platelet derivatives, platelet derivative powder, or rehydrated platelet derivatives can be characterized by the presence of CD42 on or in at least 55%, 60%, 65% or higher platelet derivatives in the population. In some embodiments, dry platelet derivative particles herein can have at least one property selected from: (a) high expression of P-selectin (CD62P), for example, at least 2 fold higher than platelets, for example, apheresis platelets, or at least 10%, 20%, 30%, 40%, 50%, 60%, 70%, or higher platelet derivative particles are positive for CD62; (b) high expression of phosphatidyl serine (PS), for example, at least 2, 3, 4, 5, 6, 7, 8, 9, 10 fold or higher than the expression on platelets, for example, apheresis platelets, or at least 25%, 30%, 40%, 50%, 60%, 70%, or higher platelet derivative particles are positive for phosphatidyl serine; (c) high expression of von Willebrand Factor (vWF), for example, at least 2, 3, 4, 5, 6, 7, 8, 9, 10 fold or higher than the expression on platelets, for example, apheresis platelets; (d) high expression of fibrinogen, for example, at least 2, 3, 4, 5, 6, 7, 8, 9, 10 fold or higher than the expression on platelets, for example, apheresis platelets; (e) high expression of thrombospondin (TSP), for example, at least 2, 3, 4, 5, 6, 7, 8, 9, 10 fold or higher than the expression on platelets, for example, apheresis platelets; (f) high expression of CD41, for example, at least 55%, 60%, 65%, 70%, 75%, 80%, 85%, 90%, or higher platelet derivative particles are positive for CD41; or (g) high expression of CD42, for example, at least 65%, 70%, 75%, 80%, 85%, 90%, 95%, or higher platelet derivative particles are positive for CD42. In some embodiments, the platelet derivative particles can have two or more properties as described herein, in illustrative embodiments, the platelet derivative particles can have all the properties as described herein.

Platelet derivatives herein, in some embodiments, are capable of generating more thrombin as compared to thrombin generated by platelets, such as fresh platelets, or apheresis platelets. For example, platelet derivatives can generate at least 2 fold, 3 fold, or more thrombin in an in vitro thrombin formation assay as compared to thrombin generated by apheresis platelets. In some embodiments, platelet derivatives can generate a maximum amount of thrombin in less time as compared to platelets, such as fresh platelets or apheresis platelets. For example, platelet derivatives herein can generate a maximum amount of thrombin in at least 10%, 20%, 30%, 40%, 50%, or 60% less time than that taken by apheresis platelets.

Compositions comprising platelets or platelet derivatives (e.g., thrombosomes) as described herein can have appropriate conditions and amounts of cellular substrates and/or metabolites, such as pH, pCO₂, PO₂, HCO₃ concentration, total carbon dioxide (TCO₂), SO₂, and lactate concentration. Lactate can be the products of glycolysis. Without being bound by any particular theory, a starting material can have high lactate concentration because it has been stored ex vivo, respirating and performing glycolysis, for a period of time (e.g., about 3 days) by the time of manufacturing. For example, in some cases, the pH can be about 5.5 to about 8.0 (e.g., about 6.0 to about 7.4, about 6.9 to about 7.5, or about 7.0 to about 7.3). As another example, the pCO₂ can be about 10 to about 20 mmHg (e.g., about 10 to about 15 mmHg, about 15 to about 20 mmHg, or about 17 to about 19 mmHg). The PO₂ can be about 140 to about 165 mmHg (e.g., about 140 to about 150 mmHg, about 150 to about 160 mmgH, or about 160 to about 165 mmHg). The HCO₃ concentration can be about 4.5 to about 6.5 mmol/L (e.g., about 5.0 to about 6.0 mmol/L). The total carbon dioxide can be about 4 to about 8 mmol/L (e.g., about 5 to about 7 mmol/L). The SO₂ can be at least about 98% (e.g., at least about 99%). The lactate concentration can be less than about 2.0 mmol/L (e.g., less than 1.5 mmol/L or 1.0 mmol/L). The lactate concentration can be about 0.4 to about 1.3 mmol/L (e.g., about 0.5 to about 0.6 mmol/L, about 0.5 to about 1.0 mmol/L, or about 0.8 to about 1.3 mmol/L).

### Membrane Integrity of the Platelet Derivatives

Platelet derivatives in certain illustrative aspects and embodiments herein are surrounded by a compromised plasma membrane. In these illustrative aspects and embodiments, the platelet derivatives lack an integrated membrane around them. Instead, the membrane has pores on them that are larger than pores observed on living cells. Not to be limited by theory, it is believed that in embodiments where platelet derivatives have a compromised membrane, such platelet derivatives have a reduced ability to, or are unable to transduce signals from the external environment into a response inside the particle that are typically transduced in living platelets. A compromised membrane can be identified through a platelet derivative's inability to retain more than 50% of lactate dehydrogenase (LDH) as compared to fresh platelets, or cold stored platelets, or cryopreserved platelets. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives exhibit an increased permeability to antibodies. In some embodiments, the antibodies can be IgG antibodies. The increased permeability can be identified by targeting IgG antibodies against a stable intracellular antigen. One non-limiting type of stable intracellular antigen is β tubulin. The compromised membrane of the platelet derivatives can also be determined by flow cytometry studies.

Platelet or platelet derivatives (e.g., thrombosomes) as described herein can retain some metabolic activity, for example, as evidenced by lactate dehydrogenase (LDH) activity. In some cases, platelets or platelet derivatives (e.g., thrombosomes) as described herein can retain at least about 10% (e.g., at least about 12%, 15%, 20%, 25%, 30%, 35%, 40%, or 45%) of the LDH activity of donor apheresis platelets. Without being bound by any particular theory, it is believed that the addition of increasing amounts of polysucrose increases the amount of LDH activity remained (e.g., products of a preparation agent with 8% polysucrose have more retained LDH activity than products of a preparation agent with 4% polysucrose). Similarly unbound by any particular theory, it is believed that thermal treatment of a lyophilized composition comprising platelets or platelet derivatives (e.g., thrombosomes) increases the amount of LDH activity retained. As another example, metabolic activity can be evidenced by retained esterase activity, such as the ability of the cells to cleave the acetate groups on carboxyfluorescein diacetate succinimidyl ester (CFDASE) to unmask a fluorophore.

### Pathogen Reduction

The reduction of pathogens is generally desirable in blood products. Without being bound by any particular theory, it is believed that some methods of pathogen reduction can cause the formation of microparticles in the treated blood product. One method of pathogen reduction involves the use of a photosensitive nucleic acid-intercalating compound to alter the nucleic acids of pathogens upon illumination with an appropriate wavelength. The INTERCEPT® system (made by Cerus) uses amotosalen, a nucleic acid intercalating compound that forms cross-links in nucleic acid upon illumination with UVA.

### Starting Material Comprising Platelets or a Platelet Composition

A final blood product (e.g., platelets, cryopreserved platelets, freeze-dried platelets (e.g., thrombosomes)) as described herein can be prepared by any appropriate method. A final blood product (e.g., platelets, cryopreserved platelets, freeze-dried platelets (e.g., thrombosomes)) as described herein can be prepared by a method as disclosed herein. In some embodiments described herein, a final blood product can be a composition that includes platelets and an aqueous medium. In some embodiments, a final blood product can be the result of freeze-drying a composition that includes platelets and an aqueous medium, as described herein. In some embodiments, a final blood product can be prepared using tangential flow filtration (TFF) of a starting material (e.g., an unprocessed blood product (e.g., donor apheresis material (e.g., pooled donor apheresis material)), or a partially processed blood product (e.g., a blood product that has undergone filtration)). In some embodiments, a final blood product can be prepared using centrifugation of a starting material (e.g., an unprocessed blood product (e.g., donor apheresis material (e.g., pooled donor apheresis material)), or a partially processed blood product (e.g., a blood product that has undergone filtration)). It will be appreciated that while the methods described herein are generally described in the context of a starting material being apheresis material, other materials, such as platelets cultured in vitro, or whole blood, may be used. In some cases, platelets may be isolated from whole blood (e.g. pooled whole blood).

A starting material can be any appropriate starting material. In some embodiments, a starting material can have a protein concentration of about 60 to about 80 mg/mL. In some embodiments, a protein concentration can be based on the protein concentration in the plasma of whole blood. In some embodiments, a protein concentration can be based on the protein concentration of donor apheresis plasma. In some embodiments, a starting material can be donor blood product (e.g., whole blood or fractionated blood). In some embodiments, the starting material can be pooled donor blood product (e.g., pooled whole blood or pooled fractionated blood). In some embodiments, a starting material can include donor apheresis plasma. In some embodiments, a starting material can be derived from donor apheresis plasma. As used herein, “donor apheresis plasma” can refer to the plasma component of apheresis material, whether or not the material contains platelets or other blood cells.

In some embodiments, a starting material can be donor apheresis material (e.g., donor platelets or a pool of donor platelets). In some embodiments, a starting material is positive for one or more of: HLA Class I antibodies, HLA Class II antibodies, and HNA antibodies based on a regulatory agency-approved assay (e.g., an FDA cleared assay). In some embodiments, starting material can test positive for HLA Class I antibodies in a regulatory agency approved assay (e.g., an FDA cleared assay). In some embodiments, a starting material can test positive for HLA Class II antibodies in a regulatory agency approved assay (e.g., an FDA cleared assay). In some embodiments, starting material can test positive for HNA antibodies in a regulatory agency approved assay (e.g., an FDA cleared assay). A regulatory agency approved assay can be any appropriate regulatory agency approved assay. In some embodiments, a regulatory agency approved test can be the LABSCREEN™ Mixed by One Lambda. In some implementations, a regulatory agency approved test can be carried out using a LUMINEX® 100/200 or a LUMINEX® XY and the HLA FUSION™ software.

In some embodiments, a starting material can undergo a pathogen reduction step, for example, a nucleic acid intercalating compound that forms cross-links in nucleic acid upon illumination with UVA.

In some embodiments, a starting material (e.g., one or more units of donor platelets) can be initially pooled into a common vessel. The starting material may or may not be initially diluted with an acidified washing buffer (e.g., a control buffer). Without being bound by any particular theory, it is believed that washing with an acidified washing buffer can reduce platelet activation during processing. In some cases, a starting material can undergo two general processing pathways; either washed with control buffer (e.g. using TFF) until a desired residual component is reached (e.g., a percentage of residual donor plasma) before being concentrated to a final concentration; or the starting material can be concentrated to a final concentration before being washed with control buffer (e.g., using TFF) until a desired residual component is reached (e.g., a percentage of residual donor plasma). TFF processed material can then be filled into vials, lyophilized and thermally treated.

### Different Steps in Processing of a Starting Material, and TFF

In some embodiments, the method can include an initial dilution step, for example, a starting material (e.g., an unprocessed blood product (e.g., donor apheresis material (e.g., pooled donor apheresis material)) can be diluted with a preparation agent (e.g., any of the preparation agents described herein) to form a diluted starting material. In some cases, the initial dilution step can include dilution with a preparation agent with a mass of preparation agent equal to at least about 10% of the mass of the starting material (e.g., at least about 15%, 25%, 50%, 75%, 100%, 150%, or 200% of the mass of the starting material. In some embodiments, an initial dilution step can be carried out using the TFF apparatus.

In some embodiments, the method can include concentrating (e.g., concentrating platelets) (e.g., concentrating a starting material or a diluted starting material) to form a concentrated platelet composition. For example, concentrated can include concentrating to a about 1000×10³ to about 4000×10³ platelets/μL (e.g., about 1000×10³ to about 2000×10³, about 2000×10³ to about 3000×10³, or about 4000×10³ platelets/μL). In some embodiments, a concentration step can be carried out using the TFF apparatus.

The concentration of platelets or platelet derivatives (e.g., thrombosomes) can be determined by any appropriate method. For example, a counter can be used to quantitate concentration of blood cells in suspension using impedance (e.g., a Beckman Coulter AcT 10 or an AcT diff 2).

In some embodiments, TFF can include diafiltering (sometimes called “washing”) of a starting material, a diluted starting material, a concentrated platelet composition, or a combination thereof. In some embodiments, diafiltering can include washing with at least 2 (e.g., at least 3, 4, 5, 6, 7, 8, 9, 10, or more) diavolumes. In some embodiments, TFF can include buffer exchange. In some embodiments, a buffer can be used in TFF. A buffer can be any appropriate buffer. In some embodiments, the buffer can be a preparation agent (e.g., any of the preparation agents described herein). In some embodiments, the buffer can be the same preparation agent as was used for dilution. In some embodiments, the buffer can be a different preparation than was used for dilution. In some embodiments, a buffer can include a lyophilizing agent, including a buffering agent, a base, a loading agent, optionally a salt, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof. A buffering agent can be any appropriate buffering agent. In some embodiments, a buffering agent can be HEPES ((4-(2-hydroxyethyl)-1-piperazineethanesulfonic acid). A base can be any appropriate base. In some embodiments, a base can be sodium bicarbonate. In some embodiments, a saccharide can be a monosaccharide. In some embodiments, a loading agent can be a saccharide. In some embodiments, a saccharide can include sucrose, maltose, trehalose, glucose (e.g., dextrose), mannose, or xylose. In some embodiments, a monosaccharide can be trehalose. In some embodiments, the loading agent can include polysucrose. A salt can be any appropriate salt. In some embodiments, a salt can be selected from the group consisting of sodium chloride (NaCl), potassium chloride (KCl), or a combination thereof.

In some embodiments, a membrane with a pore size of about 0.1 μm to about 1 μm (e.g., about 0.1 μm to about 1 μm, about 0.1 μm to about 0.5 μm, about 0.2 to about 0.45 μm, about 0.45 to about 1 μm, about 0.1 μm, about 0.2 μm, about 0.45 μm, about 0.65 μm, or about 1 μm) can be used in TFF. A membrane can be made from any appropriate material. In some cases, a membrane can be a hydrophilic membrane. In some embodiments, a membrane can be a hydrophobic membrane. In some embodiments, a membrane with a nominal molecular weight cutoff (NMWCO) of at least about 100 kDa (e.g., at least about 200 kDa, 300 kDa, 500 kDa, or 1000 kDa) can be used in TFF. The TFF can be performed with any appropriate pore size within the range of 0.1 μm to 1.0 μm with the aim of reducing the microparticles content in the composition and increasing the content of platelet derivatives in the composition. A skilled artisan can appreciate the required optimization of the pore size in order to retain the platelet derivatives and allow the microparticles to pass through the membrane. The pore size in illustrative embodiments, is such that the microparticles pass through the membrane allowing the TFF-treated composition to have less than 5% microparticles. The pore size in illustrative embodiments is such that a maximum of platelet derivatives gets retained in the process allowing the TFF-treated composition to have a concentration of the platelet derivatives in the range of 100×10³ to 20,000×10³. The pore size during the TFF process can be exploited to obtain a higher concentration of platelet derivatives in the platelet derivative composition such that a person administering the platelet derivatives to a subject in need has to rehydrate/reconstitute fewer vials, therefore, being efficient with respect to time and effort during the process of preparing such platelet derivatives for a downstream procedure, for example a method of treating provided herein. TFF can be performed at any appropriate temperature. In some embodiments, TFF can be performed at a temperature of about 20° C. to about 37° C. (e.g., about 20° C. to about 25° C., about 20° C. to about 30° C., about 25° C. to about 30° C., about 30° C. to about 35° C., about 30° C. to about 37° C., about 25° C. to about 35° C., or about 25° C. to about 37° C.). In some embodiments, TFF can be carried out at a flow rate (e.g., a circulating flow rate) of about 100 ml/min to about 800 ml/min (e.g., about 100 to about 200 ml/min, about 100 to about 400 ml/min, about 100 to about 600 ml/min, about 200 to about 400 ml/min, about 200 to about 600 ml/min, about 200 to about 800 ml/min, about 400 to about 600 ml/min, about 400 to about 800 ml/min, about 600 to about 800 ml/min, about 100 ml/min, about 200 ml/min, about 300 ml/min, about 400 ml/min, about 500 ml/min, about 600 ml/min, about 700 ml/min, or about 800 ml/min).

In some embodiments, TFF can be performed until a particular endpoint is reached, forming a TFF-treated composition. An endpoint can be any appropriate endpoint. In some embodiments, an endpoint can be a percentage of residual plasma (e.g., less than or equal to about 50%, 40%, 30%, 20%, 150%, 10%, 9%, 8%, 7%, 6%, 5%, 4%, 3%, 2%, 10%, 0.9%, 0.8%, 0.7%, 0.6%, 0.50%, 0.4%, 0.30%, 0.2%, or 0.10% of residual plasma). In some embodiments, an endpoint can be a relative absorbance at 280 nm (A280). For example, an endpoint can be an A280 (e.g., using a path length of 0.5 cm) that is less than or equal to about 50% (e.g., less than or equal to about 40%, 30%, 20%, 15%, 10%, 9%, 8%, 7%, 6%, 5%, 4%, 3%, 2%, 1%, 0.9%, 0.8%, 0.7%, 0.6%, 0.5%, 0.4%, 0.3%, 0.2%, or 0.1%) of the A280 (e.g., using a path length of 0.5 cm) prior to TFF (e.g., of a starting material or of a diluted starting material). In some embodiments, an A280 can be relative to a system that measures 7.5% plasma=1.66 AU. In some embodiments, an instrument to measure A280 can be configured as follows: a 0.5 cm gap flow cell can be attached to the filtrate line of the TFF system. The flow cell can be connected to a photometer with fiber optics cables attached to each side of the flow cell (light source cable and light detector cable). The flow cell can be made with a silica glass lens on each side of the fiber optic cables. Apart from the relative protein concentration of proteins in the aqueous medium, the protein concentration in the aqueous medium can also be measured in absolute terms. In some embodiments, the protein concentration in the aqueous medium is less than or equal to 15%, or 14%, or 13%, or 12%, or 11%, or 10%, or 9%, or 8%, or 7%, or 6%, or 5%, or 4%, or 3%, or 2%, or 1%, or 0.1%, or 0.01%. In some exemplary embodiments, the protein concentration is less than 3% or 4%. In some embodiments, the protein concentration is in the range of 0.01-15%, or 0.1-15%, or 1-15%, or 1-10%, or 0.01-10%, or 3-12%, or 5-10% in the TFF-treated composition. In some embodiments, an endpoint can be an absolute A280 (e.g., using a path length of 0.5 cm). For example, an endpoint can be an A280 that is less than or equal to 2.50 AU, 2.40 AU, 2.30 AU, 2.20 AU, 2.10 AU, 2.0 AU, 1.90 AU, 1.80 AU, or 1.70 AU (e.g., less than or equal to 1.66, 1.6, 1.5, 1.4, 1.3, 1.2, 1.1, 1.0, 0.9, 0.8, 0.7, 0.6, 0.5, 0.4, 0.3, 0.2, or 0.1 AU) (e.g., using a path length of 0.5 cm). In some embodiments, a percentage of residual plasma, a relative A280, or an A280 can be determined based on the aqueous medium of a composition comprising platelets and an aqueous medium. In some embodiments, a percentage of residual plasma can be determined based on a known correlation to an A280. In some embodiments, an endpoint can be a platelet concentration, as TFF can include concentration or dilution of a sample (e.g., using a preparation agent). For example, an endpoint can be a platelet concentration of at least about 2000×10³ platelets/μL (e.g., at least about 2050×10³, 2100×10³, 2150×10³, 2200×10³, 2250×10³, 2300×10³, 2350×10³, 2400×10³, 2450×10³, or 2500×10³ platelets/μL). As another example, an endpoint can be a platelet concentration of about 1000×10³ to about 2500 platelets/μL (e.g., about 1000×10³ to about 2000×10³, about 1500×10³ to about 2300×10³, or about 1700×10³ to about 2300×10³ platelets/μL). In some embodiments, an endpoint can be a concentration of platelets in the TFF-treated composition are at least 100×10³ platelets/μL, 200×10³ platelets/μL, 400×10³ platelets/μL, 1000×10³ platelets/μL, 1250×10³ platelets/μL, 1500×10³ platelets/μL, 1750×10³ platelets/μL, 2000×10³ platelets/μL, 2250×10³ platelets/μL, 2500×10³ platelets/μL, 2750×10³ platelets/μL, 3000×10³ platelets/μL, 3250×10³ platelets/μL, 3500×10³ platelets/μL, 3750×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, 20,000×10³ platelets/μL. In some embodiments, the platelets or platelet derivatives in the TFF-treated composition is in the range of 100×10³-20,000×10³ platelets/μL, or 1000×10³-20,000×10³ platelets/μL, or 1000×10³-10,000×10³ platelets/μL, or 500×10³-5,000×10³ platelets/μL, or 1000×10³-5,000×10³ platelets/μL, or 2000×10³-8,000×10³ platelets/μL, or 10,000×10³-20,000×10³ platelets/μL, or 15,000×10³-20,000×10³ platelets/μL.

In some embodiments, an endpoint can include more than one criterion (e.g., a percentage of residual plasma and a platelet concentration, a relative A280 and a platelet concentration, or an absolute A280 and a platelet concentration).

Typically, a TFF-treated composition is subsequently lyophilized, optionally with a thermal treatment step, to form a final blood product (e.g., platelets, cryopreserved platelets, freeze-dried platelets (e.g., thrombosomes)). However, in some cases, a TFF-treated composition can be considered to be a final blood product.

In some embodiments, a blood product can be prepared using centrifugation of a blood product (e.g., an unprocessed blood product (e.g., donor apheresis material (e.g., pooled donor apheresis material)), or a partially processed blood product (e.g., a blood product that has undergone TFF)). In some embodiments, a blood product can be prepared without centrifugation of a blood product (e.g., an unprocessed blood product (e.g., donor apheresis material), or a partially processed blood product (e.g., a blood product that has undergone TFF)). Centrifugation can include any appropriate steps. In some embodiments, centrifugation can include a slow acceleration, a slow deceleration, or a combination thereof. In some embodiments, centrifugation can include centrifugation at about 1400×g to about 1550×g (e.g., about 1400 to about 1450×g, about 1450 to about 1500×g, or 1500 to about 1550×g, about 1400×g, about 1410×g, about 1430×g, about 1450×g, about 1470×g, about 1490×g, about 1500×g, about 1510×g, about 1530×g, or about 1550×g). In some embodiments, the duration of centrifugation can be about 10 min to about 30 min (e.g., about 10 to about 20 min, about 20 to about 30 min, about 10 min, about 20 min, or about 30 min).

In some embodiments, a final blood product can be prepared using both TFF and centrifugation (e.g., TFF followed by centrifugation or centrifugation followed by TFF).

Also provided herein are compositions prepared by any of the methods described herein.

In some embodiments, a composition as described herein can be analyzed at multiple points during processing. In some embodiments, a starting material (e.g., donor apheresis material (e.g., pooled donor apheresis material)) can be analyzed for antibody content (e.g., HLA or HNA antibody content). In some embodiments, a starting material (e.g., donor apheresis material (e.g., pooled donor apheresis material)) can be analyzed for protein concentration (e.g., by absorbance at 280 nm (e.g., using a path length of 0.5 cm)). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of the starting material. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

In some embodiments, a composition as described herein can be analyzed at multiple points during processing. In some embodiments, donor apheresis plasma can be analyzed for antibody content (e.g., HLA or HNA antibody content). In some embodiments, donor apheresis plasma can be analyzed for protein concentration (e.g., by absorbance at 280 nm). In some embodiments, a composition in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of an unprocessed blood product) can be analyzed for antibody content (e.g., HLA or HNA antibody content). In some embodiments, the antibody content (e.g., HLA or HNA antibody content) of a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product (e.g., (e.g., platelets, cryopreserved platelets, freeze-dried platelets (e.g., thrombosomes)) can be analyzed for antibody content (e.g., HLA or HNA antibody content). In some embodiments described herein, a final blood product can be a composition that includes platelets and an aqueous medium. In some embodiments, the antibody content (e.g., HLA or HNA antibody content) of a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the antibody content of donor apheresis plasma. In some embodiments, a final blood product can have no detectable level of an antibody selected from the group consisting of HLA Class I antibodies, HLA Class II antibodies, and HNA antibodies. In some embodiments, the aqueous medium of a composition as described herein can be analyzed as described herein.

The protein concentration of a blood product can be measured by any appropriate method. In some embodiments, the protein concentration of a blood product can be measured using absorbance at 280 nm.

The antibody content (e.g., HLA or HNA antibody content) of a blood product can be measured by any appropriate method.

In some embodiments, a FLOWPRA™ Screening or a LABScreen Multi test kits from One Lambda, Thermo Fisher Scientific can be used as a method of HLA detection. Raw materials can be tested prior to the TFF or centrifugation processes to determine a baseline level of class I and II antibodies for Human Leukocyte Antigen (HLA) and Human Neutrophil Antigens (HNA). Testing can be repeated after processing by centrifugation or TFF to measure the removal of HLA and HNA. Additional testing points can be performed throughout the TFF procedure to maintain in-process control. Post-lyophilization and annealing, random samples can be selected from a batch and qualitative HLA/HNA antibody testing can be performed to ensure reduction and compliance with current FDA testing and acceptance requirements.

In some embodiments, the antibody content (e.g., HLA or HNA antibody content) of two blood products can be compared by determining the percentage of beads positive for a marker (e.g., HLA or HNA coated beads bound to HLA or HNA antibodies, respectively). Any appropriate comparative method can be used. In some embodiments, the antibody content of two blood products can be compared using a method as described herein. In some embodiments, such a method can be carried out as follows. An aliquot of plasma (e.g., about 1 mL) platelet-poor plasma can be obtained. In some embodiments, an aliquot of filtered (e.g., using a 0.2 μm filter) platelet-poor plasma (PPP) (e.g., about 1 mL) can be obtained. Beads coated with Class I HLA and/or beads coated with Class II HLA can be added to the plasma (e.g., about 5 μL of each type of bead to about 20 μL of PPP) to form a mixture of PPP and beads. The mixture of PPP and beads can be vortexed. The mixture of PPP and beads can be incubated to form an incubated mixture. Any appropriate incubation conditions can be used. For example, in some embodiments, incubation can occur for a time (e.g., for about 30 minutes) at a temperature (e.g., at room temperature) with other conditions (e.g., in the dark) to form an incubated mixture. In some embodiments, incubation can include agitation (e.g., gentle rocking). The beads in the incubated mixture can be washed using any appropriate conditions. In some embodiments, the beads in the incubated mixture can be washed with a wash buffer. Washed beads can be separated from the incubated mixture by any appropriate method. In some embodiments, the washed beads can be separated by centrifugation (e.g., at 9,000×g for 2 minutes) to obtain pelleted beads. In some embodiments, the washing step can be repeated. The beads can be resuspended to form a bead solution. An antibody (e.g., an antibody that will bind to the assayed antibody content (e.g., HLA or HNA antibody content)) conjugated to a detectable moiety can be added to the bead solution (e.g., an αIgG conjugated to a fluorescent reporter, such as FITC). The antibody can be incubated with the bead solution under any appropriate conditions. In some embodiments, the antibody can be incubated for a time (e.g., for about 30 minutes) at a temperature (e.g., at room temperature) with other conditions (e.g., in the dark) to form labeled beads. Labeled beads can be washed to remove unbound antibody conjugated to a detectable moiety. The labeled beads can be washed using any appropriate conditions. In some embodiments, the labeled beads can be washed with a wash buffer. Washed labeled beads can be separated by any appropriate method. In some embodiments, the washed labeled beads can be separated by centrifugation (e.g., at 9,000 g for 2 minutes) to obtain pelleted labeled beads. In some embodiments, the washing step can be repeated. Labeled beads can be detected by any appropriate method. In some embodiments, labeled beads can be detected by flow cytometry. In some embodiments, detection can include measurement of the percentage of beads that are positive for the detectable moiety as compared to a negative control. In some embodiments, a negative control can be prepared as above, using a PPP sample that is known to be negative for antibodies (e.g. HLA Class I, HLA Class II, or HNA antibodies).

In some embodiments, a blood product (e.g., platelets, cryopreserved platelets, freeze-dried platelets (e.g., thrombosomes)) can be analyzed at multiple points during processing. In some embodiments, a starting material (e.g., donor apheresis material) can be analyzed to determine the percent of positive beads (e.g., HLA or HNA coated beads). In some embodiments, a starting material (e.g., donor apheresis material) can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from a starting material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

In some embodiments, a blood product (e.g., platelets, cryopreserved platelets, freeze-dried platelets (e.g., thrombosomes)) can be analyzed at multiple points during processing. In some embodiments, donor apheresis plasma can be analyzed to determine the percent of positive beads (e.g., HLA or HNA coated beads). In some embodiments, donor apheresis plasma can be analyzed for protein concentration (e.g., by absorbance at 280 nm). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, a blood product in an intermediate step of processing (e.g., when protein concentration reduced to less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the protein concentration of a starting material) can be analyzed to determine the percent of positive beads (e.g., HLA or HNA coated beads). In some embodiments, the percent of positive beads (e.g., HLA or HNA coated beads) from a blood product in an intermediate step of processing can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis plasma. In some embodiments, the percent of positive beads (e.g., HLA or HNA coated beads) from a blood product in an intermediate step of processing can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, a final blood product (e.g., (e.g., platelets, cryopreserved platelets, freeze-dried platelets (e.g., thrombosomes)) can be analyzed to determine the percent of positive beads (e.g., HLA or HNA coated beads). In some embodiments, the percent of positive beads (e.g., HLA or HNA coated beads) from a final blood product (e.g., (e.g., platelets, cryopreserved platelets, freeze-dried platelets (e.g., thrombosomes)) can be at least 5% reduced (e.g., 10%, 20%, 30%, 40%, 50%, 60%, 70%, 75%, 80%, 85%, 90%, 95%, 96%, 97%, 98%, 99%, or more, reduced) compared to the percent of positive beads from donor apheresis material. In some embodiments, the percent of positive beads (e.g., HLA or HNA coated beads) from a final blood product can be less than or equal to 75% (e.g., less than or equal to 70%, 65%, 60%, 55%, 50%, 45%, 40%, 35%, 30%, 25%, 20%, 15%, 10%, 9%, 8%, 7%, 6%, 5%, 4%, 3%, 2%, 1%, or less) of the total amount of beads. In some embodiments, the aqueous medium of a composition as described herein can be analyzed as described herein.

A percentage of positive beads can be determined using any appropriate method. In some embodiments, positive beads can be determined compared to a negative control sample. A negative control sample can be any appropriate negative control sample. In some embodiments, a negative control sample can be used to determine positivity gating such that less than a certain percentage (e.g., between about 0.01% and about 1% (e.g., about 0.01% to about 0.05%, about 0.05% to about 0.1%, about 0.1% to about 0.5%, about 0.5% to about 1%, about 0.01%, about 0.05%, about 0.1%, about 0.5%, or about 1%)) of the negative control sample is present within the positivity gate. In some embodiments, a negative control sample can be a buffer (e.g., PBS). In some embodiments, a negative control sample can be a synthetic plasma composition. In some embodiments, a negative control sample can be a blood product known to be negative for the assayed antibodies (e.g., HLA or HNA antibodies).

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by tangential flow filtration.

Also provided herein is a method of reducing the percentage of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the amount of an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation. Also provided herein is a method of reducing the percentage of beads positive for an antibody (e.g., a HLA antibody (e.g., a HLA Class I antibody or a HLA Class II antibody) or a HNA antibody) in a composition (e.g., a blood product) comprising platelets, the method comprising filtering the composition by centrifugation.

In some embodiments of any of the methods described herein, the amount of an antibody (e.g., a HLA antibody (e.g., a HLA Class I antibody or a HLA Class II antibody) or a HNA antibody) in a composition (e.g., a blood product) can be reduced to below a reference level. A reference level can be any appropriate reference level. In some embodiments of any of the methods described herein, the percentage of beads positive an antibody (e.g., a HLA antibody (e.g., a HLA Class I antibody or a HLA Class II antibody) or a HNA antibody) in a composition (e.g., a blood product) can be reduced as compared to the blood product before undergoing the methods described herein. A percentage of beads positive for an antibody can be reduced by any appropriate amount. In some embodiments, a percentage of beads positive for an antibody can be reduced by at least 5% (e.g., reduced by at least 10%, 20%, 30%, 40%, 50%, 60%, 70%, 75%, 80%, 85%, 90%, 95%, 96%, 97%, 98%, 99%, or more) compared to the blood product before undergoing any of the methods described herein.

In some embodiments, a composition as described herein can undergo any appropriate additional processing steps. In some embodiments, a composition as described herein can be freeze-dried. In some embodiments, freeze-dried platelets can be thermally treated (e.g., at about 80° C. for about 24 hours).

For example, in some embodiments, a composition can be cryopreserved or freeze-dried. In some embodiments, a first composition (e.g., a composition comprising platelets and an aqueous medium as described herein) can be treated with a mixture. In some embodiments, a mixture can include a lyophilizing agent, including a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form a second composition comprising platelets. In some embodiments, a loading agent can be a saccharide. In some embodiments, a saccharide can be a monosaccharide. In some embodiments, a saccharide can be sucrose, maltose, trehalose, glucose (e.g., dextrose), mannose, or xylose. In some embodiments, the loading agent can be polysucrose.

In some embodiments, a first composition or a second composition can be dried. In some embodiments, a first composition or a second composition can be dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a third composition. In some embodiments, a cryoprotectant can be polysucrose.

In some embodiments, a first composition or a second composition can be freeze-dried. In some embodiments, a first composition or a second composition can be freeze-dried with a cryoprotectant. In some embodiments, a cryoprotectant can include a saccharide, optionally a base, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof to form a fourth composition. In some embodiments freeze-drying can occur at a temperature of about −40° C. to about 5° C. In some embodiments, freeze-drying can occur over a gradient (e.g., about −40° C. to about 5° C.). In some embodiments, a secondary drying step can be carried out (e.g., at about 20° C. to about 40° C.).

Also provided herein are blood products (e.g., platelets, cryopreserved platelets, freeze-dried platelets (e.g., thrombosomes)) produced by any of the methods described herein.

In some embodiments, the percentage of beads positive for an antibody selected from the group consisting of HLA Class I antibodies, HLA Class II antibodies, and HNA antibodies, as determined for a composition as described herein by flow cytometry using beads coated with Class I HLAs, Class II HLAs, or HNAs, respectively, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class I antibodies, as determined for a composition as described herein by flow cytometry using beads coated with Class I HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HLA Class II antibodies, as determined for a composition as described herein by flow cytometry using beads coated with Class II HLAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

In some embodiments, the percentage of beads positive for HNA antibodies, as determined for a composition as described herein by flow cytometry using beads coated with HNAs, is reduced by at least 10% (e.g., at least 20%, 30%, 40%, 50%, 60%, 70%, 75%, 80%, 85%, 90%, 95%, or 99%) as compared to a similar composition not prepared by a process comprising tangential flow filtration of a composition comprising platelets, centrifugation of a composition comprising platelets, or a combination thereof.

Within the process provided herein for making the compositions provided herein, optional addition of a lyophilizing agent can be the last step prior to drying. However, in some embodiments, the lyophilizing agent can be added at the same time or before other components of the composition, such as a salt, a buffer, optionally a cryoprotectant, or other components. In some embodiments, the lyophilizing agent is added to a preparation agent, thoroughly mixed to form a drying solution, dispensed into a drying vessel (e.g., a glass or plastic serum vial, a lyophilization bag), and subjected to conditions that allow for drying of a TFF-treated composition to form a dried composition.

In various embodiments, the lyophilization bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SAV ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SAV ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6, cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SAV ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, dried platelets or platelet derivatives (e.g., thrombosomes) can undergo heat treatment. Heating can be performed at a temperature above about 25° C. (e.g., greater than about 40° C., 50° C., 60° C., 70° C., 80° C. or higher). In some embodiments, heating is conducted between about 70° C. and about 85° C. (e.g., between about 75° C. and about 85° C., or at about 75° C. or 80° C.). The temperature for heating can be selected in conjunction with the length of time that heating is to be performed. Although any suitable time can be used, typically, the lyophilized platelets are heated for at least 1 hour, but not more than 36 hours. Thus, in embodiments, heating is performed for at least 2 hours, at least 6 hours, at least 12 hours, at least 18 hours, at least 20 hours, at least 24 hours, or at least 30 hours. For example, the lyophilized platelets can be heated for 18 hours, 19 hours, 20 hours, 21 hours, 22 hours, 23 hours, 24 hours, 25 hours, 26 hours, 27 hours, 28 hours, 29 hours, or 30 hours. Non-limiting exemplary combinations include: heating the dried platelets or platelet derivatives (e.g., thrombosomes) for at least 30 minutes at a temperature higher than 30° C.; heating the dried platelets or platelet derivatives (e.g., thrombosomes) for at least 10 hours at a temperature higher than 50° C.; heating the dried platelets or platelet derivatives (e.g., thrombosomes) for at least 18 hours at a temperature higher than 75° C.; and heating the dried platelets or platelet derivatives (e.g., thrombosomes) for 24 hours at 80° C. In some embodiments, heating can be performed in sealed container, such as a capped vial. In some embodiments, a sealed container be subjected to a vacuum prior to heating. The heat treatment step, particularly in the presence of a cryoprotectant such as albumin or polysucrose, has been found to improve the stability and shelf-life of the freeze-dried platelets. Indeed, advantageous results have been obtained with the particular combination of serum albumin or polysucrose and a post-lyophilization heat treatment step, as compared to those cryoprotectants without a heat treatment step. A cryoprotectant (e.g., sucrose) can be present in any appropriate amount (e.g. about 3% to about 10% by mass or by volume of the platelets or platelet derivatives (e.g., thrombosomes).

In some cases, compositions comprising platelets or platelet derivatives (e.g., thrombosomes) can be rehydrated with water (e.g., sterile water for injection) over about 10 minutes at about room temperature. In general, the rehydration volume is about equal to the volume used to fill each vial of thrombosomes prior to drying.

In some embodiments, the platelets or platelet derivatives (e.g., thrombosomes) prepared as disclosed herein have a storage stability that is at least about equal to that of the platelets prior to the preparation.

In some embodiments, the method further comprises cryopreserving the platelets or platelet derivatives prior to administering the platelets or platelet derivatives (e.g., with a preparation agent, e.g., a preparation agent described herein).

In some embodiments, the method further comprises drying a composition comprising platelets or platelet derivatives, (e.g., with a preparation agent e.g., a preparation agent described herein) prior to administering the platelets or platelet derivatives (e.g., thrombosomes). In some embodiments, the method may further comprise heating the composition following the drying step. In some embodiments, the method may further comprise rehydrating the composition following the freeze-drying step or the heating step.

In some embodiments, the method further comprises freeze-drying a composition comprising platelets or platelet derivatives (e.g., with a preparation agent e.g., a preparation agent described herein) prior to administering the platelets or platelet derivatives (e.g., thrombosomes) In some embodiments, the method may further comprise heating the composition following the freeze-drying step. In some embodiments, the method may further comprise rehydrating the composition following the freeze-drying step or the heating step.

In some embodiments, the method further comprises cold storing the platelets, platelet derivatives, or the thrombosomes prior to administering the platelets, platelet derivatives, or thrombosomes (e.g., with a preparation agent, e.g., a preparation agent described herein).

Storing conditions include, for example, standard room temperature storing (e.g., storing at a temperature ranging from about 20 to about 30° C.) or cold storing (e.g., storing at a temperature ranging from about 1 to about 10° C.). In some embodiments, the method further comprises cryopreserving, freeze-drying, thawing, rehydrating, and combinations thereof, a composition comprising platelets or platelet derivatives (e.g., thrombosomes) (e.g., with a preparation agent e.g., a preparation agent described herein) prior to administering the platelets or platelet derivatives (e.g., thrombosomes). For example, in some embodiments, the method further comprises drying (e.g., freeze-drying) a composition comprising platelets or platelet derivatives (e.g., with a preparation agent e.g., a preparation agent described herein) (e.g., to form thrombosomes) prior to administering the platelets or platelet derivatives (e.g., thrombosomes). In some embodiments, the method may further comprise rehydrating the composition obtained from the drying step.

In some embodiments, provided herein is a method for preparing a composition comprising platelets or platelet derivatives (e.g., thrombosomes). The method can include diluting a starting material comprising platelets with an approximately equal weight (±10%) of a preparation agent (e.g., Buffer A, as provided in Example 1 of U.S. Pat. No. 11,529,587 and Example 1 of PCT app no. PCT/US2022/079280), concentrating the platelets to about 2250×10³ cells/μL (+250×10³) and then washed with 2-4 diavolumes (DV) (e.g., about 2 diavolumes) of the preparation agent to form a TFF-treated composition. The residual plasma percentage can be less than about 15% relative plasma (as determined by plasma protein content). Following washing, if the concentration of the cells in the TFF-treated composition is not about 2000×10³ cells/μL (+300×10³), the cells can be diluted with the preparation agent or can be concentrated to fall within this range. The method can further include lyophilizing the TFF-treated composition and subsequently treating the lyophilized composition comprising platelets or platelet derivatives (e.g., thrombosomes) at about 80° C. for about 24 hours. In some embodiments, the method can further include a pathogen reduction step, for example, before diluting the starting material.

### Plurality of Containers Containing Platelet Derivative Composition

The platelet derivative composition as described herein can be contained in containers/vials, which further can be packed into a plurality of containers for shipping to a customer, which can be part of a commercialization process to fulfill an order for such platelet derivative composition. The containers/vessels, in certain embodiments, are 5 ml vials, 10 ml vials, 20 ml vials, 25 ml vs, 30 ml vials, 40 ml vials, 50 ml vials, 60 ml vials, 75 ml vials, 100 ml vials, 125 ml vials, 150 ml vials, 200 ml vials, or 250 ml vials. The vial(s) can be a cryovial, or a cryotube especially in illustrative embodiments where the TFF-treated composition that includes platelets is lyophilized to obtain the platelet derivative composition in the form of a powder, which further can be baked or not baked after it is lyophilized. In some embodiments, the volume of the containers in a plurality of containers (e.g. vials or tubes), which for example can be all from one lot, or from more than one lot (e.g. 2, 3, 4, 5, 6, 7, 8, 9 or 10 lots), can vary from one or more than one size between 10-100 ml. Typically, the volume of the vial/container in embodiments where the platelet derivative is a freeze-dried solid/powder, is 1× the volume of, or 1.10, 1.25, 1.5, 2, 2.5, 3, 4 or 5 times the volume of a composition that was filled in the vial before lyophilization, and/or the volume in which the powder in the vials will be rehydrated, which is an illustrative embodiment. Thus, the maximum volume of such vials can be the same or more than the volume of the composition that was filled inside prior to lyophilization or the volume in which the platelet derivative composition in the form of a powder can be rehydrated. For example, in one non-limiting embodiment, a vial with a maximum capacity of 100 ml, can be used to fill 10 ml of a TFF-treated composition that includes platelets for lyophilization. In certain embodiments, the capacity of a vial in which a TFF-treated composition that includes platelets is lyophilized, is 1-2.5 times and in other embodiments, 1-2 times, 1-3 times, 1-4 times, 1-5 times, and in certain illustrative embodiments, 1.1 to 2 times or 1.25 to 2 times the volume of a TFF-treated composition that is lyophilized therein.

The TFF-treated platelet composition before lyophilization, or in some embodiments, the platelet derivative composition obtained after the lyophilization step, with or without post-lyophilization heat treatment (baking), can be filled into a plurality of vessels or other powder and liquid-holding containers, such as vials, in a sterile manner. In some embodiments, the containers/vessels can vary in volume from 5-100 ml, 10-90 ml, 25-75 ml, or 5-40 ml. In some embodiments, the volume of containers/vessels can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml. In some embodiments, the volume of containers/vessels can be above 100 ml, for example, 125 ml, 150 ml, 175 ml, or 200 ml. The platelet derivative composition as described herein can be filled in vials of different volumes as per the commercialization requirements. A plurality (or collection) of containers/vessels having the platelet derivative composition as per any of the embodiments herein, obtained by lyophilizing the composition that includes platelets during one process (e.g. TFF or other process) for preparing a platelet derivative, can be referred to as a “batch” or a “lot”. In some embodiments, a batch/lot can have 10-500 vials, 25-450 vials, 50-350 vials, 100-300 vials, or 150-250 vials. In some embodiments, a batch/lot can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the number of vials per batch/lot can be increased to more than 500 as per the requirements, for example, 600, 700, 800, 900, or 1000 vials. In some embodiments, the number of vials can be 10-1000, 50-1000, 100-900, 200-800, 100-500, 100-400, 150-700, or 150-500 vials. The containers in a batch/lot can have a volume in the range of 5-100 ml, for example, such that a lot has several containers with the same volume or containers with different volumes. For example, 200 vials/containers in a batch/lot can have a volume of 10 ml each, 100 vials/containers in the same or a separate batch/lot can have a volume of 20 ml each, 100 vials/containers in the same or another batch/lot can have a volume of 30 ml each, or 300 vials/containers in the same or a different batch/lot can have a volume of 10 ml each. The number of containers (e.g. vials) in which a platelet derivative composition as per one of the embodiments or aspects described herein can be packed in a batch/lot can vary with manufacturing requirements, the requirements of downstream processes, for example clinical processes, and the amount of starting material comprising platelet composition.

The quantity of platelet derivatives that is present in a batch/lot can vary based on the units of starting material comprising platelets that is used to produce the platelet derivatives. Certain methods provided herein, such as the TFF methods provided herein, allow more platelet units to be used to make platelet derivatives with the characteristics provided herein than prior methods. This is the result, for example, of the ability to reduce the level of certain components in a platelet composition starting material, such as HLA antibodies, HNA antibodies, and/or microparticles, to very low levels, as provided herein. Accordingly, the starting material comprising platelets, the corresponding composition (e.g. TFF-treated composition) that is lyophilized in illustrative embodiments, and the resulting platelet derivative composition powder, can vary, such that for example, in some embodiments, the starting material, the TFF-treated composition, and/or the resulting platelet derivative composition powder can include 10-500 units of platelets or platelet derivatives (e.g. 0.5 to 2.5 μm in diameter), with one unit being 3×10¹¹ platelets or platelet derivatives. In some embodiments, the starting platelet material, the composition to be lyophilized, and/or the platelet derivative (e.g. 0.5 to 2.5 μm in diameter) composition can include, for example, 20-500 units, 30-400 units, 40-350 units, or 50-200 units of platelets or platelet derivatives, respectively. In some embodiments, the platelet units in the starting platelet composition can be a pooled platelet product from multiple donors as described herein, or multiple batches of processed platelet compositions, such as TFF-treated compositions comprising platelets, can be pooled before lyophilization. In some embodiments, there can be 1×10⁹ to 1×10¹⁶ platelets in a starting platelet composition for processing, in a platelet composition that is lyophilized, and/or of platelet derivatives in a platelet derivative composition that is produced after lyophilization, per batch/lot. In some embodiments, the platelet-containing starting composition, the platelet composition that is lyophilized, and/or the platelet derivatives that are produced, typically after lyophilization per batch/lot can vary from 1×10¹⁰ to 1×10¹⁵, 1×10¹¹ to 1×10¹⁵, 1×10¹² to 1×10¹⁶, 1×10¹³ to 1×10¹⁵ or 1×10¹³ to 1×10¹⁴.

In certain illustrative embodiments, platelet derivative compositions that are present in a liquid, or in illustrative embodiments, a solid form such as a dried powder in the plurality of containers (e.g. vials), in illustrative embodiments of a 1 or more lots, are compositions that include platelet derivatives, wherein at least 50% of the platelet derivatives are CD 41-positive platelet derivatives, wherein less than 15%, 10%, or in further, non-limiting illustrative embodiments less than 5% of the CD 41-positive platelet derivatives are microparticles having a diameter of less than 0.5 μm, and wherein the platelet derivatives have a potency of at least 0.5, 1.0 and in further, non-limiting illustrative embodiments 1.5 thrombin generation potency units (TGPU) per 10⁶ platelet derivatives. In certain illustrative embodiments, including non-limiting examples of the illustrative embodiment in the preceding sentence, the platelet derivatives are 0.5 to 2.5 um in diameter. Such platelet derivatives and platelet derivative compositions comprising the same, can have additional characteristics disclosed herein for such derivatives and compositions.

Processes provided herein for producing platelet derivative compositions, provide better lot to lot consistency than prior processes. For example, TFF methods provided herein provide improved lot to lot variability with respect to the components of compositions that include platelet derivatives prepared therein, in illustrative embodiments, compositions that include freeze-dried platelet derivates. Such freeze-dried platelet derivatives can be one of, or the main active ingredient(s). In some embodiments, a plurality of containers provided herein comprise the platelet derivative composition from at least 2 different lots in separate containers. In some embodiments, the amount of plasma protein in the powder of any two containers chosen from different lots, differs by less than 50%, 40%, 30%, 25%, or 20%, and in illustrative embodiments less than 10%, 5%, 2%, 1%, or 0.5%. The TFF process is highly controllable and can be stopped at a certain A280 for example, from 2.0 AU to 0.01 AU, or when it reaches 15% to 0.01% protein concentration in the composition that is to be lyophilized (e.g. TFF-treated composition), therefore, the plasma protein content can be very consistent not only within the containers/vials of a lot, but even between lots as well. Since different lots of platelet derivative compositions provided herein are typically prepared from platelets from different subjects or different combinations of subjects (e.g. pooled platelets from 2, 3, 4, 5, 6, 7, 8, 9, 10, 15, 20, 25, 30, 40, 50, 60, 70, 75, 80, or 100 subjects), different lots in illustrative embodiments differ in amino acid sequence of at least one (e.g., 1, 2, 3, 4, 5, 6, 7, 8, 9, 10, 1-100, or 1-10) of the proteins in, on, and/or associated with platelet derivatives of the compositions therein between the lots. In illustrative embodiments, these one or more amino acid differences occur at one or more (e.g., 1, 2, 3, 4, 5, 6, 7, 8, 9, 10, 1-100, or 1-10) of the site(s) of SNPs, in illustrative embodiments non-synonymous SNPs. In certain embodiments, such SNPs or non-synonymous SNPs have a minor allele frequency of less than or equal to 5%. In some embodiments, such pooled platelets are provided by processes provided herein, for example because HLA or HNA antibody levels can be reduced to very low or non-existent levels. Thus, not only can platelets be pooled from more subjects, before processing to form platelet derivatives, but those subjects can be males or females. As a result, platelet derivatives, in illustrative embodiments, FDPDs, of compositions herein, for example liquid or dried compositions, in some embodiments have different amino acid sequences for at least 1 or a plurality of FDPD proteins. Furthermore, as a result, in certain embodiments, within a lot or between lots, greater than 10%, 20%, 25%, 30%, or 40%, and in illustrative embodiments greater than 50%, 60%, 70%, 75%, 80%, 90%, or 95% of amino acids encoded by SNPs, in illustrative embodiments encoded by non-synonymous SNPs in one or more proteins that are bound to or otherwise associated with or part of a platelet derivative, are present for SNPs, for example with a minor allele frequency of greater than 5%, in certain embodiments including in proteins that result from expression of coding sequences comprising SNPs, in illustrative embodiments non-synonymous SNPs on a mammalian X and Y chromosome.

In some embodiments, the amount of microparticles that are less than 0.5 μm in the powder of any two containers chosen from different lots, differs in amount by less than 10%, 5%, 2%, or 1%. Since, for example, a TFF process disclosed herein is very controllable, the concentration of microparticles to be obtained in the platelet derivative composition can be optimized, for example, by performing scattering intensity studies at different time points. Once the desired level is achieved, the TFF-treated composition can be lyophilized and packed in the vials with or without the baking step.

In some embodiments, the percentage by weight of platelet derivative in the powder of any two containers chosen from different lots, differs by less than 10%, 5%, 2%, or 1%. The TFF process can be optimized to achieve a pre-determined level of platelet derivatives in the TFF-treated composition. Such a TFF-treated composition when lyophilized gives a platelet composition in the form of a powder having a certain weight percentage of platelet derivatives. Since, the TFF process is controllable, in some embodiments, there can be a minimum or a negligible variation in the weight percentages of the platelet derivatives in any two containers chosen from different lots.

In some embodiments, at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequences for at least one protein of a collection of protein gene products from a corresponding collection of encoding genes. In illustrative embodiments all, of the lots have a different amino acid sequences for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

As per one of the embodiments, a platelet derivative composition as described herein can be prepared from multiple donors of a single species, for example, mammals, such as for example canine, equine, porcine and in illustrative embodiments humans that are genetically different, in order to obtain a platelet derivative composition to prepare allogenic platelet derivatives, an allogenic platelet derivative product, and/or a composition comprising allogenic platelet derivatives. Such a platelet derivative composition can be filled in vials and a plurality of such vials can be packaged in containers, for example boxes for commercialization as described herein, to obtain a commercial product that is a composition comprising allogeneic platelet derivatives, in illustrative embodiments allogeneic freeze-dried platelet derivatives. The allogenic platelet derivatives as described herein, in some embodiments, can be a U.S. FDA-approved product comprising an allogenic platelet derivative composition. In some embodiments, a platelet derivative composition as described herein can be a European EMA-approved product comprising an allogenic platelet derivative composition. In some other embodiments, a platelet derivative composition as described herein can be a China FDA-approved product comprising an allogenic platelet derivative composition.

In some embodiments, platelets are pooled from a plurality of donors before they are used as starting material for a process for producing a platelet derivative as provided herein. Such platelets pooled from a plurality of donors can be also referred herein to as pooled platelets. In some embodiments, the donors are more than 5, such as more than 10, such as more than 20, such as more than 50, such as up to about 100 donors. In some embodiments, the donors are from 5 to 100, such as from 10 to 50, such as from 20 to 40, such as from 25 to 35. Pooled platelets can be used to make any of the platelet derivative compositions as described herein. The platelets can be pooled wherein the platelets are donated by mammalian (e.g. bovine, feline, porcine, canine, and in illustrative embodiments, human) subjects. In some embodiments, the gender of the subjects can be male or female. In some embodiments, the donor can vary from any number of male to any number of female subjects, for example, from a total of 100 donors, any number can be female donors, ranging from 0-100, 5-95, 10-90, 20-80, 30-70, or 40-60, and the rest can be male donors. In some other embodiments, the donor can be a non-human animal. In some embodiments, the donor can be a canine, equine, porcine, bovine, or feline subject.

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen.

### Platelet Derivatives Comprising an Imaging Agent

In some embodiments, provided herein is a platelet derivative composition, wherein the platelet derivatives can include an imaging agent. In some embodiments, platelets can include/be loaded with an imaging agent, for example before being dried or after being dried. In some embodiments, platelet derivatives that include an imaging agent, such as imaging agent-loaded platelet derivatives can retain the ability or properties of platelet derivatives that are not loaded with imaging agents. Loading of platelet derivatives with an agent typically facilitates imaging of the platelet derivatives in vivo. Thus, a step of detecting an imaging agent, for example an MRI agent, in a subject can be included in any method herein that includes administering or otherwise delivering platelet derivatives to the subject. The imaging agent that can be used to load the platelet derivatives as described herein can include a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ²Fe, ⁵⁵Co, ⁹⁴Tc or ⁶⁸Ga; or gamma-emitters such as ¹⁷¹Tc, ¹¹¹In, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO₃) or a dysprosium iron oxide (DyFeO₃). For example, a paramagnetic metal ion can be chelated to a polypeptide or a monocrystalline nanoparticle. For example, a gamma-emitting radioactive halogen can include, but is not limited to ¹²³I, ¹³¹I or ⁷⁷Br. For example, a positron-emitting radioactive non-metal can include, but is not limited to ¹¹C, ¹³N, ¹⁵O, ¹⁷F, ⁷⁵Br, ⁷⁶Br or ¹²⁴I. For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bis(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, an MRI agent loaded into platelets can be associated with gold or other equivalent metal particles (such as nanoparticles). For example, a metal particle system can include, but is not limited to gold nanoparticles (e.g., Nanogold™).

In some embodiments, an MRI agent loaded into platelets that is modified with an imaging agent is imaged using an imaging unit. The imaging unit can be configured to image the MRI agent loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen, Z., et. al., Advance of Molecular Imaging Technology and Targeted Imaging Agent in Imaging and Therapy, Biomed Res Mt., 819324, doi: 10.1155/2014/819324 (2014) have described various imaging techniques and which is incorporated by reference herein in its entirety. In some embodiments, an imaging agent is an MRI agent. In other embodiments, the MRI agent can be gadolinium-based compounds. In some embodiments, platelet derivatives that include an MRI agent, such as, MRI agent-loaded platelet derivatives can retain the ability or properties of platelet derivatives that are not loaded with MRI agents.

In some embodiments, an imaging agent as provided herein is loaded on platelets or platelet derivatives using a cell penetrating peptide (CPP). In some embodiments, an imaging agent can be conjugated to a CPP and further can be incubated with platelets or platelet derivatives to form imaging agent-loaded platelets or platelet derivatives. In some embodiments, an imaging agent can be an MRI agent to form MRI agent-loaded platelets or platelet derivatives. In some embodiments, a CPP can be any of the CPP well known in the art that can cross the cytoplasmic membrane efficiently. In some embodiments, CPP can be any peptide having 10 to 30 amino acids and are capable of crossing the cytoplasmic membrane. In some embodiments, CPP can be any of the peptides that are described in Kersemans et al 2008 (Kersemans, Veerle et al. “Cell penetrating peptides for in vivo molecular imaging applications.” Current pharmaceutical design vol. 14,24 (2008): 2415-47). In some embodiments, CPP can be a protein-derived CPP, a synthetic CPP, or a mixed CPP. In some embodiments, a protein-derived CPP are derived from naturally occurring protein such as, but not limiting to, TAT protein, and penetratin. In some embodiments, a synthetic CPP such as, but not limiting to polyarginines can be a CPP that is developed by known techniques, such as, phage display method. In some embodiments, a mixed CPP can be a CPP which are a combination of naturally occurring (protein derived) CPP, and synthetic CPP, such as transportan CPP, a combination of the N-terminal fragment of the neuropeptide gelanin and the membrane-interacting wasp venom peptide, mastoparan. In some embodiments, a protein-derived CPPs is selected from the group consisting of penetratin, TAT peptide (49-57 amino acids), TAT peptide (48-60 amino acids), calcitonin-derived CPP, nuclear localization sequences, new polybasic CPPs, N-terminal repetitive domain of maize gamma-zein, peptides from gp41 fusion sequence, preS2-TLM, signal-sequence hydrophobic region (SSHR), pVEC, Vpr, VP22, Human integrin b3 signal sequence, gp41 fusion sequence, Caiman crocodylus Ig(v) light chain, hCT derived peptide, Kaposi FGF signal sequences, CPP from pestivirus envelope glycoprotein, CPP derived from the prion protein, Yeast PRP6 (129-144), Phi21 N (12-29), Delta N (1-22), FHV coat (35-49), BMV Gag (7-25), HTLV-II Rex (4-16), HIV-1 Rev (9-20), RSG-1.2, Lambda-N (48-62), SV40 NLS, Bipartite, Nucleoplasmin (155-170), NLS, Herpesvirus, 8 k8 protein (124-135), Buforin-II (20-36), Magainin, PDX-1-PTD, crotamine, pIsl, SynB1, Fushi-tarazu (254-313), and Engrailed (454-513). In certain illustrative embodiments, a protein-derived CPP is penetratin. In certain illustrative embodiments, a protein-derived CPP is TAT peptide (49-57 amino acids). In certain illustrative embodiments, a protein-derived CPP is TAT peptide (48-60 amino acids). In certain illustrative embodiments, a protein-derived CPP can be any peptide described in the publication Kersemans et al 2008.

In some embodiments, a synthetic and/or mixed CPP is selected from the group consisting of transportan, polyarginine CPPs, poly-d-arginine, KLAL peptide/model amphipathic peptide (MAP), KALA model amphipathic peptide, modeled Tat peptide, Loligomer, b-sheet-forming peptide, retro-inverso forms of established CPPs, W/R penetratin, MPG, Pep-1, Signal-sequence-based peptides (I), Signal-sequence-based peptides (II), Carbamate 9, PTD-4, PTD-5, RSV-A9, CTP-512, and U2AF. In certain illustrative embodiments, a synthetic and/or mixed CPP can be any peptide described in the publication Kersemans et al 2008.

### Exemplary Embodiments

Provided in this Exemplary Embodiments section are non-limiting exemplary aspects and embodiments provided herein and further discussed throughout this specification. For the sake of brevity and convenience, all of the aspects and embodiments disclosed herein, and all of the possible combinations of the disclosed aspects and embodiments are not listed in this section. Additional embodiments and aspects are provided in other sections herein. Furthermore, it will be understood that embodiments are provided that are specific embodiments for many aspects and that can be combined with any other embodiment, for example as discussed in this entire disclosure. It is intended in view of the full disclosure herein, that any individual embodiment recited below or in this full disclosure can be combined with any aspect recited below or in this full disclosure where it is an additional element that can be added to an aspect or because it is a narrower element for an element already present in an aspect. Such combinations are sometimes provided as non-limiting exemplary combinations and/or are discussed more specifically in other sections of this detailed description.

In one aspect provided herein is a method for administering a platelet derivative composition to a subject, in certain embodiments a subject having any indication disclosed herein, in illustrative embodiments a subject having Hermansky Pudlak Syndrome (HPS) or Bernard Soulier Syndrome (BSS), comprising:
administering an effective dose of platelet derivatives in the platelet derivative composition to the subject,
wherein the platelet derivative composition comprises a population of platelet derivatives. In illustrative embodiments, anti-Human Leukocyte Antigen (HLA), and/or anti-Human Platelet Antigen (HPA) antibodies are present in blood of the subject. In some embodiments, the method further comprises determining the presence of anti-Human Leukocyte Antigen (HLA), and/or anti-Human Platelet Antigen (HPA) antibodies in blood of the subject. In some embodiments, anti-Human Leukocyte Antigen (HLA), and/or anti-Human Platelet Antigen (HPA) antibodies are detected in blood of the subject. In some embodiments, blood of the subject is known to be positive for anti-Human Leukocyte Antigen (HLA), and/or anti-Human Platelet Antigen (HPA) antibodies. In some embodiments, the method further comprises determining the presence of cross-reactive antibodies against the platelet derivatives in blood of the subject. Such administering can be performed, for example, until the bleeding of the subject stops.

In one aspect provided herein is a product comprising a vessel containing a platelet derivative composition comprising platelet derivatives, and instructions for administering the platelet derivative composition to a subject, in certain embodiments a subject having any indication disclosed herein, in illustrative embodiments a subject having Hermansky Pudlak Syndrome (HPS) or Bernard Soulier Syndrome (BSS), wherein the administering comprises administering an effective dose of the platelet derivatives in the platelet derivative composition to the subject, and wherein anti-Human Leukocyte Antigen (HLA), and/or anti-Human Platelet Antigen (HPA) antibodies are present in blood of the subject. Such administering can be performed, for example, until the bleeding of the subject stops.

In one aspect provided herein is a platelet derivative composition for use in administering to a subject having Hermansky Pudlak Syndrome (HPS), wherein the administering comprises administering an effective dose of platelet derivatives in the platelet derivative composition to the subject. In some embodiments, the platelet derivatives have at least one property of: a) have the ability to generate thrombin in vitro in the presence of tissue factor and phospholipids; b) have the ability to occlude a collagen-coated microchannel in vitro; or c) both a) and b). In illustrative embodiments, the platelet derivatives: a) have the ability to generate thrombin in vitro in the presence of tissue factor and phospholipids; b) have the ability to occlude a collagen-coated microchannel in vitro. In some embodiments, the subject is bleeding at the start of the administering. In some embodiments, the administering leads to cessation of the bleeding.

In one aspect provided herein is a platelet derivative composition for use in administering to a subject having Hermansky Pudlak Syndrome (HPS) or Bernard Soulier Syndrome (BSS), wherein the administering comprises administering an effective dose of platelet derivatives in the platelet derivative composition to the subject. In some embodiments, the subject is bleeding at the start of the administering. In some embodiments, the administering leads to cessation of the bleeding. In some embodiments, the platelet derivatives have a compromised plasma membrane, and at least one property of: (i) at least 50% of the platelet derivatives are CD 41-positive; ii) the platelet derivatives have the ability to generate thrombin in vitro in the presence of tissue factor and phospholipids; and iii) the platelet derivatives have the ability to occlude a collagen-coated microchannel in vitro.

In one aspect provided herein is a method for administering freeze-dried platelet derivatives (FDPDs) to a subject having Hermansky Pudlak Syndrome (HPS) or Bernard Soulier Syndrome (BSS), comprising administering an effective dose of the platelet derivatives in a platelet derivative composition to the subject. The platelet derivatives can have numerous characteristics provided herein, that make them well suited to restore hemostatic functions in the subject. In some embodiments, the subject is bleeding. In some embodiments, the administering leads to cessation of bleeding.

In another aspect, provided herein is a method for administering freeze-dried platelet derivatives to a subject having Hermansky Pudlak Syndrome (HPS) or Bernard Soulier Syndrome (BSS), comprising:
administering an effective dose of the freeze-dried platelet derivatives in a platelet derivative composition to the subject,
wherein the platelet derivative composition comprises a population of freeze-dried platelet derivatives (FDPDs) comprising CD 41-positive platelet derivatives, wherein less than 5% of the CD 41-positive platelet derivatives are microparticles,
and wherein the FDPDs
   a) have the ability to generate thrombin in vitro in the presence of tissue factor and phospholipids;
   b) have the ability to occlude a collagen-coated microchannel in vitro; or
   c) both a) and b). In some aspects, provided herein is a method for administering freeze-dried platelet derivatives to a subject having Hermansky Pudlak Syndrome (HPS). In some aspects, provided herein is a method for administering freeze-dried platelet derivatives to a subject having Bernard Soulier Syndrome (BSS). In some embodiments, the subject is bleeding at the start of the administering.

In another aspect, provided herein is a platelet derivative composition for use in administering to a subject having Hermansky Pudlak Syndrome (HPS) or Bernard Soulier Syndrome (BSS), wherein the administering comprises administering an effective dose of platelet derivatives in the platelet derivative composition to the subject. In some aspects, provided herein is a platelet derivative composition for use in administering to a subject having Hermansky Pudlak Syndrome (HPS). In some aspects, provided herein is a platelet derivative composition for use in administering to a subject having Bernard Soulier Syndrome (BSS). In some embodiments, the subject is bleeding at the start of the administering.

In some embodiments of any of the aspects herein that include administering, the method for administering platelet derivatives to a subject further comprises wherein the subject is being administered:
a) one or more anti-coagulants,
b) one or more anti-platelet agents,
c) aspirin,
d) two or more of a), b), or c),
e) a triple therapy comprising one of a), b), and c), or
f) a), b), and c). In some embodiments, the subject is being administered a combination of one or more anti-coagulants, and one or more anti-platelet agents. In some embodiments, the subject is being administered a combination of aspirin and one or more anti-platelet agents. In some embodiments, the subject is being administered a combination of aspirin and one or anti-coagulants.

In some embodiments, the method or platelet derivative composition for use of further comprises before the administering, rehydrating the freeze-dried platelet derivatives to form a rehydrated platelet derivative composition, and wherein the administering is administering an effective dose of the rehydrated platelets from the rehydrated platelet derivative composition to the subject.

To reiterate, any embodiments herein in this section and in this specification and associated claims, can be combined and/or used in any of the aspects herein and in combination with any of the other embodiments herein. Furthermore, a “powder” recited in any aspect or embodiment can alternatively be a solid, or a composition comprising less than 1% water content in such aspect or embodiment.

In any of the aspects and embodiments, anti-HLA antibodies are present in blood of the subject. In some embodiments, anti-HPA antibodies are present in blood of the subject. In some embodiments, anti-HLA antibodies and anti-HPA antibodies are present in blood of the subject. In some embodiments, the anti-HLA and/or anti-HPA antibodies comprise IgG antibodies. In some embodiments, the anti-HLA antibodies in blood of the subject are against one or more HLA selected from the group consisting of HLA Class-1 and HLA Class-2. In some embodiments, the anti-HPA antibodies in blood of the subject are against one or more HPA selected from the group consisting of HPA-1, HPA-2, HPA-3, HPA-4, HPA-5, and HPA-15, and sub-types thereof. In some embodiments, the anti-HPA antibodies are cross-reactive against the platelet derivatives.

In any of the aspects and embodiments, the platelet derivatives herein are capable of yielding comparable in vitro thrombin generation activity in the presence versus absence of anti-HLA and/or anti-HPA antibodies. In some embodiments, the platelet derivatives herein are capable of yielding comparable in vitro thrombin generation activity in the presence versus absence of anti-HPA antibodies. In some embodiments, the platelet derivatives herein are capable of yielding comparable in vitro thrombin generation activity in the presence versus absence of anti-HPA antibodies that are cross-reactive with the platelet derivatives. In some embodiments, the platelet derivatives are capable of yielding comparable in vitro thrombin generation activity in the presence versus absence of cross-reactive antibodies against the platelet derivatives. In some embodiments, the thrombin generation activity is selected from the group consisting of peak thrombin, lag time until thrombin generation, endogenous thrombin potential, and time to peak thrombin generation. In some embodiments, the platelet derivatives are capable of yielding +/−5%, 10%, 15%, 20%, 25%, 30%, 35%, 40%, 45%, 50%, 55%, or 60% thrombin generation activity in the presence versus absence of anti-HLA and/or anti-HPA antibodies. In some embodiments, the platelet derivatives are capable of yielding +/−5%, 10%, 15%, 20%, 25%, 30%, 35%, 40%, 45%, 50%, 55%, or 60% peak thrombin in the presence versus absence of anti-HLA and/or anti-HPA antibodies. In some embodiments, the platelet derivatives are capable of yielding +/−5%, 10%, 15%, 20%, 25%, 30%, 35%, 40%, 45%, 50%, 55%, or 60% lag time until thrombin first generated in the presence versus absence of anti-HLA and/or anti-HPA antibodies. In some embodiments, the platelet derivatives are capable of yielding +/−5%, 10%, 15%, 20%, 25%, 30%, 35%, 40%, 45%, 50%, 55%, or 60% endogenous thrombin potential in the presence versus absence of anti-HLA and/or anti-HPA antibodies. In some embodiments, the platelet derivatives are capable of yielding +/−5%, 10%, 15%, 20%, 25%, 30%, 35%, 40%, 45%, 50%, 55%, or 60% time to peak in the presence versus absence of anti-HLA and/or anti-HPA antibodies.

In any of the aspects and embodiments, the platelet derivatives are capable of yielding comparable hemostatic activity in an in vitro T-TAS assay in the presence versus absence of anti-HLA and/or anti-HPA antibodies. In some embodiments, the platelet derivatives are capable of yielding comparable hemostatic activity in an in vitro T-TAS assay in the presence versus absence of anti-HPA antibodies. In some embodiments, the platelet derivatives are capable of yielding comparable hemostatic activity in an in vitro T-TAS assay in the presence versus absence of anti-HPA antibodies that are cross-reactive with the platelet derivatives. In some embodiments, the platelet derivatives are capable of yielding +/−5%, 10%, 15%, 20%, 25%, 30%, 35%, 40%, 45%, 50%, 55%, or 60% occlusion start time in an in vitro T-TAS assay in the presence versus absence of anti-HLA and/or anti-HPA antibodies. In some embodiments, the platelet derivatives are capable of yielding +/−5%, 10%, 15%, 20%, 25%, 30%, 35%, 40%, 45%, 50%, 55%, or 60% are under the curve (AUC) in an in vitro T-TAS assay in the presence versus absence of anti-HLA and/or anti-HPA antibodies. In some embodiments, the platelet derivatives are capable of yielding comparable hemostatic activity in an in vitro T-TAS assay in the presence versus absence of cross-reactive antibodies against the platelet derivatives. In some embodiments, the platelet derivative composition has the ability to occlude, and wherein the ability to occlude is determined by a thrombus-formation analysis system (T-TAS) assay. In some embodiments, when at a concentration of 70×10³ particles/μL, the platelet derivative composition produce an occlusion time of less than 20 minutes in a T-TAS assay.

In any of the aspects and embodiments, the platelet derivatives have less amount of CD42 as compared to the amount of CD42 in fresh platelets or apheresis platelets. In some embodiments, the platelet derivatives have 12% to 64%, 20% to 64%, 20% to 60%, 20% to 50%, or 25% to 50% amount of CD42 as compared to the amount of CD42 in fresh platelets or apheresis platelets. In some embodiments, the platelet derivatives have no detectable exogenous cross-linking on the membranes via proteins and/or lipids present on the membranes. In some embodiments, the platelet derivatives have no exogenous cross-linking on the membranes via proteins and/or lipids present on the membranes. In some embodiments, the platelet derivatives have less than 5%, 4%, 3%, 2%, 1%, 0.5%, 0.3%, 0.1%, 0.05%, or 0.01% cross-linking on the membranes via proteins and/or lipids present on the membranes. In some embodiments, the platelet derivatives have between 0.001-5%, 0.001-3%, 0.001-1%, 0.001-0.5%, 0.001-0.4%, 0.001-0.3%, 0.001-0.2%, 0.001-0.1%, or 0.001-0.01% cross-linking on the membranes via proteins and/or lipids present on the membranes.

In any of the aspects and embodiments, the subject is bleeding at the start of the administering. In some embodiments, the administering leads to cessation of the bleeding. In some embodiments, the subject has HPS. In some embodiments, the subject has BSS. In some embodiments, the subject is refractory to platelet transfusion. In some embodiments, the subject can be refractory to platelet transfusion and has one or more indication discloses elsewhere herein. In some embodiments, the subject has HPS and is refractory to platelet transfusion. In some embodiments, the subject has BSS and is refractory to platelet transfusion. In some embodiments, the subject has BSS, wherein the administering is performed to treat the subject, and wherein the ability to occlude a collagen-coated microchannel in vitro is restored or improved in the subject after the administering compared to before the administering. In some embodiments, the administering of the platelet derivatives leads to an improvement in thrombin generation in the subject as compared to the subject before the administering and/or an improvement in clot formation in the subject as compared to the subject before the administering. In some embodiments, the administering of the platelet derivatives leads to an improvement in clot formation in the subject as compared to the subject after being administered apheresis platelets, but before the administering of the platelet derivatives. In some embodiments, the administering of the platelet derivatives is performed to treat the subject, and wherein normal levels of hemostasis and/or HPS-related biomarker abnormalities are maintained in the subject. In some embodiments, the subject is taking a) one or more anti-coagulants, b) one or more anti-platelet agents, or both a) and b). In some embodiments, the subject is bleeding at the start of administering, and the administering leads to a decrease in bleeding within 15, 30, 45 minutes, 1, 2, 3, 4, 5, 6, 8, 10, 12, 14, 16, 18, 20, 22, or 24 hours after the start of the administering. In some embodiments, the administering is performed until the bleeding stops. In some embodiments, the administering is performed for more than 2, 3, 4, 5, 6, 8, 10, 12, 14, 16, 18, 20, 22, or 24 hours from the start of the first dose of the platelet derivatives. In some embodiments, the administering is performed for less than 6, 8, 10, 12, 14, 16, 18, 20, 22, or 24 hours from the start of the first dose of the platelet derivatives. In some embodiments, the administering is performed until there is cessation of bleeding at a primary bleeding site. In some embodiments, the administering is performed until there is cessation of bleeding at a bleeding site other than the primary bleeding site. In some embodiments, the administering is performed until there is cessation of bleeding at all of the bleeding sites. In some embodiments, the administering is performed until there is a decrease in bleeding to less than WHO grade 2A at a primary bleeding site. In some embodiments, the administering is performed until there is a decrease in bleeding to less than WHO grade 2A at a bleeding site other than the primary bleeding site. In some embodiments, the administering is performed until there is a decrease in bleeding to less than WHO grade 2A at all the bleeding site. In some embodiments, the administering is performed until there is a decrease in bleeding to less than WHO grade 2A at least one of the bleeding site. In some embodiments, the administering leads to a decrease in bleeding to less than WHO grade 2A, or cessation of bleeding in a primary bleeding site, or any site other than the primary bleeding site within 15, 30, 45, 60 minutes, 1, 2, 4, 6, 8, 10, 12, 14, 16, 18, 20, 22, or 24 hours after the administering of the platelet derivatives.

In some embodiments of any of the aspects or embodiments herein that include platelet derivatives, method for preparing platelet derivatives, platelet derivatives for use, or a method for administering the platelet derivatives, the platelet derivatives have at least one, two, three, or in illustrative embodiments all of the following: (i) less than 5% of CD 41-positive platelet derivatives are microparticles having a diameter of less than 0.5 μm, (ii) at least 65% of the platelet derivates are CD 42 positive, (iii) the composition comprises a population of platelet derivatives having a reduced propensity to aggregate such that no more than 10% of the platelet derivatives in the population aggregate under aggregation conditions comprising an agonist but no platelets, and no divalent cation; (iv) at least 50% of the platelet derivatives in the composition are at least 0.5 μm in diameter by scattering intensity; and (v) at least 50% of the platelet derivatives in the composition are between 0.5 μm and 25 μm in diameter by scattering intensity.

In some embodiments of any of the aspects and embodiments herein that include platelet derivatives, the platelet derivatives have a reduced propensity to aggregate, wherein no more than 25%, 10%, 5%, 4%, 3%, or in illustrative embodiments, no more than 2%, of the platelet derivatives in the population aggregate under aggregation conditions comprising an agonist but no platelets, and in illustrative embodiments in the absence of a divalent cation; and in certain illustrative embodiments have one or more, two or more, or all of the following characteristics of a super-activated platelet selected from: a. the presence of thrombospondin (TSP) on their surface at a level that is greater than on the surface of resting platelets; b. the presence of von Willebrand factor (vWF) on their surface at a level that is greater than on the surface of resting platelets; c. an inability to increase expression of a platelet activation marker in the presence of an agonist as compared to the expression of the platelet activation marker in the absence of an agonist.

In some embodiments of any of the aspects and embodiments herein that include a composition comprising platelet derivatives, less than 15%, and in certain non-limiting illustrative embodiments less than 5% of the CD 41-positive platelet derivatives are microparticles, in non-limiting illustrative embodiments having a diameter of less than 1 μm, and in certain non-limiting illustrative embodiments less than 0.5 μm, and the platelet derivatives are capable of generating thrombin.

In some embodiments of any of the aspects and embodiments herein that include platelet derivatives in a platelet derivative composition in the form of a powder, the powder comprises trehalose in the range of 20-35% by weight, polysucrose in the range of 45-60% by weight, and platelet derivatives in the range of 0.5-20% by weight, and in illustrative embodiments wherein the platelet derivatives to microparticles have a numerical ratio of at least 95:1 in the platelet derivative composition, and wherein the platelet derivatives are capable of generating thrombin in an in vitro assay.

In some embodiments of any of the aspects and embodiments herein that include platelet derivatives in a platelet derivative composition in the form of a powder, The composition comprises trehalose in the range of 20-35% by weight, polysucrose in the range of 45-60% by weight, and platelet derivatives in the range of 0.5-20% by weight, wherein the platelet derivative composition comprises a population of platelet derivatives having a reduced propensity to aggregate such that no more than 25%, 10%, 5%, 4%, 3%, or in illustrative embodiments, no more than 2%, of the platelet derivatives in the population aggregate under aggregation conditions comprising an agonist but no platelets and in illustrative embodiments, no divalent cations, and wherein platelet derivatives therein further have one or both of: the presence of thrombospondin (TSP) on their surface at a level that is greater than on the surface of resting platelets; and the presence of von Willebrand factor (vWF) on their surface at a level that is greater than on the surface of resting platelets.

In some embodiments of any of the aspects and embodiments herein that include a process for preparing freeze-dried platelets, the process comprises a) preparing a composition comprising platelets and an aqueous medium using the process comprising: tangential flow filtration (TFF) of a starting material comprising platelets, a diluted starting material comprising platelets, a concentrated platelet composition, or a combination thereof, thereby preparing a composition comprising platelets or platelet derivatives and aqueous medium, wherein the aqueous medium has a protein concentration less than or equal to 50% of the protein concentration of donor apheresis plasma; and optionally b) freeze-drying the composition comprising platelets and the aqueous medium.

In some embodiments of any of the aspects and embodiments herein that include a process for preparing a composition, the process comprises: diluting a starting material comprising platelets to form a diluted starting material; concentrating the diluted starting material such that the platelets have a concentration of about 2250×10³ cells/μL (±250×10³) to form a concentrated platelet composition; and washing the concentrated platelet composition with at least 2 diavolumes (DV) of a preparation agent to form a TFF-treated composition.

In some embodiments of any of the aspects and embodiments herein that include a process for preparing a platelet derivative composition, performing tangential flow filtration (TFF) of a platelet composition with a preparation agent, in a preparation agent, or in a solution that optionally can have components of the preparation agent or can be a different solution, the TFF is performed to at least partially exchange the solution with the preparation agent having a pH in the range of 5.5 to 8.0 and comprising 0.4 to 35% trehalose and 2% to 8% polysucrose, wherein said TFF is performed using a 0.3 to 1 micron filter, to at least partially or fully exchange the platelets into the preparation agent, thereby preparing a TFF-treated composition comprising 100×10³ to 20,000×10³ platelets/μl, in certain illustrative embodiments between 10,000×10³ to 20,000×10³ platelets/μl, in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm; freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried composition to form the platelet derivative composition, wherein the platelet derivatives in the platelet derivative composition are capable of generating thrombin. In illustrative embodiments the platelet derivatives are capable of generating thrombin

In some embodiments of any of the aspects and embodiments herein that include a process for preparing a platelet derivative composition, the TFF is performed to at least partially exchange the solution with the preparation agent having a pH in the range of 5.5 to 8.0 and comprising 0.4 to 35% trehalose and 2% to 8% polysucrose, wherein the TFF is performed using a 0.3 to 1 micron filter, to at least partially or fully exchange the platelets into the preparation agent, thereby preparing a TFF-treated composition comprising 100×10³ to 20,000×10³ platelets/μl, in certain illustrative embodiments between 10,000×10³ to 20,000×10³ platelets/μl, in an aqueous medium having less than or equal to 15% plasma protein, and having less than 15%, and in certain non-limiting illustrative embodiments less than 5.0% microparticles by scattering intensity having a diameter of less than 1 μm, and in certain non-limiting illustrative embodiments less than 0.5 μm, freeze drying the TFF-treated composition comprising platelets in the aqueous medium to form a freeze-dried composition comprising platelet derivatives; and heating the freeze-dried composition at a temperature in the range of 60° C. to 90° C. for at least 1 hour to not more than 36 hours to thermally treat the platelet derivatives in the freeze-dried platelet composition to form the platelet derivative composition, wherein the platelet derivative composition is: a) negative for HLA Class I antibodies based on a regulatory agency approved test for HLA Class I antibodies; b) negative for HLA Class II antibodies based on a regulatory agency approved test for HLA Class II antibodies; and c) negative for HNA antibodies based on a regulatory agency approved test for HNA antibodies. The platelets can be diluted 1:0.5, 1:1, 1:2, 1:5, or 1:10 in a solution or in the preparation agent before performing the TFF.

In some embodiments of any aspects and embodiments herein that include a composition, the platelet derivatives have a compromised plasma membrane and a reduced propensity to aggregate such that no more than 2%, 3%, 4%, 5%, 6%, 7%, 8%, 9%, 10%, or 25% of the platelet derivatives in the population aggregate under aggregation conditions comprising an agonist but no platelets, and in illustrative embodiments no divalent cations, wherein at least 30%, 40%, 50%, 60%, 70%, or 80% of the platelet derivatives are CD 41-positive platelet derivatives, wherein less than 1%, 2%, 3%, 4%, or 5% of the CD 41-positive platelet derivatives are microparticles having a diameter or radius of less than 0.3 μm, 0.4 μm, 0.5 μm, 0.7 μm, or 1 μm and wherein the platelet derivatives are capable of generating thrombin in vitro.

In certain illustrative embodiments of a composition, or in some compositions used in or formed by a process, the platelet derivatives in a composition, as a non-limited example a powder, and/or formed by a process disclosed herein, are surrounded by a compromised plasma membrane, are positive for CD 41, and/or are 0.5 to 25.0 μm, 20.0 μm, 15.0 μm, 12.5 μm, 10.0 μm, or 2.5 μm in radius or diameter. In some embodiments, the composition comprises platelet derivatives such that at least 95% platelet derivatives positive for CD 41 have a radius or diameter in the range of 0.5 to 25.0 μm, 20.0 μm, 15.0 μm, 12.5 μm, 10.0 μm, or 2.5 μm. Such radius or diameter can be measured, for example by flow cytometry technique as known to a skilled artisan in the art.

In some embodiments of any of the aspects and embodiments herein that include platelet derivatives in a hydrated or rehydrated form, in illustrative wherein tangential flow is included in a method for producing the platelet derivatives, the protein concentration, or plasma protein concentration, is in the range of 0.01%-50%, 5%-50%, 5%-30%, 5-15%, 8%-10%, 7%-10%, or 3-7% of the protein concentration of donor apheresis plasma. In some embodiments of a composition or in some compositions used in or formed by a process herein, the protein concentration, or plasma protein concentration is less than or equal to 30%, 25%, 20%, 15%, 10%, 5%, 4%, 3%, 2%, or 1% of the protein concentration of donor apheresis plasma. In some embodiments of a composition or a process herein, the protein concentration, or plasma protein concentration is less than or equal to 15%, 14%, 13%, 12%, 11%, 10%, 9%, 8%, 7%, 6%, 5%, 4%, 3%, 2%, 1%, 0.1%, or 0.01%. In some exemplary embodiments, the protein concentration, or plasma protein concentration is less than 3% or 4%. In some embodiments, the protein concentration, or plasma protein concentration is between 0.01% and 20%, 0.01% and 15%, 0.01% and 10%, 0.01% and 5%, 0.1% and 20%, 0.1% and 15%, 0.1% and 10%, 0.1% and 5%, 1% and 20%, 1% and 15%, 1% and 10%, 1% and 5%, 2% and 10%, 2% and 5%, 2.5% and 5%, 2.5% and 7.5%, or between 3% and 5%. In some embodiments of a composition or a process herein, the protein concentration is in the range of 0.01-15%, 0.1-15%, 1-15%, 1-10%, 0.01-10%, 3-12%, or 5-10%. In some embodiments, the absorbance at 280 nm is less than or equal to 2.0 AU, or 1.90 AU, or 1.80 AU, or 1.7 AU, or 1.66 AU, or 1.6 AU when measured using a path length of 0.5 cm.

In some embodiments of any of the aspects and embodiments herein that include platelet derivatives in a powdered form, the protein concentration is in the range of 0.01-15%, 0.1-15%, 1-15%, 1-10%, 0.01-10%, 3-12%, or 5-10%. In some embodiments, the protein concentration is less than or equal to 25%, 20%, 15%, 10%, 7.5%, 5%, 2.5%, 1%, or 0.1%.

In some embodiments of any of the aspects and embodiments herein that include a process for preparing a platelet derivative composition, the process comprises performing TFF of a platelet composition in a solution to at least partially exchange the solution with a preparation agent having a pH in the range of 5.5 to 8.0 and comprising trehalose and polysucrose, wherein said TFF is performed using a 0.3 to 1 micron filter, thereby preparing a TFF-treated composition.

In some embodiments of any of the aspects and embodiments herein that include a composition or in some compositions used in or formed by a process that includes a population of platelet derivatives in a hydrated or rehydrated form, the composition comprises less than 10%, 7.5%, 7%, 6%, 5%, 4.5%, 4%, 3.5%, 3%, 2.5%, 2%, 1.5%, 1.0%, 0.75%, 0.5%, 0.25%, or 0.1% (by scattering intensity) microparticles. In some embodiments, the composition comprises microparticles (by scattering intensity) in the range of 0.01%-10%, 0.01%-7.5%, 0.01%-5%, 0.1%-10%, 0.1%-5%, 0.1%-4.9%, 0.5%-4.5%, 1%-10%, 1%-5%, 0.01%-4%, −0.1%-4%, 1%-4%, 1.5%-3%, 0.1%-3%, or 1%-3%. In some embodiments, the microparticles have a diameter less than 1 μm. In illustrative embodiments, the microparticles have a radius or diameter less than 0.5 μm. In some embodiments, the microparticles have a radius or diameter in the range of 0.01-0.5 μm, 0.1-0.5 μm, or 0.1-0.49 μm, 0.1-0.47 μm, or 0.1-0.45 μm, or 0.1-0.4 μm, or 0.2-0.49 μm, or 0.25-0.49 μm, or 0.3-0.47 μm. In some embodiments, the radius or diameter of the microparticles is measured using flow cytometry.

In some embodiments of any of the aspects and embodiments herein that include a platelet derivative composition in a powdered form, the platelet derivative composition comprises a population of platelet derivatives comprising CD41-positive platelet derivatives, wherein less than 5%, 4.5%, 4%, 3.5%, 3%, 2.5%, 2%, 1.5%, 1%, 0.5%, or 0.1% of the CD41-positive platelet derivatives are microparticles having a diameter of less than 1 μm, 0.9 μm, 0.8 μm, 0.7 μm, 0.6 μm, 0.5 μm 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-41-positive platelet derivatives are microparticles. In some embodiments, the platelet derivative composition comprises a population of platelet derivatives comprising CD42-positive platelet derivatives, wherein less than 5%, 4.5%, 4%, 3.5%, 3%, 2.5%, 2%, 1.5%, 1%, 0.5%, or 0.1% of the CD42-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-42-positive platelet derivatives are microparticles. In some embodiments, the platelet derivative composition comprises a population of platelet derivatives comprising CD61-positive platelet derivatives, wherein less than 5%, 4.5%, 4%, 3.5%, 3%, 2.5%, 2%, 1.5%, 1%, 0.5%, or 0.1% of the CD61-positive platelet derivatives are microparticles having a diameter of less than 1 μm, 0.9 μm, 0.8 μm, 0.7 μm, 0.6 μm, 0.5 μm, 0.4 μm, 0.3 μm, 0.2 μm, or 0.1 μm. In some embodiments, 0.01%-4.9%, 0.1%-4.9%, 0.5%-4.5%, 0.01%-4%, 0.1%-4%, 1%-4%, 1.5%-3%, 0.1%-3%, or 1%-3% of the CD-62-positive platelet derivatives are microparticles. In some illustrative embodiments, the microparticles are having a diameter of less than 0.5 μm. In some embodiments of any of the aspects and embodiments herein that include a platelet derivative composition in a powdered form, the diameter of the microparticles is determined after rehydrating the platelet derivative composition with an appropriate solution. In some embodiments, the amount of solution for rehydrating the platelet derivative composition is equal to the amount of buffer or preparation agent present at the step of freeze-drying. In some embodiments, the diameter of the microparticles is determined by flow cytometry.

In some embodiments of any of the aspects and embodiments herein that include a composition or in some compositions used in or formed by a process, the platelet derivatives have a radius or diameter greater than 0.25 μm, greater than 0.3 μm, greater than 0.4 μm, or in illustrative embodiments, greater than 0.5 μm. In some embodiments, the platelet derivatives have a radius or diameter greater than 0.75 μm. In some embodiments, the platelet derivatives have a radius or diameter in the range of 0.25-4 μm, 0.27-3.5 μm, 0.3-3.25 μm, 0.35-3.50 μm, or 0.4-3 μm. In illustrative embodiments, the platelet derivatives have a radius or diameter of at least 0.5 μm, for example in the range of 0.5 μm on the low end of the range to 25.0 μm, 20.0 μm, 15.0 μm, 12.5 μm, 10.0 μm, 5.0 μm or 2.5 μm on the high end of the range. In some embodiments, the diameter of the platelet derivatives is measured using flow cytometry.

In some embodiments of any of the aspects and embodiments herein that include a composition or in some compositions used in or formed by a process, the platelet derivatives are CD-41 positive. In some embodiments, at least 40%, 45%, 50%, 55%, 60%, 65%, 70%, or 75% of the platelet derivatives are CD-41 positive. In some embodiments, the platelet derivatives in the range of 35-97%, 40-97%, 50-97%, 60-97%, 40-95%, 45-90%, 50-95%, 60-90%, or 75-95% are positive for CD-41. In some embodiments, at least 50% of the platelet derivatives are CD 41-positive platelet derivatives, wherein less than 5% of the CD 41-positive platelet derivatives are microparticles having a diameter of less than 0.5 μm, and wherein the platelet derivatives have a potency of at least 1.5 thrombin generation potency units (TGPU) per 10⁶ platelet derivatives.

In some embodiments of any of the aspects and embodiments herein that include a platelet derivative composition in a powdered form, the platelet derivatives in the platelet derivative composition have a weight percentage of at least 0.5%, 0.75%, 1%, 1.5%, 2%, 2.5% 3%, 3.5%, 4%, 4.5%, 5%, 5.5%, 6%, 6.5%, 7%, 7.5%, 8%, 8.5%, 9%, 9.5%, or 10%. In some embodiments, the platelet derivatives in the platelet derivative composition have a weight percentage in the range of 0.5 to 25%, 0.5% to 20%, 1% to 20%, 2.5% to 20%, 5% to 20%, 5% to 10%, 2.5% to 20%, 2.5% to 15%, 2.5% to 10%, or 2.5% to 7.5%.

In some embodiments of the aspects and embodiments herein that include a platelet derivative composition in a powdered form, a platelet derivative composition is devoid of plasma protein. In some embodiments, the plasma protein is in the range of 0.01-15%, 0.1-15%, 1-10%, 2-15%, 3-9%, 1-5%, 1-3%, 0.1-3%, 0.5-2%, or 0.25-2%.

In some embodiments of the aspects and embodiments herein that include a platelet derivative composition in a powdered form, a platelet derivative composition comprises a buffering agent in the range of 0.5-3%, 0.75-2.75%, 1-2.5%, or 1.5-2.5%. In some embodiments, the buffering agent is HEPES.

In some embodiments of any of the aspects and embodiments herein that include a composition or in some compositions used in or formed by a process, the platelet derivatives have an inability to increase expression of a platelet activation marker in the presence of an agonist as compared to the expression of the platelet activation marker in the absence of the agonist. In some embodiments, the platelet activation marker is selected from the group consisting of Annexin V, and CD 62. In some embodiments, the platelet activation marker is Annexin V. In some embodiments, the platelet activation marker is CD 62. In some embodiments, the agonist is selected from the group consisting of collagen, epinephrine, ristocetin, arachidonic acid, adenosine di-phosphate, and thrombin receptor associated protein (TRAP).

In some embodiments of any of the aspects and embodiments herein that include a composition, or in some compositions used in or formed by a process, the aqueous medium has a concentration of human leukocyte antigen (HLA) Class I antibodies that is less than 30%, 10%, 5%, 3%, or 1% of the human leukocyte antigen (HLA) Class I antibody concentration in donor apheresis plasma. In some embodiments, the aqueous medium has a concentration of human leukocyte antigen (HLA) Class II antibodies that is less than 30%, 10%, 5%, 3%, or 1% of the human leukocyte antigen (HLA) Class II antibody concentration in donor apheresis plasma. In some embodiments, the composition is negative for HLA Class I antibodies based on a regulatory agency approved test. In some embodiments, the composition is negative for HLA Class II antibodies based on a regulatory agency approved test. In some embodiments of the composition, a percentage of beads positive for HLA Class I antibodies, as determined for the composition by flow cytometry using beads coated with Class I HLAs, is less than 5%, 3%, or 1%. In some embodiments of the composition, a percentage of beads positive for HLA Class II antibodies, as determined for the composition by flow cytometry using beads coated with Class II HLAs is less than 5%, 3%, or 1%.

In some embodiments of any of the aspects and embodiments herein that include a composition, or in some compositions used in or formed by a process, the aqueous medium has a concentration of human neutrophil antigen (HNA) antibodies that is less than 30%, 10%, 5%, 3%, or 1% of the HNA antibody concentration in donor apheresis plasma. In some embodiments, the composition is negative for HNA antibodies based on a regulatory agency approved test. In some embodiments of the composition, a percentage of beads positive for HNA antibodies, as determined for the composition by flow cytometry using beads coated with HNAs is less than 5%, 3%, or 1%.

In some embodiments of any of the aspects and embodiments herein that include a composition, or in some compositions used in or formed by a process, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments of the composition, a percentage of beads positive for HLA Class I antibodies, HLA Class II antibodies, and HNA antibodies, as determined for the composition by flow cytometry using beads coated with Class I HLAs, Class II HLAs, or HNAs, respectively, is less than 5%, 3%, or 1%. In some embodiments, the composition is negative for the antibodies selected from the group consisting of HLA Class I antibodies, HLA Class II antibodies, and HNA antibodies based on a regulatory agency approved test for the respective antibodies. In some embodiments, the composition is negative for HLA Class I antibodies, HLA Class II antibodies, and HNA antibodies based on a regulatory agency approved test for the respective antibodies.

In some embodiments of any of the aspects and embodiments herein that include a composition, or in some compositions used in or formed by a process that includes a population of platelet derivatives in a hydrated or rehydrated form, the platelets or platelet derivatives in a composition are at least 100×10³ platelets/μL, or 200×10³ platelets/μL, or 400×10³ platelets/μL, or 1000×10³ platelets/μL, or 1250×10³ platelets/μL, or 1500×10³ platelets/μL, or 1750×10³ platelets/μL, 2000×10³ platelets/μL, or 2250×10³ platelets/μL, or 2500×10³ platelets/μL, or 2750×10³ platelets/μL, or 3000×10³ platelets/μL, 3250×10³ platelets/μL, 3500×10³ platelets/μL, 3750×10³ platelets/μL, 4000×10³ platelets/μL, or 4250×10³ platelets/μL, or 4500×10³ platelets/μL, or 4750×10³ platelets/μL, or 5000×10³ platelets/μL, or 5250×10³ platelets/μL, or 5500×10³ platelets/μL, or 5750×10³ platelets/μL, or 6000×10³ platelets/μL, or 7000×10³ platelets/μL, or 8000×10³ platelets/μL, or 9000×10³ platelets/μL, or 10,000×10³ platelets/μL, or 11,000×10³ platelets/μL, or 12,000×10³ platelets/μL, or 13,000×10³ platelets/μL, or 14,000×10³ platelets/μL, or 15,000×10³ platelets/μL, or 16,000×10³ platelets/μL, or 17,000×10³ platelets/μL, or 18,000×10³ platelets/μL, or 19,000×10³ platelets/μL, or 20,000×10³ platelets/μL. In some embodiments of the composition, the platelets or platelet derivatives in the composition is in the range of 100×10³-20,000×10³ platelets/μL, 1000×10³-20,000×10³ platelets/μL, 1000×10³-10,000×10³ platelets/μL, 500×10³-5,000×10³ platelets/μL, 1000×10³-5,000×10³ platelets/μL, 2000×10³-8,000×10³ platelets/μL, 10,000×10³-20,000×10³ platelets/μL, 15,000×10³-20,000×10³ platelets/μL, 5000×10³ to 20,000×10³ platelets/μl, 6000×10³ to 18,000×10³ platelets/μl or 6000×10³ to 15,000×10³ platelets/μl.

In some embodiments, the above concentrations are at any point in a process herein, such as in the volume that is freeze dried. In some embodiments, the above concentrations are for platelet-derivatives herein. It is contemplated that the platelet derivative composition in the form of a powder has to be rehydrated with a solution to determine the platelet-derivative concentration, typically in the intended volume for rehydration of a powder, e.g. freeze-dried, composition, which in illustrative embodiments is a recommended volume of a container containing the powder and/or a same volume as the composition was in before it was dried to form the powder. In some embodiments, the solution for rehydrating can be water. In some embodiments, the solution for rehydrating can be a well-known buffer. In some embodiments, the amount of solution for rehydrating the platelet derivative composition is equal to the amount of buffer or preparation agent present at the step of freeze-drying. In some embodiments, the platelet concentration is in

In some embodiments of any of the aspects and embodiments herein that include a composition, or in some compositions used in or formed by a process, the composition(s) comprises a population of platelet derivatives having a reduced propensity to aggregate. In certain embodiments, no more than 25%, 22.5%, 20%, 17.5%, 12.5%, 10%, 7.5%, 5%, 4%, 3%, 2%, or 1% of the platelet derivatives in the population aggregate under aggregation conditions. In an illustrative embodiment no more than 10% of the platelet derivatives in the population aggregate under aggregation conditions. Illustrative embodiments of exemplary aggregation conditions are provided herein. For example, in illustrative embodiments such aggregation conditions comprise an agonist but no platelets and no divalent cations are present in the aggregation conditions. In some embodiments, the agonist is selected from the group consisting of collagen, epinephrine, ristocetin, arachidonic acid, adenosine di-phosphate, and thrombin receptor associated protein (TRAP). In some embodiments, the population of platelet derivatives aggregate in the range of 2-30%, 5-25%, 10-30%, 10-25%, 12.5-25%, 2-10%, 2-8%, 2-7.5%, 2-5%, 2-4%, 0-1%, 0-2%, 0-3%, 0-4%, 0-5%, 0-7.5%, or 0-10%, or in illustrative embodiments 0 to about 1% of the platelet derivatives under aggregation conditions comprising an agonist but no platelets. It can be contemplated that aggregation conditions involve rehydrating the platelet derivative composition in an appropriate amount of water or an appropriate buffer.

In some embodiments of any of the aspects and embodiments herein that include a composition, or in some compositions used in or formed by a process, comprises erythrocytes in an amount lesser than 0.2×10⁶ erythrocytes/μL, or 0.1×10⁶ erythrocytes/μL, or 0.5×10⁵ erythrocytes/μL, or 0.1×10⁷ erythrocytes/μL. In some embodiments, the erythrocytes in the composition is in the range of 0.1×10⁵ erythrocytes/μL to 0.2×10⁶ erythrocytes/μL, or 0.5×10⁷ erythrocytes/μL to 0.1×10⁶ erythrocytes/μL.

In some embodiments of any of the aspects and embodiments herein that include a composition, or in some compositions used in or formed by a process, the aqueous medium further comprises a buffering agent, a base, a loading agent, optionally a salt, and optionally at least one organic solvent. In some embodiments, the buffering agent is HEPES (4-(2-hydroxyethyl)-1-piperazineethanesulfonic acid). In some embodiments, the base is sodium bicarbonate. In some embodiments, the loading agent is a monosaccharide, a polysaccharide, or a combination thereof. In some embodiments, the monosaccharaide is selected from the group consisting of sucrose, maltose, trehalose, glucose, mannose, and xylose. In some embodiments, the monosaccharide is trehalose. In some embodiments, the polysaccharide is polysucrose. In some embodiments, the salt is sodium chloride, potassium chloride, or a combination thereof. In some embodiments, the organic solvent is selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

In some embodiments of any of the aspects and embodiments herein that include a composition, or in some compositions used in or formed by a process that includes a population of platelet derivatives in a hydrated or rehydrated form, comprises trehalose in the range of 0.4-35%, or 1-35%, or 2-30%, 1-20%, or 1-10%, or 1-5%, or 0.5-5%. In an exemplary embodiment, the composition comprises 3.5% trehalose.

In some embodiments of any of the aspects and embodiments herein that include a composition, or in some compositions used in or formed by a process that includes a platelet composition in a powdered form, comprises trehalose having a weight percentage in the range of 10-60%, 15-55%, 20-60%, 20-50%, 25-60%, 25-50%, 10-50%, 20-40%, 20-35%, or 1-20%. In some embodiments, the weight percentage of trehalose can vary on the weight percentage of other components in the composition like, polysucrose, platelet derivatives, plasma protein, and buffering agents.

In some embodiments of any of the aspects and embodiments herein that include a composition, or in some compositions used in or formed by a process that includes a population of platelet derivatives in a hydrated or rehydrated form, comprises polysucrose in the range of 2-8%, 2.25-7.75%, 2.5-7.5%, or 2.5-6.5%. In an exemplary embodiment, the composition comprises 3% polysucrose. In another exemplary embodiment, the composition comprises 6% polysucrose.

In some embodiments of any of the aspects and embodiments herein that include a composition, or in some compositions used in or formed by a process that includes a platelet composition in a powdered form, comprises polysucrose having a weight percentage in the range of 20-80%, 25-75%, 30-70%, 35-65%, 30-80%, or 45-60%. In some embodiments, the weight percentage of trehalose can vary on the weight percentage of other components in the composition like, trehalose, platelet derivatives, plasma protein, and buffering agents.

In some embodiments of any of the aspects and embodiments herein that include a composition, or in some compositions used in or formed by a process that includes a platelet composition in a powdered form, comprises trehalose and polysucrose having a combined weight percentage in the range of 30-95%, 35-95%, 40-90%, 40-90%, 45-90%, or 60-95%.

In some embodiments of any of the aspects and embodiments herein that include a composition or in some compositions used in or formed by a process herein, comprises polysucrose, the polysucrose is a cationic form of polysucrose. In some embodiments, the cationic form of polysucrose is diethylaminoethyl (DEAE)-polysucrose. In some embodiments, the polysucrose is an anionic form of polysucrose. In some embodiments, the anionic form of polysucrose is carboxymethyl-polysucrose. In some embodiments of the composition, polysucrose has a molecular weight in the range of 70,000 MW to 400,000 MW, 100,000 MW to 400,000 MW, 200,00 MW to 400,000 MW, 80,000 MW to 350,000 MW, 100,000 MW to 300,00 MW, 100,000 MW to 200,000 MW, 120,000 MW to 200,000 MW. In some exemplary embodiments, polysucrose has a molecular weight of 150,000 MW, 160,000 MW, 170,000 MW, 180,000 MW, 190,000 MW, or 200,000 MW.

In some embodiments of any of the aspects and embodiments herein that include a composition or in some compositions used in or formed by a process herein, comprises platelet derivatives that are positive for at least one platelet activation marker selected from the group consisting of Annexin V, and CD 62. In some embodiments, the platelet derivatives are positive for at least one platelet marker selected from the group consisting of CD 41, CD 42, and CD 61. In some embodiments, the platelet derivatives are positive for CD 47. In some embodiments, the platelet derivatives are positive for Annexin V. In some embodiments, at least 25%, 50%, or 75% of the platelet derivatives in the platelet derivative composition are Annexin V positive. In some embodiments, the platelet derivatives are positive for CD 41. In some embodiments, at least 30%, 35%, 40%, 45%, 50%, 55%, 60%, or 65% of the platelet derivatives in the platelet derivative composition are CD41 positive. In some embodiments, at least 90%, 91%, 92%, 93%, 94%, 95% 96%, 97%, 98%, or 99% platelet derivatives that are positive for CD 41 have a size in the range of 0.5-2.5 μm. In some exemplary embodiments, at least 95% platelet derivatives that are positive for CD 41 have a size in the range of 0.5-2.5 μm. In some embodiments, the platelet derivatives are positive for CD 42. In some embodiments, at least 65%, 80%, or 90% of the platelet derivatives in the platelet derivative composition are CD42 positive. In some embodiments, the platelet derivatives are positive for CD 47. In some embodiments, at least 8%, 10%, 15%, or 20% of the platelet derivatives in the platelet derivative composition are CD47 positive. In some embodiments, the platelet derivatives are positive for CD 62. In some embodiments, at least 10%, 50%, 65%, 80%, or 90% of the platelet derivatives in the platelet derivative composition are CD62 positive. In some embodiments, the platelet derivatives in the platelet derivative composition are positive for CD41, CD62, and Annexin V. In some embodiments, the platelet derivatives in the platelet derivative composition are at least 50% platelet derivatives are positive for CD41, at least 70% platelet derivatives are positive for CD62, and at least 70% platelet derivatives are positive for Annexin V.

In some embodiments of any of the aspects and embodiments herein that include a composition or in some compositions used in or formed by a process herein, the platelet derivatives have fibrinogen associated with their cell membrane. In some embodiments, the platelet derivatives have at least 25%, 30%, 35%, 40%, 45%, 50%, 55%, 60%, 65%, 70%, 75%, 80%, 85%, 90%, 95%, or 100% higher fibrinogen on their surface as compared to resting platelets, or activated platelets, or fixed platelets. In some embodiments, the platelet derivatives when analyzed for the binding of anti-fibrinogen antibody to the platelet derivatives using flow cytometry exhibit at least 10, 15, 20, 25, 30, 35, or 40 folds higher mean fluorescent intensity (MFI) in the absence of an agonist as compared to the MFI of binding of anti-fibrinogen antibody to the fixed platelets.

In some embodiments of any of the aspects and embodiments herein that include a composition or in some compositions used in or formed by a process herein that includes a population of platelet derivatives in a hydrated or rehydrated form, the platelet derivatives in the platelet derivative composition retain at least 10%, or 15%, or 20% of the lactate dehydrogenase activity of donor apheresis platelets. In some embodiments, the aqueous medium has a lactate concentration of less than 2.0 mmol/L, or 1.5 mmol/L. In some embodiments, the lactate concentration is in the range of 0.4 to 1.3 mmol/L, or 0.5 to 1.0 mmol/L.

In some embodiments of any of the aspects and embodiments herein that include a composition or in some compositions used in or formed by a process herein that includes a population of platelet derivatives in a hydrated or rehydrated form, the platelet derivatives, when at a concentration of about 4.8×10³ particles/μL generate a thrombin peak height (TPH) of at least 25 nM, at least 50 nM, at least 75 nM, or at least 100 nM when in the presence of a reagent containing tissue factor and phospholipids. In some embodiments, the platelet derivatives, when at a concentration of about 4.8×10³ particles/μL generate a thrombin peak height (TPH) in the range of 25-100 nM, 30-80 nM, or 25-75 nM.

In some embodiments of any of the aspects and embodiments herein that include a composition or in some compositions used in or formed by a process, the platelet derivatives, have a potency of at least 1.25, at least 1.5, at least 1.75, at least 2.0, at least 2.25, at least 2.5 thrombin generation potency units (TGPU) per 10⁶ particles. In some embodiments, the platelet derivatives have a potency in the range of 1.2 to 2.5, 1.2 to 2.0, 1.3 to 1.5, 1.5 to 2.25, 2 to 2.5, or 2.25 to 2.5 TGPU per 10⁶ particles.

In some embodiments of any of the aspects and embodiments herein that include a composition or in some compositions used in or formed by a process, the platelet derivatives have the presence of thrombospondin (TSP-1) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of thrombospondin (TSP-1) on their surface at a level that is more than 100% higher than on the surface of resting platelets, or lyophilized fixed platelets. In some embodiments, the platelet derivatives have the presence of thrombospondin (TSP-1) on their surface at a level that is at least 50%, 60%, 70%, 80%, 90%, or 100% higher than on the surface of activated platelets, or lyophilized fixed platelets. In some embodiments, the platelet derivatives have the presence of thrombospondin (TSP-1) on their surface at a level that is more than 100% higher than on the surface of activated platelets, or lyophilized fixed platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, 40 folds, 50 folds, 60 folds, 70 folds, 80 folds, 90 folds, or 100 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the lyophilized fixed platelets. In some embodiments, the platelet derivatives when analyzed for the binding of anti-thrombospondin (TSP) antibody to the platelet derivatives using flow cytometry exhibit 10-800 folds, 20-800 folds, 100-700 folds, 150-700 folds, 200-700 folds, or 250-500 folds higher mean fluorescent intensity (MFI) in the absence of an agonist as compared to the MFI of binding of anti-TSP antibody to the resting platelets. In some embodiments, the platelet derivatives when analyzed for the binding of anti-thrombospondin (TSP) antibody to the platelet derivatives using flow cytometry exhibit at least 2 folds, 5 folds, 7 folds, 10 folds, 20 folds, 30 folds, or 40 folds higher mean fluorescent intensity (MFI) in the absence of an agonist as compared to the MFI of binding of anti-TSP antibody to the active platelets. In some embodiments, the platelet derivatives when analyzed for the binding of anti-thrombospondin (TSP) antibody to the platelet derivatives using flow cytometry exhibit 2-40 folds, 5-40 folds, 5-35 folds, 10-35 folds, or 10-30 folds higher mean fluorescent intensity (MFI) in the absence of an agonist as compared to the MFI of binding of anti-TSP antibody to the active platelets.

In some embodiments of any of the aspects and embodiments herein that include a composition or in some compositions used in or formed by a process, the platelet derivatives have the presence of von Willebrand factor (vWF) on their surface at a level that is at least 10%, 20%, 25%, 30%, 50%, 60%, 70%, 80%, 90%, or 100% higher than on the surface of resting platelets, or lyophilized fixed platelets. In some embodiments, the platelet derivatives have the presence of von Willebrand factor (vWF) on their surface at a level that is more than 100% higher than on the surface of resting platelets, or lyophilized fixed platelets. In some embodiments, the platelet derivatives when analyzed for the binding of anti-von Willebrand factor (vWF) antibody to the platelet derivatives using flow cytometry exhibits at least 1.5 folds, 2 folds, or 3 folds, or 4 folds higher mean fluorescent intensity (MFI) in the absence of an agonist as compared to the MFI of binding of anti-vWF antibody to the resting platelets, or lyophilized fixed platelets. In some embodiments, the platelet derivatives when analyzed for the binding of anti-von Willebrand factor (vWF) antibody to the platelet derivatives using flow cytometry exhibits 2-4 folds, or 2.5-3.5 higher mean fluorescent intensity (MFI) in the absence of an agonist as compared to the MFI of binding of anti-vWF antibody to the resting platelets, or lyophilized fixed platelets.

In some embodiments of any of the aspects and embodiments herein that include a composition or in some compositions used in or formed by a process, the platelet derivatives lack an integrated membrane as compared to platelets. In some embodiments, the platelet derivatives are surrounded by a compromised plasma membrane. In some embodiments, the platelet derivatives are incapable of retaining more than 35%, 40%, 45%, 50%, 55%, 60%, 65%, 70%, or 75% of lactate dehydrogenase as compared to lactate dehydrogenase retained in fresh platelets, or cold stored platelets, or cryopreserved platelets. In some embodiments, the platelet derivatives can retain 35%-75%, 40-70%, 45-65%, or 35-50% lactate dehydrogenase as compared to fresh platelets, or cold stored platelets, or cryopreserved platelets. In some embodiments, the platelet derivatives exhibit an increased permeability to antibodies. In some embodiments, the antibodies can be IgG antibodies.

In some embodiments of any of the aspects and embodiments herein that include a composition or in some compositions used in or formed by a process, the platelet derivatives, when at a concentration of at least about 70×10³ particles/μL, produce an occlusion time of less than 14 minutes, or less than 12 minutes in a total thrombus-formation analysis system (T-TAS) assay. In some embodiments, the occlusion time is in the range of 1 to 13 minutes, 1 to 11 minutes, 1 to 10 minutes, or 1 to 7 minutes.

In some embodiments of any of the aspects and embodiments herein that include a composition or in some compositions used in or formed by a process, the platelet derivatives in the platelet derivative composition comprise thrombosomes. In some embodiments, the platelet derivatives comprise freeze-dried platelets. In some embodiments, the platelet derivatives comprise thermally-treated freeze-dried platelets.

In some embodiments of any of the aspects and embodiments herein that include a process, comprises tangential flow filtration (TFF), centrifugation of a starting material comprising platelet composition, or a combination thereof. In some embodiments of the process, the starting material comprising platelet composition is: a) positive for HLA Class I antibodies based on a regulatory agency approved test; or b) positive for HLA Class II antibodies based on a regulatory agency approved test; or c) positive for HNA antibodies based on a regulatory agency approved test; or two or more of a), b), and c). In some embodiments, the starting material comprising platelet composition has a protein concentration in the range of 60 to 80 mg/mL, or 65 to 75 mg/mL. In some embodiments, the starting material comprising platelet composition comprises donor blood product. In some embodiments, the donor blood product is pooled donor blood product. In some embodiments, the starting material comprising platelet composition comprises donor apheresis material.

In some embodiments of any of the aspects and embodiments herein that include a process, that does not comprise centrifugation of the starting material comprising platelets or platelet composition, the diluted starting material comprising platelets or platelet composition, the concentrated platelet composition, the TFF-treated composition, or the combination thereof. In some embodiments, the process does not comprise centrifugation of a composition comprising platelets or platelet derivatives.

In some embodiments of any of the aspects and embodiments herein that include a process, the TFF comprises concentrating. In some embodiments, the TFF comprises diafiltering. In some embodiments, the diafiltering comprises diafiltering with at least two diavolumes. In some embodiments, the diafiltering is done with at least three diavolumes, or four diavolumes, or five diavolumes, or six diavolumes. In some embodiments, the diafiltering is done with diavolumes in the range of two to ten. In some embodiments, the TFF comprises buffer exchange.

In some embodiments of any of the aspects and embodiments herein that include a process, diluting comprises diluting with an approximately equal weight (±10%) of the preparation agent.

In some embodiments of the process of any of the aspects and embodiments herein that include a process, further comprises a pathogen reduction step. In some embodiments, the pathogen reduction step occurs before the diluting of the starting material. In some embodiments, the pathogen reduction step precedes TFF.

In some embodiments of any of the aspects and embodiments herein that include a process, wherein following washing if the concentration of the platelets or cells in the TFF-treated composition is not 2000×10³ cells/μL (+300×10³), 3000×10³ cells/μL (+300×10³), 4000×10³ cells/μL (+300×10³ 5000×10³ cells/μL (+300×10³), 6000×10³ cells/μL (+300×10³), 7000×10³ cells/μL (+300×10³), 8000×10³ cells/μL (+300×10³), 10000×10³ cells/μL (+300×10³), 12000×10³ cells/μL (+300×10³), 14000×10³ cells/μL (+300×10³), 16000×10³ cells/μL (+300×10³), 18000×10³ cells/μL (+300×10³ or 20000×10³ cells/μL (+300×10³), diluting the preparation agent or concentrating the platelets or the cells to fall within this range.

In some embodiments of any of the aspects and embodiments herein that include a process, further comprises lyophilizing or freeze-drying the TFF-treated composition to form a lyophilized composition. In some embodiments, a process further comprises treating the lyophilized composition at a temperature in the range of 60-90° C., or 65-85° C., or 70-90° C. for a time period in the range of 1-36 hours, or 5-30 hours, or 10-25 hours.

In some embodiments of any of the aspects and embodiments herein that include a process, the TFF is carried out using a membrane with a pore size in the range of 0.2 μm to 1 μm. In some embodiments the TFF is carried out using a membrane with pore size in the range of 0.3 μm to 1 μm, or 0.4 μm to 1 μm, or 0.4 μm to 0.8 μm, or 0.4 μm to 0.7 μm. In illustrative embodiments, the TFF is carried out using a membrane with a pore size of 0.45 μm, or 0.65 μm.

In some embodiments of any of the aspects and embodiments herein that include a process, the TFF is carried out until the absorbance at 280 nm of the aqueous medium is less than or equal to 50%, or 30%, or 10%, or 5%, or 3%, or 1% of the absorbance at 280 nm of the starting material comprising platelet composition, using a path length of 0.5 cm. In some embodiments, the TFF is carried out until the protein concentration or plasma protein concentration in the aqueous medium is less than or equal to 20%, 15%, 12.5%, 10%, 7.5%, 5%, 2.5%, or 1%. In some embodiments, the TFF is carried out until the protein concentration or plasma protein concentration is in between 0.01% and 20%, 0.01% and 15%, 0.01% and 10%, 0.010% and 5%, 0.10% and 20%, 0.10% and 15%, 0.10% and 10%, 0.10% and 5%, 10% and 20%, 10% and 15%, 1% and 10%, 1% and 5%, 2% and 10%, 2% and 5%, 2.5% and 5%, 2.5% and 7.5%, or between 3% and 5%. In some embodiments, the TFF is carried out until the absorbance at 280 nm of the aqueous medium is less than or equal to 2.0 AU, or 1.90 AU, or 1.80 AU, or 1.70 AU, or 1.66 AU, or 1.60 AU, using a path length of 0.5 cm. In some embodiments, the TFF is carried out until the platelet concentration is at least 2000×10³ platelets/μL, 2250×10³ platelets/μL, 3000×10³ platelets/μL, 3250×10³ platelets/μL, 3500×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, or 20,000×10³ platelets/μL.

In some embodiments of any of the aspects and embodiments herein that include a process, the TFF-treated composition comprises at least 1000×10³ platelets/μL, 2000×10³ platelets/μL, 2250×10³ platelets/μL, 3000×10³ platelets/μL, 3250×10³ platelets/μL, 3500×10³ platelets/μL, 4000×10³ platelets/μL, 4250×10³ platelets/μL, 4500×10³ platelets/μL, 4750×10³ platelets/μL, 5000×10³ platelets/μL, 5250×10³ platelets/μL, 5500×10³ platelets/μL, 5750×10³ platelets/μL, 6000×10³ platelets/μL, 7000×10³ platelets/μL, 8000×10³ platelets/μL, 9000×10³ platelets/μL, 10,000×10³ platelets/μL, 11,000×10³ platelets/μL, 12,000×10³ platelets/μL, 13,000×10³ platelets/μL, 14,000×10³ platelets/μL, 15,000×10³ platelets/μL, 16,000×10³ platelets/μL, 17,000×10³ platelets/μL, 18,000×10³ platelets/μL, 19,000×10³ platelets/μL, or 20,000×10³ platelets/μL. In some embodiments, the TFF-treated composition comprises 1000×10³ platelets/μL to 20,000×10³ platelets/μL, 10,000×10³ platelets/μL to 20,000×10³ platelets/μL, 5000×10³ platelets/μL to 20,000×10³ platelets/μL, or 5000×10³ platelets/μL to 10,000×10³ platelets/μL.

In some embodiments of any of the aspects and embodiments herein that include a process, the TFF comprises diafiltering with a preparation agent comprising a buffering agent, a base, a loading agent, optionally a salt, and optionally at least one organic solvent. In some embodiments of the process, the TFF comprises buffer exchange into a preparation agent comprising a buffering agent, a base, a loading agent, optionally a salt, and optionally at least one organic solvent. In some embodiments, the buffering agent is HEPES (4-(2-hydroxyethyl)-1-piperazineethanesulfonic acid). In some embodiments, the base is sodium bicarbonate. In some embodiments, the loading agent is a monosaccharide, a polysaccharide, or a combination thereof. In some embodiments, the monosaccharide is selected from the group consisting of sucrose, maltose, trehalose, glucose, mannose, xylose, and combinations thereof. In some embodiments, the monosaccharide is trehalose. In some embodiments, the polysaccharide is polysucrose. In some embodiments, the salt is sodium chloride, potassium chloride, or a combination thereof. In some embodiments, the organic solvent is selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

In some embodiments of any of the aspects and embodiments herein that include a process, the preparation agent has a pH in the range of 5.5 to 8.0, or 6.0 to 8.0, or 6.0 to 7.5. In an illustrative embodiment, the preparation agent has a pH of 6.5. In another illustrative embodiment, the preparation agent has a pH of 7.4.

In some embodiments of any of the aspects and embodiments herein that include a process, that does not comprise a step for fixing the platelets, or platelet derivatives. In some embodiments, the process does not comprise fixing the platelets, or platelet derivatives using a fixative agent known in the art for fixing the platelets or platelet derivatives. In some embodiments, the process does not comprise contacting the platelets, or platelet derivatives with at least one fixative agent. In some embodiments, the fixative agent is an aldehyde. In some embodiments, the fixative agent is an alcohol. In illustrative embodiments, the fixative agent is selected from the group consisting of formaldehyde, paraformaldehyde, glutaraldehyde, and isopropanol.

In some embodiments of any of the aspects and embodiments herein that include a process, further comprises lyophilizing the composition comprising platelets or platelet derivatives.

In some embodiments of any of the aspects and embodiments herein that include a process, further comprises cryopreserving the composition comprising platelets or platelet derivatives.

In some embodiments of any of the aspects and embodiments herein that include a process, further comprises thermally treating the composition comprising platelets or platelet derivatives.

In some of the embodiments of any of the aspects and embodiments herein that include a process, the TFF is performed at a temperature in the range of 20° C. to 37° C., or 25° C. to 37° C., or 20° C. to 35° C., or 25° C. to 35° C.

In some embodiments of any of the aspects and embodiments herein that include a process, a percentage of beads positive for an antibody selected from the group consisting of HLA Class I antibodies, HLA Class II antibodies, and HNA antibodies, as determined for the composition by flow cytometry using beads coated with Class I HLAs, Class II HLAs, or HNAs, respectively, is reduced by at least 50%, or by at least 75%, or by at least 90%, or by at least 95%, as compared to a similar composition not prepared by a process comprising tangential flow filtration of a blood product composition, centrifugation of a blood product composition, or a combination thereof.

In some embodiments of any of the aspects and embodiments herein that include a composition, or in some compositions used in or formed by a process, the platelet derivatives are derived from human platelets and are positive for at least one marker selected from the group consisting of CD 41, CD 42, and CD 61. In some embodiments, the platelet derivatives are derived from human platelets that are positive for CD 41. In some embodiments, embodiments, the platelet derivatives are derived from human platelets that are positive for CD 42. In some embodiments, embodiments, the platelet derivatives are derived from human platelets that are positive for CD 61. In some illustrative embodiments, the platelet derivatives are derived from human platelets that are positive for CD 41, CD 42, and CD 61.

In some embodiments of any of the aspects and embodiments herein that include a composition, or in some compositions used in or formed by a process, the platelet derivatives are derived from a non-human animal. In some embodiments, the non-human animal is selected from the group consisting of canines, equines, and felines. In some exemplary embodiments, the platelet derivatives are derived from canines.

In some embodiments of any of the aspects and embodiments herein that include a composition, or in some compositions used in or formed by a process that includes a population of platelet derivatives a powdered form, the platelet derivative composition comprises no more than 0.1%, 0.2%, 0.3%, 0.4%, 0.5%, 0.6%, 0.7%, 0.8% r 0.9%, 1%, 1.5%, 2%, 2.5%, 3%, 3.5%, 4.0%, 4.5%, or 4.9% residual moisture. In some embodiments, wherein the platelet derivative composition is in a powdered form, the platelet derivative composition comprises residual moisture in the range of 0.1-2%, 0.2-1.5%, 0.5-1.5%, 0.75-1.25%, 2-3%, 2.5-4.9%, 3-4.5%, 1.5-3%, or 1-2% residual moisture. In some illustrative embodiments, the platelet derivative composition comprises no more than 0.5% residual moisture.

In some embodiments of any of the aspects and embodiments herein that include a composition, or in some compositions used in or formed by a process that includes a plurality of containers each filled with a platelet derivative composition in the form of a powder, the platelet derivative composition in at least one of the plurality of containers comprises or is associated with a first protein from a first gene that has a different amino acid sequence than found in all the versions of the first protein from the first gene in the platelet derivative composition in one or more other containers of the plurality.

In some embodiments of any of the aspects and embodiments herein that include a composition, or in some compositions used in or formed by a process that includes a plurality of containers each filled with a platelet derivative composition in the form of a powder, the at least one container comprises a first lot of platelet derivatives and the one or more other containers comprise a second lot of platelet derivatives. In some embodiments, plurality of containers comprises the platelet derivative composition from at least 2, 3, 4, 5, 6, 7, 8, 9, or 10 different lots, wherein the platelet derivative composition in at least 2 of the lots have a different amino acid sequences for at least one protein of a collection of protein gene products from a corresponding collection of encoding genes. In illustrative embodiments all, of the lots have a different amino acid sequences for at least one protein of a collection of protein gene products from a corresponding collection of encoding genes. In some embodiments, the amino acid difference(s) is at one or more residues corresponding to amino acid residues encoded by a non-synonymous single nucleotide polymorphism (SNP).

In some embodiments of any of the aspects and embodiments herein that include a composition, or in some compositions used in or formed by a process that includes a plurality of containers each filled with a platelet derivative composition in the form of a powder, each of the plurality of containers are purged with at least one inert gas. In some embodiments, the inert gas can be argon, or nitrogen.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one protein of a collection of protein gene products from a corresponding collection of encoding genes. In some embodiments, the different amino acid sequences differ at one or more residues corresponding to amino acid residues encoded by a non-synonymous single nucleotide polymorphism (SNP). In some embodiments, the platelet derivative composition is in a container, and wherein the container is filled with at least one inert gas.

In some embodiments of any of the aspects and embodiments herein that include a composition, or in some compositions used in or formed by a process that includes a plurality of containers each filled with a platelet derivative composition in the form of a powder, the amount of plasma protein in the powder of any two containers chosen from different lots, differs by less than 50%, 40%, 30%, 20%, 1%, 50%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the amount of microparticles that are less than 0.5 μm in the powder of any two containers chosen from different lots, differs by less than 50%, 40%, 30%, 20%, 10%, 5%, 2%, 1%, or 0.5%.

In some embodiments of any of the aspects and embodiments herein that include a composition, or in some compositions used in or formed by a process that includes a plurality of containers each filled with a platelet derivative composition in the form of a powder, the platelet derivative composition from the at least 2 lots have different amino acid sequences for at least one, two, three, four, or five protein of a collection of protein gene products from a corresponding collection of encoding genes. In some embodiments, the different amino acid sequences differ at one or more residues corresponding to amino acid residues encoded by a non-synonymous single nucleotide polymorphism (SNP).

In some embodiments of any of the aspects and embodiments herein that includes a plurality of containers each filled with a platelet derivative composition in the form of a powder, the containers can vary in volume from 5-100 ml, 10-90 ml, 25-75 ml, or 5-40 ml. In some embodiments, the volume of containers can be 5 ml, 10 ml, 15 ml, 20 ml, 25 ml, 30 ml, 35 ml, 40 ml, 45 ml, 50 ml, 55 ml, 60 ml, 65 ml, 70 ml, 75 ml, 80 ml, 85 ml, 90 ml, 95 ml, or 100 ml. In some embodiments, the volume of containers can be above 100 ml, for example, 125 ml, 150 ml, 175 ml, or 200 ml. In some illustrative embodiments, the volume of vials is 30 ml. In some other illustrative embodiments, the volume of vials is 10 ml. In some embodiments, the plurality of containers can have 10-500 vials, 25-450 vials, 50-350 vials, 100-300 vials, or 150-250 vials. In some embodiments, the plurality of containers can have 10, 20, 30, 40, 50, 60, 70, 80, 90, 100, 110, 120, 130, 140, 150, 160, 170, 180, 190, 200, 250, 300, 350, 400, 450, or 500 vials. In some embodiments, the plurality of containers can be increased to more than 500 as per the requirements, for example, 600, 700, 800, 900, or 1000 vials. In some embodiments, the number of vials can be 10-1000, 50-1000, 100-900, 200-800, or 150-700, or 150-500 vials. The number of vials in which a platelet derivative composition as per one of the embodiments, or aspects described herein can be filled and/or present can vary with the manufacturing requirements and the amount of starting material comprising platelets.

In some embodiments of any of the aspects and embodiments herein that includes a plurality of containers each filled with a platelet derivative composition in the form of a powder, the amount of platelet derivatives when the plurality of containers is taken as a whole can be 1×10⁹ to 1×10¹⁶, 1×10¹⁰ to 1×10¹⁵, 1×10 to 1×10¹⁵, 1×10¹² to 1×10¹⁶, or 1×10¹³ to 1×10¹⁵.

In some embodiments of any of the aspects and embodiments herein that include a composition, or in some compositions used in or formed by a process, the platelet derivatives are allogenic platelet derivatives. In some embodiments, the platelet derivatives are allogenic platelet derivative product. In some embodiments, a platelet derivative composition as per any of the embodiments or aspects herein, is a composition comprising allogenic platelet derivatives. In some embodiments, a platelet derivative composition as described herein is a U.S. FDA-approved product comprising allogenic platelet derivative composition. In some embodiments, a platelet derivative composition as described herein is a European EMA-approved product comprising allogenic platelet derivative composition. In some embodiments, a platelet derivative composition as described herein is a China FDA-approved product comprising an allogenic platelet derivative composition.

In some embodiments of the aspects and embodiments herein that include a composition, or in some compositions used in or formed by a process, or a process for preparing a platelet derivative composition, the platelets in the starting material can be donated from a human subject. In some embodiments, the human subject can be a male, or a female. In some embodiments, the platelets can be a pooled product from a number of male and female donors. In some embodiments, from a total of 100 donors, any number can be female donors, ranging from 0-100, 5-95, 10-90, 20-80, 30-70, or 40-60, and the rest can be male donors.

In some embodiments of the aspects and embodiments herein that include a composition, or in some compositions used in or formed by a process, or a process for preparing a platelet derivative composition, a starting material can comprise 10-500 units of platelets. In some embodiments, the starting material can comprise 20-500 units, 30-400 units, 40-350 units, or 50-200 units. In some embodiments, the units can be a pooled platelet product from multiple donors as described herein.

In some embodiments of any of the aspects and embodiments herein that include a method for treating a clotting-related disorder in a subject, said method comprising administering to the subject a therapeutically effective amount of the platelet derivative composition of any of the aspects or embodiments herein, or the platelet derivative composition prepared by any of the process described in the aspects or embodiments herein. In some embodiments, the clotting-related disorder is selected from the group consisting of Von Willebrand Disease, hemophilia, thrombasthenia, thrombocytopenia, thrombocytopenic purpura, trauma, or a combination thereof. In some embodiments, the composition is passed through a filter of 18 μm before administering to the subject.

In some embodiments, the platelet derivative composition of any of the aspects or embodiments herein is provided for use in the treatment of a disorder selected from the group consisting of alopecia areata, Von Willebrand Disease, hemophilia, thrombasthenia, thrombocytopenia, thrombocytopenic purpura, trauma, or a combination thereof.

In some embodiments, the platelet derivatives as described herein can be used for healing wounds in a subject. In some embodiments, there is provided a method for healing a wound in a subject, comprising administering a therapeutically effective amount of a platelet derivative composition of any of the aspects or embodiments herein, or the platelet derivative composition prepared by any of the process described in the aspects or embodiments herein, to the subject. In some embodiments, the platelet derivative composition of any of the aspects or embodiments herein is provided for use in wound healing in a subject.

In some embodiments, the platelet derivative composition of any of the aspects or embodiments herein can be used in treating a coagulopathy in a subject that has been administered or is being administered an antiplatelet agent. In some embodiments, the platelet derivative composition of any of the aspects or embodiments herein is provided for use an anti-platelet reversal agent. In some embodiments, the platelet derivative composition of any of the aspects or embodiments herein can be used in treating a coagulopathy in a subject that has been administered or is being administered an anticoagulant agent. In some embodiments, the platelet derivative composition of any of the aspects or embodiments herein is provided for use an anti-coagulant reversal agent.

In some embodiments, there is provided a method of treating a subject, the method comprising administering to the subject in need thereof, an effective amount of any of the platelet derivative compositions disclosed herein, or the platelet derivative composition prepared by any of the methods disclosed herein, wherein the subject has been treated or is being treated with an antiplatelet agent and/or an anti-coagulant, and wherein the method for treating is a) a method for restoring normal hemostasis in the subject; b) for treating a coagulopathy in the subject; or c) for preparing the subject for surgery. In some embodiments, the method of treating is the method of restoring normal hemostasis in the subject. In some embodiments, the method of treating is the method for treating the coagulopathy in the subject. In some embodiments, the method of treating is the method for preparing the subject for surgery. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, a supplement, and a combination thereof. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, and a combination thereof. In some embodiments of any of the methods for treating aspects herein, wherein the subject is being treated with an antiplatelet agent, the antiplatelet agent is selected from the group consisting of aspirin, cangrelor, ticagrelor, clopidogrel, prasugrel, eptifibatide, tirofiban, abciximab, terutroban, picotamide, elinogrel, ticlopidine, ibuprofen, vorapaxar, atopaxar, cilostazol, prostaglandin E1, epoprostenol, dipyridamole, treprostinil sodium, sarpogrelate, and a combination thereof.

In some embodiments of any of the methods for treating aspects herein, wherein the subject is being treated with an anticoagulant, the anticoagulant is selected from the group consisting of dabigatran, argatroban, hirudin, rivaroxaban, apixaban, edoxaban, fondaparinux, warfarin, heparin, a low molecular weight heparin, a supplement, and a combination thereof. In some embodiments of any of the methods for treating aspects herein, wherein the subject is being treated with an anticoagulant, the anticoagulant is selected from the group consisting of dabigatran, argatroban, hirudin, rivaroxaban, apixaban, edoxaban, fondaparinux, warfarin, heparin, low molecular weight heparins, tifacogin, Factor VIIai, SB249417, pegnivacogin (with or without anivamersen), TTP889, idraparinux, idrabiotaparinux, SR23781A, apixaban, betrixaban, lepirudin, bivalirudin, ximelagatran, phenprocoumon, acenocoumarol, indandiones, fluindione, a supplement, and a combination thereof. In some embodiments of any of the methods for treating aspects herein, the administering comprises administering topically, parenterally, intravenously, intramuscularly, intrathecally, subcutaneously or intraperitoneally. In some embodiments of any of the methods for treating aspects herein, where the composition is in the form of a powder, the method further includes, before the administering, rehydrating the composition. In some embodiments provided herein is use of any of the platelet derivative compositions herein, in the manufacture of a kit for performing any of the methods of treating provided herein.

In some embodiments, there is provided a composition comprising platelets or platelet derivatives prepared by any of the process described in any of the aspects or embodiments herein. In some embodiments, there is provided a composition comprising platelets or platelet derivatives and an aqueous medium prepared by any of the process described in any of the aspects or embodiments herein. In some of the embodiments, there is provided a composition comprising freeze-dried platelets prepared by any of the process described in any of the aspects or embodiments herein. In some of the embodiments, there is provided a composition comprising thrombosomes prepared by any of the aspects or embodiments herein. In some of the embodiments of any of the aspects and embodiments herein that include a composition, or in some compositions used in or formed by a process, a composition prepared by a process comprising tangential flow filtration (TFF) of a starting material comprising platelets, centrifugation of a starting material comprising platelets, or a combination thereof. In some embodiments, the centrifugation comprises centrifugation at 1400×g to 1550×g, or 1450×g to 1500×g. In some embodiments, the composition is prepared by a process that does not comprise centrifugation.

In some embodiments of the aspects and embodiments herein that include a platelet derivative composition, or in some compositions used in or formed by a process, or a process for preparing a platelet derivative composition, or a method for treating a subject, or a platelet derivative composition comprising platelet derivatives for use as a medicament in treating a subject, a therapeutically effective dose of platelet derivatives is based on units of thrombin generation activity administered per kilogram of body weight of the subject. In further embodiments of these embodiments the effective dose is not based on the number of platelet derivatives delivered to the subject. In some embodiments of any aspect or embodiment herein the effective dose is based on both A) units of thrombin generation activity administered per kilogram of body weight of the subject; and B) the number of platelet derivatives administered to the subject. In some embodiments of any aspect or embodiment herein the effective dose is based on the weight of the subject.

In some embodiments of any aspect or embodiment herein the subject is suffering from a condition, or a disease selected from the group including only thrombocytopenia, hematologic malignancy, bone marrow aplasia, myeloproliferative disorders, myelodysplastic syndromes, and platelet refractoriness. In some embodiments, the subject is suffering from thrombocytopenia. In some embodiments, the subject is suffering from hematologic malignancy. In some embodiments, the subject is suffering from bone marrow aplasia. In some embodiments, the subject is suffering from myeloproliferative disorders. In some embodiments, the subject is suffering from myelodysplastic syndromes. In some embodiments, the subject is suffering from platelet refractoriness. In some embodiments, the subject is suffering from two or more of the disease or condition selected from the group consisting of thrombocytopenia, hematologic malignancy, bone marrow aplasia, myeloproliferative disorders, myelodysplastic syndromes, and platelet refractoriness. In some embodiments, the subject is refractory to platelet transfusion.

In some embodiments of any aspect or embodiment herein a therapeutically effective dose or amount of the platelet derivatives in a platelet derivative composition is in the range of 1.0×10⁷ to 1.0×10¹²/kg of the subject.

In some embodiments of any aspect or embodiment herein a therapeutically effective dose or amount of the platelet derivatives is an amount that has a potency in the range of 250 to 5000 TGPU per kg of the subject.

In some embodiments of any aspect or embodiment herein, a method of administering, a method of treatment or a composition for use as a medicament as described herein, a method or a medicament leads to cessation or decrease in bleeding, in illustrative embodiments in a subject having HPS or BSS, at a primary bleeding site at or within 5 minutes, 10 minutes, 15 minutes, 20 minutes, 30 minutes 1 hour, 2 hours, 3 hours, 4 hours, 6 hours, 8 hours, 12 hours, 24 hours, 48 hours, 72 hours, 4 days, 5 days, 6 days, and/or 7 days, or in between 5 minutes on the low end of the range, and 72, 48 hours, 24 hours, 12 hours, 8 hours, 6 hours, 4 hours, 3 hours, 2 hours, 1 hour, 30 minutes, 15 minutes, or 10 minutes after administering the platelet derivative composition. In some embodiments, the primary bleeding site is based upon the most severe bleeding location of the subject within 12 hours prior to administering the platelet derivative composition. In some embodiments, the administering involves infusing a platelet derivative composition. In some embodiments, a platelet derivative composition is administered on Day 1 of the treatment. In some embodiments, the cessation or decrease is evidenced by an ordinal change in WHO bleeding score of the subject evaluated at 24 hours after administering the platelet derivative composition to the subject. In some embodiments, a method or a medicament as described herein leads to cessation or decrease in bleeding at bleeding sites other than primary bleeding site at 12 hours, 24 hours, 48 hours, 72 hours, 4 days, 5 days, 6 days, and 7 days after administering the platelet derivative composition. For example, the bleeding can be caused by trauma, for example, a blunt trauma, for example, a car accident, or a penetrating trauma, for example, being cut by a sharp knife, a gun-shot wound or a stabbing wound. The bleeding can be or can include internal bleeding.

In some embodiments of any aspect or embodiment herein, a method of treatment or a composition for use as a medicament as described herein, a method or a medicament leads to an increase in platelet count in the subject at 12 hours, 24 hours, 48 hours, 72 hours, 4 days, 5 days, 6 days, and 7 days after administering the platelet derivative composition. In some embodiments, the increase is at least 500 platelets/μl, 1000 platelets/μl, 2000 platelets/μl, 3000 platelets/μl, 4000 platelets/μl, 5000 platelets/μl, 6000 platelets/μl, 7000 platelets/μl, 8000 platelets/μl, 9000 platelets/μl, or 10000 platelets/μl in the subject. In some embodiments, the increase is in the range of 500 to 10000 platelets/μl, 1000 to 10000 platelets/μl, 2000 to 8000 platelets/μl, or 3000 to 7000 platelets/μl in the subject.

In some embodiments of any aspect or embodiment herein, a method of treatment or a composition for use as a medicament as described herein, a method or a medicament leads to changes, or in some embodiments, does not lead to changes, in one or more markers of endothelial cell injury in the subject from a pre-administration time through 12 hours to 35 days, 24 hours to 32 days, 24 hours to 30 days, or 48 hours to 28 days after administering the platelet derivative composition. In some embodiments, the method or the medicament leads to changes, or in some embodiments, does not lead to changes, in one or more markers of endothelial cell injury in the subject at 72 hours after administering the platelet derivative composition. In some embodiments, the one or more markers of endothelial cell injury is selected from the group consisting of Syndecan-1, hyaluronan, thrombomodulin, vascular endothelial growth factor (VEGF), interleukin 6, and sVE cadherin. In some embodiments, the method or the medicament leads to changes in two or more markers, three or markers, four or more markers, five or more markers, or all of the markers selected from the group consisting of Syndecan-1, hyaluronan, thrombomodulin, vascular endothelial growth factor (VEGF), interleukin 6, and sVE cadherin. In some embodiments, the changes can be an increase or a decrease in the markers of endothelial cell injury in the subject as compared to a control.

In some embodiments of any aspect or embodiment herein, a method of treatment or a composition for use as a medicament as described herein, a method or a medicament leads to acceptable measures of coagulation in the subject at 12 hours to 35, 24 hours to 32 days, 24 hours to 30 days, or 48 hours to 28 days after administering the platelet derivative composition. In some embodiments, a method or a medicament leads to acceptable measures of coagulation in the subject at 72 hours after administering the platelet derivative composition. In some embodiments, the acceptable measure of coagulation includes one or more, two or more, three or more, four or more, five or more, or all of prothrombin time (PT), international normalized ratio (INR), fibrinogen, D-dimer, activated partial thromboplastin time (aPTT), and thromboelastography (TEG) or rotational thromboelastometry (ROTEM). In some embodiments, a method or a medicament leads to an increase or a decrease in the acceptable measure of coagulation in the subject as compared to a control.

In some embodiments of any aspect or embodiment herein, a method of treatment or a composition for use as a medicament as described herein, a method or a medicament leads to acceptable measures of hematology in the subject from a pre-administration time through 12 hours to 35 days, 24 hours to 32 days, 24 hours to 30 days, or 48 hours to 28 days after administering the platelet derivative composition. In some embodiments, the acceptable measures of hematology are one or more, two or more, three or more, four or more, five or more, or all selected from the group consisting of Prothrombin Fragment 1+2, thrombin generation assay (TGA), Thrombopoietin, activated Protein C, tissue plasminogen activator (TPA), and/or plasminogen activator inhibitor (PAI). In some embodiments, the acceptable measures of hematology can be an increase or a decrease in the subject as compared to a control.

In some embodiments of any aspect or embodiment herein, a method of treatment or a composition for use as a medicament as described herein, a method or a medicament leads to survival of the subject without WHO Grade 2A or greater bleeding during the first 3, 4, 5, 6, 7, 8, 9, or 10 days after administering of a platelet derivative composition.

In some embodiments of any aspect or embodiment herein, a method of treatment or a composition for use as a medicament as described herein, administering is performed in a maximum of 2, 3, 4, 5, 6, 7, 8, 9, or 10 doses in a 72-hour period of treatment. In some embodiments, the subject has a count of total circulating platelets (TCP) between 5,000 to 100,000 platelets/μl, 10,000 to 90,000 platelets/μl, 10,000 to 80,000 platelets/μl, or 10,000 to 70,000 platelets/μl of blood at the time of administering. In some embodiments, the subject is undergoing one or more, two or more, three or more, or all of chemotherapy, immunotherapy, radiation therapy or hematopoietic stem cell transplantation at the time of administering. In some embodiments, the subject is refractory to platelet transfusion, wherein refractory is a two 1-hour CCI [corrected count increment] of <5000 on consecutive transfusions of liquid stored platelets. In some embodiments, the subject has a WHO bleeding score of 2 excluding cutaneous bleeding.

In some embodiments of any aspect or embodiment herein the subject at the time of administering has two or more, or all of: confirmed diagnosis of hematologic malignancy, myeloproliferative disorder, myelodysplastic syndrome, or aplasia; undergoing chemotherapy, immunotherapy, radiation therapy or hematopoietic stem cell transplantation; or refractory to platelet transfusion wherein refractory is a two 1-hour CCI of <5000 on consecutive transfusions of liquid stored platelets.

In some embodiments of any aspect or embodiment herein the administering confers an improved survival at 10, 15, 20, 25, 30, 35, 40, 45, or 50 days after administering the platelet derivatives. In some embodiments of any aspect or embodiment herein the administering leads to a decrease in administration of secondary blood products, platelets, or platelet derivatives to the subject for the first 5, 6, 7, 8, 9, or 10 days after the administering of the platelet derivatives.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, the subject is having an indication selected from the group consisting of Von Willebrand disease, immune thrombocytopenia, Intracranial hemorrhage (ICH), Traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, acute lymphoblastic lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof. In some embodiments, an indication is Von Willebrand disease. In some embodiments, an indication is Immune thrombocytopenia. In some embodiments, an indication is Chemotherapy induced thrombocytopenia (CIT). In some embodiments, an indication is Fetal and neonatal alloimmune thrombocytopenia.

In some embodiments of any aspect or embodiments herein that include an indication, the indication is selected from the group consisting of Von Willebrand disease, Immune thrombocytopenia, Intracranial hemorrhage (ICH), Traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, Glanzmann thrombasthenia, Myelodysplastic syndrome, Hemorrhagic Shock, Coronary thrombosis (myocardial infarction), Ischemic Stroke, Arterial Thromboembolism, Wiskott Aldrich Syndrome, Venous Thromboembolism, MYH9 related disease, Acute Lymphoblastic Lymphoma (ALL), Acute Coronary Syndrome, Chronic Lymphocytic Leukemia (CLL), Acute Promyelocytic Leukemia, Cerebral Venous Sinus Thrombosis (CVST), Liver Cirrhosis, Factor V Deficiency (Owren Parahemophilia), Thrombocytopenia absent radius syndrome, Kasabach Merritt syndrome, Gray platelet syndrome, Aplastic anemia, or combinations thereof.

In some embodiments of any aspect or embodiments herein that include an indication, the indication is selected from the group consisting of Chronic Liver Disease, Acute radiation syndrome, Dengue Hemorrhagic Fever, Pre-Eclampsia, Snakebite envenomation, HELLP syndrome, Haemorrhagic Cystitis, Multiple Myeloma, Disseminated Intravascular Coagulation, Heparin Induced Thrombocytopenia, Pre-Eclampsia, Labor And Delivery, Hemophilia, Cerebral (Fatal) Malaria, Alexander's Disease (Factor VII Deficiency), Hemophilia C (Factor XI Deficiency), Familial hemophagocytic lymphohistiocytosis, Acute lung injury, Hemolytic Uremic Syndrome, Menorrhagia, Chronic myeloid leukemia, or any combinations thereof.

In some embodiments of any aspect or embodiments herein that include an indication, the indication is selected from the group consisting of Fetal and neonatal alloimmune thrombocytopenia, intracranial hemorrhage (ICH), traumatic brain injury (TBI), Von Willebrand disease, Immune thrombocytopenia, and the indication is not treatable by administering unmodified platelets. In some embodiments, the indication is Von Willebrand disease, and wherein the indication is not treatable by administering unmodified platelets. In some embodiments, the indication is Immune thrombocytopenia, and wherein the indication is not treatable by administering unmodified platelets. In some embodiments, the indication is Fetal and neonatal alloimmune thrombocytopenia, wherein the indication is not treatable by administering unmodified platelets. In some embodiments, the indication is intracranial hemorrhage (ICH), and wherein the indication is not treatable by administering unmodified platelets. In some embodiments, the indication is traumatic brain injury (TBI), and wherein the indication is not treatable by administering unmodified platelets.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, the subject is having an indication that, typically cannot be treated with unmodified platelet preparation but can be treated with a platelet derivative composition as described herein. In some embodiments, such type of indication is Von Willebrand disease, Immune thrombocytopenia, Intracranial hemorrhage (ICH), Traumatic brain injury (TBI), Hermansky Pudlak Syndrome (HPS), Chemotherapy induced thrombocytopenia (CIT), Scott syndrome, Evans syndrome, Hematopoietic Stem Cell Transplantation, Fetal and neonatal alloimmune thrombocytopenia, Bernard Soulier syndrome, Acute myeloid leukemia, or combinations thereof.

In some aspects, provided herein is a method of treating immune thrombocytopenia in a subject, the method comprises administering an effective dose of freeze-dried platelet derivatives, in a platelet derivative composition, to the subject. In some aspects, provided herein is a method of treating intracranial hemorrhage (ICH) in a subject, the method comprises administering an effective dose of freeze-dried platelet derivatives, in a platelet derivative composition, to the subject. In some aspects, provided herein is a method of treating traumatic brain injury (TBI) in a subject, the method comprises administering an effective dose of freeze-dried platelet derivatives, in a platelet derivative composition, to the subject.

In some embodiments of any aspect or embodiment herein that include delivering platelet derivatives to a subject, or administering platelet derivatives to a subject, or treating a subject, or use of platelet derivative composition or platelet derivatives as described in any of the aspects or embodiments, platelet derivatives can have an effective dose or a therapeutically effective dose in the range of 1.0×10⁷ to 1.0×10¹¹ particles/kg of the subject. In some embodiments, platelet derivatives can have an effective dose or a therapeutically effective dose in the range 250 and 5000 TGPU per kg of the subject.

In some embodiments of any of the aspects or embodiments herein that include administering, the administering increases the levels of at least one platelet biomarker selected from CD62P, PAC-1, and CD63 for endogenous platelets of the subject (recipient) as compared to before the administering. In some embodiments, the administering increases the level of CD62P for endogenous platelets of the subject (recipient) as compared to before the administering. In some embodiments, the administering increases the level of PAC-1 for endogenous platelets of the subject (recipient) as compared to before the administering. In some embodiments, the administering increases the level of CD63 for endogenous platelets of the subject (recipient) as compared to before the administering. In illustrative embodiments, levels of both CD62P and PAC-1 are increased in the subject after the administering. In some embodiments, the subject (recipient) is having HPS.

In some aspects, or embodiments of any of the aspects or embodiments herein that include administering, the administering is performed to treat the subject, wherein either a) at least one HPS-related hemostatic abnormality and/or HPS-related biomarker abnormality observed in the subject is improved in the subject after the administering compared to before the administering; or b) normal levels of hemostasis and/or the HPS-related biomarker abnormalities are maintained in the subject. In some embodiments, the administering is performed to treat the subject such that at least one HPS-related hemostatic abnormality and/or HPS-related biomarker abnormality observed in the subject is improved in the subject after the administering compared to before the administering. In some embodiments, the administering is performed to treat the subject such that normal levels of hemostasis are maintained in the subject. In some embodiments, the administering is performed to treat the subject such that the HPS-related biomarker abnormalities are maintained to that of the normal levels in the subject. In some embodiments, the administering increases the levels of at least one platelet biomarker selected from CD62P, PAC-1, and CD63 for endogenous platelets of the subject having HPS as compared to before the administering. In some embodiments, the administering increases the levels of both CD62P and PAC-1 for endogenous platelets of the subject having HPS as compared to before the administering. In some embodiments, the administering increases the levels of all the platelet biomarkers CD62P, PAC-1, and CD63 for endogenous platelets of the subject having HPS as compared to before the administering. In some embodiments, the administering increases the levels of CD62P for endogenous platelets of the subject having HPS as compared to before the administering. In some embodiments, the administering increases the levels of PAC-1 for endogenous platelets of the subject having HPS as compared to before the administering. In some embodiments, the administering increases the levels of CD63 for endogenous platelets of the subject having HPS as compared to before the administering. In some embodiments, the administering leads to an improvement in thrombin generation in the subject having HPS as compared to the subject before the administering. In some embodiments, the administering leads to an improvement in clot formation in the subject having HPS as compared to the subject before the administering. In some embodiments, the administering leads to an improvement in clot formation in the subject having HPS as compared to the improvement in clot formation after administering platelets, such as apheresis platelets or fresh platelets.

In some embodiments, the administering is performed until the bleeding potential of the subject is reduced as compared to the bleeding potential before the administering. In some embodiments, the administering is performed until the bleeding stops. In some embodiments, the subject that has bleeding potential has HPS. In some embodiments, the subject that has bleeding potential has BSS.

In some embodiments, of any aspects or embodiments herein that include a method or a composition, or a composition for use, the platelet derivatives have the ability to generate thrombin in an in vitro thrombin formation assay, in illustrative embodiments, in the presence of a tissue factor, and phospholipids. In some embodiments, platelet derivatives have the ability to occlude a collagen-coated microchannel in vitro.

In some embodiments of any of the aspects or embodiments herein that include platelet derivatives or a platelet derivative composition, the platelet derivative composition comprises a population of platelet derivatives having a compromised plasma membrane and CD 41-positive platelet derivatives, wherein less than 5%, 4%, 3%, 2%, or 1% of the CD 41-positive platelet derivatives in the platelet derivative composition are microparticles. In some embodiments, the platelet derivative a) have the ability to generate thrombin in vitro in the presence of tissue factor and phospholipids; b) have the ability to occlude a collagen-coated microchannel in vitro; or c) both a) and b).

In some embodiments, of any aspects or embodiments herein that include a method of treating, or administering, or use of a composition, the subject is a mammal. In some embodiments, the subject is a human.

In some embodiments of any aspects or embodiments herein that include a method or platelet derivative composition for use, the platelet derivative composition is in the form of a powder, and before the administering, rehydrating the platelet derivatives to form a rehydrated platelet derivative composition, and the administering is administering an effective dose of the rehydrated platelet derivatives from the rehydrated platelet derivative composition to the subject. In some embodiments herein that include a method or platelet derivative composition for use, the platelet derivatives have a comprised immune plasma membrane.

In some embodiments of any aspect or embodiment herein that includes a method or platelet derivative composition for use, at least 55% of the platelet derivatives are positive for CD41. In some embodiments, wherein at least 55% of the platelet derivatives are positive for CD41 and have the size in the range of 0.5-2.5 μm. In some embodiments, at least 65% of the platelet derivatives are positive for CD42, and in some embodiments, at least 65% of the platelet derivatives are positive for CD42 and have the size in the range of 0.5-2.5 μm in diameter.

In some embodiments of any aspects or embodiments herein that include administering platelet derivatives, or use of a composition, the effective dose of the platelet derivatives is at least 10¹¹/kg, for example, at least 10¹²/kg. In some embodiments, the effective dose of the platelet derivatives is in the range of 3.0×10⁹ to 1.0×10¹²/kg, 3.0×10⁹ to 5.0×10¹¹/kg, 1×10¹⁰ to 1.0×10¹²/kg, or 5.0×10⁹ to 1.0×10¹²/kg of the subject. In some embodiments, a dose or an effective dose of the platelet derivatives can be 1.5×10⁷ to 1.1×10¹⁰/kg of the subject, or 1.5×10⁹ to 1.0×10¹²/kg of the subject.

In any of the aspects and embodiment herein that includes administering platelet derivatives to a recipient or a subject herein, a dose, an effective dose, dose for continuous infusion procedure (/min), therapeutically effective dose or amount of the platelet derivatives in a platelet derivative composition or for administering is in the range of 1.0×10⁷ to 1.0×10¹²/kg of the subject. In some embodiments, the administering can be as a continuous infusion procedure and the dose can be in the range of 1.0×10⁷ to 1.0×10¹²/kg/min of the subject. In some embodiments, the administering can be the effective amount or the dose is in the range of 1.0×10⁹ to 1.0×10¹², 1.2×10⁹ to 1.0×10¹², 1.4×10⁹ to 1.0×10¹², 1.6×10⁹ to 1.0×10¹², 1.8×10⁹ to 1.0×10¹², 2.0×10⁹ to 1.0×10¹², 3.0×10⁹ to 1.0×10¹², 3.5×10⁹ to 1.0×10¹², 4.0×10⁹ to 1.0×10¹², 5.0×10⁹ to 1.0×10¹², 5.5×10⁹ to 1.0×10¹², 6.0×10⁹ to 1.0×10¹², 6.2×10⁹ to 1.0×10¹², 6.4×10⁹ to 1.0×10¹², or 6.5×10⁹ to 1.0×10¹²/kg of the subject.

In any of the aspects and embodiment herein that includes administering platelet derivatives, a dose, an effective dose, dose for continuous infusion procedure (/min), or a therapeutically effective dose can be in the range of 1.0×10⁵ to 1.0×10¹/kg of the subject. In some embodiments, administering can be done as a continuous infusion procedure, and the dose can be in the range of 1.0×10⁵ to 1.0×10¹²/kg/min. In some embodiments, a continuous infusion procedure using any dose herein, for example, a dose from 1.0×10⁵ to 1.0×10¹²/kg/min can be done for at least 15 minutes, 30 minutes, 1 hour, 2 hours, 3 hours, 4 hours, 5 hours, 6 hours, 8 hours, 10 hours, 12 hours, 18 hours, 24 hours. In some embodiments, continuous infusion using any dose herein, for example, a dose from 1.0×10⁵ to 1.0×10¹²/kg/min can be done for a time period in the range of 30 minutes to 72 hours, 30 minutes to 48 hours, 30 minutes to 24 hours, 30 minutes to 18 hours, 30 minutes to 12 hours, or 30 minutes to 6 hours. In some embodiments continuous infusion procedure can be done with platelet derivatives at a dose in the range of 1.0×10⁵ to 1.0×10⁷/kg/min, 1.0×10⁵ to 1.0×10⁸/kg/min, 1.0×10⁵ to 1.0×10⁹/kg/min, 1.0×10⁵ to 1.0×10¹⁰/kg/min, 1.0×10⁵ to 1.0×10¹¹/kg/min, 1.0×10⁶ to 1.0×10¹²/kg/min, 1.0×10⁸ to 1.0×10¹²/kg/min, or 1.0×10¹⁰ to 1.0×10¹²/kg/min. In some embodiments, administering can be done multiple times by administering a single, double, triple, or more doses, in illustrative embodiments between 1.0×10⁵ to 1.0×10¹²/kg at a frequency, for example, at every 2 minutes, 3 minutes, 4 minutes, 5 minutes, 6 minutes, 7 minutes, 8 minutes, 9 minutes, 10 minutes, 15 minutes, 20 minutes, 30 minutes, 1 hour, 2 hours, 3 hours, 4 hours, 5 hours, 6 hours, 7 hours, 8 hours, 9 hours, 10 hours, 11 hours, 12 hours, or 24 hours. In certain illustrative embodiments, dosing is performed at a time interval between every 5 minutes and every 60 minutes. In some embodiments, a single, double, triple, or more doses can be administered at a frequency of a time within the range of 2 minutes to 24 hours, 2 minutes to 12 hours, 2 minutes to 6 hours, 2 minutes to 3 hours, 2 minutes to 2 hours, 2 minutes to 1 hour, 2 minutes to 45 minutes, 2 minutes to 30 minutes, 2 minutes to 20 minutes, 2 minutes to 15 minutes, 2 minutes to 10 minutes, or 2 minutes to 5 minutes. In some embodiments, the dosage in a single, double, triple or more doses can vary as per the time from the administration of the first dose. In some embodiments, the number of doses administered over a frequency of time herein can vary from 1 dose to 10 doses, 1 dose to 8 doses, 1 dose to 6 doses, 1 dose to 4 doses, or 1 dose to 3 doses. In some embodiments, administering can be done in multiple times, and the dose can vary in the range of 1.0×10¹ to 1.0×10¹⁰/kg, 1.0×10⁵ to 1.0×10⁸/kg, 1.0×10⁵ to 1.0×10⁷/kg, 1.0×10⁸ to 1.0×10¹²/kg, 1.0×10⁹ to 1.0×10¹²/kg, 1.0×10¹⁰ to 1.0×10¹²/kg, or 1.0×10¹¹ to 1.0×10¹²/kg of the subject. In some embodiments, the continuous infusion procedure or providing doses at a time frequency herein can be done depending on whether bleeding is reduced to a satisfactory level, for example such that it is no longer considered life-threatening, or no longer considered severe or serious, or continued until the bleeding is mild, or stops. For example, administering can be performed at a frequency of at least one dose every 2 to 5 minutes or more frequently starting from the first dose until the bleeding potential of the subject is reduced as compared to the bleeding potential before the administering. In some embodiments, administering of platelet derivatives, FDPDs, or FPH herein can be performed as a mixed procedure in which the continuous infusion can be interrupted with a specific dose of platelet derivatives followed by a specific interval as per the requirement, for example, status of bleeding of the subject or the recipient. In some embodiments, administering platelet derivatives, FDPDs, or FPH as a continuous infusion procedure, or providing single or multiple doses as per a time frequency as disclosed herein can be done as a part of a surgical procedure. In other words, a continuous infusion procedure or single or multiple dose administration can be done at a time frequency disclosed herein for a subject undergoing surgery. For example, administering platelet derivatives, FDPDs, or FPH can be done as a continuous infusion procedure during a surgery, such that any dose as disclosed herein can be infused 10, 20, 30, 40, 50, or 60 minutes, within 1 hour before, during, or within one hour after a surgery, or before a surgery is scheduled to end. In other embodiments, any dose can be provided as a continuous infusion procedure starting 60, 50, 40, 30, 20, or 10 minutes within 1 hour before, during or within 1 hour after a surgery, or before a surgery is scheduled to end. In some embodiments, the continuous infusion procedure can be done during the entire duration of the surgery. In some embodiments, the continuous infusion procedure can be done whenever there might be a risk of increased bleeding during the surgery. In some embodiments, platelet derivatives, FDPDs, or FPH can be administered every 2, 3, 4, 5, 6, 7, 8, or 9 minutes within 1 hour before, during, or within 1 hour after the surgery. In some embodiments, platelet derivatives, FDPDs, or FPH can be administered every 10, 20, 30, 40, 50, or 60 minutes within 1 hour before, during, or within 1 hour after the surgery.

In some embodiments, provided herein is a platelet derivative composition comprising platelet derivatives of any aspect provided herein, or a platelet derivative composition prepared by the process of any method for preparing or making provided herein, wherein the platelet derivatives comprise an imaging agent, to form imaging agent-loaded platelet derivatives. In some embodiments, the platelet derivatives in imaging agent-loaded platelet derivatives are freeze-dried platelet derivatives (FDPDs) that retain one, two, three, or more properties of FDPDs that are not loaded as described herein. In some embodiments, the imaging agent can be an MRI agent, such as gadolinium. In some embodiments, platelet derivatives comprise an MRI agent, termed as MRI agent-loaded platelet derivatives. In some embodiments, MRI agent-loaded platelet derivatives are freeze-dried platelet derivatives (FDPDs) that retain one, two, three, or more properties of FDPDs that are not loaded as described herein.

In some embodiments, provided herein is imaging agent-loaded platelets, cryopreserved platelets, or platelet derivatives. In some embodiments, provided herein is MRI agent-loaded platelets, cryopreserved platelets, or platelet derivatives. In some embodiments, imaging agent-loaded or MRI agent-loaded platelets, cryopreserved platelets, or platelet derivatives comprise a cell penetrating peptide (CPP). A CPP is coupled to an imaging agent or an MRI agent that facilitates the imaging agent or MRI agent to load onto platelets, cryopreserved platelets, or platelet derivatives. In illustrative embodiments, a CPP is a TAT peptide.

In some embodiments, provided herein is a method of delivering an imaging agent to a subject. In some embodiments, platelet derivatives or platelets as described herein are loaded with an imaging agent to form imaging agent-loaded platelet derivatives or platelets, such imaging agent-loaded platelet derivatives or platelets are administered to a subject. In some embodiments, platelet derivatives or platelets as described herein are loaded with an MRI agent to form MRI agent-loaded platelet derivatives or platelets, such MRI agent-loaded platelet derivatives or platelets are administered to a subject.

Also provided herein are compositions produced by any of the methods described herein. In some embodiments, any of the compositions provided herein can be made by the methods described herein. Specific embodiments disclosed herein may be further limited in the claims using “consisting of” or “consisting essentially of” language.

The following non-limiting examples are provided purely by way of illustration of exemplary embodiments, and in no way limit the scope and spirit of the present disclosure.

### EXAMPLES

### Example 1. Freeze-Dried Platelet Derivatives (FDPDs) Improve Thrombin Generation in Hermansky Pudlak Syndrome (HPS) Patients

Analysis of thrombin generation by Platelet Rich Plasma (PRP) obtained from blood of patients with HPS and further treated with varying doses of FDPDs, was performed. Human FDPDs were prepared according to the method as described in Example 1 of U.S. Pat. No. 11,529,587 B2, incorporated herein by reference in its entirety. The FDPDs were rehydrated with sterile water equivalent to the fill volume prior to lyophilization.

Whole blood from patients with HPS (n=4) and healthy donors (n=21) were collected in 3.2% sodium citrate tubes (BD #363083). Platelet rich plasma (PRP) was generated by centrifugation of whole blood for 10 minutes at a Relative Centrifugal Force (RCF) of 180×g and removal of the top PRP layer. The remainder of the tube was centrifuged for 10 minutes at a RCF of 1800×g and the top platelet poor plasma (PPP) layer was removed. The PRP was adjusted to 200k platelets/μL with PPP (k=10³>.

Thrombin calibrator reagent (Stago #86192) was prepared according to manufacturer's guidelines (Stago, available on the internet at stago.com). 20 μL of the thrombin calibrator reagent was added to each calibration well, and 20 μL of PBS was added to each thrombin generation well. PRP was aliquoted into microcentrifuge tubes and treated with FDPDs at 5, 20, and 50 k/μL of FDPDs. Samples were diluted 1:10 in OCTOPLAS® and a multi-channel pipette was used to add 80 μL of the diluted sample to each of the assay wells of an assay plate (calibration and thrombin generator wells). Three wells per sample were used for the thrombin calibrator and three wells were used for thrombin generation wells. Octaplas® plasma is a solvent/detergent treated, pooled human plasma available from Octapharma USA, Inc.

The assay plate was inserted into Thrombinoscope (Stago) connected to a computer with CAT software running. The assay plate was incubated at 37° C. for 10 min. During plate incubation Fluo-substrate and Fluo-buffer (Stago #86197) were combined according to manufacturer's guidelines to obtain FluCa buffer. ADP (Chronolog #384) and PGE1 (Cayman #13010) were added to FluCa buffer. ADP was added to FluCa buffer to obtain a final concentration of 1p M of ADP in assay wells and PGE1 was added to FluCa buffer to obtain a final concentration of 20 nM of PGE1 in the assay wells. After plate incubation, 20 μL FluCa solution was injected into each well. Thrombin generation was read for 180 min at 40 second intervals. Data analysis was performed in GraphPad Prism (GraphPad Software, San Diego, CA, graphpad.com).

Results of this experiment as shown in FIG. 1A and FIG. 1B. FIG. 1A and FIG. 1B show a reduced lag time and reduced time to peak of thrombin production with the addition of FDPDs to the PRP obtained from HPS patient blood, respectively. FIG. 1B shows approximately 2 fold reduction of time to peak thrombin production with the addition of FDPDs as compared to the HPS patient blood. Thus, these ex vivo results support that FDPDs can be effective in improving hemostasis in HPS patients.

### Example 2. FDPDs Improve Clot Formation of HPS Patients as Shown in Thromboelastography (TEG) Method

Analysis of blood coagulation factors (via clot formation) from blood obtained from normal patients, HPS patients, and HPS patients after ex vivo addition of 50 k/μL (k=10³) of FDPDs, was performed. Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization.

TEG assays monitor clot formation. Clot formation parameters are measured including time to clot initiation (r time), the rate of clot formation (k time), the angle of the clot, the maximum amplitude “MA” or “size of the clot”, and the percent platelet activation normalized to the citrated kaolin sample. The instrument measures the resistance to movement in a pin submerged in the sample as the cup of sample moves.

Whole blood was retrieved from HPS patients and healthy patients via sodium citrate vacutainers (BD ref #363083) and lithium heparin vacutainers (BD ref #367884) and used within two hours to perform the experiment. Citrate tubes were kept on a rocker and heparin tubes were kept upright and mixed by inversion prior to using them. Whole blood for HPS patients was tested untreated and treated by ex vivo addition of 50 k/μL of FDPDs. The TEG® PlateletMapping® Assay (Haemonetics cat #07-014, Boston, MA, haemonetics.com) was run according to manufacturer's instructions on TEG 5000 instrument. This required 2000 μL of citrated blood and 2,160 μL of heparin blood. For the samples treated with FDPDs, the test article was added to a microcentrifuge tube, blood to which FDPDs had been added ex vivo, was added to the tube and slowly mixed with a pipette, and then the run was immediately carried out according to the manufacturer's instructions. The results were returned automatically by the TEG software. Statistical analysis was carried out in GraphPad Prism (GraphPad Software, San Diego, CA, graphpad.com) using one-way ANOVA with Tukey's post-hoc test.

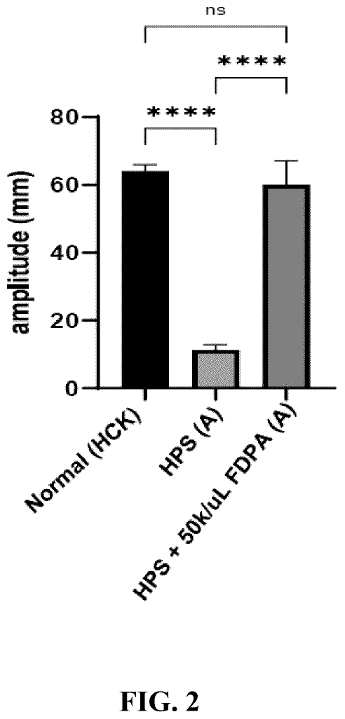

FIG. 2 shows the maximum amplitude of the clot of normal patient, HPS patient, and HPS patient blood treated with 50 k/μL of FDPDs. FIG. 2 confirms that HPS patients have reduced maximum amplitude of the clot and shows that the addition of 50 k/μL of FDPDs to HPS patient blood restores clot amplitude to near normal amplitude. The TEG® PlateletMapping® Assay clearly indicates that the additional of FDPDs to HPS blood ex vivo improves coagulation via clot formation amplitude improvement. FIG. 2 shows that the addition of FPDPs to HPS patient blood increases the clot amplitude to approximately 6 fold as compared to the HPS patient blood without the addition of FDPDs.

### Example 3. FDPDs Further Improve Clot Formation of HPS Patients in Comparison to Apheresis Platelets as Shown in Collagen Modified Thromboelastography (TEG) Method

Analysis of blood coagulation factors (via clot formation) from blood obtained from normal patients (n=5), HPS patients (n=3), and HPS patients after ex vivo addition of 50 k/μL (k=10³) (n=3) of FDPDs or 50 k/μL of apheresis platelets (n=1), was performed. Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization. TEG assays monitors clot formation. Clot formation parameters are measured including time to clot initiation (r time), the rate of clot formation (k time), the angle of the clot, the maximum amplitude “MA” or “size of the clot”, and the percent platelet activation normalized to the citrated kaolin sample. The instrument measures the resistance to movement in a pin submerged in the sample as the cup of sample moves.

Whole blood was retrieved from HPS patients and healthy patients via sodium citrate vacutainers (BD ref #363083) and lithium heparin vacutainers (BD ref #367884) and used within two hours to perform the experiment. Citrate tubes were kept on a rocker and heparin tubes were kept upright and mixed by inversion prior to using them. Whole blood for HPS patients was tested untreated and treated with 50 k/μL of FDPDs or 50 k/μL of apheresis platelets.

FIG. 3A, shows the maximum amplitude of the clot of blood from normal patients, HPS patients, HPS patients treated with 50 k/μL of FDPDs, and an HPS patient treated with 50 k/μL of apheresis platelets. FIG. 3B shows the rate of clot formation (k time) for blood from normal patients, HPS patients, HPS patients treated with 50 k/μL of FDPDs, and an HPS patient treated with 50 k/μL of apheresis platelets. The results illustrated in FIG. 3A confirm that HPS patients have reduced maximum amplitude of the clot and surprisingly show that the addition of 50 k/μL of FDPDs to HPS patient blood restores clot amplitude better than the same dose of apheresis platelets. Similarly, the results shown in FIG. 3B surprisingly show that 50 k/μL of FDPDs reduced the k time better than the same dose of apheresis platelets in this ex vivo assay. These results using the collagen modified TEG PlateletMapping Assay clearly indicate that the additional of FDPDs to HPS blood improves coagulation via clot formation amplitude and reduced rate of clot formation. FIG. 3A shows that the addition of FPDPs to HPS patient blood increases the clot amplitude to approximately 2 fold as compared to the HPS patient blood without the addition of FDPDs but with the addition of apheresis platelets.

### Example 4. FDPDs Contribute to an Increase of Endogenous PAC-1 Expression for HPS Patient Blood

The results in Examples 4 to 7 demonstrate that FDPDs contribute to an increase in levels of platelet activation biomarkers for endogenous platelets in HPS patient blood. Analysis of the first procaspase activating compound (PAC-1) expression from blood obtained from normal patients, HPS patients, and HPS patients after ex vivo addition of 20 k/μL (k=10³) of FDPDs was performed. Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization.

Whole blood was retrieved from HPS patients (n=3) and normal healthy patients (n=6) via sodium citrate vacutainers (BD ref #363083). Normal whole blood was left untreated and used as a control for reagents and instruments used in the assay. 1 mL of the HPS whole blood from the sodium citrate tube was transferred to two tubes. The first tube of HPS blood was left untreated and the second tube of HPS blood was treated by the addition of 20 k/μL of FDPDs. The tubes were inverted slowly three times and used immediately for PAC-1 staining.

For PAC-1 staining, 50 μL of each sample was stained in a well of a 96 well U-bottom plate with the following conditions: (1) 70 μL of HEPES-modified Tyrode's Albumin buffer (HMTA), 20 μL of muse-antihuman CD42b-FTIC (BD Biosciences Cat #555472), and 5 μL of mouse anti-human PAC-1AF647 (Biolegend 3628206), (2) 75 μL HMTA, 20 μL of mouse IgG1k-FITC (BD Cat #555748), and (3) 93.5 μL of HMTA, 1.5 μL of mouse IgM-AF647 (Biolegend Cat #401618).

To compensate for any color bleeding or any spillover of the different stains, single color controls were created. Single color controls are essential for any multicolor experiment. The single color controls reveal the level of spillover of the fluorophore into the other detectors. The spill over is mathematically removed, ensuring only specific signals are used in the final analysis.

Single color controls were created by combining 10 μL of each blood sample and adding 5 μL of the mixture into the wells with the following conditions: (1) 5 μL of PAC-1-AF647 in 90 μL of HMTA, (2) 20 μL CD42b-FITC in 75 μL of HMTA, and (3) 95 μL of HMTA.

Samples were stained in a dark room for 20 minutes at room temperature and then diluted 1:40 in HMTA and run on the Novocyte Quanteon flow cytometer (Agilent Santa Clara, CA, agilent.com). The flow cytometer was set up to collect events in either CD42b positive or CD41 positive to include only platelets in the analysis. The flow rate was set to medium and compensation was automatically calculated by the instrument and applied to all samples. To study only the endogenous HPS population, the contribution of FDPDs to the CD42b positive or CD41 positive was removed by gating on the negative population for FDPDs specific marker.

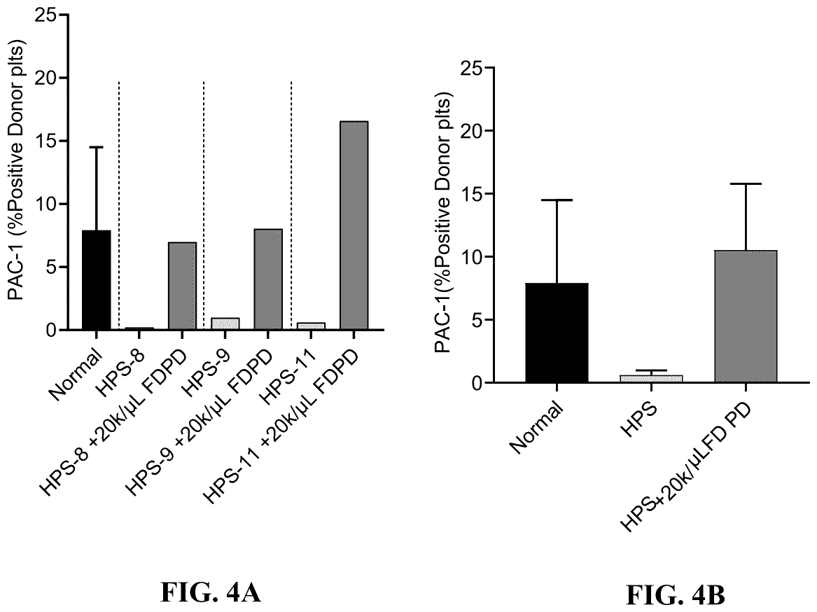

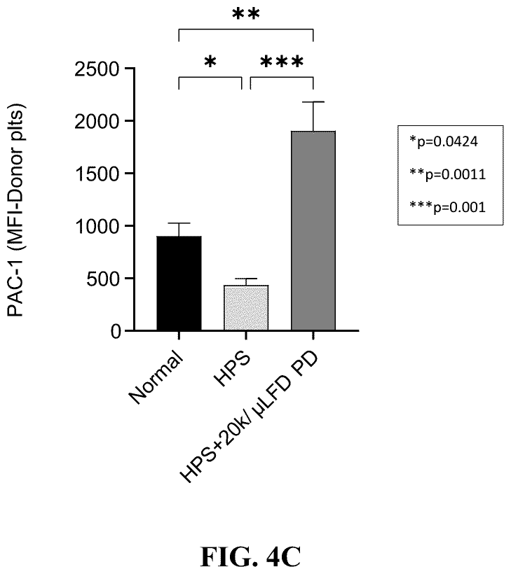

FIG. 4A and FIG. 4B show PAC-1 percent positivity of normal patients, HPS untreated patients, and HPS patients treated with 20 k/μL dose of FDPDs. FIG. 4A shows individual HPS patients separated by dashed vertical lines in comparison to normal and untreated HPS donor blood, FIG. 4B shows the same data as the mean and standard error of the mean of the HPS patients' data. Both FIG. 4A and FIG. 4B show that the addition of 20 k/μL of FDPDs to HPS blood is able to restore the percentage of endogenous platelets that are PAC-1 back to normal levels. FIG. 4C is the MFI representation of the same data as FIG. 4B and demonstrates the ability of exposure of HPS patient blood to FDPDs to increase the levels of PAC-1 in the HPS patient blood. FIG. 4A shows that addition of FDPDs exhibit positivity of PAC-1 of approx. 8% (an increase of more than 7% as compared to HPS blood before the addition of FDPD), 9% (an increase of more than 6-7% as compared to HPS blood before the addition of FDPD), and 20% (an increase of at least 18% as compared to HPS blood before the addition of FDPD).

### Example 5. FDPDs Contribute to an Increase of Endogenous CD62P Expression for HPS Patient Blood

Analysis of CD62P expression from blood obtained from normal patients, HPS patients, and HPS patients after ex vivo addition of 20 k/μL (k=10³) of FDPDs was performed. Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization.

Whole blood was retrieved from HPS patients (n=3) and normal healthy patients (n=6) via sodium citrate vacutainers (BD ref #363083). Normal whole blood was left untreated and used as a control for reagents and instruments used in the assay. 1 mL of the HPS whole blood from the sodium citrate tube was transferred to two tubes. The first tube of HPS blood was left untreated and the second tube of HPS blood was treated by the addition of 20 k/μL of FDPDs. The tubes were inverted slowly three times and incubated on the rocker for an additional 10 minutes. After 10 minutes on the rocker, 50 μL of each blood sample was combined with 50 μL of Thrombofix (Beckman Coulter Cat #6607130). The blood samples were left with the fixation solution for at least 1 hour followed by staining for CD62P (P-selectin).

For CD62P staining 5 μL of each sample was stained in a well of a 96 well U-bottom plate with the following conditions: (1) 70 μL of HMTA, 5 μL CD41-PE (Beckman Coulter Cat #1M1416U, and 20 μL CD62P-PECy5 (BD Biosciences Cat #551142), (2) 75 μL HMTA buffer containing 20 μL of mouse IgG1k-PECy5 (BD Biosciences Cat #555750), and (3) 90 μL of HMTA buffer containing 5 μL of mouse IgG1-PE (Beckman Coulter IM0670U).

Single color controls were created by combining 10 μL of each blood sample and adding 5 μL of the mixture into the wells with the following conditions: (1) 20 μL CD62P-PECy5 in 75 μL of HMTA, (2) 5 μL CD41-PE in 90 μL of HMTA, and (3) 95 μL of HMTA.

Samples were stained in a dark room for 20 minutes at room temperature and then diluted 1:40 in HMTA and run on the Novocyte Quanteon flow cytometer (Agilent Santa Clara, CA, agilent.com). The flow cytometer was set up to collect events in either CD42b positive or CD41 positive to include only platelets in the analysis. The flow rate was set to medium and compensation was automatically calculated by the instrument and applied to all samples. To study only the endogenous HPS population, the contribution of FDPDs to the CD42b positive or CD41 positive was removed by gating on the negative population for FDPDs specific marker.

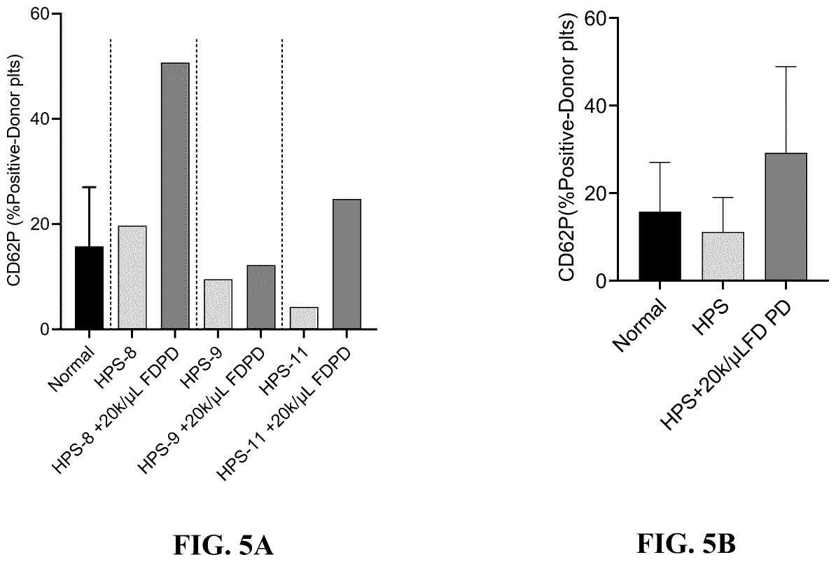

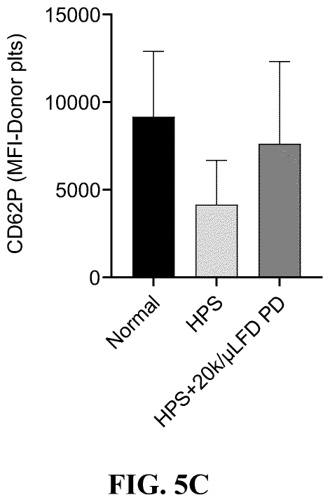

FIG. 5A and FIG. 5B show CD62P-percent positivity when analyzing blood of normal patients, HPS patients without addition of FDPDs, and HPS patients in which 20 k/μL of FDPDs had been added before performing the analysis. FIG. 5A shows each individual HPS patients separated by dashed vertical lines in comparison to normal and untreated HPS donor blood, FIG. 5B shows the same data as mean and standard deviation error of the mean of the HPS patients' data. Both FIG. 5A and FIG. 5B show that the addition of 20 k/μL of FDPDs to HPS blood restores the percentage of positivity of endogenous platelets that are CD62P back to normal levels. FIG. 5C is the MFI representation of the same data as FIG. 5B and demonstrate the ability of exposure of HPS patient blood to FDPDs to increase the levels of CD62P in the HPS patient blood. FIG. 5A shows that addition of FDPDs exhibit positivity of CD62P of approx. 50% (an increase of around 30% as compared to HPS blood before the addition of FDPD), and 30% (an increase of at least 20% as compared to HPS blood before the addition of FDPD).

### Example 6. FDPDs Contribute to an Increase of Endogenous CD63 Expression for HPS Patient Blood

Analysis of CD63 expression from blood obtained from normal patients, HPS patients, and HPS patients after ex vivo addition of 20 k/μL (k=10³) of FDPDs was performed. Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization.

Whole blood was retrieved from HPS patients (n=3) and normal healthy patients (n=4) via sodium citrate vacutainers (BD ref #363083). Normal whole blood was left untreated and used as a control for reagents and instruments used in the assay. 1 mL of the HPS whole blood from the sodium citrate tube was transferred to two tubes, the first tube of HPS blood was left untreated and the second tube of HPS blood was treated with 20 k/μL of FDPDs. The tubes were inverted slowly three times and incubated on the rocker for an additional 10 minutes. After 10 minutes on the rocker, 50 μL of each blood sample was combined with 50 μL of Thrombofix (Beckman Coulter Cat #6607130). The blood samples were left with the fixation solution for at least 1 hour followed by staining for CD63.

For CD63 staining 5 μL of each sample was stained in a well of a 96 well U-bottom plate with the following conditions: (1) 85 μL of HMTA, 5 μL of mouse anti-human CD41-PE (Beckman Coulter Cat #1M1416U) and 5 μL of mouse anti-humanCD63-APC (Biolegend Cat #353008), (2) 90 μL HMTA, 5 μL of mouse IgG1-PE (Beckman Coulter IM0670U), and (3) 90 μL of HMTA, 5 μL of mouse IgG1-APC (Biolegend Cat #400122).

Single color controls were created by combining 10 μL of each blood sample and adding 5 μL of the mixture into the wells with the following conditions: (1) 5 μL CD63-APC in 90 μL of HMTA, (2) 5 μL CD41-PE in 90 μL of HMTA, and (3) 95 μL of HMTA.

Samples were stained in a dark room for 20 minutes at room temperature and then diluted 1:40 in HMTA and run on the Novocyte Quanteon flow cytometer (Agilent Santa Clara, CA, agilent.com). The flow cytometer was set up to collect events in either CD42b positive or CD41 positive to include only platelets in the analysis. The flow rate was set to medium, and compensation was automatically calculated by the instrument and applied to all samples. To study only the endogenous population of platelets in the HPS blood treated with FDPDs, the contribution of FDPDs to the CD42b positive or CD41 positive was removed by gating on the negative population for FDPDs specific marker.

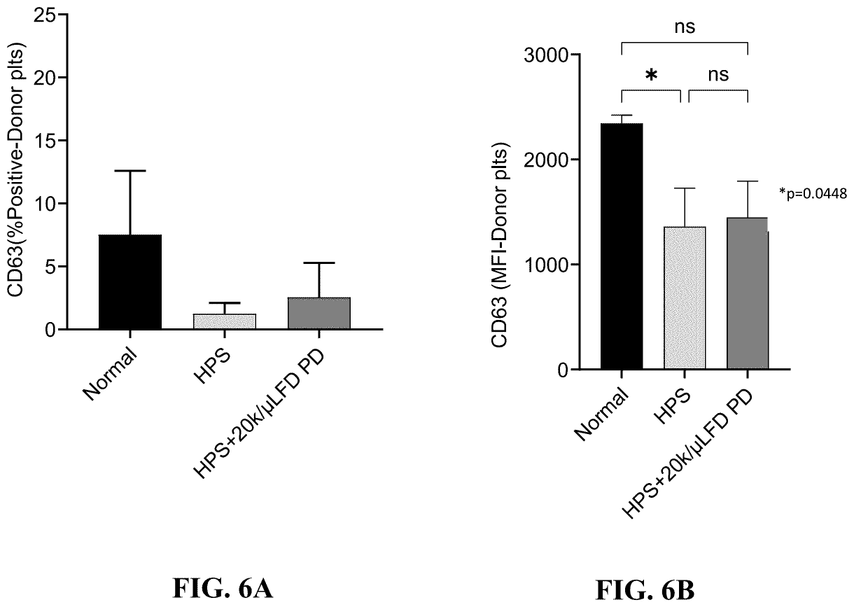

FIG. 6A shows cumulative data for CD63-percent positivity of normal patients, HPS patients, and HPS patients after ex vivo addition of 20 k/μL of FDPDs. FIG. 6A shows that the addition of 20 k/μL of FDPDs to HPS blood increases the number of cells that are positive for CD63 in comparison to the untreated HPS blood, bringing the percent positive cell numbers in the FDPD-treated sample closer to the normal sample. FIG. 6B is the MFI representation of the same data as FIG. 6A.

### Example 7. Crossmatching Assay to Detect Level of IgG Via Flow Cytometry

Platelet antibodies in plasma or serum controls were detected by incubating samples with FDPDs to perform a crossmatching assay. Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization.

HPS citrated plasma was retrieved from HPS whole blood. 60 μL of 30 k/μL FDPDs were added to 60 μL of HPS plasma and incubated for 20 minutes at room temperature in a shaker. A positive serum and negative human serum were included and treated as the HPS plasma. After incubation cells were recovered by centrifugation at RCF of 1,100×g for 10 minutes. The supernatant was removed, and the pellet was washed twice with a (FWS) flow wash buffer (1×PBS and 5% Heat-inactivated normal goat serum). The cell pellet was resuspended in FWS at a concentration of 100K cells/μL. The staining of the samples was done by incubating with PE-labeled anti-human IgG (GOAT, F(ab′)₂ fragment) (Jackson Immuneresearch Laboratories, Inc, West Grove PA, jacksonimmuno.com) at room temperature for 20 minutes away from light. After the staining incubation, 500 μL of 1×PBS was added to all samples.

Analysis was performed by transferring 100 μL of the samples to a 96 well plate using a NovoCyte flow cytometer (Agilent, Santa Clara, CA, agilent.com). Thirty thousand events in the relevant gate surrounding FDPD population were collected. Fluorescent intensity from the gated population was used to calculate IgG levels.

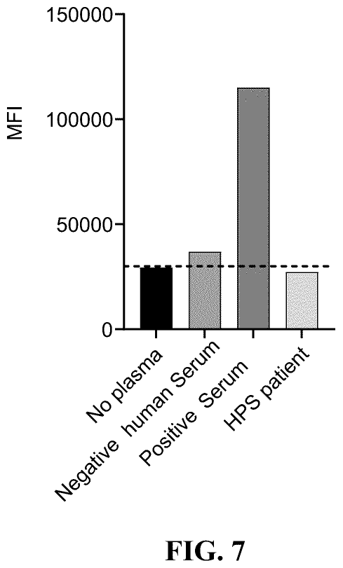

As shown in FIG. 7, anti-platelet IgG antibodies were detected in the positive serum. However, anti-platelet IgG antibodies were not detected in the HPS patient with ex vivo addition of FDPDs. Therefore, HPS patient with ex vivo addition of FDPDs does not lead to production of IgG antibodies against the FDPDs.

### Example 8. Addition of FDPDs to HPS Patient Blood Shows Dose Dependent Improvement of the Occlusion Time in PRP

Analysis of occlusion time with the addition of FDPDs to HPS PRP patient sample was measured on the Total Thrombin formation Analysis System (T-TAS® 01) using HD chips (collagen and tissue factor stimulant with higher shear speed). Human FDPDs were prepared according to the method as described in EXAMPLE 1 of U.S. Pat. No. 11,529,587 B2, incorporated herein by reference in its entirety. The FDPDs were rehydrated with sterile water equivalent to the fill volume prior to lyophilization.

Analysis samples were prepared from HPS whole blood collected in acid citrate dextrose (ACD) collection tubes (BD vacutainers Ref #364606). Platelet rich plasma (PRP) was prepared by centrifugation of the ACD whole blood at a RFC of 180×g for 10 minutes and removal of the top PRP layer. The remainder of the tube was centrifugated for 10 minutes at a RCF of 1800×g and the top platelet poor plasma (PPP) layer was removed. The PRP was adjust to 75,000/μL with PPP.

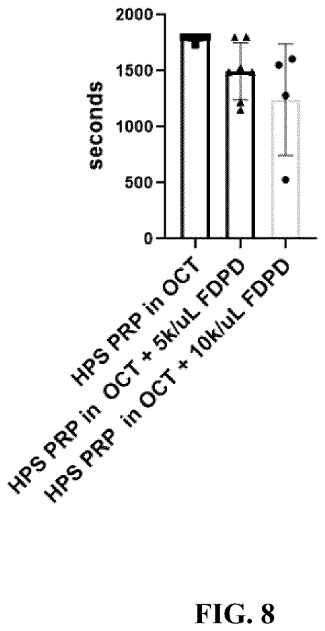

T-TAS® 01 (Zacros, zacros.co.jp) instrument was loaded with the HD chip and prepared for use according to the manufacturer's instructions. Three samples were run on the T-TAS® 01 machine, (1) HPS PRP and Octaplas®, (2) HPS PRP, Octaplas®, and 5k/μL of FDPDs, and (3) HPS PRP, Octaplas®, and 10 k/μL of FDPDs. Octaplas® plasma is a solvent/detergent treated, pooled human plasma available from Octapharma USA, Inc. For each sample 480 μL of the prepared sample in the Octaplas® plasma was placed into a microcentrifuge tube with 20 μL of provided calcium CTI (CaCTI) reagent (Zacros, zacros.co.jp). The mixture was gently pipetted, loaded into T-TAS® 01 reservoir, and run on machine. FIG. 8 shows that addition of FDPDs to HPS PRP improves the time to occlude within the HD channel in comparison to untreated HPS PRP. Furthermore, a high dose of FDPDs is shown to further reduce the occlusion time.

### Example 9: In Vitro Surrogate Model for Bernard Soulier Syndrome (BSS)

An in vitro surrogate model of BSS was created and tested to confirm BSS phenotype using flow cytometry and light transmission aggregometry (LTA). The defect in BSS platelets is due to abnormalities in the Gp1b-V-IX complex affecting the binding to von Willebrand Factor (vWF) when exposed to vascular injury on subendothelial surfaces and thus interfering with primary hemostatic plug.

Single donor apheresis platelet units were purchased from LifeShare Blood Center. Apheresis platelets were treated with anti-CD42b antibody targeting the ligand binding domain region GPIbα and blocking the binding of vWF. Anti-CD42b clone AK2 (Invitrogen, Thermo Fisher Scientific, thermofisher.com) was selected for the blocking experiment (treated platelets). The treated platelets as obtained herein can also be called as a surrogate BSS model. A control untreated sample (untreated or control platelets) was used for comparison, the control was washed and incubated along with the treated platelets to assess the effect of platelet manipulation. Platelets were quantified using Act Diff2 (Beckman Coulter, beckmancoulter.com) and aliquots for treatment were prepared using 250,000/μL as target concentration. Platelets were washed using platelet wash buffer. Platelet wash buffer comprises 128 mM NaCl, 4.26 mM Na2HPO4, 7.46 mM NaH2PO4, 4.77 mM Trisodium Citrate, 2.35 mM Citric Acid, 5.5 mM Glucose, and 0.35% BSA, pH 6.5 After washing with platelet wash buffer platelets were recovered by centrifugation at 1,000 g×10 min. The pellet were then resuspended in HEPES buffer (145 mM NaCl, 10 mM HEPES, 0.5 mM Na2HPO4, 5 mM KCl, 2 mM MgCl2, 1% Glucose, 0.35% BSA, 0.02 U/mL Apyrase, pH 7.4) and 0.025 μg/μL of anti-CD42b antibody was added. Samples were incubated for 30 min at room temperature in the incubator. After incubation platelet wash buffer was added to the sample and the platelets were recovered by centrifugation at 1,000 g for 10 min. Platelet pellets were either resuspended in either flow wash buffer (1×PBS, 5% Heat inactivated Normal Mouse Serum (Innovative Research, innov-research.com)) or in Octoplas® (Octopharma, octapharmausa.com) and counted using Act Diff2 with a final concentration that was adjusted based on the requirements of the follow-up assay.

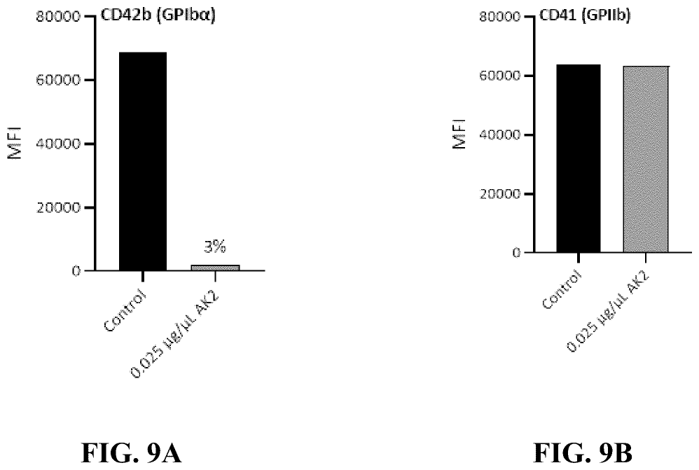

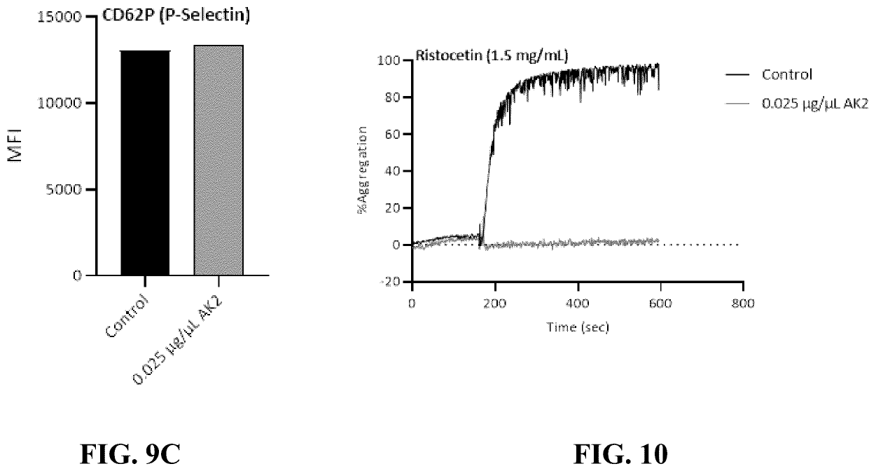

The apheresis platelets samples (treated and untreated) were first tested to confirm inhibition of the Gp1b-V-IX complex and expression of platelet activation surface markers using flow cytometry. For the flow cytometry assay the samples were resuspended in flow wash buffer and adjusted to 100,000/μL. Levels of GPIbα, CD41, and CD62P were detected and quantified using FITC labelled anti-CD42b HIP1 antibody (Becton Dickinson Biosciences, bdbiosciences.com), PE-labelled anti-CD41 P2 antibody (Beckman Coulter, beckmancoulter.com), and PE-labelled anti-CD62P AC1.2 antibody (Becton Dickinson Biosciences, bdbiosciences.com), respectively. For each marker to be tested, 10 μL of each sample was transferred into a well of a 96-well U-bottom plate (Immulon 2-HB), staining reactions were performed at a final volume of 30 μL and adjusted using flow wash buffer. Isotype controls for each antibody were included to establish background levels. Samples were stained for 20 min in the dark at room temperature. Before acquisition, samples were diluted 1:20 in 1×PBS, 100 μL of each sample was transferred to a well of a 96-well U-bottom plate and run on the Novocyte Quanteon flow (Agilent, agilent.com). The flow cytometer was set up to collect 30,000 events total. Data was analyzed by first applying a size gate for the platelet population. Within that gate, the markers were analyzed using the isotype controls as guides for gate placement. Data was reported as percentage positive platelets and mean fluorescent intensity (MFI). FIGS. 9A-9C show the surface marker expression for CD42b, CD41, and CD62P, respectively. FIG. 9A confirms the inhibition of GPIbα in the treated platelets (surrogate BSS model) (0.025 μg/μl AK2) using the AK2 antibody, and thereby confirming that in the treated platelets (surrogate BSS model) the binding of vWF is blocked. Further FIGS. 9B and 9C show that the inhibition of GPIbα in the treated platelets did not affect the expression of the activation markers of the treated platelets. The levels of surface expression for CD41 (FIG. 9B) and CD62P (FIG. 9C) in the treated platelets are similar to the control untreated sample.

Next the effect of the AK2 antibody treatment was evaluated by Light Transmission Aggregometry (LTA). In a clinical setting, diagnosis of BSS is based on the aggregation exhibited by platelets in the subject using ristocetin induced platelet aggregation (RIPA) assay. It is known that platelets of a subject diagnosed with BSS exhibit less aggregation in RIPA as compared to platelets of a healthy subject. Treated platelets (surrogate BSS model) and untreated platelets (control) were tested with RIPA assay, samples were counted and adjusted to 200,000/μL in Octoplas. RIPA was performed by adding 1.5 mg/mL of ristocetin (Helena Bioscience, helena-biosciences.com) to treated platelets and untreated control platelets. Data collection was performed in the PAP8 Platelet Aggregometer. 225 μL of adjusted platelets were added to the corresponding cuvettes, and incubated for 2 min with stirring at 37° C. After the initial incubation, 25 μL of ristocetin was added to the corresponding cuvette. After the run was complete, the data was exported and processed using GraphPad, and was reported as percent aggregation. FIG. 10 shows lack of aggregation exhibited by the treated platelets (surrogate BSS model) (0.025 μg/μl AK2) in the presence of ristocetin as compared to the aggregation exhibited by the control platelets (control) in the presence of ristocetin (1.5 mg/ml), thereby, confirming the in vitro BSS surrogate model.

### Example 10: FPH Treated In Vitro BSS Surrogate Model

Total thrombus formation analysis system (T-TAS) assay was performed to determine the effect of the addition of FPH on the surrogate BSS model platelets. A composition comprising thrombosomes having hemostatic properties, also referred to herein as a freeze-dried platelet-derived hemostat (FPH) was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

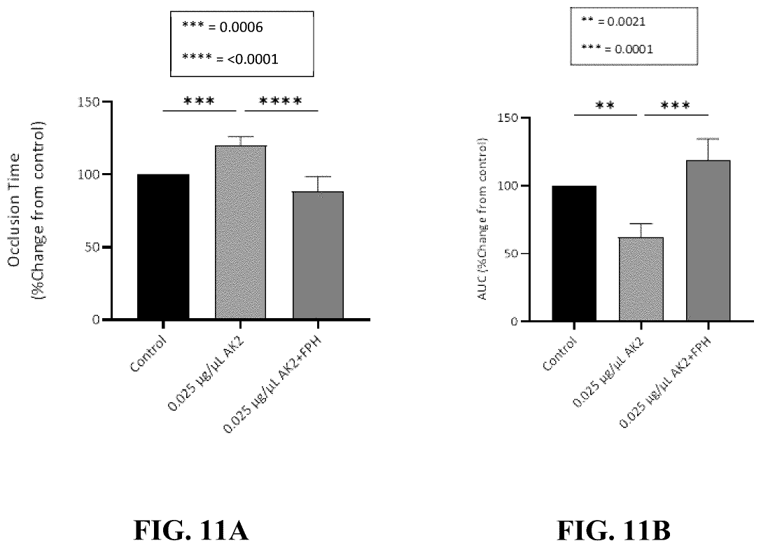

Treated platelets and untreated control platelet samples of Example 9 were counted and adjusted to 75,000/μL with Octoplas and ran on the T-TAS®01 (Zacros, zacros.com) using an HD chip (Zacros, zacros.com). HD chip is a high shear channel with collagen and tissue thromboplastin activators. 480 μL of corresponding sample was transferred into a 1.7 mL microcentrifuge tube with 20 μL of provided Calcium CTI plus reagent (Zacros, zacros.com). Measurement was initiated by transferring 450 μL of sample into T-TAS®01 reservoir. Samples were run twice. To assess the effect of FPH, 50,000/μL of FPH was added to the microcentrifuge tubes with the platelet samples (treated and untreated control). Data analysis was performed using GraphPad and reported as mean with standard deviation of the mean of the vessel occlusion time and area under the curve (AUC). FIG. 11A and FIG. 11B show the occlusion time and area under the curve of the untreated control platelet sample, surrogate BSS model platelet sample, and surrogate BSS model platelet sample treated with FPH at 50,000/μL, respectively. In FIG. 11A, it is observed that the addition of FPH to surrogate BSS model platelets (0.025 μg/μl AK2+FPH) reduces the occlusion time as compared to that of only surrogate BSS model platelets (0.025 μg/μl AK2) and brings it to a level that is similar to that observed for the control sample. In FIG. 11B, it is observed that the addition of FPH to surrogate BSS model platelets (0.025 μg/μl AK2+FPH) improves the AUC to levels that is greater than that of only surrogate BSS model platelets (0.025 μg/μl AK2), and restores the AUC back to normal untreated control platelet sample.

### Example 11: Crossmatching Assay to Detect Level of IgG Via Flow Cytometry

Platelet antibodies in HPS patients' plasma and serum controls were detected by incubating samples with FPH to perform crossmatching assay. The test was run with 32 HPS patient samples and compared with a control serum and a known reactive serum. Flow cytometry detection of platelet reactive antibodies is an effective method to detect alloantibodies bound to target platelets or lymphocytes.

Single donor apheresis platelet units (APU) were purchased from LifeShare Blood Center. FPH was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

HPS patient blood samples that have been tested for the presence of anti-HLA and anti-HPA antibodies by high resolution HLA Class 1 typing and HLA Class 1 antibody screening were received from the American Red Cross. Of the 32 HPS patient blood samples (2004-001-HPS0001 to 2004-001-HPS0032), 10 samples were identified as positive for anti-HLA or anti-HPA antibodies, namely, 2004-001-HPS0001, 2004-001-HPS0004, 2004-001-HPS0007, 2004-001-HPS0008, 2004-001-HPS0014, 2004-001-HPS0015, 2004-001-HPS0016, 2004-001-HPS0020, 2004-001-HPS0027, and 2004-001-HPS0029. Out of the 10 samples, 4 of the samples were identified as positive for anti-HPA antibodies, namely, 2004-001-HPS0001, 2004-001-HPS0007, 2004-001-HPS0008, and 2004-001-HPS0029. HPS citrated plasma was retrieved from HPS patient samples.

Each HPS patient serum sample was incubated with three different lots of FPH (Lot A, Lot B, Lot C). FPH was adjusted to a count of 30 k/L in flow wash buffer (FWB) (1×PBS and 5% Heat-inactivated normal goat serum, Jackson Immuneresearch Laboratories, Inc, West Grove PA, jacksonimmuno.com). 200 μL of diluted FPH suspension was added to a microcentrifuge tube plus 200 μL of FWB for each individual test.

Samples were centrifuged at RCF of 1,100×g for 10 minutes at room temperature in a microcentrifuge to pellet the FPH. Supernatant was discarded and the pellet was resuspended in 60 μL of FWB plus 60 μL of HPS patient serum or control serum and incubated for 20 minutes at room temperature on a rocker. Controls consisted of a known positive sample (University of Michigan, serum 6) and normal human serum (negative control) (Innovative Research, Novi MI, innov-research.com). FPH was incubated with FWB to create the no serum control. After incubation, cells were recovered by centrifugation at RCF of 1,100×g for 10 minutes, the supernatant was removed, and the pellet was resuspended with 500 μL FWB. The cells were centrifuged two more times, and finally resuspended with 30 μL of FWB. For staining, 10 μL of cells were incubated with 0.2 μg PE-labeled anti-Human IgG (Goat, F(ab′)₂ fragment) (Jackson ImmunoResearch Lab) and 62.5 ng/mL FITC-labeled CD61 (BD Biosciences 348093) and brought up to a final volume of 50 μL with HMTA. Cells were stained at room temperature for 20 minutes away from open light. After incubation, samples were diluted 1:20 with PBS and acquired on the NovoCyte Quanteon flow cytometer (Agilent, Santa Clara, CA, agilent.com). Mean fluorescence intensity (MFI) of IgG on the surface of FPH within the CD61-positive population was analyzed using NovoExpress software.

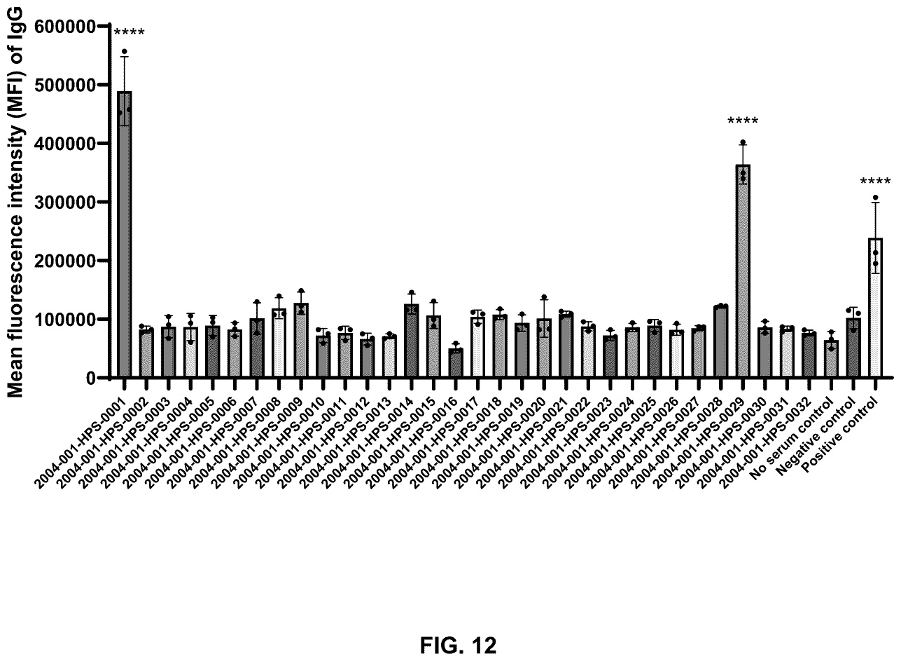

FIG. 12 shows the average MFI from the three FPH lots, with error bars representing standard deviation. One-Way ANOVA with Dunnett's test was performed in GraphPad Prism and used to determine statistical difference compared to the normal plasma sample. ****(p<0.0001). The negative and positive controls behaved as expected (average MFI of 101,465±17,928 vs 238,094±58,597, respectively). Two samples, HPS-0001 and HPS-0029, showed cross-reactivity to the three lots of FPH, both samples were previously identified as positive for anti-HLA and anti-HPA antibodies. A comparison of the compiled data for the three lots of FPH for HPS-0001 serum and the negative control shows a significant increase on IgG detected on the FPH surface (489,936±60,633 vs 101,465±17,928, p<0.0001). Increased levels of IgG were detected with HPS-0029 serum sample. Comparison of the compiled data for the three lots of FPH for HPS-0029 serum and the negative control shows a significant increase on IgG detected on the FPH surface (354,363±25,585 vs 101,465±17,928, p<0.0001).

### Example 12: FPH Ability to Generate Thrombin is not Affected by HPS Patient Plasma Samples

FPH ability to maintain thrombin generation ability (TGA) parameters compared to a normal plasma control was shown in HPS plasma samples overall, as well as HPS plasma samples positive for anti-HLA or anti-HPA antibodies and HPS plasma samples with cross reactivity to FPH.

Single donor apheresis platelet units (APU) were purchased from LifeShare Blood Center. FPH was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

HPS patient blood samples that have been tested for the presence of anti-HLA and anti-HPA antibodies by high resolution HLA Class 1 typing and HLA Class 1 antibody screening were received from the American Red Cross. Of the 32 HPS patient blood samples, 10 samples were identified as positive for anti-HLA or anti-HPA antibodies. HPS citrated plasma was retrieved from HPS patient samples.

FPH were adjusted to a count of 30,000 cells/μL in FWB (Jackson ImmuneResearch Laboratories, Inc). 200 μL of diluted FPH suspension was added to a microcentrifuge tube plus 200 μL of FWB for each individual test. Samples were centrifuged at RCF of 1,100×g for 10 minutes at room temperature in a microcentrifuge to pellet the FPH. Supernatant was discarded and the pellet was resuspended in 60 μL of FWB plus 60 μL of HPS patient serum or control serum and incubated for 20 minutes at room temperature on a rocker. After the incubation of FPH with plasma, each sample was diluted to a final concentration of 7,200 FPH/μL in 30% Octoplas®/70% TGA dilution buffer. Each sample was assayed in triplicate by adding 80 μL of sample per well with either 20 μL of PRP reagent or calibrator (Stago, stago.com), as per the manufacture's protocol. Cephalin control was prepared by diluting UPTT reagent (Bio/Data Corporation #105997, Horsham PA, biodatacorp.com) 1:50 in 30% Octaplas®/70% TGA dilution buffer. Lactadherin control was prepared by centrifuging FPH as above and resuspending the pellet in 120 uL FWB with 0.59 μg lactadherin (Haematologic Technologies #BLAC-1200, goprolytox.com). The sample was then incubated and diluted as above. The “no plasma control” was prepared by incubating with FWB in place of plasma. The normal human plasma control was prepared by pooling plasma from 8 normal donors and incubating the pooled sample with FPH as above. The run was started by injecting 20 μL of FluCa reagent (Stago, stago.com) on a Fluoroskan plate reader (Thermo Fisher Scientific, thermofisher.com) according to the manufacturer's protocols. The assay was set to run for 75 minutes, with measurements taken every 60 seconds. Peak thrombin, lag time, endogenous thrombin potential, time to peak, and velocity were automatically calculated by the Thrombinoscope software. Data is plotted as the average from the three FPH lots, with error bars representing standard deviation. One-Way ANOVA with Dunnett's test was performed in GraphPad Prism and used to determine statistical difference compared to the normal plasma sample. *(p<0.05), **(p<0.01), ***(p<0.001), ****(p<0.0001).

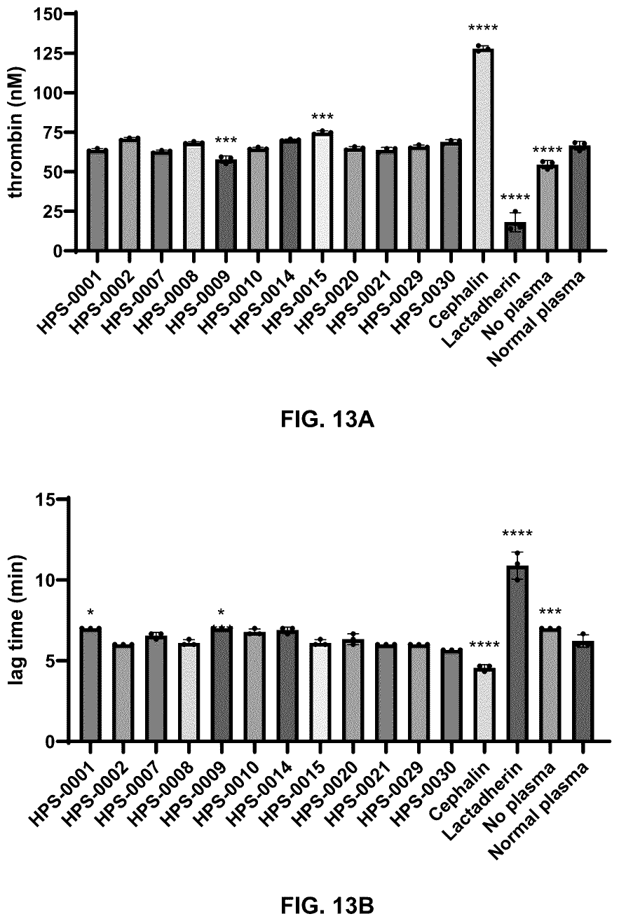

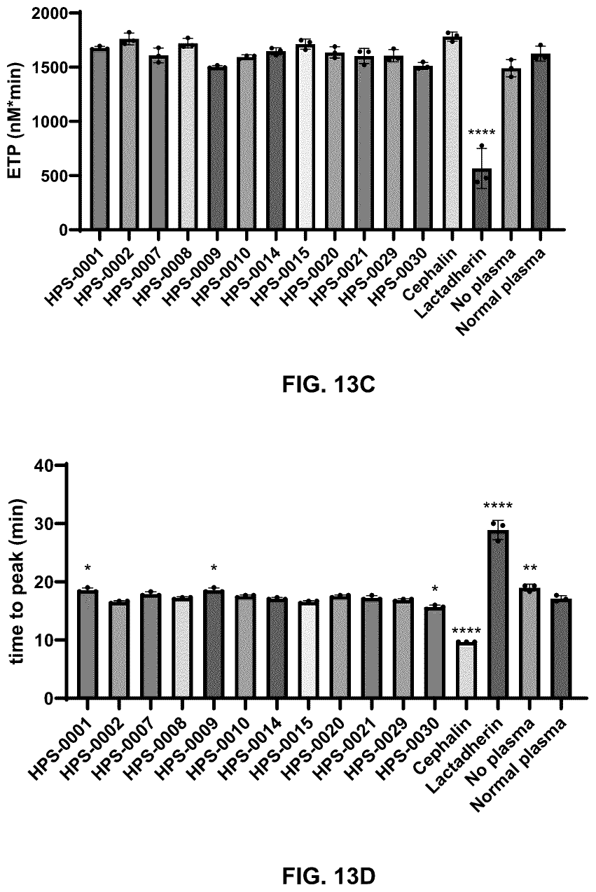

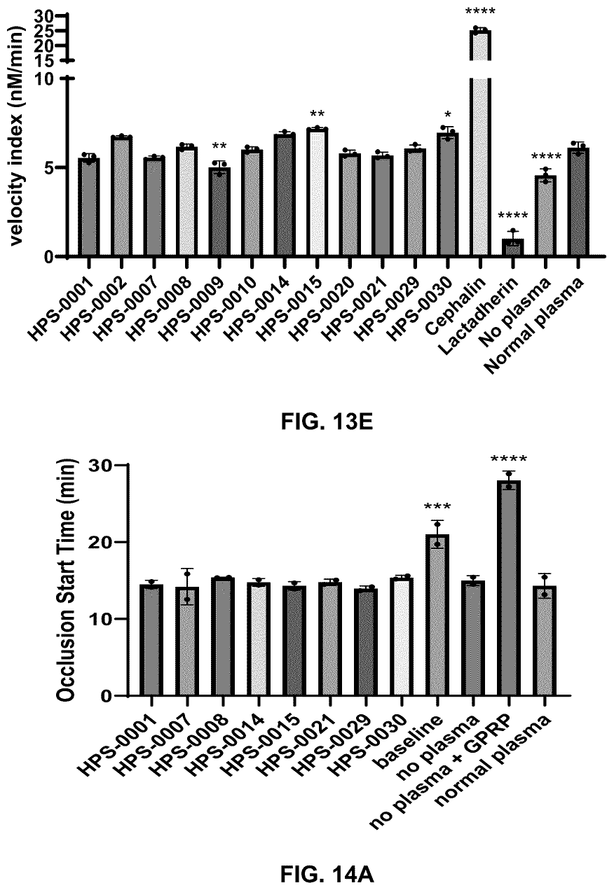

FIG. 13A shows the peak thrombin for the different HPS serum samples when incubated with FPH. FIG. 13B shows the lag time, time until thrombin first generated for the different HPS samples when incubated with FPH. FIG. 13C shows the endogenous thrombin potential or the net amount of thrombin that HPS plasma samples can generate when incubated with FPH. FIG. 13D shows the time to peak or the time for maximum concentration of thrombin generated. FIG. 13E shows the velocity index or the slope of the thrombin potential between the lag time and the time to peak.

Although some HPS plasma samples had statistically significant differences in TGA parameters compared to the normal plasma control, none of these differences are likely to be biologically meaningful rather they likely represent a variability within the human population. The only patient sample that demonstrated a consistent difference from normal plasma was HPS-0009, a sample negative for anti-HLA or anti-HPA antibodies. Patient sample HPS-0015, positive for anti-HLA antibodies, had a higher peak thrombin (74.8±1.06 nM vs. 66.5±2.76 nM; p=0.0004) and higher velocity index than normal (7.2±0.06 nM/min vs. 6.1±0.32 nM/min; p=0.0034).

Patient sample HPS-0001, positive for anti-HLA and anti-HPA antibodies (anti-GPIIbIIIa antibodies), was highly cross-reactive with FPH, had a longer lag time (7.0±0.0 min. vs. 6.2±0.4 min.; p=0.0157) and a longer time to peak (19±0.39 min. vs. 17±0.51 min; p=0.0188), but no change in peak thrombin, velocity, or ETP. Patient sample HPS-0029, having high FPH cross-reactivity, was not significantly different from the control for any other parameter.

These results indicate that the ability of FPH to generate thrombin was not significantly altered in the presence of HPS plasma, HPS plasma containing anti-HLA or anti-HPA antibodies, or HPS plasma with antibodies that cross-react to the FPH. The ability of FPH to generate thrombin is maintained regardless of the HPS plasma specification as shown in the TGA assay.

### Example 13: FPH Hemostatic Activity is Maintained on the T-TAS® when Incubated with Different HPS Plasma Samples

FPH pre-incubated with HPS plasma or normal plasma was tested on the T-TAS using a model of whole blood containing a low concentration of endogenous platelets. FPH hemostatic activity on the T-TAS is not altered due to incubation with HPS plasma, HPS plasma containing anti-HLA or anti-HPA antibodies, or FPH-cross-reactive HPS plasma.

Single donor apheresis platelet units (APU) were purchased from LifeShare Blood Center. FPH was prepared according to the method as described in EXAMPLE 1 of U.S. Pat. No. 11,529,587 B2, incorporated herein by reference in its entirety. FPH were rehydrated with sterile water equivalent to the fill volume prior to lyophilization.

HPS patient blood samples that have tested for the presence of anti-HLA and anti-HPA antibodies by high resolution HLA Class 1 typing and HLA Class 1 antibody screening were received from the American Red Cross. Of the 32 HPS patient blood samples, 10 samples were identified as positive for anti-HLA or anti-HPA antibodies. HPS citrated plasma was retrieved from HPS patient samples.

Normal donor blood was obtained on the day of the experiment and drawn into 3.2% sodium citrate vacutainers. Tubes were placed on a rocker for 30 minutes and then centrifuged at RCF of 180×g for 10 minutes and PRP was removed to a fresh tube. The buffy coat was removed and discarded. The tubes were centrifuged again at RCF of 1800×g for 10 minutes and the PPP was removed and discarded. The red blood cells (RBCs) were pipetted into a fresh tube. Octaplas® was combined with RBCs and PRP to achieve a platelet count between 30,000-50,000/μL, and a hematocrit of 40%+/−2%, as measured by the ActDiff2 hematology analyzer. The reassembled blood was tested using the T-TAS01® (Zacros, zacrosamerica.com) with HD Chip and CaCTI reagent for T-TASO1 (Zacros, zacrosamerica.com), following the manufacturer's recommendations. The platelet count was adjusted until the sample occluded at 20 minutes or longer, at which point a stock blood sample was prepared. The “No FPH” sample was prepared by adding 460 μL blood sample+20 μL HMTA+20 μL CaCTI reagent and running on the HD chip. To prepare the other samples, FPH were incubated with HPS patient plasma, normal plasma, or buffer (No plasma). FPH were diluted in HMTA±plasma to reach a total of 6.94×10\hat{ }6 cells at a ratio of 100,000 cells/μL plasma in 140 μL total volume. Samples were incubated for 20 minutes, then centrifuged at 1100×g, 10 minutes. The supernatant was discarded, and the pellet resuspended in 20 μL HMTA. 460 μL of the blood sample+20 μL CaCTI was added and the sample was run on the HD chip. For the GPRP control, an additional no plasma sample was created, and immediately before starting the run, 0.625 mM GPRP (BaChem) was added to the sample. Two replicates were performed for each sample on a single lot of FPH. Data is plotted as averages of the duplicate measurements for occlusion start time (OST), occlusion time (OT), and area under the curve (AUC), with error bars representing the standard deviation. One-Way ANOVA with Dunnett's test was performed in GraphPad Prism and used to determine statistical difference compared to the normal plasma sample. *(p<0.05), **(p<0.01), ***(p<0.001), ****(p<0.0001).

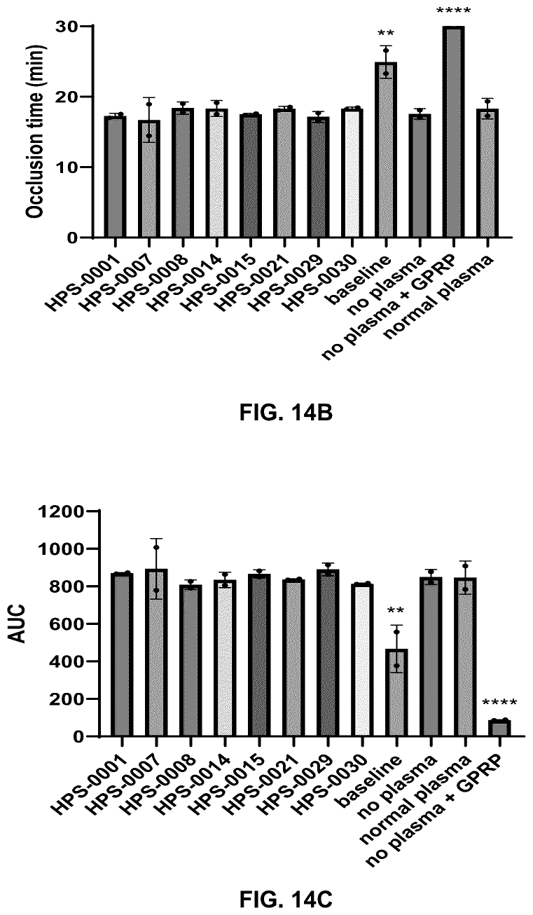

FIG. 14A, FIG. 14B, and FIG. 14C show the occlusion start time, the occlusion time, and the area under the curve (AUC), respectively, of the different samples in the HD chip. The T-TAS parameters for all HPS plasma samples were not statistically significant compared to normal plasma. In fact, two samples (HPS-001 and HPS-0029) that were determined to be cross reactive to FPH were not different from normal plasma control sample in occlusion start time (14±0.53 min, p>0.9999 and 14±0.35 min, p>0.9999, versus 14±1.6 min, respectively), occlusion time (17±0.36 min, p>0.9824 and 17±0.78 min, p>0.9598 versus 18±1.5 min, respectively), and AUC (869±4.0, p>0.9999 and 890±35, p>0.9950, versus 846±89, respectively). There was no statistical significance difference between any HPS sample and normal plasma sample regardless of the fact the sample was positive or negative for anti-HLA or anti-HPA antibodies or was cross reactive to FPH. Therefore, it can be concluded that the FPH hemostatic activity parameters in a T-TAS assay are maintained in all HPS patient plasma samples.

The disclosed embodiments, examples and experiments are not intended to limit the scope of the disclosure or to represent that the experiments below are all or the only experiments performed. Efforts have been made to ensure accuracy with respect to numbers used (e.g., amounts, temperature, etc.) but some experimental errors and deviations should be accounted for. It should be understood that variations in the methods as described may be made without changing the fundamental aspects that the experiments are meant to illustrate.

Those skilled in the art can devise many modifications and other embodiments within the scope and spirit of the present disclosure. Indeed, variations in the materials, methods, drawings, experiments, examples, and embodiments described may be made by skilled artisans without changing the fundamental aspects of the present disclosure. Any of the disclosed embodiments can be used in combination with any other disclosed embodiment.

In some instances, some concepts have been described with reference to specific embodiments. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of invention.

What is claimed is:

1. A method for administering a platelet derivative composition to a subject having Hermansky Pudlak Syndrome (HPS), comprising:
administering an effective dose of the platelet derivatives in a platelet derivative composition to the subject, wherein the platelet derivative composition comprises a population of platelet derivatives,
wherein the platelet derivatives have a compromised plasma membrane,
wherein at least 70% of the platelet derivatives in the platelet derivative composition are CD41 positive platelet derivatives when measured using flow cytometry, and less than 5% of the CD41 positive platelet derivatives are microparticles,
wherein at least 70% of the platelet derivatives in the platelet derivative composition are CD42 positive platelet derivatives, when measured using flow cytometry,
wherein at least 70% of the platelet derivatives in the platelet derivative composition are CD62 positive platelet derivatives, when measured using flow cytometry,
wherein the platelet derivatives are capable of generating thrombin in an in vitro thrombin formation assay, and
wherein the platelet derivatives show an inability to increase expression of a platelet activation marker in the presence of an agonist as compared to the expression of the platelet activation marker in the absence of an agonist, and wherein the platelet activation marker is Annexin V, CD62, or a combination thereof.

2. The method of claim 1, wherein the subject is bleeding at the start of the administering.

3. The method of claim 2, wherein the administering leads to cessation of the bleeding.

4. A method for administering a platelet derivative composition to a subject having Bernard Soulier Syndrome (BSS), comprising:
administering an effective dose of the platelet derivatives in a platelet derivative composition to the subject, wherein the platelet derivative composition comprises a population of platelet derivatives,
wherein the platelet derivatives have a compromised plasma membrane,
wherein at least 70% of the platelet derivatives in the platelet derivative composition are CD41 positive platelet derivatives when measured using flow cytometry, and less than 5% of the CD41 positive platelet derivatives are microparticles,
wherein at least 70% of the platelet derivatives in the platelet derivative composition are CD42 positive platelet derivatives, when measured using flow cytometry,
wherein at least 70% of the platelet derivatives in the platelet derivative composition are CD62 positive platelet derivatives, when measured using flow cytometry,
wherein the platelet derivatives are capable of generating thrombin in an in vitro thrombin formation assay, and
wherein the platelet derivatives show an inability to increase expression of a platelet activation marker in the presence of an agonist as compared to the expression of the platelet activation marker in the absence of an agonist, and wherein the platelet activation marker is Annexin V, CD62, or a combination thereof.

5. The method of claim 4, wherein the subject is bleeding at the start of the administering.

6. The method of claim 5, wherein the administering leads to cessation of the bleeding.

7. The method of any one of claims 1 or 4, wherein at least 75% of the platelet derivatives are CD62 positive platelet derivatives, and wherein at least 75% of the platelet derivatives are CD 41-positive.

8. A method for administering a platelet derivative composition to a subject having Hermansky Pudlak Syndrome (HPS) or Bernard Soulier Syndrome (BSS), comprising:
administering an effective dose of the platelet derivatives in a platelet derivative composition to the subject, wherein the platelet derivative composition comprises a population of platelet derivatives.

9. The method of claim 8, wherein the subject is bleeding at the start of the administering.

10. The method of claim 8, wherein the administering leads to cessation of the bleeding.

11. The method of claim 8, wherein the platelet derivatives have a compromised plasma membrane, wherein at least 50% of the platelet derivatives are CD 41-positive,
and wherein the platelet derivatives
a) have the ability to generate thrombin in vitro in the presence of tissue factor and phospholipids;
b) have the ability to occlude a collagen-coated microchannel in vitro; or
c) both a) and b).

12. The method of any one of claims 8 to 11, wherein the subject has Hermansky Pudlak Syndrome (HPS).

13. The method of claim 12, wherein the administering increases the levels of at least one platelet biomarker selected from CD62P, PAC-1, and CD63 for endogenous platelets of the subject as compared to before the administering.

14. The method of claim 13, wherein levels of the platelet biomarkers CD62P and PAC-1 are increased.

15. The method of any one of claims 8 to 11, wherein the subject has Bernard Soulier syndrome (BSS).

16. The method of any one of claims 8 to 11, wherein the administering increases the levels of at least one platelet biomarker selected from CD62P, PAC-1, and CD63 for endogenous platelets of the subject as compared to before the administering.

17. The method of any one of claims 8 to 11, wherein the administering increases the levels of at least two platelet biomarkers selected from CD62P, PAC-1, and CD63 for endogenous platelets in the subject as compared to the subject before the administering.

18. The method of claim 17, wherein levels of the platelet biomarkers CD62P and PAC-1 are increased.

19. The method of any one of claims 8 to 11, wherein the administering increases the levels of all the three platelet biomarkers selected from CD62P, PAC-1, and CD63 for endogenous platelets in the subject as compared to the subject before the administering.

20. The method of any one of claims 8 to 11, wherein the subject has HPS, wherein the administering is performed to treat the subject, and wherein either
a) at least one HPS-related hemostatic abnormality and/or HPS-related biomarker abnormality observed in the subject is improved in the subject after the administering compared to before the administering; or
b) normal levels of hemostasis and/or the HPS-related biomarker abnormalities are maintained in the subject.

21. The method of any one of claims 8 to 11, wherein the subject has BSS, wherein the administering is performed to treat the subject, and wherein the ability to occlude a collagen-coated microchannel in vitro is restored or improved in the subject after the administering compared to before the administering.

22. The method of any one of claims 8 to 11, wherein the administering leads to an improvement in thrombin generation in the subject as compared to the subject before the administering and/or an improvement in clot formation in the subject as compared to the subject before the administering.

23. The method of any one of claims 8 to 11, wherein the administering leads to an improvement in clot formation in the subject as compared to the subject after being administered apheresis platelets, but before the administering of the platelet derivatives.

24. The method of any one of claims 8 to 11, wherein the administering is performed to treat the subject, and wherein normal levels of hemostasis and/or HPS-related biomarker abnormalities are maintained in the subject.

25. The method of any one of claims 8 to 13, wherein the subject is taking a) one or more anti-coagulants, b) one or more anti-platelet agents, or both a) and b).

26. The method of any one of claims 8 to 11, wherein no more than 5% of the platelet derivatives in the population aggregate under aggregation conditions comprising an agonist but no platelets, and in the absence of a divalent cation.

27. The method of claim 26, wherein the agonist is selected from the group consisting of collagen, epinephrine, ristocetin, arachidonic acid, adenosine di-phosphate, and thrombin receptor associated protein (TRAP).

28. The method of any one of claims 8 to 11, wherein the platelet derivatives are freeze-dried platelet derivatives.

29. The method of claim 28, wherein the subject is bleeding at the start of administering, and the administering leads to a decrease in bleeding within 24 hours after the start of the administering.

30. The method of claim 29, wherein the administering is performed until the bleeding stops.

31. The method of any one of claims 8 to 11, wherein the administering is performed for more than 24 hours.

32. The method of any one of claims 8 to 11, wherein the administering is performed for 24 hours or less.

33. The method of any one of claims 8 to 11, wherein the administering is performed until there is cessation of bleeding at a primary bleeding site.

34. The method of any one of claims 8 to 11, wherein the administering leads to cessation of bleeding within 24 hours after the administering.

35. The method of any one of claims 8 to 11, wherein the platelet derivative composition is in the form of a powder, and the use or the method further comprises before the administering, rehydrating the platelet derivatives to form a rehydrated platelet derivative composition, and wherein the administering is administering an effective dose of the rehydrated platelet derivatives from the rehydrated platelet derivative composition to the subject.

36. The method of any one of claims 8 to 11, wherein the platelet derivatives have a compromised plasma membrane, and wherein the platelet derivative composition
a) has the ability to generate thrombin in vitro in the presence of tissue factor and phospholipids; and
b) has the ability to occlude a collagen-coated microchannel in vitro.

37. The method of claim 36, wherein the platelet derivative composition has the ability to occlude, and wherein the ability to occlude is determined by a thrombus-formation analysis system (T-TAS) assay.

38. The method of claim 37, wherein when at a concentration of 70×10³ particles/μL, the platelet derivative composition produce an occlusion time of less than 20 minutes in a T-TAS assay.

39. The method of any one of claims 8 to 11, wherein at least 50% of the platelet derivatives have a diameter in the range of 0.5-2.5 μm by flow cytometry, and wherein at least 65% of the platelet derivatives are CD42 positive.

40. The method of any one of claims 8 to 11, wherein the composition when rehydrated comprises less than or equal to 15% plasma protein, and wherein the platelet derivatives have less than 5.0% microparticles having a diameter less than 0.5 μm by scattering intensity.

41. The method of any one of claims 8 to 11, wherein one or more of the following:
less than 5% of CD 41-positive platelet derivatives are microparticles having a diameter of less than 0.5 μm,
at least 65% of the platelet derivates are CD 42 positive,
the composition comprises a population of platelet derivatives having a reduced propensity to aggregate such that no more than 10% of the platelet derivatives in the population aggregate under aggregation conditions comprising an agonist but no platelets, and no divalent cation,
at least 50% of the platelet derivatives in the composition are at least 0.5 μm in diameter by scattering intensity; and
at least 50% of the platelet derivatives in the composition are between 0.5 μm and 25 μm in diameter by scattering intensity.

42. The method of any one of claims 8 to 11, wherein two or more of the following:
less than 5% of CD 41-positive platelet derivatives are microparticles having a diameter of less than 0.5 μm,
at least 65% of the platelet derivates are CD 42 positive,
the composition comprises a population of platelet derivatives having a reduced propensity to aggregate such that no more than 10% of the platelet derivatives in the population aggregate under aggregation conditions comprising an agonist but no platelets, and no divalent cation,
at least 50% of the platelet derivatives in the composition are at least 0.5 μm in diameter by scattering intensity; and
at least 50% of the platelet derivatives in the composition are between 0.5 μm and 25 μm in diameter by scattering intensity.

43. The method of any one of claims 8 to 11, wherein,
less than 5% of CD 41-positive platelet derivatives are microparticles having a diameter of less than 0.5 μm,
at least 65% of the platelet derivates are CD 42 positive,
the composition comprises a population of platelet derivatives having a reduced propensity to aggregate such that no more than 10% of the platelet derivatives in the population aggregate under aggregation conditions comprising an agonist but no platelets, and no divalent cation,
at least 50% of the platelet derivatives in the composition are at least 0.5 μm in diameter by scattering intensity; and
at least 50% of the platelet derivatives in the composition are between 0.5 μm and 25 μm in diameter by scattering intensity.

44. The method of any one of claims 8 to 11, wherein:
at least 65% of the platelet derivates are CD 42 positive. and
the composition comprises a population of platelet derivatives having a reduced propensity to aggregate such that no more than 10% of the platelet derivatives in the population aggregate under aggregation conditions comprising an agonist but no platelets, and no divalent cation.

45. The method of any one of claims 8 to 11, wherein the effective dose of the platelet derivatives is in the range of 1.0×10⁷ to 1.0×10¹²/kg of the subject.

46. The method of any one of claims 8 to 11, wherein the effective dose of the platelet derivatives is in the range of 1.0×10⁷ to 1.0×10¹¹/kg of the subject.

47. The method of any one of claims 8 to 11, wherein the effective dose of the platelet derivatives is in the range of 1.5×10⁷ to 1.1×10¹⁰/kg of the subject.

48. The method of any one of claims 8 to 11, wherein the effective dose of the platelet derivatives is in the range of 1.5×10⁹ to 1.0×10¹²/kg of the subject.

49. The method of any one of the preceding claims, wherein the subject has anti-HLA and/or anti-HPA antibodies.

50. The method of any one of the preceding claims, wherein the subject has cross-reactive antibodies against the platelet derivatives.

51. The method of any one of the preceding claims, wherein the method leads to a decrease or cessation in bleeding at a primary site within 2 hours after the administering of the platelet derivatives.

52. The method of any one of the preceding claims, wherein the administering comprises administering a dose of the platelet derivatives between 2 and 10 times within 7 days from a first dose of the platelet derivatives.
